(19) United States
(12) Patent Application Publication
(10) Pub. No.: US 2023/0322870 A1
(43) Pub. Date: Oct. 12, 2023

(54) ACTINOBACILLUS PLEUROPNEUMONIAE VACCINES

(71) Applicants: IMPERIAL COLLEGE INNOVATIONS LIMITED, London (GB); Ceva Sante Animale, Liboume (FR)

(72) Inventors: Janine T Bosse, London (GB); Paul Richard Langford, London (GB); Yanwen LI, London (GB); Andrew Nelson Rycroft, Hatfield (GB); Sebatian Stentzel, Libourne (FR); Fabian Deutskens, Libourne (FR)

(21) Appl. No.: 18/018,526

(22) PCT Filed: Jul. 30, 2021
(86) PCT No.: PCT/IB2021/000549

§ 371 Date: Jan. 27, 2023

(30) Foreign Application Priority Data
Jul. 30, 2020 (GB) 2011902.0

(51) Int. Cl. C07K 14/285; A61K 39/102; C12N 1/20

(52) U.S. Cl. CPC C07K 14/285; A61K 39/102; C12N 1/20; A61K 2039/523

(57) ABSTRACT

The present invention relates to microorganisms comprising each of each of an ApxIA, ApxIIA and ApxIIIA toxin, related vaccines and methods of production thereof, as well as uses thereof for the immunisation and protection of mammals.

### FIELD OF THE INVENTION

The present invention relates to microorganisms comprising each of an ApxIA, ApxIIA and ApxIIIA toxin, related vaccines and methods of production thereof, as well as uses thereof for the immunisation and protection of mammals.

### BACKGROUND OF THE INVENTION

Actinobacillus pleuropneumoniae (APP) is a Gram-negative bacterium and a member of the family Pasteurellaceae. APP is the etiological agent of porcine pleuropneumonia, a severe pulmonary disease of pigs that causes high economic losses in pig production worldwide. The disease is often characterised by haemorrhagic, fibrinous and necrotic lung lesions. Pigs surviving the disease often become asymptomatic carriers of APP and are the main cause of bacterial dissemination.

To date, 19 serotypes of APP have been identified on the basis of the antigenic properties of capsular polysaccharides (CPS) and as a result of genetic analysis. Main virulence factors of APP are exotoxins, CPS, lipopolysaccharide (LPS) and membrane proteins. The most important virulence factors are Apx-exotoxins, which belong to the pore-forming repeats in toxin (RTX) family. The toxins are known to be highly immunogenic and are very important to obtain a protective immunity against APP-related pleuropneumonia. At least four different Apx toxins are produced by APP, designated ApxI, ApxII, ApxIII and ApxIV. ApxI shows strong haemolytic activity, whereas ApxII shows low haemolytic activity. Both are cytotoxic and active against a broad range of cells of different host species. ApxIII is non-haemolytic but strongly cytotoxic, with porcine alveolar macrophages and neutrophils as major targets. ApxIV shows no cytotoxic activity and only weak haemolytic activity. No serotypes of APP produce all four Apx toxins, or even all three of ApxI, ApxII and ApxIII. All serotypes produce ApxIV and one or two of ApxI-III. The pattern of Apx toxin production is associated with virulence, with serotypes 1, 5, 9 and 11 producing ApxI and ApxII being the most virulent.

At least four genes are responsible for production and secretion of active Apx toxins. Gene A encodes the structural toxin. Gene C encodes an acyltransferase which is required for post-translational activation of the toxin. Genes B and D encode two membrane proteins that are required for secretion of the mature protein. The Apx genes are organised as operons. Operons of ApxI and ApxIII consist of genes CABD, whilst the ApxII operon contains only the genes CA (FIG. 1). Secretion of ApxIIA therefore depends on active genes B and D of the ApxI operon.

Presently, pleuropneumonia resulting from APP infection of pigs is usually treated with antibiotics. However, it has been found that APP often exhibits antibiotic resistance against at least one of the antibiotics commonly used to treat APP infection.

Vaccination against APP is a promising prophylactic strategy to prevent pleuropneumonia. Several vaccines have been commercialised. Commercially available vaccines are either chemically inactivated whole cell vaccines or subunit vaccines or a combination of both. The immunological reactions of animals vaccinated with whole cell vaccines is directed mainly against surface structures such as CPS and LPS. The absence of secreted proteins such as Apx toxins which are known to be highly immunogenic and essential for protection explains the limited protection observed with APP whole cell vaccines. Furthermore, APP whole cell vaccines confer only homologous protection against the serotype used to prepare the vaccine.

The commercially available subunit vaccine (described in EP 0453024 B1) contains chemically inactivated ApxA toxins as well as an outer membrane protein. The inactivation of ApxA toxins with denaturising substances such as formaldehyde potentially leads to a decreased immunogenicity of the toxoids. The disadvantages of such a vaccine are insufficient protection due to denaturising the toxins, as well as concomitant serious side reactions, probably due to residual toxicity from incomplete inactivation of ApxA toxins. Subsequently, due to decreased immunogenicity after inactivation, higher amounts of toxins need to be used. This can result in increased amounts of contaminating LPS in the vaccines. High LPS content can also cause side effects, as seen with the commercial APP subunit vaccines.

Thus, the current commercially available APP vaccines do not possess satisfactory safety and or efficacy profiles against APP infection.

Other experimental approaches for APP vaccines are also under development.

WO 2004/045639 discloses a live attenuated vaccine against porcine pleuropneumonia containing an APP strain which is modified in transmembrane domains of genes encoding the toxins ApxIA and ApxIIA. To test the degree of attenuation, three-month old pigs were inoculated with modified live APP strains. Seven days after the inoculation the animals were sacrificed, and macroscopic lesions were recorded in the respiratory organs. All animals showed a modification of their behaviour and lung lesions at necropsy. The efficacy of this live vaccine was not examined.

EP 0810283(A2) and EP0861319(B1) describe live attenuated APP strains having deletions in ApxA activator (apxC) genes. The modified APP strain does not produce the ApxC activator proteins in a functional form and therefore the toxins ApxIA and ApxIIA were not activated by acylation. Mice were vaccinated with a ΔapxC strain and challenged with virulent APP field strains. Vaccinated mice were protected against homologous challenge and partially protected against heterologous challenge. One study in pigs was performed. One out of six vaccinated pigs had lung lesions after heterologous challenge and necropsy. These attenuated live vaccines seem to be efficacious, but bear a significant safety risk. The vaccine strains produce Apx toxins which are not activated by acylation due to lacking apxC. However, these toxins have the original amino acid sequence of toxic ApxA. Very likely heterologous acyltransferases can acylate the ApxA converting it into its active, toxic form. In most pig farms worldwide, there are asymptomatic carriers of virulent APP strains as well as pigs infected with low virulent APP strains. If such pigs were vaccinated with a ΔapxC vaccine strain, the non-activated ApxA toxins of the vaccine strain could be activated by functional ApxC proteins of field strains. Furthermore, there is the possibility of complementation of the apxC deletion by uptake of functional apxC genes. Therefore, there is the possibility that the attenuated strains could revert to virulence, causing disease in the vaccinated animals.

The present inventors have previously developed recombinant ApxIA, ApxIIA and ApxIIIA toxins expressed in Escherichia coli, which are modified at their acylation sites to create inactive but fully immunogenic toxoid forms of these proteins.

To-date, the issue of providing protection against heterologous serotypes, particularly for live attenuated and inactivated whole cell vaccines, remains. Further, even with subunit vaccines this remains problematic from a commercial perspective, as it is costly to grow sufficient volumes of multiple different serotypes and then purify their respective ApxA proteins to produce subunit vaccines that provide protection against all known APP serotypes.

Accordingly, there is a need for improved vaccines against APP which are safe, can be produced at scale by an economically viable process, and which are safe and able to induce cross-protection against all relevant APP serotypes in pigs and/or young piglets.

It is therefore an object of the invention to provide microorganisms comprising each of each of an ApxIA, ApxIIA and ApxIII toxin, related vaccines and methods of production, as well as uses thereof for the immunisation and protection of mammals.

### SUMMARY OF THE INVENTION

The present inventors are the first to produce APP bacteria expressing all three of ApxIA, ApxIIA and ApxIIIA in a single strain. In particular, the inventors have constructed APP strains that produce non-functional forms of each of ApxIA, ApxIIA and ApxIIIA, with the genes encoding each modified toxin integrated into the APP chromosome. These APP strains have been generated by the introduction of unmarked mutations using two-step natural transformation. The advantage of the inventors' modified APP strains is that these triple mutants can be used as a single live attenuated vaccine strain, that will induce antibodies against all three of ApxIA, ApxIIA and ApxIIIA, and hence that will give protection against all known serovars of APP. In addition, these strains can be used to streamline the production of Apx toxoid vaccines, enabling a single APP strain to be used to produce all three of ApxIA, ApxIIA and ApxIIIA. Using the inventors' methodology it would be equally possible to produce APP strains producing all three of ApxIA, ApxIIA and ApxIIIA, either in wild-type or modified forms for the production of inactivated whole cell or subunit vaccines, wherein either the bacteria or the individual ApxIA, ApxIIA and ApxIIIA can be inactivated using suitable inactivants for use as vaccines.

Accordingly, the present invention provides a microorganism comprising: (a) a nucleic acid sequence encoding ApxIA of Actinobacillus pleuropneumoniae; (b) a nucleic acid sequence encoding ApxIIA of A. pleuropneumoniae; and (c) a nucleic acid sequence encoding ApxIIIA of A. pleuropneumoniae.

The nucleic acid sequences of (a), (b) and/or (c) may be: (i) comprised within the genome of the microorganism; or (ii) comprised extra-chromosomally.

The ApxIA, ApxIIA and ApxIIIA may be: (a) inactive ApxIA, ApxIIA and ApxIIIA which have common antigenic cross-reactivity with wild-type ApxIA, ApxIIA and ApxIIIA; or (b) wild-type ApxIA, ApxIIA and ApxIIIA. In particular, the microorganism may comprise: (a) (i) the inactive ApxIA has an amino acid sequence corresponding to the wild-type ApxIA amino acid sequence of SEQ ID NO: 1, modified in at least one amino acid selected from the group consisting of K560 and K686, or a variant or fragment thereof which is at least 90% homologous to said inactive ApxIA amino acid sequence, said fragment comprising at least 30% of the consecutive amino acids of said inactive ApxIA amino acid sequence, wherein said variant or fragment comprises the at least one modified amino acid; (ii) the inactive ApxIIA has an amino acid sequence corresponding to the wild-type ApxIIA amino acid sequence of SEQ ID NO: 2, modified in at least one amino acid selected from the group consisting of K557 and N687, or a variant or fragment thereof which is at least 90% homologous to said inactive ApxIIA amino acid sequence, said fragment comprising at least 30% of the consecutive amino acids of said inactive ApxIIA amino acid sequence, wherein said variant or fragment comprises the at least one modified amino acid; and (iii) the inactive ApxIIIA has an amino acid sequence corresponding to the wild-type ApxIIIA amino acid sequence of SEQ ID NO: 3, modified in at least one amino acid selected from the group consisting of K571 and K702, or a variant or fragment thereof which is at least 90% homologous to said inactive ApxIIIA amino acid sequence, said fragment comprising at least 30% of the consecutive amino acids of said inactive ApxIIIA amino acid sequence, wherein said variant or fragment comprises the at least one modified amino acid; and the at least one modified amino acid is substituted by an amino acid not susceptible to acylation; or (b) (i) the inactive ApxIA has an amino acid sequence corresponding to the wild-type ApxIA amino acid sequence of SEQ ID NO: 1, containing deletions comprising at least one amino acid selected from the group consisting of K560 and K686, or a variant or fragment thereof which is at least 90% homologous to said inactive ApxIA amino acid sequence, said fragment comprising at least 30% of the consecutive amino acids of said inactive ApxIA amino acid sequence, wherein said variant or fragment comprises the deletion; (ii) the inactive ApxIIA has an amino acid sequence corresponding to the wild-type ApxIIA amino acid sequence of SEQ ID NO: 2, containing deletions comprising at least one amino acid selected from the group consisting of K557 and N687, or a variant or fragment thereof which is at least 90% homologous to said inactive ApxIIA amino acid sequence, said fragment comprising at least 30% of the consecutive amino acids of said inactive ApxIIA amino acid sequence, wherein said variant or fragment comprises the deletion; and (iii) the inactive ApxIIIA has an amino acid sequence corresponding to the wild-type ApxIIIA amino acid sequence of SEQ ID NO: 3, containing deletions comprising at least one amino acid selected from the group consisting of K571 and K702, or a variant or fragment thereof which is at least 90% homologous to said inactive ApxIIIA amino acid sequence, said fragment comprising at least 30% of the consecutive amino acids of said inactive ApxIIIA amino acid sequence, wherein said variant or fragment comprises the deletion. Wherein either or both of the acylation sites are substituted by amino acids not susceptible to acylation, each amino acid not susceptible to acylation may be independently selected from the group consisting of alanine, glycine, isoleucine, leucine, methionine, valine, serine, threonine, asparagine, glutamine, aspartic acid, histidine, aspartic acid, cysteine, proline, phenylalanine, tyrosine, tryptophan and glutamic acid; preferably selected from the group consisting of alanine, glycine, serine, isoleucine and leucine, valine and threonine; most preferably selected from the group consisting of alanine, glycine and serine. The inactive ApxIA may have substitutions at both K560 and K686. The inactive ApxIIA may have substitutions at both K557 and N687. The inactive ApxIIIA may have substitutions at both K571 and K702. The inactive ApxIA may comprise the amino acid sequence of SEQ ID NO: 4. The inactive ApxIIA may comprise the amino acid sequence of SEQ ID NO: 5. The inactive ApxIIIA may comprise the amino acid sequence of SEQ ID NO: 6. Wherein the acylation sites are deleted: (i) the inactive ApxIA has deletions at both K560 and K686; (ii) the inactive ApxIIA has deletions at both K557 and N687; and (iii) the inactive ApxIIIA has deletions at both K571 and K702.

Wherein the microorganism comprises wild-type ApxA polypeptides: (a) the wild-type ApxIA has an amino acid sequence corresponding to SEQ ID NO: 1, or a variant or fragment thereof which is at least 90% homologous to said wild-type ApxIA amino acid sequence, said fragment comprising at least 30% of the consecutive amino acids of said wild-type ApxIA amino acid sequence; (b) the wild-type ApxIIA has an amino acid sequence corresponding to SEQ ID NO: 2, or a variant or fragment thereof which is at least 90% homologous to said wild-type ApxIIA amino acid sequence, said fragment comprising at least 30% of the consecutive amino acids of said wild-type ApxIIA amino acid sequence; and (c) the wild-type ApxIIIA has an amino acid sequence corresponding to SEQ ID NO: 3, or a variant or fragment thereof which is at least 90% homologous to said wild-type ApxIIIA amino acid sequence, said fragment comprising at least 30% of the consecutive amino acids of said wild-type ApxIIIA amino acid sequence.

The microorganism of the invention may be an Escherichia coli strain or an Actinobacillus strain, preferably an Actinobacillus pleuropneumoniae strain. The A. pleuropneumoniae strain may be produced from: (a) an A. pleuropneumoniae strain which expresses an endogenous ApxIIA and ApxIIIA, preferably a serotype 2, 8, or 15 strain; or (b) an A. pleuropneumoniae strain which expresses an endogenous ApxIA and ApxIIA, preferably a serotype 1, 5 or 9 strain.

The microorganism may be an A. pleuropneumoniae strain in which at least one additional gene is modified, wherein preferably: (a) said one or more additional gene is selected from the group consisting of apxIVA, sxy, nlpD and/or ssrA; and/or (b) said modification results in the inactivation of said one or more additional gene. The at least one additional gene which is modified maybe (i) apxIVA; (ii) sxy; or (iii) apxIVA and sxy, wherein preferably: (a) the apxIVA gene is modified by an unmarked in-frame deletion of an N-terminal immunogenic domain sequence; and/or (b) the sxy gene is deleted.

The invention also provides a vaccine composition comprising a microorganism of the invention and at least a pharmaceutical carrier, a diluent and/or an adjuvant. Said vaccine may be a live vaccine, wherein preferably: (a) the microorganism is an Actinobacillus pleuropneumoniae strain; and/or (b) the ApxIA, ApxIIA and ApxIIIA are inactive ApxIA, ApxIIA and ApxIIIA which have common antigenic cross-reactivity with wild-type ApxIA, ApxIIA and ApxIIIA. Said vaccine may be an inactivated vaccine, wherein preferably: (a) the microorganism is an Actinobacillus pleuropneumoniae strain; and/or (b) the ApxIA, ApxIIA and ApxIIIA are wild-type ApxIA, ApxIIA and ApxIIIA which have been subsequently inactivated, preferably by chemical and/or heat treatment.

The invention also provides a method of producing a live vaccine composition of the invention, comprising: (a) culturing a microorganism of the invention, wherein the ApxIA, ApxIIA and ApxIIIA are inactive ApxIA, ApxIIA and ApxIIIA which have common antigenic cross-reactivity with wild-type ApxIA, ApxIIA and ApxIIIA; (b) isolating the microorganism; and (c) formulating the microorganism with a pharmaceutical carrier, a diluent and/or an adjuvant.

The invention further provides a method of producing an inactivated vaccine composition of the invention, comprising: (a) culturing a microorganism of the invention, wherein the ApxIA, ApxIIA and ApxIIIA are wild-type ApxIA, ApxIIA and ApxIIIA; (b) isolating the microorganism; (c) inactivating the microorganism, preferably by chemical and/or heat treatment; and (d) formulating the inactivated microorganism with a pharmaceutical carrier, a diluent and/or an adjuvant.

The invention also provides a method of producing a subunit vaccine composition, comprising: (a) (i) culturing a microorganism of the invention, wherein the ApxIA, ApxIIA and ApxIIIA are inactive ApxIA, ApxIIA and ApxIIIA which have common antigenic cross-reactivity with wild-type ApxIA, ApxIIA and ApxIIIA; (ii) isolating the inactive ApxIA, ApxIIA and ApxIIIA from the cultured microorganism; and (iii) formulating the inactive ApxIA, ApxIIA and ApxIIIA with a pharmaceutical carrier, a diluent and/or an adjuvant; or (b) (i) culturing a microorganism of the invention, wherein the ApxIA, ApxIIA and ApxIIIA are wild-type ApxIA, ApxIIA and ApxIIIA; (ii) isolating the wild-type ApxIA, ApxIIA and ApxIIIA from the cultured microorganism; (iii) inactivating the wild-type ApxIA, ApxIIA and ApxIIIA, preferably by chemical and/or heat treatment; and (iv) formulating the inactivated wild-type ApxIA, ApxIIA and ApxIIIA with a pharmaceutical carrier, a diluent and/or an adjuvant.

The invention also provides a vaccine composition of the invention for use in a method of prophylactic, metaphylactic or therapeutic treatment of a pneumonia, a pleurisy or a pleuropneumonia, in particular, of a pneumonia, a pleurisy or a pleuropneumonia caused by Actinobacillus pleuropneumoniae, wherein optionally the vaccine composition is to be administered intramuscularly, intradermally, intravenously, subcutaneously, or by mucosal administration.

The invention further provides an expression system comprising a microorganism of the invention, further comprising at least one additional nucleic acid which encodes one or more additional swine pathogen antigen, wherein preferably the at least one additional nucleic acid is comprised within the genome of the microorganism.

The invention further provides a vector or set of vectors comprising nucleic acids encoding for: (a) wild-type ApxIA, ApxIIA and ApxIIIA as defined herein; or (b) inactive ApxIA, ApxIIA and ApxIIIA which have common antigenic cross-reactivity with wild-type ApxIA, ApxIIA and ApxIIIA as defined herein.

### BRIEF DESCRIPTION OF THE DRAWINGS

FIG. 1: Organisation of the apx operons in Actinobacillus pleuropneumoniae. A) the complete apxI operon, found in serotypes 1, 5, 9, 11, 14 and 16; B) the apxII operon, found in most serotypes except 10 and 14. This operon lacks genes encoding a cognate secretion system and has only a truncated apxIIB* sequence; C) the apxIII operon, found in serotypes 2, 3, 4, 6, 8 and 15; D) the truncated apxI operon found in all serotypes (except serotype 3) lacking the complete apxI operon. A partial (3′ end only) apxIA gene is present upstream of the apxIBD genes and is indicated as apxIA*. For all, the apxC genes encode the acyltransferases required for toxin activation; the apxA genes encode the toxin protein (the location of codons for acylation site amino acids K or N are marked for each, as appropriate); the apxBD genes encode the secretion system required for each toxin, with ApxII using the secretion system encoded by apxIBD.

FIG. 2: Schematic representation of sequences for generation of gene replacement cassettes used in the first round of natural transformation when generating unmarked mutations in Actinobacillus pleuropneumoniae. For each gene replacement, approximately 500 bases upstream (i.e. the left flank sequence; shown upper left) and approximately 500 bases downstream (i.e. the right flank sequence; shown lower right) of the target sequence to be replaced are synthetically generated with universal priming sites (i.e. left_flank_forward and tri_OE_rev, or sac_OE_for and right_flank_rev, as appropriate) to allow fusion by overlap PCR to the synthetic dfrAsacB cassette (shown in the centre), which has sites complementary to the 3′ end of the left and 5′ end of the right flank sequences (i.e. tri_OE_for and sac_OE_rev, respectively; which can be used to amplify the dfrAsacB cassette by PCR). See main text for specific sequences of all primers. Sizes of sequences shown by rulers with tick marks indicating every 100 bp.

FIG. 3: Schematic representation of sequences used in a two-step natural transformation method for replacement of acylation site codons in apxIIA, resulting in non-active ApxII toxin with K557A and N687A mutations. A) Wild-type apxIIA sequence showing location of the two acylation site codons (AAA at 1669-1671 bp, encoding K; AAT at 2059-2061 bp, encoding N). B) Construct used in first round of natural transformation to replace the central region of apxIIA (containing both acylation sites) with a counter-selectable cassette (dfrA14sacB); C) Synthetic construct used to replace the dfrA14sacB cassette in the second round of natural transformation, leaving altered codons (GCA at 1669-1671 bp, and GCT at 2059-2061 bp, both encoding A residues) resulting in a non-active ApxIIA protein. Sizes of sequences shown by rulers with tick marks indicating every 100 bp.

FIG. 4: (A) SDS PAGE was conducted to determine ApxII and ApxIII expression in inactive form from APP ST8 and ST15 strains compared to supernatants of respective wild-type (WT) strains ST8 and ST15 of APP. Arrows indicate ApxII (lower band) and ApxIII (upper band). No difference in expression levels was observed between wild-type (WT) and inactive (MUT) forms. (B) Supernatants of ST8 and ST15 containing ApxII and ApxIII in active (WT) and inactive form (MUT) were serially diluted in PBS. 6M urea served as control and was also serially diluted in PBS. The dilutions were incubated with BL3 cells and cytotoxicity determined using WST-1 substrate by measuring absorption at 450 nm. Whereas ST8 MUT and ST15 MUT showed similar pattern as 6M urea control and did not induce cell death in dilutions 1:32, the APP 8 WT and APP 15 WT remained cytotoxic properties even in dilution 1:1024 and above.

FIG. 5: (A) Western Blot to confirm expression of ApxI, ApxII and Apx Ill in inactive form from the same APP. Supernatants of the same ST8 (lanes: ST8) and ST15 (lanes: ST15) were applied. Monoclonal antibodies raised against and specific for ApxI, ApxII and ApxIII were used to detect expression of the respective toxins. ApxI (lane: ApxI), truncated form of ApxII (lane: ApxIIt) and ApxIII (lane: ApxIII) recombinantly expressed in E. coli served as positive control to demonstrate specificity of monoclonal antibodies. None of the monoclonal antibodies cross-reacted with the other Apx-toxins (data not shown). (B) Supernatants of ST8 and ST15 expressing ApxI, ApxII and ApxIII in active (wild-type, WT) and inactive form (MUT) were serially diluted in PBS. 6M urea served as control and was also serially diluted in PBS. The dilutions were incubated with BL3 cells and cytotoxicity determined using WST-1 substrate by measuring absorption at 450 nm. Whereas ST8 MUT and ST15 MUT showed similar pattern as 6M urea control and did not induce cell death in dilutions 1:32, the APP 8 WT and APP 15 WT remained cytotoxic properties even in dilution 1:1024 and above.

FIG. 6: Schematic representation of sequences for generation of an unmarked sxy mutation in Actinobacillus pleuropneumoniae. A) Sequences used to generate a construct to allow insertion of the dfrA14sacB cassette downstream of sxy. Complementary sequences (tri_OE_rev and tri_OE_for, as well as sac_OE_rev and sac_OE_for) allow fusion of the left and right flanking sequences to the dfrA14sacB cassette by OE-PCR. The resulting product of the OE-PCR is used in the first round of natural transformation. B) Synthetic construct used to replace the dfrA14sacB cassette along with the entire sxy gene in the second round of natural transformation. Sizes of sequences shown by rulers with tick marks indicating every 100 bp.

FIG. 7: Schematic representation of the genomic region flanking the sxy gene found in A. pleuropneumoniae. A) wild type region showing the sxy gene flanked by rpsI and fumC; B) a knock-in mutant having the dfrA14sacB cassette introduced downstream of sxy; C) a clean deletion mutant with the entire sxy gene removed. Sizes of sequences shown by rulers with tick marks indicating every 100 bp.

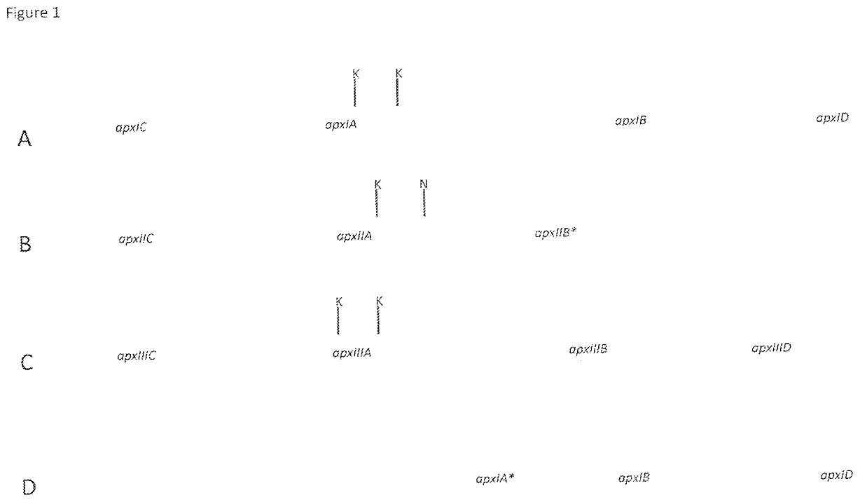

### DETAILED DESCRIPTION OF THE INVENTION

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure. The meaning and scope of the terms should be clear; however, in the event of any latent ambiguity, definitions provided herein take precedent over any dictionary or extrinsic definition. It should be understood that this invention is not limited to the particular methodology, protocols, and reagents, etc., described herein and as such can vary.

This disclosure is not limited by the exemplary methods and materials disclosed herein, and any methods and materials similar or equivalent to those described herein can be used in the practice or testing of embodiments of this disclosure. The terminology used herein is for the purpose of describing particular embodiments only, and is not intended to limit the scope of the present invention, which is defined solely by the claims.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

As used herein, the term “capable of” when used with a verb, encompasses or means the action of the corresponding verb. For example, “capable of interacting” also means interacting, “capable of cleaving” also means cleaves, “capable of binding” also means binds and “capable of specifically targeting . . . ” also means specifically targets.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

As used herein, the articles “a” and “an” may refer to one or to more than one (e.g. to at least one) of the grammatical object of the article. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ±4%, ±3%, ±2%, ±1%, ±0.5%, ±0.1%, of the numerical value of the number with which it is being used.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention (i.e. inactive or non-immunogenic ingredients).

Embodiments described herein as “comprising” one or more features may also be considered as disclosure of the corresponding embodiments “consisting of” and/or “consisting essentially of” such features.

The term “pharmaceutically acceptable” as used herein means approved by a regulatory agency of the Federal or a state government, or listed in the U.S. Pharmacopeia, European Pharmacopeia or other generally recognized pharmacopeia for use in animals, and more particularly in pigs.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

Amino acids are referred to herein using the name of the amino acid, the three-letter abbreviation or the single letter abbreviation.

As used herein, the terms “protein” and “polypeptide” are used interchangeably herein to designate a series of amino acid residues, connected to each other by peptide bonds between the alpha-amino and carboxyl groups of adjacent residues. The terms “protein”, and “polypeptide” refer to a polymer of amino acids, including modified amino acids (e.g., phosphorylated, glycated, glycosylated, etc.) and amino acid analogues, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogues of the foregoing.

Minor variations in the amino acid sequences of proteins of the invention are contemplated as being encompassed by the present invention, providing that the variations in the amino acid sequence(s) maintain at least 60%, at least 70%, more preferably at least 80%, at least 85%, at least 90%, at least 95%, and most preferably at least 97% or at least 99% sequence identity to the proteins of the invention or an immunogenic fragment thereof as defined anywhere herein. The term homology is used herein to mean identity. As such, the sequence of a variant or analogue sequence of a protein of the invention may differ on the basis of substitution (typically conservative substitution) deletion or insertion.

Proteins of the invention may include variants in which amino acid residues from one species are substituted for the corresponding residue in another species, either at the conserved or non-conserved positions. Variants of protein molecules disclosed herein may be produced and used in the present invention. Following the lead of computational chemistry in applying multivariate data analysis techniques to the structure/property-activity relationships [see for example, Wold, et al. Multivariate data analysis in chemistry. Chemometrics-Mathematics and Statistics in Chemistry (Ed.: B. Kowalski); D. Reidel Publishing Company, Dordrecht, Holland, 1984 (ISBN 90-277-1846-6] quantitative activity-property relationships of proteins can be derived using well-known mathematical techniques, such as statistical regression, pattern recognition and classification [see for example Norman et al. Applied Regression Analysis. Wiley-Interscience; 3rd edition (April 1998) ISBN: 0471170828; Kandel, Abraham et al. Computer-Assisted Reasoning in Cluster Analysis. Prentice Hall PTR, (May 11, 1995), ISBN: 0133418847; Krzanowski, Wojtek. Principles of Multivariate Analysis: A User's Perspective (Oxford Statistical Science Series, No 22 (Paper)). Oxford University Press; (December 2000), ISBN: 0198507089; Witten, Ian H. et al Data Mining: Practical Machine Learning Tools and Techniques with Java Implementations. Morgan Kaufmann; (Oct. 11, 1999), ISBN:1558605525; Denison David G. T. (Editor) et al Bayesian Methods for Nonlinear Classification and Regression (Wiley Series in Probability and Statistics). John Wiley & Sons; (July 2002), ISBN: 0471490369; Ghose, Arup K. et al. Combinatorial Library Design and Evaluation Principles, Software, Tools, and Applications in Drug Discovery. ISBN: 0-8247-0487-8]. The properties of proteins can be derived from empirical and theoretical models (for example, analysis of likely contact residues or calculated physicochemical property) of proteins sequence, functional and three-dimensional structures and these properties can be considered individually and in combination.

Amino acids are referred to herein using the name of the amino acid, the three-letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Amino acid residues at non-conserved positions may be substituted with conservative or non-conservative residues. In particular, conservative amino acid replacements are contemplated.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in a protein of the invention does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

“Insertions” or “deletions” are typically in the range of about 1, 2, or 3 amino acids. The variation allowed may be experimentally determined by systematically introducing insertions or deletions of amino acids in a protein using recombinant DNA techniques and assaying the resulting recombinant variants for activity. This does not require more than routine experiments for a skilled person.

A “fragment” of a polypeptide comprises at least 50%, at least 60%, at least 70%, at least 80%, at least 90%, at least 95%, at least 97% or more of the original polypeptide. These fragments may be used as active ingredients in APP vaccines as described herein.

The proteins of the invention, or immunogenic fragments thereof, include both intact and modified forms of the proteins disclosed herein. For example, a protein of the invention or immunogenic fragment thereof can be functionally linked (e.g. by chemical coupling, genetic fusion, noncovalent association, or otherwise) to one or more other molecular entities, such as a pharmaceutical agent, a detection agent, and/or a protein or peptide that can mediate association of a binding molecule disclosed herein with another molecule (e.g. a streptavidin core region or a polyhistidine tag) Non-limiting examples of detection agents include: enzymes, such as alkaline phosphatase, glucose-6-phosphate dehydrogenase (“G6PDH”), alpha-D-galactosidase, glucose oxydase, glucose amylase, carbonic anhydrase, acetylcholinesterase, lysozyme, malate dehydrogenase and peroxidase, e.g., horseradish peroxidase; dyes; fluorescent labels or fluorescers, such as fluorescein and its derivatives, fluorochrome, rhodamine compounds and derivatives, GFP (GFP for “Green Fluorescent Protein”), dansyl, umbelliferone, phycoerythrin, phycocyanin, allophycocyanin, o-phthaldehyde, and fluorescamine; fluorophores such as lanthanide cryptates and chelates, e.g., Europium etc., (Perkin Elmer and Cis Biointernational); chemoluminescent labels or chemiluminescers, such as isoluminol, luminol and the dioxetanes; bio-luminescent labels, such as luciferase and luciferin; sensitizers; coenzymes; enzyme substrates; radiolabels, including but not limited to, bromine⁷⁷, carbon¹⁴, cobalt⁵⁷, fluorine⁸, gallium⁶⁷, gallium⁶⁸, hydrogen³ (tritium), indium¹¹¹, indium^{113m}, iodine^{123m}, iodine125, iodine¹²⁶, iodine¹³¹, iodine¹³³, mercury¹⁰⁷, mercury²⁰³, phosphorous³², rhenium^{99m}, rhenium¹⁰¹, rhenium¹⁰⁵, ruthenium⁹⁵, ruthenium⁹⁷, ruthenium¹⁰³, ruthenium¹⁰⁵, scandium⁴⁷, selenium⁷⁵, sulphur³⁵, technetium⁹⁹, technetium^{99m}, tellurium^{121m}, tellurium^{122m}, tellurium^{126m}, thulium¹⁶⁵, thulium¹⁶⁷, thulium¹⁶⁸ and yttrium¹⁹⁹; particles, such as latex or carbon particles, metal sol, crystallite, liposomes, cells, etc., which may be further labelled with a dye, catalyst or other detectable group; molecules such as biotin, digoxygenin or 5-bromodeoxyuridine; toxin moieties, such as for example a toxin moiety selected from a group of Pseudomonas exotoxin (PE or a cytotoxic fragment or mutant thereof), Diptheria toxin or a cytotoxic fragment or mutant thereof, a Botulinum toxin A, B, C, D, E or F, ricin or a cytotoxic fragment thereof e.g. ricin A, abrin or a cytotoxic fragment thereof, saporin or a cytotoxic fragment thereof, pokeweed antiviral toxin or a cytotoxic fragment thereof and bryodin 1 or a cytotoxic fragment thereof.

The proteins of the invention or immunogenic fragments thereof also include derivatives that are modified (e.g., by the covalent attachment of any type of molecule to the protein) such that covalent attachment does not prevent the protein from binding to antibodies specific for said protein, or otherwise impair the biological activity of the protein. Examples of suitable derivatives include, but are not limited to fucosylated proteins, glycosylated proteins, acetylated proteins, PEGylated proteins, phosphorylated proteins, and amidated proteins.

As used herein, the terms “polynucleotides”, “nucleic acid” and “nucleic acid sequence” refers to any molecule, preferably a polymeric molecule, incorporating units of ribonucleic acid, deoxyribonucleic acid or an analogue thereof. The nucleic acid can be either single-stranded or double-stranded. A single-stranded nucleic acid can be one nucleic acid strand of a denatured double-stranded DNA Alternatively, it can be a single-stranded nucleic acid not derived from any double-stranded DNA. In one aspect, the nucleic acid can be DNA. In another aspect, the nucleic acid can be RNA Suitable nucleic acid molecules are DNA, including genomic DNA or cDNA. Other suitable nucleic acid molecules are RNA, including mRNA.

The terms “decrease”, “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. The terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

References herein to the level of a particular molecule (specifically any of the Apx proteins described herein) encompass the actual amount of the molecule, such as the mass, molar amount, concentration or molarity of the molecule. Preferably in the context of the invention, references to the level of a particular molecule refer to the concentration of the molecule.

The level of a molecule may be determined in any appropriate physiological compartment. Preferred physiological compartments include plasma, blood and/or bronchoalveolar lavage (BAL). The level of a molecule may be determined from any appropriate sample from a patient, e.g. a plasma sample, a blood sample, a serum sample and/or a BAL sample. Other non-limiting examples of samples which may be tested are tissue or fluid samples urine and biopsy samples. Thus, by way of non-limiting example, the invention may reference the level (e.g. concentration) of a molecule (e.g. an antibody to ApxIA, ApxIIA or ApxIIIA) in the plasma and/or BAL of a subject. The level of a molecule pre-treatment with a vaccine of the invention may be interchangeably referred to as the “baseline”.

The level of a molecule may be measured directly or indirectly, and may be determined using any appropriate technique. Suitable standard techniques are known in the art, for example Western blotting and enzyme-linked immunosorbent assays (ELISAs).

A subject can be one who has been previously diagnosed with or identified as suffering from or having a condition in need of treatment or one or more complications related to such a condition, and optionally, have already undergone treatment for a condition as defined herein or the one or more complications related to said condition. Alternatively, a subject can also be one who has not been previously diagnosed as having a condition as defined herein or one or more complications related to said condition. For example, a subject can be one who exhibits one or more risk factors for a condition, or one or more complications related to said condition or a subject who does not exhibit risk factors.

A “subject in need” of treatment for a particular condition can be a subject having that condition, diagnosed as having that condition, or at risk of developing that condition.

A “subject” may be any mammal, particularly a pig. A “subject” may be an adult, juvenile or infant, such as a pig or piglet. A “subject” may be male or female.

As used herein, the term “vaccine” is used to refer to a composition which induces an immune response. For example, the composition may induce an immune response in a subject to which it is administered. Unless explicitly stated, the term “vaccine” includes live vaccines (attenuated and vectored), inactivated vaccines (including whole cell inactivated vaccines and inactivated subunit vaccines) and subunit vaccines.

A live attenuated vaccine comprises whole bacteria which are capable of infecting and replicating in host cells, but have been modified in some way so that they do not cause disease.

A live vectored vaccine comprises a live vector, which is typically non-pathogenic, that has been modified to express one or more antigen from the bacteria against which an immune response is to be raised. Typically, the one or more antigen is a key antigen against which an immune response would be generated if a subject were exposed to the wild-type bacterium (i.e. is infected with the disease) or vaccinated with a live attenuated or inactivated vaccine. The antigen may be a protein antigen, or fragment thereof, or a polysaccharide antigen, or fragment thereof. The antigen may be expressed recombinantly or as a conjugate or fusion protein.

An inactivated whole cell vaccine comprises whole bacteria which have been killed or inactivated (e.g. by heat or chemical treatment). Inactivated bacteria are not capable of infecting or replicating in host cells and do not cause disease.

A subunit vaccine comprises one or more component of the bacterium against which an immune response is to be raised. Typically, the one or more component is a key antigen against which an immune response would be generated if a patient were exposed to the wild-type bacteria (i.e. is infected with the disease) or vaccinated with a live attenuated or inactivated vaccine. The component may be a protein antigen, or fragment thereof, or a polysaccharide antigen, or fragment thereof. The component may be expressed recombinantly or as a conjugate or fusion protein. In the case of subunit vaccines comprising toxin components, these may either be (i) modified such that the toxin no longer has toxic (e.g. cytotoxic or haemolytic activity), or (ii) wild-type toxins which have been inactivated (e.g. by heat or chemical treatment).

As used herein, the terms Apx polypeptides or Apx toxins are used interchangeably and encompass any one, two, or three of ApxIA, ApxIIA and ApxIIIA (e.g. ApxIA; ApxIIA; ApxIIIA; ApxIA and ApxIIA; ApxIA and ApxIIA; ApxIIA and ApxIIIA; and/or ApxIA, ApxIIA and ApxIIIA), unless expressly stated to the contrary. Typically, reference herein to Apx polypeptides or Apx toxins encompasses all of ApxIA, ApxIIA and ApxIIIA unless expressly stated otherwise.

A wild-type APP “toxin” is a polypeptide that consists of the amino acid sequence of ApxI, ApxIIA or ApxIIIA (e.g. as set forth in SEQ ID Nos: 1 to 3 respectively) and exhibits cytolytic and/or haemolytic activity. A “toxoid” in this disclosure is a polypeptide that is a modified form of the “toxin” wherein the modification is achieved by the replacement or deletion of one or more amino acid that is susceptible to acylation in vivo in APP, said toxoid does not exhibit any cytotoxic or haemolytic activity.

The genus Actinobacillus comprises Gram-negative, non-spore forming and predominantly encapsulated bacterial species that colonise mucosal surfaces of the respiratory and urogenital tracts. Relevant veterinary species are for example APP, Actinobacillus suis, Actinobacillus equuli and Actinobacillus lignieresii which are the preferred Actinobacillus spp. of the disclosure. Actinobacillus spp. normally show strong host species specificity. Preferred APP serotypes are serotypes 1, 5, 7, 8, 9 and 11.

The terms “strain”, “serovar” and “serotype” are used interchangeably herein to describe a distinct group or classification of APP.

The publications discussed herein are provided solely for their disclosure prior to the filing date of the present application. Nothing herein is to be construed as an admission that such publications constitute prior art to the claims appended hereto.

### ApxIA, ApxIIA and ApxIIIA Polypeptides

The present invention relates to microorganisms which express one of each of ApxIA, ApxIIA and ApxIIIA polypeptides (hereafter collectively and exchangeably designated ApxA toxins or polypeptides for brevity). Whilst conventional approaches have been able to produce a series of contructs each expressing individual ApxA toxins (for example Hur et al. J. Vet. Med. Sci. 77(12):1693-1696, 2015; and Hur and Lee Vet. Res. Commun. 38:87-91, 2014; which are herein incorporated by reference in its entirely), this is very different to providing a single microorganism expressing one each of ApxIA, ApxIIA and ApxIIIA polypeptides. The former is technically straightforward, whereas the present inventors have pioneered the natural transformation methodology and so are the first to provide a technique by which microorganisms expressing one of each of ApxIA, ApxIIA and ApxIIIA polypeptides can be produced. Linear template DNA is used in natural transformation, ensuring that allele exchange occurs via a double-cross over event, resulting in correct directional insertion of the gene replacement without incorporation of any extra (e.g. plasmid backbone) DNA. Other approaches described in the art are also unsuitable for the production of microorganisms according to the invention, and are typically associated with one or more disadvantages. For example, some prior art techniques are dependent on the particular APP strain, or rely on serial single cross-over events which do not reliably result in the production of the desired gene in a predictable manner (e.g., Oswald et al. FEMS Microbiol. Lett. 179:153-160, 1999, which is herein incorporated by reference in its entirety).

The ApxIA, ApxIIA and ApxIIIA polypeptides expressed by microorganisms of the invention may be wild-type ApxA polypeptides as described herein. The ApxIA, ApxIIA and ApxIIIA polypeptides may be variants of wild-type ApxA polypeptides as described herein which retain the cytotoxic and/or haemolytic activity of the wild-type ApxA polypeptide from which they are derived. The ApxIA, ApxIIA and ApxIIIA polypeptides may be modified ApxA polypeptides which have reduced cytotoxic and/or haemolytic activity compared with the wild-type ApxA polypeptide from which they are derived. In particular, the ApxIA, ApxIIA and ApxIIIA polypeptides may be modified ApxA polypeptides as described herein. Typically, all three of ApxIA, ApxIIA and ApxIIIA polypeptides are either wild-type ApxA polypeptides or modified ApxA polypeptides.

One or more of the ApxA polypeptides expressed by a microorganism of the invention are typically in their native conformation, preferably all of the ApxA polypeptides expressed by a microorganism of the invention are in their native conformation.

The ApxA polypeptides of the invention can induce a humoral and/or cellular immunological response against one or more serotypes of AAP in a mammal, in particular a pig, when administered to said mammal. The ApxA polypeptides can induce a humoral and/or cellular immunological response against at least one, at least two, at least three, at least four, at least five, at least six, at least seven, at least eight, at least nine, at least ten, at least 12, at least 15 or more, up to all known serotypes (currently 19) of APP in a mammal, in particular a pig, when administered to said mammal. Preferably the ApxA polypeptides of the invention induce a sterile immunity (i.e. provide complete protection) against APP, APP strains, APP serotypes or APP serovars.

### Wild-Type ApxA Polypeptides

The microorganisms, vectors and vaccines of the invention may comprise wild-type ApxIA, ApxIIA and ApxIIIA polypeptides, or a fragment or variant thereof, provided that said variants do not encompass modifications at either acylation site as described herein in the context of modified ApxIA, ApxIIA and ApxIIIA polypeptides of the invention (i.e. amino acids corresponding to K560 and/or K686 in ApxIA; K557 and/or N687 in ApxIIA; and K571 and/or K702 in ApxIIIA).

A wild-type ApxIA polypeptide typically has an amino acid sequence corresponding to SEQ ID NO: 1. A variant of this wild-type ApxIA polypeptide may have at least 60%, at least 70%, more preferably at least 80%, at least 85%, at least 90%, at least 95%, and most preferably at least 97% or at least 99% sequence identity with the wild-type ApxIA sequence (e.g. SEQ ID NO: 1). By way of non-limiting example, a variant of an ApxIA wild-type polypeptide is at least 90% homologous to the wild-type ApxIA amino acid sequence. A fragment of the wild-type ApxIA comprises at least 30%, at least 40%, at least 50%, at least 60%, at least 70%, at least 80%, at least 90%, at least 95%, at least 97% or more of the wild-type ApxIA polypeptide from which it is derived (e.g. SEQ ID NO: 1).

A wild-type ApxIIA polypeptide typically has an amino acid sequence corresponding to SEQ ID NO: 2. A variant of this wild-type ApxIIA polypeptide may have at least 60%, at least 70%, more preferably at least 80%, at least 85%, at least 90%, at least 95%, and most preferably at least 97% or at least 99% sequence identity with the wild-type ApxIIA sequence (e.g. SEQ ID NO: 2). By way of non-limiting example, a variant of an ApxIIA wild-type polypeptide is at least 90% homologous to the wild-type ApxIIA amino acid sequence. A fragment of the wild-type ApxIIA comprises at least 30%, at least 40%, at least 50%, at least 60%, at least 70%, at least 80%, at least 90%, at least 95%, at least 97% or more of the wild-type ApxIIA polypeptide from which it is derived (e.g. SEQ ID NO: 2). A particular example of a fragment of a wild-type ApxIIA polypeptide is set out in SEQ ID NO: 7. Approximately 62% of the full-length wild-type ApxIIA sequence has been deleted to produce this wild-type ApxIIA fragment.

A wild-type ApxIIIA polypeptide typically has an amino acid sequence corresponding to SEQ ID NO: 3. A variant of this wild-type ApxIIIA polypeptide may have at least 60%, at least 70%, more preferably at least 80%, at least 85%, at least 90%, at least 95%, and most preferably at least 97% or at least 99% sequence identity with the wild-type ApxIIIA sequence (e.g. SEQ ID NO: 3). By way of non-limiting example, a variant of an ApxIIIA wild-type polypeptide is at least 90% homologous to the wild-type ApxIIIA amino acid sequence. A fragment of the wild-type ApxIIIA comprises at least 30%, at least 40%, at least 50%, at least 60%, at least 70%, at least 80%, at least 90%, at least 95%, at least 97% or more of the wild-type ApxIIIA polypeptide from which it is derived (e.g. SEQ ID NO: 3).

Variants of the wild-type ApxA polypeptides typically comprise conservative substitutions or deletions as defined in the general definitions section above. These variants do not comprise substitutions or deletions which reduce or abrogate the cytotoxicity and/or haemolytic activity of the wild-type ApxA polypeptides (instead, ApxA polypeptides with reduced or abrogated cytotoxicity and/or haemolytic activity are encompassed by the modified ApxA polypeptides of the invention, described herein).

In particular, variants of the wild-type ApxA polypeptides do not comprise conservative substitutions or deletions of either amino acid susceptible to acylation (i.e. amino acids corresponding to K560 and/or K686 in ApxIA; K557 and/or N687 in ApxIIA; and K571 and/or K702 in ApxIIIA). In other words, variants of the wild-type ApxA polypeptides comprise both amino acids susceptible to acylation. Thus, variants of the wild-type ApxA polypeptides may comprise substitutions and/or deletions provided the one or two amino acids susceptible to acylation (or any other amino acids required for cytotoxic and haemolytic activity) are not substituted and/or deleted.

The wild-type ApxA polypeptide variants may comprise any number of substitutions or deletions, provided the cytotoxic and/or haemolytic activity of the wild-type ApxA polypeptides is retained. Typically, the wild-type ApxA polypeptide variants will comprise less than ten amino acid deletions, nine amino acid deletions, eight amino acid deletions, seven amino acid deletions, six amino acid deletions, five amino acid deletions, four amino acid deletions, three amino acid deletions, two amino acid deletions or one amino acid deletion. Preferably, the wild-type ApxA polypeptide variants will comprise only one, two or three amino acid deletions. Typically the wild-type ApxA polypeptide variants will comprise less than ten conservative amino acid substitutions, nine conservative amino acid substitutions, eight conservative amino acid substitutions, seven conservative amino acid substitutions, six conservative amino acid substitutions, five conservative amino acid substitutions, four conservative amino acid substitutions, three conservative amino acid substitutions, two conservative amino acid substitutions or one conservative amino acid substitution. Preferably, the wild-type ApxA polypeptide variants will comprise only one, two or three conservative amino acid substitutions. The wild-type ApxA polypeptide variants may comprise less than ten conservative amino acid substitutions and deletions in total, nine conservative amino acid substitutions and deletions in total, eight conservative amino acid substitutions and deletions in total, seven conservative amino acid substitutions and deletions in total, six conservative amino acid substitutions and deletions in total, five conservative amino acid substitutions and deletions in total, four conservative amino acid substitutions and deletions in total, three conservative amino acid substitutions and deletions in total, two conservative amino acid substitutions and deletions in total or one conservative amino acid substitution or deletion.

Fragments of the wild-type ApxA polypeptides also comprise both amino acids susceptible to acylation.

Any combination of these wild-type ApxA polypeptides may be used together, provided that each of an ApxIA, ApxIIA and ApxIIIA polypeptide is used.

### Modified ApxA Polypeptides

The present inventors have previously developed modified forms of ApxIA, ApxIIA and ApxIIIA which may be used in the present invention.

These modified ApxA toxins have been modified at at least one of the two acylation sites of ApxIA, ApxIIA and ApxIIIA (typically amino acids corresponding to K560 and/or K686 in ApxIA; K557 and/or N687 in ApxIIA; and K571 and/or K702 in ApxIIIA), creating inactive but fully immunogenic toxoid forms of these proteins. The modification at either or both acylation sites may be by amino acid substitution or deletion at the position of the amino acid susceptible to acylation in the wild-type polypeptide. The substitution or deletion of the amino acids at either or both of the two acylation sites prevents them from being acylated by any endogenous or exogeneous acyltransferase (e.g. an ApxC of APP). Preferably, both of the acylation sites of ApxIA, ApxIIA and/or ApxIIIA are substituted or deleted.

As a result of this inability to be acylated (whether by substitution or deletion of either or both of the amino acids at the acylation positions within ApxA polypeptides), these modified Apx toxins cannot initiate binding to the target cell membrane and consequently have substantial pathological effect, particularly no cytotoxic or haemolytic activity. These inactive ApxA polypeptides are safer to use in vaccine compositions than vaccines in which the acyltransferase ApxC is deleted because in the acyltransferase deletion vaccines the Apx polypeptides remain pathological if an acyltransferase is provided exogenously, either by a naturally occurring strain of APP or any other source of acyltransferase in vivo. The modified ApxA proteins of the invention typically elicit fewer side effects (e.g. fever, vomiting, apathy) when used to vaccinate a mammal whilst conferring immunological protection against APP. These modified ApxA may therefore be considered as inactivated toxins (also referred to as toxoids). A further benefit is that these modified Apx toxins, whether in subunit or whole cell vaccine form do not need to be chemically inactivated, resulting in highly immunogenic vaccines, such that lower doses may be used.

Accordingly, the microorganisms, vectors and vaccines of the invention may comprise modified ApxIA, ApxIIA and ApxIIIA polypeptides, or a fragment or variant thereof, provided that said modified ApxA polypeptides comprise a modification at either or both acylation site as described. These modified ApxA polypeptides are also referred interchangeably herein as inactive ApxA polypeptides. These modified ApxA polypeptides typically retain common antigenic cross-reactivity with the corresponding wild-type ApxA polypeptide from which they are derived.

An inactive ApxIA typically has an amino acid sequence corresponding to the wild-type ApxIA amino acid sequence of SEQ ID NO: 1, modified by an amino acid substitution at at least one amino acid selected from the group consisting of K560 and K686. Preferably the inactive ApxIA comprises substitutions at both K560 and K686. Variants of this inactive ApxIA are also encompassed. A variant of this inactive ApxIA polypeptide may have at least 60%, at least 70%, more preferably at least 80%, at least 85%, at least 90%, at least 95%, and most preferably at least 97% or at least 99% sequence identity with the inactive ApxIA sequence, provided that said variant comprises the at least one modified (substituted) amino acid. By way of non-limiting example, a variant of an inactive ApxIA polypeptide is at least 90% homologous to the inactive ApxIA amino acid sequence, wherein said variant comprises an amino acid substitution at position K560 and/or K686. A fragment of the inactive ApxIA comprises at least 30%, at least 40%, at least 50%, at least 60%, at least 70%, at least 80%, at least 90%, at least 95%, at least 97% or more of the inactive ApxIA polypeptide from which it is derived, provided that said variant comprises the at least one modified (substituted) amino acid. Preferably variants and/or fragments of the inactive ApxIA comprise substitutions at both K560 and K686.

An inactive ApxIIA typically has an amino acid sequence corresponding to the wild-type ApxIIA amino acid sequence of SEQ ID NO: 2, modified by an amino acid substitution at least one amino acid selected from the group consisting of K557 and N687. Preferably the inactive ApxIIA comprises substitutions at both K557 and N687. Variants of this inactive ApxIIA are also encompassed. A variant of this inactive ApxIIA polypeptide may have at least 60%, at least 70%, more preferably at least 80%, at least 85%, at least 90%, at least 95%, and most preferably at least 97% or at least 99% sequence identity with the inactive ApxIIA sequence, provided that said variant comprises the at least one modified (substituted) amino acid. By way of non-limiting example, a variant of an inactive ApxIIA polypeptide is at least 90% homologous to the inactive ApxIIA amino acid sequence, wherein said variant comprises an amino acid substitution at position K557 and/or N687. A fragment of the inactive ApxIIA comprises at least 30%, at least 40%, at least 50%, at least 60%, at least 70%, at least 80%, at least 90%, at least 95%, at least 97% or more of the inactive ApxIIA polypeptide from which it is derived, provided that said variant comprises the at least one modified (substituted) amino acid. Preferably variants and/or fragments of the inactive ApxIIA comprise substitutions at both K557 and N687. A particular example of a fragment of an inactive ApxIIA polypeptide is set out in SEQ ID NO: 8. Approximately 62% of the full-length inactive ApxIIA sequence has been deleted to produce this inactive ApxIIA fragment.

An inactive ApxIIIA typically has an amino acid sequence corresponding to the wild-type ApxIIA amino acid sequence of SEQ ID NO: 3, modified by an amino acid substitution at least one amino acid selected from the group consisting of K571 and K702. Preferably the inactive ApxIIIA comprises substitutions at both K571 and K702. Variants of this inactive ApxIIIA are also encompassed. A variant of this inactive ApxIIIA polypeptide may have at least 60%, at least 70%, more preferably at least 80%, at least 85%, at least 90%, at least 95%, and most preferably at least 97% or at least 99% sequence identity with the inactive ApxIIIA sequence, provided that said variant comprises the at least one modified (substituted) amino acid. By way of non-limiting example, a variant of an inactive ApxIIIA polypeptide is at least 90% homologous to the inactive ApxIIIA amino acid sequence, wherein said variant comprises an amino acid substitution at position K571 and/or K702. A fragment of the inactive ApxIIIA comprises at least 30%, at least 40%, at least 50%, at least 60%, at least 70%, at least 80%, at least 90%, at least 95%, at least 97% or more of the inactive ApxIIIA polypeptide from which it is derived, provided that said variant comprises the at least one modified (substituted) amino acid. Preferably variants and/or fragments of the inactive ApxIIIA comprise substitutions at both K571 and K702.

An exemplary inactive ApxIA polypeptide of the invention comprises the amino acid sequence of SEQ ID NO: 4.

An exemplary inactive ApxIIA polypeptide of the invention comprises the amino acid sequence of SEQ ID NO: 5.

An exemplary inactive ApxIIIA polypeptide of the invention comprises the amino acid sequence of SEQ ID NO: 6.

An inactive ApxIA may have an amino acid sequence corresponding to the wild-type ApxIA amino acid sequence of SEQ ID NO: 1, modified by a deletion at at least one amino acid selected from the group consisting of K560 and K686. Preferably the inactive ApxIA comprises deletions at both K560 and K686. Variants of this inactive ApxIA are also encompassed. A variant of this inactive ApxIA polypeptide may have at least 60%, at least 70%, more preferably at least 80%, at least 85%, at least 90%, at least 95%, and most preferably at least 97% or at least 99% sequence identity with the inactive ApxIA sequence, provided that said variant comprises the at least one deletion. By way of non-limiting example, a variant of an inactive ApxIA polypeptide is at least 90% homologous to the inactive ApxIA amino acid sequence, wherein said variant comprises a deletion at position K560 and/or K686. A fragment of the inactive ApxIA comprises at least 30%, at least 40%, at least 50%, at least 60%, at least 70%, at least 80%, at least 90%, at least 95%, at least 97% or more of the inactive ApxIA polypeptide from which it is derived, provided that said variant comprises the at least one deletion. Preferably variants and/or fragments of the inactive ApxIA comprise deletions at both K560 and K686.

An inactive ApxIIA typically has an amino acid sequence corresponding to the wild-type ApxIIA amino acid sequence of SEQ ID NO: 2, modified by a deletion at at least one amino acid selected from the group consisting of K557 and N687. Preferably the inactive ApxIIA comprises deletions at both K557 and N687. Variants of this inactive ApxIIA are also encompassed. A variant of this inactive ApxIIA polypeptide may have at least 60%, at least 70%, more preferably at least 80%, at least 85%, at least 90%, at least 95%, and most preferably at least 97% or at least 99% sequence identity with the inactive ApxIIA sequence, provided that said variant comprises the at least one deletion. By way of non-limiting example, a variant of an inactive ApxIIA polypeptide is at least 90% homologous to the inactive ApxIIA amino acid sequence, wherein said variant comprises a deletion at position K557 and/or N687. A fragment of the inactive ApxIIA comprises at least 30%, at least 40%, at least 50%, at least 60%, at least 70%, at least 80%, at least 90%, at least 95%, at least 97% or more of the inactive ApxIIA polypeptide from which it is derived, provided that said variant comprises the at least one deletion. Preferably variants and/or fragments of the inactive ApxIIA comprise deletions at both K557 and N687.

An inactive ApxIIIA typically has an amino acid sequence corresponding to the wild-type ApxIIA amino acid sequence of SEQ ID NO: 3, modified by a deletion at at least one amino acid selected from the group consisting of K571 and K702. Preferably the inactive ApxIIIA comprises deletions at both K571 and K702. Variants of this inactive ApxIIIA are also encompassed. A variant of this inactive ApxIIIA polypeptide may have at least 60%, at least 70%, more preferably at least 80%, at least 85%, at least 90%, at least 95%, and most preferably at least 97% or at least 99% sequence identity with the inactive ApxIIIA sequence, provided that said variant comprises the at least one deletion. By way of non-limiting example, a variant of an inactive ApxIIIA polypeptide is at least 90% homologous to the inactive ApxIIIA amino acid sequence, wherein said variant comprises a deletion at position K571 and/or K702. A fragment of the inactive ApxIIIA comprises at least 30%, at least 40%, at least 50%, at least 60%, at least 70%, at least 80%, at least 90%, at least 95%, at least 97% or more of the inactive ApxIIIA polypeptide from which it is derived, provided that said variant comprises the at least one deletion. Preferably variants and/or fragments of the inactive ApxIIIA comprise deletions at both K571 and K702.

In inactive ApxA polypeptides, where one or both of the amino acids which are susceptible to acylation (i.e. the acylation sites) are deleted, the deletion may comprise point deletions where only either the one or the two amino acids susceptible to acylation in each wild-type ApxA sequence are deleted. Alternatively, the deletions may also delete amino acids in an area adjacent to the one or two amino acids susceptible to acylation. Thus, the respective deletions may comprise a deletion of two, three, four, five, six, seven, eight, nine, ten, 20, 30, 40, 50, 60, 70, 80, 90, 100, 110, 120, 130, 140, 150, 200, 300, 350 or 400 amino acids, provided that the deletion comprises one of the two amino acids susceptible to acylation. When both of the amino acids which are susceptible to acylation are deleted, the size of the deletion for each amino acid susceptible to acylation may be independent from each other, or the two deletions may be the same size. Deletions which cover a consecutive stretch of amino acids between the two amino acids susceptible to acylation are also disclosed.

Whether either or both of the amino acids which are susceptible to acylation are deleted, the deletion(s) do not delete more than 70% of the corresponding wild-type amino acid sequence.

Other modified ApxA polypeptides may be used in the present invention. The methods of the present invention may be used to produce a microorganism which expresses inactive forms of each of ApxIA, ApxIIA and ApxIIIA. By way of non-limiting example, WO 2013/068629 (herein incorporated by reference in its entirety) describes modified apxIIA and ApxIIIA genes which are mutated in their transmembrane domains and the encoded modified ApxIIA and ApxIIIA retain their immunogenicity whilst being haemolytically and cytotoxically inactive. These modified apxIIA and ApxIIIA genes may be used in the present invention as alternatives to or in combination with the modified ApxA polypeptides with one or more deleted/substituted acylation site.

Variants of the inactive ApxA polypeptides typically comprise conservative substitutions or deletions as defined in the general definitions section above. Thus, variants of the inactive ApxA polypeptides may comprise further deletions outside the deletion region comprising the one or two amino acids susceptible to acylation.

The inactive ApxA polypeptide variants may comprise any number of substitutions or deletions, provided the cytotoxic and/or haemolytic activity of the wild-type ApxA polypeptides is still abrogated or reduced. Typically, the inactive ApxA polypeptide variants will comprise less than ten amino acid deletions, nine amino acid deletions, eight amino acid deletions, seven amino acid deletions, six amino acid deletions, five amino acid deletions, four amino acid deletions, three amino acid deletions, two amino acid deletions or one amino acid deletion. Preferably, the inactive ApxA polypeptide variants will comprise only one, two or three amino acid deletions. Typically the inactive ApxA polypeptide variants will comprise less than ten conservative amino acid substitutions, nine conservative amino acid substitutions, eight conservative amino acid substitutions, seven conservative amino acid substitutions, six conservative amino acid substitutions, five conservative amino acid substitutions, four conservative amino acid substitutions, three conservative amino acid substitutions, two conservative amino acid substitutions or one conservative amino acid substitution. Preferably, the inactive ApxA polypeptide variants will comprise only one, two or three conservative amino acid substitutions. The inactive ApxA polypeptide variants may comprise less than ten conservative amino acid substitutions and deletions in total, nine conservative amino acid substitutions and deletions in total, eight conservative amino acid substitutions and deletions in total, seven conservative amino acid substitutions and deletions in total, six conservative amino acid substitutions and deletions in total, five conservative amino acid substitutions and deletions in total, four conservative amino acid substitutions and deletions in total, three conservative amino acid substitutions and deletions in total, two conservative amino acid substitutions and deletions in total or one conservative amino acid substitution or deletion.

Any combination of these inactive ApxA polypeptides may be used together, provided that each of an ApxIA, ApxIIA and ApxIIIA polypeptide is used.

In an inactive ApxIA, ApxIIA or ApxIIIA polypeptide of the invention, the one or two amino acids susceptible to acylation may be each be independently substituted with any amino acid that is not susceptible to acylation.

Amino acids susceptible to acylation are naturally occurring amino acids such as lysine and/or asparagine. Amino acids not susceptible to acylation are known to the skilled person and can be used to substitute one or both of the amino acids susceptible to acylation. The amino acid to be substituted at each amino acid susceptible to acylation in the wild-type ApxA polypeptides, i.e. amino acids corresponding to K560 and/or K686 in ApxIA; K557 and/or N687 in ApxIIA; and K571 and/or K702 in ApxIIIA, may be independently selected from the group consisting of alanine, glycine, isoleucine, leucine, methionine, valine, serine, threonine, asparagine, glutamine, aspartic acid, histidine, cysteine, proline, phenylalanine, tyrosine, tryptophan and glutamic acid. More preferably, each amino acid to be substituted at each amino acid susceptible to acylation in the wild-type ApxA polypeptides, i.e. K560 and/or K686 in ApxIA; K557 and/or N687 in ApxIIA; and K571 and/or K702 in ApxIIIA, may be independently selected from the group consisting of alanine, glycine, serine, isoleucine and leucine, valine and threonine. Even more preferably, each amino acid to be substituted at each amino acid susceptible to acylation in the wild-type ApxA polypeptides, i.e. K560 and/or K686 in ApxIA; K557 and/or N687 in ApxIIA; and K571 and/or K702 in ApxIIIA, may be independently selected from the group consisting of alanine, glycine and serine. The most preferred amino acid not susceptible to acylation (for each of ApxIA, ApxIIA and ApxIIIA) is alanine.

Preferably, for each inactive ApxA polypeptide, i.e. each of ApxIA, ApxIIA and ApxIIIA, both of the two amino acids susceptible to acylation are modified. Accordingly, the wild-type sequence of ApxIA (exemplified by SEQ ID NO: 1) is modified at amino acids corresponding to K560 and K686. The wild-type sequence of ApxIIA (exemplified by SEQ ID NO: 2) is modified at amino acids corresponding to K557 and N687. The wild-type sequence of ApxIIIA (exemplified by SEQ ID NO: 3) is modified at amino acids corresponding to K571 and K702. Preferably, both of the two amino acids susceptible to acylation in each of ApxIA, ApxIIA and ApxIIIA are substituted with alanine. Thus, preferred inactive ApxA polypeptides have the amino acid sequence set forth in SEQ ID: 4 (inactive ApxIA); SEQ ID NO: 5 (inactive ApxIIA) and SEQ ID NO: 6 (inactive ApxIIIA). Variants and fragments of these sequences are also encompassed as described above.

### Nucleic Acids

Nucleic acids comprising a nucleic acid sequence capable of coding for the above described wild-type and inactive ApxA polypeptides are also disclosed. The disclosed nucleic acid can be cDNA, DNA, RNA, cRNA or PNA (peptide nucleic acid). The term “nucleic acid sequence” refers to a heteropolymer of nucleotides or the sequence of these nucleotides. The nucleic acid can comprise a nucleic acid as set forth in SEQ ID NO: 9, 10, or 11 (for wild-type apxIA, apxIIA and apxIIIA respectively) or 12, 13 or 14 (for inactive apxIA, apxIIA and apxIIIA respectively). Variants and fragments of said nucleic acids which encode variants and fragments of wild-type and inactive ApxA polypeptides as disclosed herein are also encompassed.

Said nucleic acids may be comprised in a microorganism of the invention, as described herein.

The nucleic acid may be comprised in a vector suitable for cloning or expressing the nucleic acids of the disclosure. Exemplary vectors are pEX-A258 (SEQ ID NO: 15), pQE-80L (SEQ ID NO: 16) and/or pQE-60 (SEQ ID NO: 17). The nucleic acids or vectors may comprise additional regulatory non-coding elements like inducible or non-inducible promoters, operators (e.g. lac-operator) or nucleic acids coding for other APP proteins.

One or more nucleic acids of the invention may encode for each of an ApxIA, an ApxIIA and an ApxIIIA polypeptide (wild-type or inactive) as disclosed herein. All three ApxA polypeptides may be encoded by a single nucleic acid. Alternatively, each ApxA polypeptide may be encoded by a separate nucleic acid. Alternatively, any two of the ApxA polypeptides may be encoded by a first nucleic acid, with the remaining ApxA polypeptide being encoded by a second nucleic acid. By way of non-limiting example, ApxIA and ApxIIA polypeptides of the invention may be encoded by a first nucleic acid, with ApxIIIA encoded by a second nucleic acid. By way of a further non-limiting example, ApxIA and ApxIIIA polypeptides of the invention may be encoded by a first nucleic acid, with ApxIIA encoded by a second nucleic acid. By way of a further non-limiting example, ApxIIA and ApxIIIA polypeptides of the invention may be encoded by a first nucleic acid, with ApxIA encoded by a second nucleic acid. Thus, the present invention provides a nucleic acid or set of nucleic acids (i.e. one or more nucleic acid) encoding for an ApxIA, an ApxIIA and an ApxIIIA polypeptide (wild-type or inactive) of the invention.

The one or more nucleic acid may be integrated into one or more vector, wherein the one or more nucleic acid is operably linked to an expression control region of the vector(s). Each nucleic acid may be operably linked to a separate expression control region, or the nucleic acids may be operably linked to the same expression control region, forming a polycistronic cassette. Thus, expression vectors are also disclosed, wherein the expression vector preferably comprises one or more regulatory sequences in addition to the nucleic acid(s) encoding for the ApxIA, ApxIIA and ApxIIIA polypeptides. The present invention therefore provides a vector or set of vectors (i.e. one or more vector) encoding for an ApxIA, an ApxIIA and an ApxIIIA polypeptide (wild-type or inactive) of the invention.

One or more vector of the invention may encode for each of an ApxIA, an ApxIIA and an ApxIIIA polypeptide (wild-type or inactive) as disclosed herein. All three ApxA polypeptides may be encoded by a vector. Alternatively, each ApxA polypeptide may be encoded by a separate vector. Alternatively, any two of the ApxA polypeptides may be encoded by a first vector, with the remaining ApxA polypeptide being encoded by a second vector. By way of non-limiting example, ApxIA and ApxIIA polypeptides of the invention may be encoded by a first vector, with ApxIIIA encoded by a second vector. By way of a further non-limiting example, ApxIA and ApxIIIA polypeptides of the invention may be encoded by a first vector, with ApxIIA encoded by a second vector. By way of a further non-limiting example, ApxIIA and ApxIIIA polypeptides of the invention may be encoded by a first vector, with ApxIA encoded by a second vector. The nucleic acid encoding each ApxA polypeptide in said one or more vector may be operably linked to the same expression control region as described herein, or maybe operably linked to separate expression control regions.

The term “expression vector” generally refers to a plasmid, phage, virus or vector for expressing a polypeptide from a DNA (RNA) sequence. An expression vector may comprise a transcriptional unit comprising an assembly of (1) a genetic element or elements having a regulatory role in gene expression, for example promoters or enhancers; (2) a structural or coding sequence which is transcribed into mRNA and translated into protein; and (3) appropriate transcription initiation and termination sequences. Structural units intended for use in yeast or eukaryotic expression systems preferably include a leader sequence enabling extracellular secretion of translated protein by a host cell. Alternatively, where recombinant protein is expressed without a leader or transport sequence, it may include an N-terminal methionine residue. This residue may or may not be subsequently cleaved from the expressed recombinant protein to provide a final product.

Any of these one or more nucleic acid combinations or one or more vector combinations may be comprised in a vaccine composition of the invention. Preferably for such vaccine combinations the one or more nucleic acid is integrated into one or more vector as disclosed herein.

### Microorganisms

The present invention particularly relates to microorganisms which comprise each of an ApxIA, an ApxIIA and an ApxIIIA polypeptide. These ApxA polypeptides may be wild-type or inactive ApxA polypeptides as described herein. Preferably, the microorganism comprises either all wild-type ApxA polypeptides or all inactive ApxA polypeptides as disclosed herein. As used herein, references to a microorganism “comprising” (wild-type or inactive) ApxA encompass microorganisms producing, encoding or expressing said ApxA.

The APP ApxA polypeptides may be provided via nucleic acids or vectors of the invention. Accordingly, the invention provides a microorganism comprising: (a) a nucleic acid sequence encoding ApxIA; (b) a nucleic acid sequence encoding ApxIIA; and (c) a nucleic acid sequence encoding ApxIIIA. Said nucleic acids may be comprised within one or more vector as described herein.

The nucleic acid encoding each of the ApxIA, ApxIIA and ApxIIIA polypeptides may be comprised within the genome of the microorganism. This may involve integration of the nucleic acid within the genome of the microorganism, such as by using molecular biological techniques. The nucleic acid encoding each of the ApxIA, ApxIIA and ApxIIIA polypeptides may be comprised separately (e.g. extra-chromosomally) from the genome of the microorganism. In such instances, expression of the ApxIA, ApxIIA and/or ApxIIIA polypeptides expressed by an extra-chromosomal nucleic acid may be transient. By way of non-limiting example, extra-chromosomal expression of one or more of the ApxIA, ApxIIA and ApxIIIA polypeptides may be achieved when the nucleic acid encoding one or more of the ApxIA, ApxIIA and ApxIIIA polypeptides is part of a (non-integrating) plasmid. One benefit of integrating the ApxA polypeptides (wild-type or inactive) into the genome of the microorganism is that expression of the ApxA polypeptides is stable and does not require antibiotic selection for maintenance. Introduction of ApxA polypeptides according to the invention, particularly when the apxA genes are stably integrated into the genome of the microorganism, such as when introduced via natural transformation, is typically not associated with a selection marker (e.g. antimicrobial or antibiotic-resistance marker). Instead, in such embodiments, the apxA genes are typically unmarked within the chromosome of the microorganism.

Any combination of ApxA polypeptides encoding nucleic acids comprised with the genome of a microorganism or comprised separately from the genome is encompassed herein. By way of non-limiting example, a microorganism may comprise one or more nucleic acid encoding for ApxIA and ApxIIA within its genome, with a nucleic acid encoding for ApxIIIA comprised separately from its genome (e.g. in a separate plasmid). By way of a further non-limiting example, a microorganism may comprise one or more nucleic acid encoding for ApxIIA and ApxIIIA within its genome, with a nucleic acid encoding for ApxIA comprised separately from its genome (e.g. in a separate plasmid). By way of a further non-limiting example, a microorganism may comprise one or more nucleic acid encoding for ApxIA and ApxIIIA within its genome, with a nucleic acid encoding for ApxIIA comprised separately from its genome (e.g. in a separate plasmid).

The microorganism may be any appropriate bacterial species. Non-limiting examples include Actinobacillus species, for example APP, Actinobacillus suis, an Actinobacillus species strain, for example a strain of APP or a strain A. suis, or a particular serotype (ST) of an Actinobacillus species, such a strain of a serotype of APP or a strain of a serotype of A. suis. Other examples of appropriate bacteria include E. coli, for example an E. coli strain, particularly E. coli Top10F′ strain. Preferably the microorganism is an APP or an APP strain, e.g. serotype 2 (ST2 e.g. APP23 or 07/07), serotype 5 (ST5, e.g. DZY47), serotype 7 (ST7, e.g. DZY33) or serotype 8 (ST8, e.g. DZY49). References herein to an Actinobacillus species encompass references to strains and serotypes (also called serovars) of said Actinobacillus species. For example, references herein to APP also encompass references to strains, serotypes/serovars of APP.

The microorganism may be an APP strain which is produced by modification of an existing APP strain, such as naturally occurring APP strains. The resulting microorganism may comprise (express/produce) wild-type or inactive ApxA polypeptides as described herein.

A microorganism of the invention may be produced from an APP strain which expresses only one of ApxIA, ApxIIA and ApxIIIA endogenously. In such instances, the additional two ApxA polypeptides may be introduced to the microorganism, either in wild-type form if the endogenous ApxA is retained in wild-type form, or in inactive form if the endogenous ApxA is replaced or modified to produce an inactive form.

A microorganism of the invention may be produced from an APP strain which expresses only two of ApxIA, ApxIIA and ApxIIIA endogenously. In such instances, the additional ApxA polypeptide may be introduced to the microorganism, either in wild-type form if the endogenous ApxA are retained in wild-type form, or in inactive form if the endogenous ApxA are replaced or modified to produce the inactive forms.

A microorganism of the invention may be produced from an APP strain which expresses endogenous ApxIIA and ApxIIIA polypeptides, such as a serotype 2, 8 or 15 strain. By way of non-limiting example, a nucleic acid encoding a wild-type ApxIA polypeptide as disclosed herein may be introduced to said APP strain to produce a microorganism according to the invention (the nucleic acid encoding the wild-type ApxIA polypeptide may be integrated into the genome of the APP strain, or may be present extra-chromosomally within the microorganism).

A microorganism of the invention may be produced from an APP strain which expresses endogenous ApxIA and ApxIIA polypeptides, such as a serotype 1, 5 or 9 strain. By way of non-limiting example, a nucleic acid encoding a wild-type ApxIIIA polypeptide as disclosed herein may be introduced to said APP strain to produce a microorganism according to the invention (the nucleic acid encoding the wild-type ApxIIIA polypeptide may be integrated into the genome of the APP strain, or may be present extra-chromosomally within the microorganism).

A microorganism of the invention may be produced from an APP strain which expresses endogenous ApxIIA and ApxIIIA polypeptides, such as a serotype 2, 8 or 15 strain, and the endogenous ApxIIA and ApxIIIA polypeptides may be replaced by or modified to form inactive ApxIIA and ApxIIIA polypeptides. A nucleic acid encoding an inactive ApxIA polypeptide may be introduced to produce the microorganism of the invention. By way of non-limiting example, a nucleic acid encoding an inactive ApxIA polypeptide as disclosed herein may be introduced to said APP strain to produce a microorganism according to the invention (the nucleic acid encoding the inactive ApxIA, ApxIIA and/or ApxIIIA polypeptides may be integrated into the genome of the APP strain, or may be present extra-chromosomally within the microorganism).

A microorganism of the invention may be produced from an APP strain which expresses ApxIA and ApxIIA polypeptides, such as a serotype 1, 5 or 9 strain, and the endogenous ApxIA and ApxIIA polypeptides may be replaced by or modified to form inactive ApxIA and ApxIIA polypeptides. A nucleic acid encoding an inactive ApxIIIA polypeptide may be introduced to produce the microorganism of the invention. By way of non-limiting example, a nucleic acid encoding an inactive ApxIIIA polypeptide as disclosed herein may be introduced to said APP strain to produce a microorganism according to the invention (the nucleic acid encoding the inactive ApxIA, ApxIIA and/or ApxIIIA polypeptides may be integrated into the genome of the APP strain, or may be present extra-chromosomally within the microorganism).

The introduction, replacement or modification of a nucleic acid encoding an ApxIA, ApxIIA and/or ApxIIIA polypeptide may be carried out by any appropriate technique. Non-limiting examples of suitable techniques include those described in Baltes et al. (FEMS Microbiol. Lets. (2003b) 220(1):41-48), the single-step transconjugation system described in Oswald et al. (FEMS Microbiol. Lets. (1999) 179(1):153-160) and the allele exchange methodology used in Sheehan et al. (Infect Immun (2000) 68(8):4778-478), each of which is herein incorporated by reference in its entirety. Preferably, the introduction, replacement or modification of a nucleic acid encoding an ApxIA, ApxIIA and/or ApxIIIA polypeptide is carried out using natural transformation. This technique is preferred as it allows for the production of precise APP mutant strains. Exemplary natural transformation methodology is described in Bosse et al. FEMS Microbiol Lett. 2004 Apr. 15; 233(2):277-81 and Bosse et al. 2014 PLoS ONE 9(11): e111252, which are herein incorporated by reference in its entirety. Typically, a two-step natural transformation protocol is used, such as that exemplified herein. One example of a cassette that may be used in the first step of such a two-step natural transformation protocol is the dfrA14sacB cassette (SEQ ID NO: 18), as exemplified herein. This preferred dfrA14sacB cassette consists of the trimethoprim resistance allele dfrA14 (identified in endogenous APP plasmids), preceded by the promoter for the sodC gene of APP, followed by a 9-bp sequence required for uptake of DNA during natural transformation by APP, and the sucrose sensitivity gene, sacB. Gene replacement and mutation/deletion constructs (along with all of the primer sequences used in generation of these constructs) for the preferred natural transformation method are given in SEQ ID Nos: 19 to 46 in the sequence information section below.

The invention therefore provides a method for the production of an APP strain producing all three ApxA toxins (ApxI, ApxII, ApxIII) as described herein. Said method typically involves the introduction of one or more apxA gene into a microorganism by natural transformation, typically by two-step natural transformation. The dfrA14sacB cassette described herein is an exemplary, non-limiting cassette that may be used in such methods. Non-limiting examples of production methods are described in more detail below. The Examples herein provide non-limiting descriptions of methods according to the invention.

The methods of the invention can be used to generate APP strains producing all three ApxA toxins (ApxI, ApxII and ApxIII), in either wild-type or inactive form, regardless of the original apx gene profile of the APP strain. By way of non-limiting example, for a transformable APP isolate producing ApxII and ApxIII, to produce a strain comprising inactive forms of all three of ApxI, ApxII and ApxIII according to the invention, the appropriate mutations/deletions to remove or inactivate the one or both acylation sites in the respective toxin apxIIA and apxIIIA genes (as described herein) will be introduced, together with a mutated apxI operon (comprising a deletion/modification of one or both acylation sites as described herein) using natural transformation, such as the two-step transformation process described in the Examples. This can be done by amplifying the entire mutated apxI operon and 500 bp of flanking sequence and transforming this sequence into the strain in which the apxIIA and apxIIIA genes have already been mutated. The 500 bp flanking sequence to either side of the operon may be modified as appropriate to target a desired insertion site. As another non-limiting example, for a transformable APP isolate producing ApxI and ApxII, to produce a strain comprising inactive forms of all three of ApxI, ApxII and ApxIII according to the invention, the appropriate mutations/deletions to remove the one or both acylation sites in the respective toxin apxIA and apxIIA genes (as described herein) will be introduced, together with a mutated apxIII operon (comprising a deletion/modification of one or both acylation sites as described herein) using natural transformation, such as the two-step transformation process described in the Examples. This may easily be done by amplifying the entire mutated apxIII operon and 500 bp of flanking sequence (e.g. from one of the serotype 8 or 15 mutants) and transforming this sequence into the strain in which the apxIA and apxIIA genes have already been mutated. By way of further non-limiting example, if using a strain that normally only possesses genes for one of the ApxA toxins, the other two operons (with one or both acylation sites mutated or deleted in the respective toxin genes) could be introduced using this same method.

Similarly, this two-step method may be used to generate a microorganism in which all three ApxIA, ApxIIA and ApxIIIA are present in wild-type form. By way of non-limiting example, whether starting from an APP strain which endogenously expresses wild-type ApxIIA and ApxIIIA, two step-transformation may be carried out by amplifying the entire wild-type apxI operon and 500 bp of flanking sequence and transforming this sequence into the strain already comprising wild-type apxIIA and apxIIIA genes. As a further non-limiting example, if the starting strain endogenously expresses ApxIA and ApxIIA, the two-step natural transformation process involves the amplification of the entire wild-type apxIII operon and 500 bp of flanking sequence and transforming this sequence into the strain already comprising wild-type apxIA and apxIIA genes. The 500 bp flanking sequence to either side of the operon may be modified as appropriate to target a desired insertion site.

Microorganisms of the present invention may also comprise nucleic acids and/or vectors encoding one or more additional genes.

The one or more additional antigen may be from APP or may be from one or more other swine pathogens. Non-limiting examples of other swine pathogens and antigens therefrom that may be expressed using microorganisms, nucleic acids and/or vectors of the invention include bacterial antigens from: Bordetella bronchiseptica, Brachyspira hyodysenteriae, Brachyspira pilosicoli, Brucella suis, Clostridium difficile, Clostridium perfringens, Escherichia coli [e.g Heat labile (LT)-toxin, heat-stable (ST)-toxins], Lawsonia intracellularis Shigella-like toxin type II variant (SLT-Ile), verotoxin, cell wall (O antigens) and fimbriae (F antigens), Erysipelothrix rhusiopathiae, Haemophilus parasuis, Leptospira spp., Mycoplasma hyopneumoniae, Mycoplasma hyosynoviae, Mycoplasma hyorhinis, Pasteurella multocida, Salmonella spp, Staphylococcus hyicus, Streptococcus suis (e.g. IdeS).

Non-limiting examples of other swine pathogens and antigens therefrom that may be expressed using microorganisms, nucleic acids and/or vectors of the invention include viral antigens from: African Swine Fever Virus (ASFV), Atypical Porcine Pestivirus (APPV, e.g. E1 and or E2), Classical Swine Fever Virus (CSFV, e.g. E1 and or E2), Foot and Mouth Disease Virus (FMDV, e.g. VP1, VP2, VP3, VP4, P2A and/or 3C), Porcine Epidemic Diarrhea Virus (PEDV, e.g. spike protein), Encephalomyocarditis virus, Parvovirus (e.g. VP2), Porcine Circovirus (PCV1, PCV2 or PCV2, e.g. ORF2 or cap protein respectively), Porcine Reproductive and Respiratory Syndrome Virus (PRRSV), Suid Herpes Virus, Rotavirus Type A and C (RVA, RVC, e.g. VP4 and or Vp7), Swine Herpes Virus, Swine Influenza Virus (SIV, e.g. Haemmagglutinin (HA) and or Neuraminidase NA), Swine Pox Virus, Swine Vesicular Disease Virus, Transmissible Gastroenteritis Virus (TGEV).

Microorganisms of the present invention may also comprise one or more additional modification or deletion to inactivate/knock out at least one additional polypeptide within the microorganism. Such additional modifications are typically comprised in microorganisms comprising inactive ApxA polypeptides as disclosed herein, particularly wherein said additional modifications provide a further means to attenuate the microorganism. Inactivation/deletion of at least one additional polypeptide is therefore preferable in the context of attenuated vaccines as described herein. Without being bound by theory, it is believed that combining additional modifications with the microorganisms of the invention, particularly those with inactive ApxIA, ApxIIA and ApxIIIA, as described herein will result in a synergistic attenuation of APP. Modification or deletion of ApxIVA as described herein may be used for either live (attenuated) microorganisms comprising inactive ApxA polypeptides or microorganisms comprising wild-type ApxA polypeptides, as in either the deleted/modified ApxIVA polypeptide may be used as a marker for a DIVA vaccine as described herein.

Non-limiting examples of other genes which may be modified according to the invention include apxIVA, sxy (e.g. as encoded by DRF63_RS09615, version as of 30 Jul. 2020), ssrA (e.g. as encoded by DRF63_RS10030, version as of 16 Jul. 2020) and nlpD (also known as DRF63_RS10540, version as of 16 Jul. 2020), which is the gene encoding a LysM peptidoglycan-binding domain containing protein. Any combination of these genes may be modified. For example, apxIVA and sxy may be modified, apxIVA, sxy and ssrA may be modified, apxIVA, sxy and nlpD may be modified, apxIVA, sxy, ssrA and nlpD may be modified or nlpD and ssrA may be modified.

Typically, where the sxy gene is modified according to the invention, the sxy gene product is inactivated or deleted, preferably deleted. Inactivation or deletion of sxy prevents natural transformation. Thus, when producing microorganisms of the invention, inactivation or deletion of sxy is typically the last modification made to the microorganism, as further modification via natural transformation will not be possible once sxy is inactivated or deleted. Inactivation or deletion of sxy is particularly preferred when a microorganism of the invention comprises inactive ApxA (ApxIA, ApxIIA and ApxIIIA) polypeptides, as deletion of sxy prevents the microorganism reacquiring a wild-type ApxA polypeptide by natural transformation and so regaining virulence. Particularly preferred are microorganisms in which (i) both amino acids that are susceptible to acylation in each ApxA (ApxIA, ApxIIA and ApxIIIA) polypeptide have been substituted for amino acids that are not susceptible to acylation or deleted; and (ii) sxy has been inactivated or deleted, preferably deleted. This combination effectively precludes the possibility of the microorganism reverting to wild-type and hence virulence.

Microorganisms of the invention where the apxIVA gene is modified according to the invention allow for the differentiation of infected from vaccinated animals. Vaccines comprising appropriately modified apxIVA may therefore be described as DIVA vaccines.

ApxIV polypeptide is a weakly-haemolytic toxin that is unique to APP. In vivo it is expressed by all serotypes and can therefore be used to assign species and as an antigen for serological surveillance. Use of a modified ApxIV polypeptide (or nucleic acid encoding therefor) has the potential to act as a marker for live attenuated vaccine strains (or for subunit vaccines which comprise an ApxIV component), via a DIVA strategy. DIVA vaccines have at least one less antigenic protein than the corresponding wild-type microorganism. The ability to differentiate between subjects which have been immunised with the vaccine and subjects which have been exposed to the pathogenic form of the microorganism are based on detecting the serological response either toward a protein (or epitope) whose gene (or part thereof) has been deleted in the vaccine strain. Thus, subjects which have bene exposed to the pathogenic form of the microorganism exhibit a positive serological response to the antigen or epitope, whereas subjects which have been immunised with the vaccine do not. ApxIVA can therefore be used as a marker for a DIVA vaccine according to the invention.

Typically, when the apxIVA gene is modified according to the invention, the apxIVA gene is deleted or modified by an unmarked in-frame deletion of a sequence encoding an N-terminal immunogenic domain in the ApxIVA protein. One non-limiting example of such a deletion is the 2586 base pair (bp) deletion described in the Examples herein. An exemplary wild-type ApxIVA polypeptide (serotype 8) is given in SEQ ID NO: 47. The exemplified N-terminal in-frame deletion is given in SEQ ID NO: 48. Vaccinated subjects will not exhibit a serological response to the N-terminal immunogenic domain of ApxIVA.

A microorganism of the invention (comprising either wild-type or inactive ApxA polypeptides as described herein) may preferably comprise a deletion of the sxy gene and/or a modification of the apxIVA gene, such as an unmarked in-frame deletion of an N-terminal immunogenic domain sequence in the apxIVA as exemplified herein (or a deletion of the apxIVA gene). Most preferably the microorganism may comprise both a deletion of the sxy gene and a modification of the apxIVA gene, such as an unmarked in-frame deletion of an N-terminal immunogenic domain sequence in the apxIVA as exemplified herein (or a deletion of the apxIVA gene).

A microorganism of the invention, particularly an APP may comprises one or at least two of the following additional modifications (e.g. single or multiple deletions): ΔtpbA, ΔtonB2, ΔsodC, ΔdsbA, Δfur, ΔmlcA, ΔmglA, ΔexbB, ΔureC, double mutant ΔexbBΔureC, double mutant ΔfhuAΔhlyX, double mutant ΔapxICΔapxIIC, triple mutant ΔapxICΔapxIICΔorf1, hexamutant ΔapxIIAΔureCΔdmsAΔhybBΔaspAΔfur, double mutant ΔapxIIIBΔapxIIID, double mutant ΔclpPΔapxIIC, ΔznuA, ΔapfA, double mutant ΔapxIIAΔureC, pentamutant ΔapxICΔapxIICΔorf1ΔcpxARΔarcA, double mutant ΔapxICΔompP2, double mutant ΔapxIICΔapxIVA, inactivated apxIIC, inactivated apxIC, Δlip40, ΔcpxA/cpxR, ΔpotD2, ΔtolC2, ΔsapA and/or ΔpdxS/pdxT. These modifications are described in Baltes et al. FEMS Microbiol Let (2002) 209(2):283-287; Sheehan et al. Infect Immun (2003) 71(7):3960-3970; Baltes et al Infect. Immun (2001) 69(1):472-478; Jaques Can J Vet Res (2004) 68(2):81-85; Baltes et al. Infect Immun (2005) 73(8):4614-4619; Lin et al. FEMS Microbiol Let (2007) 274(1):55-62; Yuan et al. Current Microbiol (2011) 63(6):574-580; Maas et al. Infect Immun (2006) 74(7):4124-4132; Park et al. J Vet Med Sci (2009) 71(10:1317-1323; Xie et al. BMC Vet Res (2017) 13(1)p14; Yuan et al. Vet Microbiol (2014) 174(3-4):531-539; Zhou et al. Clin Vaccine Immunol (2013) 20(2):287-294; Tonpitak et al. Infect Immun (2002) 70(12):7120-7125; Yuan et al. Vaccine (2018) 36(14):1830-1836; Liu et al. Onderstepoort J of Vet Res (2013) 80(1):519; Liu et al. Vaccine (2007) 25(44):7696-7705; Bei et al. FEMS Microbiol Let (2005) 243(1):21-37; Xu et al. Acta Microbiologica Sinca (2007) 47(5):923-927; Prideaux et al. Infect Immun (1999) 67(4):1962-1966; Liu Front Microbiol 2018 Jul. 3:9:1472; Li et al. Front Cell Infect Microbiol 2018 Mar. 20:8:72; Zhu Antonie Van Leeuwenhoek (2017) 110(12):1647-1657; Li J Med Microbiol (2017) DOI: 10/1099/imm.0.000544; and Xie Front Microbiol 2017 May 10:8:911; Xie PLoS One (2017) 12(4):e0176374; each of which is herein incorporated by reference in its entirety.

It is expected that the combination of the microorganisms, particularly those with inactive ApxIA, ApxIIA and ApxIIIA, as described herein will result in a synergistic attenuation of APP.

### Vaccine Compositions

Also disclosed herein is a vaccine composition comprising one or more microorganism of the invention, one or more nucleic acid of the invention or one or more vector of the invention. In particular, the present invention provides live (attenuated) vaccines and whole cell inactivated vaccines comprising microorganisms of the invention.

Live (attenuated) vaccines typically comprise microorganisms comprising inactive ApxA polypeptides of the invention as described herein. Thus, whilst the microorganisms of live (attenuated) vaccines are able to infect and replicate in host cells, they have substantially no haemolytic and/or cytotoxic activity. In live (attenuated) vaccines of the invention preferably (a) the microorganism is an APP strain; and/or (b) the ApxIA, ApxIIA and ApxIIIA are inactive ApxIA, ApxIIA and ApxIIIA which have common antigenic cross-reactivity with wild-type ApxIA, ApxIIA and ApxIIIA as described herein.

Whole cell inactivated vaccines typically comprise microorganisms comprising wild-type ApxA polypeptides as described herein, wherein the microorganisms are subsequently inactivated by a suitable means (such as chemical or thermal inactivation). Thus, the microorganisms in whole cell inactivated vaccines are immunogenic, are unable to infect or replicate in host cells. In whole cell inactivated vaccines of the invention preferably (a) the microorganism is an APP strain; and/or (b) the ApxIA, ApxIIA and ApxIIIA are wild-type ApxIA, ApxIIA and ApxIIIA which have been subsequently inactivated, preferably by chemical and/or heat treatment.

One advantage of the vaccine compositions of the invention is that a single microorganism, particularly a single strain of a microorganism may be used to provide all three of ApxIA, ApxIIA and ApxIIA, and hence to provide protection against all APP serovars. This is the case for both live (attenuated) vaccines and whole cell inactivated vaccines. The invention also allows for the production of subunit vaccines against APP using a single microorganism, or single microorganism strain as described herein.

The microorganism comprised in a vaccine of the invention may be of any bacterial species as described herein. Actinobacillus species (e.g. APP and A. suis), including strains, serotypes/serovars thereof are preferred. APP and strains, serotypes/serovars thereof are particularly preferred. Typically, a vaccine of the invention comprises a single microorganism, or strain, species or serotype/serovar thereof. As a non-limiting example, a vaccine may comprise a single APP strain, serotype/serovar thereof. This is because the microorganism comprises each of ApxIA, ApxIIA and ApxIIIA, providing protection against all APP strains, serotypes/serovars, and avoiding the need for multiple APP strains, serotypes/serovars to be included in the vaccine.

The microorganism (comprising either wild-type or inactive ApxA polypeptides as described herein) comprised in a vaccine of the invention may comprise one or more additional modification as described herein. The microorganism (comprising either wild-type or inactive ApxA polypeptides as described herein) comprised in a vaccine of the invention preferably comprise a deletion of the sxy gene and/or a modification or deletion of the apxIVA gene as described herein. Most preferably the microorganism may comprise both a deletion of the sxy gene and a modification of the apxIVA gene, such as an unmarked in-frame deletion of an N-terminal immunogenic domain sequence in the apxIVA as exemplified herein (or a deletion of the apxIVA gene).

A vaccine composition of the invention may comprise at least a pharmaceutical carrier, a diluent and/or an adjuvant.

Non-limiting examples of pharmaceutically acceptable carriers, diluents or adjuvants which may be used in accordance with the invention include: mineral salt adjuvants (e.g. alum-, calcium-, iron- and zirconium-based adjuvants), tensoactive adjuvants (e.g. Quil A, QS-21 and other saponins), bacterial-derived adjuvants (e.g. N-acetyl muramyl-L-alanyl-D-isoglutamine (MDP), lipopolysaccharide (LPS), monophosphoryl lipid A, trehalose dimycolate (TDM), DNA, CpGs and bacterial toxins), adjuvant emulsions (e.g. FIA, Montande, Adjuvant 65, Lipovant), liposome adjuvants, polymeric adjuvants and carriers, cytokines (e.g. Granulocyte-macrophage colony stimulating factor (GM-CSF)), carbohydrate adjuvants, living antigen delivery systems (e.g. bacteria, especially modified APP). Furthermore, carriers can also comprise dry formulations such as coated patches made from titan or polymer. Techniques for formulation and administration of the vaccines of the present application may also be found in “Remington, The Science and Practice of Pharmacy”, 22^{nd} edition.

The vaccine compositions as a unit composition may comprise 0.001-2.0 mg of protein, 0.001-2.0 mg of nucleic acid, or 0.5-200 mg (or 1×10⁴-1×10⁹ colony forming units (CFU)) of microorganism. The required active amount of the protein, nucleic acid or microorganism may be determined by routine testing methods by the skilled person, e.g. in pigs or piglets.

A vaccine composition of the invention may substantially only contain one or more nucleic acid or one or more vector of the invention. By way of non-limiting example, the vaccine may be a DNA vaccine. DNA vaccines are third generation vaccines. Nucleic acid or DNA APP vaccines contain DNA/nucleic acid that encodes specific proteins from APP, particularly ApxA polypeptides. DNA/nucleic acid vectors of the invention typically contain one or more nucleic acid of the invention or one or more vector of the invention which encode for all three of ApxIA, ApxIIA and ApxIIIA, preferably in inactive form as described herein. DNA/nucleic acid vaccines are administered to a mammalian subject (typically by injection) and the DNA/nucleic acid is taken up by subject's cells, whose normal metabolic processes synthesise proteins based on the genetic code in the DNA/nucleic acid of the vaccine which they have taken up. Because these proteins contain regions of amino acid sequences that are characteristic of APP, they are recognised as foreign when they are processed by the host cells and displayed on their surface, altering the subject's immune system and triggering an immune response. When the APP proteins encoded by a DNA/nucleic acid vaccine are inactive ApxA polypeptides, an immune response is triggered, but the ApxA polypeptides do not have any haemolytic or cytotoxic activity, and so are themselves non-pathogenic.

DNA/nucleic acid vaccines may be encapsulated in protein to facilitate entry to the mammalian subject's cells. If this capsid protein is comprised within the DNA/nucleic acid of the DNA/nucleic acid vaccine, the resulting vaccine can combine the potency of a live vaccine without reversion risks.

Standard methods and techniques for the production of vaccines are known in the art and are described in handbooks known to the person of skill in the art. One advantage provided by vaccines of the present invention is the simplification of the production protocol, with the consequent reduction in cost. This simplification and cost saving typically results from the fact that a single microorganism can be used to produce all three of ApxIA, ApxIIA and ApxIIIA, and thus provide protection against all known serovars of APP. Conventional production protocols require at least two APP strains, which requires multiple production steps (such as the culturing and purification of the at least two APP strains, or the ApxA polypeptides therefrom), and hence increased production costs.

Accordingly, the invention provides a method of producing a live (attenuated) vaccine composition of the invention, comprising: (a) culturing a microorganism of the invention, wherein the ApxIA, ApxIIA and ApxIIIA are inactive ApxIA, ApxIIA and ApxIIIA which have common antigenic cross-reactivity with wild-type ApxIA, ApxIIA and ApxIIIA; (b) isolating the microorganism; and (c) formulating the microorganism with a pharmaceutical carrier, a diluent and/or an adjuvant.

The invention also provides a method of producing an inactivated vaccine composition of the invention, said method comprising: (a) culturing a microorganism as defined herein, wherein the ApxIA, ApxIIA and ApxIIIA are wild-type ApxIA, ApxIIA and ApxIIIA; (b) isolating the microorganism; (c) inactivating the microorganism, preferably by chemical and/or heat treatment; and (d) formulating the inactivated microorganism with a pharmaceutical carrier, a diluent and/or an adjuvant. Standard means and protocols for inactivating microorganisms, such as by heat (thermal inactivation) and/or chemical inactivation are known in the art and would be routine to one of skill in the art.

The invention also provides a method of producing subunit vaccine comprising each of ApxIA, ApxIIA and ApxIIIA using a single microorganism or strain thereof. The ApxIA, ApxIIA and ApxIIIA may be produced as wild-type polypeptides (as described herein) and subsequently inactivated. Alternatively, the ApxIA, ApxIIA and ApxIIIA may be produced in an inactive form (as described herein), such that they do not require further inactivation (e.g. chemical or thermal) prior to use.

Accordingly, the invention provides a method of producing a subunit vaccine composition, comprising: (a) culturing a microorganism of the invention which comprises inactive ApxIA, ApxIIA and ApxIIIA which have common antigenic cross-reactivity with wild-type ApxIA, ApxIIA and ApxIIIA; (b) isolating the inactive ApxIA, ApxIIA and ApxIIIA from the cultured microorganism; and (c) formulating the inactive ApxIA, ApxIIA and ApxIIIA with a pharmaceutical carrier, a diluent and/or an adjuvant.

Alternatively, the invention provides a method of producing a subunit vaccine composition, comprising: (a) culturing a microorganism of the invention which comprises wild-type ApxIA, ApxIIA and ApxIIIA; (b) isolating the wild-type ApxIA, ApxIIA and ApxIIIA from the cultured microorganism; (c) inactivating the wild-type ApxIA, ApxIIA and ApxIIIA; and (d) formulating the inactivated wild-type ApxIA, ApxIIA and ApxIIIA with a pharmaceutical carrier, a diluent and/or an adjuvant. Standard means and protocols for inactivating microorganisms, such as by heat (thermal inactivation) and/or chemical inactivation are known in the art and would be routine to one of skill in the art.

Any appropriate culture conditions, media and/or protocols may be used in the production methods of the invention. Standard culture conditions, media and protocols are known in the art. Any appropriate means may be used to isolate the microorganism. Again, routine isolation means and protocols are also known in the art and would be routine to one of skill in the art.

The production methods of the invention preferably relate to the production of a microorganism that is an Actinobacillus species (e.g. APP and A. suis), including strains, serotypes and serovars thereof are particularly preferred. In addition, microorganisms comprising one or more additional modifications are preferred, particularly microorganisms (even more particularly an Actinobacillus species (e.g. APP)) comprising the modifications/deletions sxy and/or apxIVA as described herein.

The invention also encompasses vaccines (particularly live attenuated vaccines) which comprise multiple different microorganisms, each of which provides one or more inactive ApxA poplypeptide of the invention. Each microorganism typically does not express any wild-type ApxA polypeptides. Further, where multiple different microorganisms are used, each microorganism will also comprise modification of sxy and ApxIVA as described herein. Accordingly, the invention provides a vaccine comprising three different microorganisms, each which expresses an inactive form of one of ApxIA, ApxIIA and ApxIIIA, wherein each microorganism also encompasses the the modifications/deletions sxy and/or apxIVA as described herein.

The invention also provides a vaccine comprising two different microorganisms; a first microorganism which expresses inactive forms of any two of ApxIA, ApxIIA and ApxIIIA polypeptides, and a second microorganism which expresses at least an inactive form of the ApxA polypeptide not expressed by the first microorganism (and may also express inactive forms of other ApxA polypeptides), wherein both the first and second microorganism also encompass the modifications/deletions of sxy and apxIVA as described herein. By way of non-limiting example, a vaccine may comprise: (i) a first microorganism (e.g. a serotype 2 APP strain) expressing inactive forms of ApxIIA and ApxIIIA, a modified apxIVA and a deleted sxy; and (ii) a second microorganism (e.g. a serotype 9 APP strain) expressing inactive forms of ApxIA and ApxIIA, a modified apxIVA and a deleted sxy. By way of further non-limiting example, a vaccine may comprise: (i) a first microorganism (e.g. a serotype 2 APP strain) expressing inactive forms of ApxIIA and ApxIIIA, a modified apxIVA and a deleted sxy; and (ii) a second microorganism (e.g. a serotype 14 APP strain) expressing an inactive form of ApxIA, a modified apxIVA and a deleted sxy.

Any and all disclosure herein in relation to microorganisms in the context of a single strain provide all three of ApxIA, ApxIIA and ApxIIA applies equally and without restriction to vaccines comprising multiple different microorganisms. For example, the microorganisms may each be independently any bacterial species as described herein, preferably each independently selected from an Actinobacillus species, more preferably each independently selected from APP strains.

### Medical Uses or Methods

The disclosed vaccine compositions may be used in the prophylactic, metaphylactic and/or therapeutic treatment of a pneumonia, a pleurisy or a pleuropneumonia, in particular a pneumonia, a pleurisy or a pleuropneumonia caused by APP in a subject. The subject to be treated is typically a mammal, particularly a pig.

The vaccine composition may be administered by any appropriate means. Non-limiting examples of suitable means of administration include intramuscular, intradermal, intravenous, subcutaneous and/or mucosal (e.g. intranasal) administration.

The vaccine composition may be administered via at least one, for example one or two administrations using a unit composition as described above. In particular, the composition may be administered for the first time on the day of birth of the subject, within three days, one week, two weeks, four weeks, six weeks, eight weeks, ten weeks or 12 weeks of the birth of the subject. Accordingly, the vaccine, or the first administration thereof, may be advantageously administered at an early point in time of the life of the subject. Alternatively, the vaccine may be administered (including second or subsequent administrations) at any time point in the life of the subject.

The vaccine composition may be administered for a second time or subsequent time, wherein the time period between the two administrations (e.g. the first and second administrations) may be between one and four weeks, between one and three weeks, or between one and two weeks. Preferably, a vaccine composition comprising a microorganism of the invention is to be administered once only. The invention encompasses the passive immunisation of piglets through the colostrum of sows who have been vaccinated according to the present invention. The invention also encompasses the vaccination of piglets by maternally-derived antibodies from sows who have been vaccinated according to the present invention. The invention further encompasses vaccination of piglets having maternally-derived antibodies at the time of vaccination.

### Expression Systems

The microorganisms, nucleic acids and/or vectors of the invention may be used as a means to express one or more additional antigen from a swine pathogen. Thus, the invention provides an expression system for antigens from other swine pathogens. This expression system may be used to produce the swine pathogen antigen in vitro for subsequent clinical application (e.g. to produce an (additional) component for a subunit vaccine) or research use. Alternatively, this expression system may be used in vivo as a vaccine against said one or more additional swine pathogen. In this way, subjects could be immunised against multiple swine pathogens using a single vaccine comprising a single microorganism or strain thereof.

Accordingly, the invention provides an expression system comprising a microorganism of the invention which comprises each of ApxIA, ApxIIA and ApxIIIA (either in wild-type or inactive form), further comprising at least one additional nucleic acid which encodes one or more additional swine pathogen antigen. The at least one additional nucleic acid may be comprised within the genome of the microorganism or be present extra-chromosomally, as described herein in the context of nucleic acids encoding for the (wild-type or inactive) ApxIA, ApxIIA and ApxIIIA polypeptides. That disclosure applies equally and without restriction to the at least one additional nucleic acid encoding one or more additional swine pathogen antigen. Preferably the at least one additional nucleic acid is comprised within the genome of the microorganism

The one or more additional antigen may be from APP or may be from one or more other swine pathogens. Non-limiting examples of other swine pathogens and antigens therefrom that may be expressed using microorganisms, nucleic acids and/or vectors of the invention include bacterial antigens from: Bordetella bronchiseptica, Brachyspira hyodysenteriae, Brachyspira pilosicoli, Brucella suis, Clostridium difficile, Clostridium perfringens, Escherichia coli [e.g Heat labile (LT)-toxin, heat-stable (ST)-toxins], Lawsonia intracellularis Shigella-like toxin type II variant (SLT-Ile), verotoxin, cell wall (O antigens) and fimbriae (F antigens), Erysipelothrix rhusiopathiae, Haemophilus parasuis, Leptospira spp., Mycoplasma hyopneumoniae, Mycoplasma hyosynoviae, Mycoplasma hyorhinis, Pasteurella multocida, Salmonella spp, Staphylococcus hyicus, Streptococcus suis (e.g. IdeS).

Non-limiting examples of other swine pathogens and antigens therefrom that may be expressed using microorganisms, nucleic acids and/or vectors of the invention include viral antigens from: African Swine Fever Virus (ASFV), Atypical Porcine Pestivirus (APPV, e.g. E1 and or E2), Classical Swine Fever Virus (CSFV, e.g. E1 and or E2), Foot and Mouth Disease Virus (FMDV, e.g. VP1, VP2, VP3, VP4, P2A and/or 3C), Porcine Epidemic Diarrhea Virus (PEDV, e.g. spike protein), Encephalomyocarditis virus, Parvovirus (e.g. VP2), Porcine Circovirus (PCV1, PCV2 or PCV2, e.g. ORF2 or cap protein respectively), Porcine Reproductive and Respiratory Syndrome Virus (PRRSV), Suid Herpes Virus, Rotavirus Type A and C (RVA, RVC, e.g. VP4 and or Vp7), Swine Herpes Virus, Swine Influenza Virus (SIV, e.g. Haemmagglutinin (HA) and or Neuraminidase NA), Swine Pox Virus, Swine Vesicular Disease Virus, Transmissible Gastroenteritis Virus (TGEV).

Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics:1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, 15% identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{[ length of the longer sequence plus the number of gaps introduced into the longer sequence in order to align the two sequences ]} \times 100$$

Substantially homologous polypeptides are characterized as having one or more amino acid substitutions, deletions or additions. These changes are preferably of a minor nature, that is conservative amino acid substitutions (as described herein) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

### Sequence Information

ApxIA wild type
SEQ ID NO: 1
Met Ala Asn Ser Gln Leu Asp Arg Val Lys Gly Leu Ile Asp Ser Leu Asn Gln His Thr Lys
Ser Ala Ala Lys Ser Gly Ala Gly Ala Leu Lys Asn Gly Leu Gly Gln Val Lys Gln Ala Gly
Gln Lys Leu Ile Leu Tyr Ile Pro Lys Asp Tyr Gln Ala Ser Thr Gly Ser Ser Leu Asn Asp
Leu Val Lys Ala Ala Glu Ala Leu Gly Ile Glu Val His Arg Ser Glu Lys Asn Gly Thr Ala
Leu Ala Lys Glu Leu Phe Gly Thr Thr Glu Lys Leu Leu Gly Phe Ser Glu Arg Gly Ile Ala
Leu Phe Ala Pro Gln Phe Asp Lys Leu Leu Asn Lys Asn Gln Lys Leu Ser Lys Ser Leu Gly
Gly Ser Ser Glu Ala Leu Gly Gln Arg Leu Asn Lys Thr Gln Thr Ala Leu Ser Ala Leu Gln
Ser Phe Leu Gly Thr Ala Ile Ala Gly Met Asp Leu Asp Ser Leu Leu Arg Arg Arg Arg Asn
Gly Glu Asp Val Ser Gly Ser Glu Leu Ala Lys Ala Gly Val Asp Leu Ala Ala Gln Leu Val
Asp Asn Ile Ala Ser Ala Thr Gly Thr Val Asp Ala Phe Ala Glu Gln Leu Gly Lys Leu Gly
Asn Ala Leu Ser Asn Thr Arg Leu Ser Gly Leu Ala Ser Lys Leu Asn Asn Leu Pro Asp Leu
Ser Leu Ala Gly Pro Gly Phe Asp Ala Val Ser Gly Ile Leu Ser Val Val Ser Ala Ser Phe
Ile Leu Ser Asn Lys Asp Ala Asp Ala Gly Thr Lys Ala Ala Ala Gly Ile Glu Ile Ser Thr
Lys Ile Leu Gly Asn Ile Gly Lys Ala Val Ser Gln Tyr Ile Ile Ala Gln Arg Val Ala Ala
Gly Leu Ser Thr Thr Ala Ala Thr Gly Gly Leu Ile Gly Ser Val Val Ala Leu Ala Ile Ser
Pro Leu Ser Phe Leu Asn Val Ala Asp Lys Phe Glu Arg Ala Lys Gln Leu Glu Gln Tyr Ser
Glu Arg Phe Lys Lys Phe Gly Tyr Glu Gly Asp Ser Leu Leu Ala Ser Phe Tyr Arg Gly Thr
Gly Ala Ile Glu Ala Ala Leu Thr Thr Ile Asn Ser Val Leu Ser Ala Ala Ser Ala Gly Val
Gly Ala Ala Ala Thr Gly Ser Leu Val Gly Ala Pro Val Ala Ala Leu Val Ser Ala Ile Thr
Gly Ile Ile Ser Gly Ile Leu Asp Ala Ser Lys Gln Ala Ile Phe Glu Arg Val Ala Thr Lys
Leu Ala Asn Lys Ile Asp Glu Trp Glu Lys Lys His Gly Lys Asn Tyr Phe Glu Asn Gly Tyr
Asp Ala Arg His Ser Ala Phe Leu Glu Asp Thr Phe Glu Leu Leu Ser Gln Tyr Asn Lys Glu
Tyr Ser Val Glu Arg Val Val Ala Ile Thr Gln Gln Arg Trp Asp Val Asn Ile Gly Glu Leu
Ala Gly Ile Thr Arg Lys Gly Ala Asp Ala Lys Ser Gly Lys Ala Tyr Val Asp Phe Phe Glu
Glu Gly Lys Leu Leu Glu Lys Asp Pro Asp Arg Phe Asp Lys Lys Val Phe Asp Pro Leu Glu
Gly Lys Ile Asp Leu Ser Ser Ile Asn Lys Thr Thr Leu Leu Lys Phe Ile Thr Pro Val Phe
Thr Ala Gly Glu Glu Ile Arg Glu Arg Lys Gln Thr Gly Lys Tyr Glu Tyr Met Thr Glu Leu
Phe Val Lys Gly Lys Glu Lys Trp Val Val Thr Gly Val Gln Ser His Asn Ala Ile Tyr Asp
Tyr Thr Asn Leu Ile Gln Leu Ala Ile Asp Lys Lys Gly Glu Lys Arg Gln Val Thr Ile Glu
Ser His Leu Gly Glu Lys Asn Asp Arg Ile Tyr Leu Ser Ser Gly Ser Ser Ile Val Tyr Ala
Gly Asn Gly His Asp Val Ala Tyr Tyr Asp Lys Thr Asp Thr Gly Tyr Leu Thr Phe Asp Gly
Gln Ser Ala Gln Lys Ala Gly Glu Tyr Ile Val Thr Lys Glu Leu Lys Ala Asp Val Lys Val
Leu Lys Glu Val Val Lys Thr Gln Asp Ile Ser Val Gly Lys Arg Ser Glu Lys Leu Glu Tyr
Arg Asp Tyr Glu Leu Ser Pro Phe Glu Leu Gly Asn Gly Ile Arg Ala Lys Asp Glu Leu His
Ser Val Glu Glu Ile Ile Gly Ser Asn Arg Lys Asp Lys Phe Phe Gly Ser Arg Phe Thr Asp
Ile Phe His Gly Ala Lys Gly Asp Asp Glu Ile Tyr Gly Asn Asp Gly His Asp Ile Leu Tyr
Gly Asp Asp Gly Asn Asp Val Ile His Gly Gly Asp Gly Asn Asp His Leu Val Gly Gly Asn
Gly Asn Asp Arg Leu Ile Gly Gly Lys Gly Asn Asn Phe Leu Asn Gly Gly Asp Gly Asp Asp
Glu Leu Gln Val Phe Glu Gly Gln Tyr Asn Val Leu Leu Gly Gly Ala Gly Asn Asp Ile Leu
Tyr Gly Ser Asp Gly Thr Asn Leu Phe Asp Gly Gly Val Gly Asn Asp Lys Ile Tyr Gly Gly
Leu Gly Lys Asp Ile Tyr Arg Tyr Ser Lys Glu Tyr Gly Arg His Ile Ile Ile Glu Lys Gly
Gly Asp Asp Asp Thr Leu Leu Leu Ser Asp Leu Ser Phe Lys Asp Val Gly Phe Ile Arg Ile
Gly Asp Asp Leu Leu Val Asn Lys Arg Ile Gly Gly Thr Leu Tyr Tyr His Glu Asp Tyr Asn
Gly Asn Ala Leu Thr Ile Lys Asp Trp Phe Lys Glu Gly Lys Glu Gly Gln Asn Asn Lys Ile
Glu Lys Ile Val Asp Lys Asp Gly Ala Tyr Val Leu Ser Gln Tyr Leu Thr Glu Leu Thr Ala
Pro Gly Arg Gly Ile Asn Tyr Phe Asn Gly Leu Glu Glu Lys Leu Tyr Tyr Gly Glu Gly Tyr
Asn Ala Leu Pro Gln Leu Arg Lys Asp Ile Glu Gln Ile Ile Ser Ser Thr Gly Ala Leu Thr
Gly Glu His Gly Gln Val Leu Val Gly Ala Gly Gly Pro Leu Ala Tyr Ser Asn Ser Pro
Asn Ser Ile Pro Asn Ala Phe Ser Asn Tyr Leu Thr Gln Ser Ala
ApxIIA wild type
SEQ ID NO: 2
Met Ser Lys Ile Thr Leu Ser Ser Leu Lys Ser Ser Leu Gln Gln Gly Leu Lys Asn Gly Lys
Asn Lys Leu Asn Gln Ala Gly Thr Thr Leu Lys Asn Gly Leu Thr Gln Thr Gly His Ser Leu
Gln Asn Gly Ala Lys Lys Leu Ile Leu Tyr Ile Pro Gln Gly Tyr Asp Ser Gly Gln Gly Asn
Gly Val Gln Asp Leu Val Lys Ala Ala Asn Asp Leu Gly Ile Glu Val Trp Arg Glu Glu Arg
Ser Asn Leu Asp Ile Ala Lys Thr Ser Phe Asp Thr Thr Gln Lys Ile Leu Gly Phe Thr Asp
Arg Gly Ile Val Leu Phe Ala Pro Gln Leu Asp Asn Leu Leu Lys Lys Asn Pro Lys Ile Gly
Asn Thr Leu Gly Ser Ala Ser Ser Ile Ser Gln Asn Ile Gly Lys Ala Asn Thr Val Leu Gly
Gly Ile Gln Ser Ile Leu Gly Ser Val Leu Ser Gly Val Asn Leu Asn Glu Leu Leu Gln Asn
Lys Asp Pro Asn Gln Leu Glu Leu Ala Lys Ala Gly Leu Glu Leu Thr Asn Glu Leu Val Gly
Asn Ile Ala Ser Ser Val Gln Thr Val Asp Ala Phe Ala Glu Gln Ile Ser Lys Leu Gly Ser
His Leu Gln Asn Val Lys Gly Leu Gly Gly Leu Ser Asn Lys Leu Gln Asn Leu Pro Asp Leu
Gly Lys Ala Ser Leu Gly Leu Asp Ile Ile Ser Gly Leu Leu Ser Gly Ala Ser Ala Gly Leu
Ile Leu Ala Asp Lys Glu Ala Ser Thr Glu Lys Lys Ala Ala Ala Gly Val Glu Phe Ala Asn
Gln Ile Ile Gly Asn Val Thr Lys Ala Val Ser Ser Tyr Ile Leu Ala Gln Arg Val Ala Ser
Gly Leu Ser Ser Thr Gly Pro Val Ala Ala Leu Ile Ala Ser Thr Val Ala Leu Ala Val Ser
Pro Leu Ser Phe Leu Asn Val Ala Asp Lys Phe Lys Gln Ala Asp Leu Ile Lys Ser Tyr Ser
Glu Arg Phe Gln Lys Leu Gly Tyr Asp Gly Asp Arg Leu Leu Ala Asp Phe His Arg Glu Thr
Gly Thr Ile Asp Ala Ser Val Thr Thr Ile Asn Thr Ala Leu Ala Ala Ile Ser Gly Gly Val
Gly Ala Ala Ser Ala Gly Ser Leu Val Gly Ala Pro Val Ala Leu Leu Val Ala Gly Val Thr
Gly Leu Ile Thr Thr Ile Leu Glu Tyr Ser Lys Gln Ala Met Phe Glu His Val Ala Asn Lys
Val His Asp Arg Ile Val Glu Trp Glu Lys Lys His Asn Lys Asn Tyr Phe Glu Gln Gly Tyr
Asp Ser Arg His Leu Ala Asp Leu Gln Asp Asn Met Lys Phe Leu Ile Asn Leu Asn Lys Glu
Leu Gln Ala Glu Arg Val Val Ala Ile Thr Gln Gln Arg Trp Asp Asn Gln Ile Gly Asp Leu
Ala Ala Ile Ser Arg Arg Thr Asp Lys Ile Ser Ser Gly Lys Ala Tyr Val Asp Ala Phe Glu
Glu Gly Gln His Gln Ser Tyr Asp Ser Ser Val Gln Leu Asp Asn Lys Asn Gly Ile Ile Asn
Ile Ser Asn Thr Asn Arg Lys Thr Gln Ser Val Leu Phe Arg Thr Pro Leu Leu Thr Pro Gly
Glu Glu Asn Arg Glu Arg Ile Gln Glu Gly Lys Asn Ser Tyr Ile Thr Lys Leu His Ile Gln
Arg Val Asp Ser Trp Thr Val Thr Asp Gly Asp Ala Ser Ser Ser Val Asp Phe Thr Asn Val
Val Gln Arg Ile Ala Val Lys Phe Asp Asp Ala Gly Asn Ile Ile Glu Ser Lys Asp Thr Lys
Ile Ile Ala Asn Leu Gly Ala Gly Asn Asp Asn Val Phe Val Gly Ser Ser Thr Thr Val Ile
Asp Gly Gly Asp Gly His Asp Arg Val His Tyr Ser Arg Gly Glu Tyr Gly Ala Leu Val Ile
Asp Ala Thr Ala Glu Thr Glu Lys Gly Ser Tyr Ser Val Lys Arg Tyr Val Gly Asp Ser Lys
Ala Leu His Glu Thr Ile Ala Thr His Gln Thr Asn Val Gly Asn Arg Glu Glu Lys Ile Glu
Tyr Arg Arg Glu Asp Asp Arg Phe His Thr Gly Tyr Thr Val Thr Asp Ser Leu Lys Ser Val
Glu Glu Ile Ile Gly Ser Gln Phe Asn Asp Ile Phe Lys Gly Ser Gln Phe Asp Asp Val Phe
His Gly Gly Asn Gly Val Asp Thr Ile Asp Gly Asn Asp Gly Asp Asp His Leu Phe Gly Gly
Ala Gly Asp Asp Val Ile Asp Gly Gly Asn Gly Asn Asn Phe Leu Val Gly Gly Thr Gly Asn
Asp Ile Ile Ser Gly Gly Lys Asp Asn Asp Ile Tyr Val His Lys Thr Gly Asp Gly Asn Asp
Ser Ile Thr Asp Ser Gly Gly Gln Asp Lys Leu Ala Phe Ser Asp Val Asn Leu Lys Asp Leu
Thr Phe Lys Lys Val Asp Ser Ser Leu Glu Ile Ile Asn Gln Lys Gly Glu Lys Val Arg Ile
Gly Asn Trp Phe Leu Glu Asp Asp Leu Ala Ser Thr Val Ala Asn Tyr Lys Ala Thr Asn Asp
Arg Lys Ile Glu Glu Ile Ile Gly Lys Gly Gly Glu Arg Ile Thr Ser Glu Gln Val Asp Lys
Leu Ile Lys Glu Gly Asn Asn Gln Ile Ser Ala Glu Ala Leu Ser Lys Val Val Asn Asp Tyr
Asn Thr Ser Lys Asp Arg Gln Asn Val Ser Asn Ser Leu Ala Lys Leu Ile Ser Ser Val Gly
Ser Phe Thr Ser Ser Ser Asp Phe Arg Asn Asn Leu Gly Thr Tyr Val Pro Ser Ser Ile Asp
Val Ser Asn Asn Ile Gln Leu Ala Arg Ala Ala
ApxIIIA wild type
SEQ ID NO: 3
Met Ser Thr Trp Ser Ser Met Leu Ala Asp Leu Lys Lys Arg Ala Glu Glu Ala Lys Arg Gln
Ala Lys Lys Gly Tyr Asp Val Thr Lys Asn Gly Leu Gln Tyr Gly Val Ser Gln Ala Lys Leu
Gln Ala Leu Ala Ala Gly Lys Ala Val Gln Lys Tyr Gly Asn Lys Leu Val Leu Val Ile Pro
Lys Glu Tyr Asp Gly Ser Val Gly Asn Gly Phe Phe Asp Leu Val Lys Ala Ala Glu Glu Leu
Gly Ile Gln Val Lys Tyr Val Asn Arg Asn Glu Leu Glu Val Ala His Lys Ser Leu Gly Thr
Ala Asp Gln Phe Leu Gly Leu Thr Glu Arg Gly Leu Thr Leu Phe Ala Pro Gln Leu Asp Gln
Phe Leu Gln Lys His Ser Lys Ile Ser Asn Val Val Gly Ser Ser Thr Gly Asp Ala Val Ser
Lys Leu Ala Lys Ser Gln Thr Ile Ile Ser Gly Ile Gln Ser Val Leu Gly Thr Val Leu Ala
Gly Ile Asn Leu Asn Glu Ala Ile Ile Ser Gly Gly Ser Glu Leu Glu Leu Ala Glu Ala Gly
Val Ser Leu Ala Ser Glu Leu Val Ser Asn Ile Ala Lys Gly Thr Thr Thr Ile Asp Ala Phe
Thr Thr Gln Ile Gln Asn Phe Gly Lys Leu Val Glu Asn Ala Lys Gly Leu Gly Gly Val Gly
Arg Gln Leu Gln Asn Ile Ser Gly Ser Ala Leu Ser Lys Thr Gly Leu Gly Leu Asp Ile Ile
Ser Ser Leu Leu Ser Gly Val Thr Ala Ser Phe Ala Leu Ala Asn Lys Asn Ala Ser Thr Ser
Thr Lys Val Ala Ala Gly Phe Glu Leu Ser Asn Gln Val Ile Gly Gly Ile Thr Lys Ala Val
Ser Ser Tyr Ile Leu Ala Gln Arg Leu Ala Ala Gly Leu Ser Thr Thr Gly Pro Ala Ala Ala
Leu Ile Ala Ser Ser Ile Ser Leu Ala Ile Ser Pro Leu Ala Phe Leu Arg Val Ala Asp Asn
Phe Asn Arg Ser Lys Glu Ile Gly Glu Phe Ala Glu Arg Phe Lys Lys Leu Gly Tyr Asp Gly
Asp Lys Leu Leu Ser Glu Phe Tyr His Glu Ala Gly Thr Ile Asp Ala Ser Ile Thr Thr Ile
Ser Thr Ala Leu Ser Ala Ile Ala Ala Gly Thr Ala Ala Ala Ser Ala Gly Ala Leu Val Gly
Ala Pro Ile Thr Leu Leu Val Thr Gly Ile Thr Gly Leu Ile Ser Gly Ile Leu Glu Phe Ser
Lys Gln Pro Met Leu Asp His Val Ala Ser Lys Ile Gly Asn Lys Ile Asp Glu Trp Glu Lys
Lys Tyr Gly Lys Asn Tyr Phe Glu Asn Gly Tyr Asp Ala Arg His Lys Ala Phe Leu Glu Asp
Ser Phe Ser Leu Leu Ser Ser Phe Asn Lys Gln Tyr Glu Thr Glu Arg Ala Val Leu Ile Thr
Gln Gln Arg Trp Asp Glu Tyr Ile Gly Glu Leu Ala Gly Ile Thr Gly Lys Gly Asp Lys Leu
Ser Ser Gly Lys Ala Tyr Val Asp Tyr Phe Gln Glu Gly Lys Leu Leu Glu Lys Lys Pro Asp
Asp Phe Ser Lys Val Val Phe Asp Pro Thr Lys Gly Glu Ile Asp Ile Ser Asn Ser Gln Thr
Ser Thr Leu Leu Lys Phe Val Thr Pro Leu Leu Thr Pro Gly Thr Glu Ser Arg Glu Arg Thr
Gln Thr Gly Lys Tyr Glu Tyr Ile Thr Lys Leu Val Val Lys Gly Lys Asp Lys Trp Val Val
Asn Gly Val Lys Asp Lys Gly Ala Val Tyr Asp Tyr Thr Asn Leu Ile Gln His Ala His Ile
Ser Ser Ser Val Ala Arg Gly Glu Glu Tyr Arg Glu Val Arg Leu Val Ser His Leu Gly Asn
Gly Asn Asp Lys Val Phe Leu Ala Ala Gly Ser Ala Glu Ile His Ala Gly Glu Gly His Asp
Val Val Tyr Tyr Asp Lys Thr Asp Thr Gly Leu Leu Val Ile Asp Gly Thr Lys Ala Thr Glu
Gln Gly Arg Tyr Ser Val Thr Arg Glu Leu Ser Gly Ala Thr Lys Ile Leu Arg Glu Val Ile
Lys Asn Gln Lys Ser Ala Val Gly Lys Arg Glu Glu Thr Leu Glu Tyr Arg Asp Tyr Glu Leu
Thr Gln Ser Gly Asn Ser Asn Leu Lys Ala His Asp Glu Leu His Ser Val Glu Glu Ile Ile
Gly Ser Asn Gln Arg Asp Glu Phe Lys Gly Ser Lys Phe Arg Asp Ile Phe His Gly Ala Asp
Gly Asp Asp Leu Leu Asn Gly Asn Asp Gly Asp Asp Ile Leu Tyr Gly Asp Lys Gly Asn Asp
Glu Leu Arg Gly Asp Asn Gly Asn Asp Gln Leu Tyr Gly Gly Glu Gly Asn Asp Lys Leu Leu
Gly Gly Asn Gly Asn Asn Tyr Leu Ser Gly Gly Asp Gly Asn Asp Glu Leu Gln Val Leu Gly
Asn Gly Phe Asn Val Leu Arg Gly Gly Lys Gly Asp Asp Lys Leu Tyr Gly Ser Ser Gly Ser
Asp Leu Leu Asp Gly Gly Glu Gly Asn Asp Tyr Leu Glu Gly Gly Asp Gly Ser Asp Phe Tyr
Val Tyr Arg Ser Thr Ser Gly Asn His Thr Ile Tyr Asp Gln Gly Lys Ser Ser Asp Leu Asp
Lys Leu Tyr Leu Ser Asp Phe Ser Phe Asp Arg Leu Leu Val Glu Lys Val Asp Asp Asn Leu
Val Leu Arg Ser Asn Glu Ser Ser His Asn Asn Gly Val Leu Thr Ile Lys Asp Trp Phe Lys
Glu Gly Asn Lys Tyr Asn His Lys Ile Glu Gln Ile Val Asp Lys Asn Gly Arg Lys Leu Thr
Ala Glu Asn Leu Gly Thr Tyr Phe Lys Asn Ala Pro Lys Ala Asp Asn Leu Leu Asn Tyr Ala
Thr Lys Glu Asp Gln Asn Glu Ser Asn Leu Ser Ser Leu Lys Thr Glu Leu Ser Lys Ile Ile
Thr Asn Ala Gly Asn Phe Gly Val Ala Lys Gln Gly Asn Thr Gly Ile Asn Thr Ala Ala
Leu Asn Asn Glu Val Asn Lys Ile Ile Ser Ser Ala Asn Thr Phe Ala Thr Ser Gln Leu
Gly Gly Ser Gly Met Gly Thr Leu Pro Ser Thr Asn Val Asn Ser Met Met Leu Gly Asn
Leu Ala Arg Ala Ala
APP ApxIA K560A K686A
SEQ ID NO: 4
Met Ala Asn Ser Gln Leu Asp Arg Val Lys Gly Leu Ile Asp Ser Leu Asn Gln His Thr Lys
Ser Ala Ala Lys Ser Gly Ala Gly Ala Leu Lys Asn Gly Leu Gly Gln Val Lys Gln Ala Gly
Gln Lys Leu Ile Leu Tyr Ile Pro Lys Asp Tyr Gln Ala Ser Thr Gly Ser Ser Leu Asn Asp
Leu Val Lys Ala Ala Glu Ala Leu Gly Ile Glu Val His Arg Ser Glu Lys Asn Gly Thr Ala
Leu Ala Lys Glu Leu Phe Gly Thr Thr Glu Lys Leu Leu Gly Phe Ser Glu Arg Gly Ile Ala
Leu Phe Ala Pro Gln Phe Asp Lys Leu Leu Asn Lys Asn Gln Lys Leu Ser Lys Ser Leu Gly
Gly Ser Ser Glu Ala Leu Gly Gln Arg Leu Asn Lys Thr Gln Thr Ala Leu Ser Ala Leu Gln
Ser Phe Leu Gly Thr Ala Ile Ala Gly Met Asp Leu Asp Ser Leu Leu Arg Arg Arg Arg Asn
Gly Glu Asp Val Ser Gly Ser Glu Leu Ala Lys Ala Gly Val Asp Leu Ala Ala Gln Leu Val
Asp Asn Ile Ala Ser Ala Thr Gly Thr Val Asp Ala Phe Ala Glu Gln Leu Gly Lys Leu Gly
Asn Ala Leu Ser Asn Thr Arg Leu Ser Gly Leu Ala Ser Lys Leu Asn Asn Leu Pro Asp Leu
Ser Leu Ala Gly Pro Gly Phe Asp Ala Val Ser Gly Ile Leu Ser Val Val Ser Ala Ser Phe
Ile Leu Ser Asn Lys Asp Ala Asp Ala Gly Thr Lys Ala Ala Ala Gly Ile Glu Ile Ser
Thr Lys Ile Leu Gly Asn Ile Gly Lys Ala Val Ser Gln Tyr Ile Ile Ala Gln Arg Val Ala
Ala Gly Leu Ser Thr Thr Ala Ala Thr Gly Gly Leu Ile Gly Ser Val Val Ala Leu Ala Ile
Ser Pro Leu Ser Phe Leu Asn Val Ala Asp Lys Phe Glu Arg Ala Lys Gln Leu Glu Gln Tyr
Ser Glu Arg Phe Lys Lys Phe Gly Tyr Glu Gly Asp Ser Leu Leu Ala Ser Phe Tyr Arg Glu
Thr Gly Ala Ile Glu Ala Ala Leu Thr Thr Ile Asn Ser Val Leu Ser Ala Ala Ser Ala Gly
Val Gly Ala Ala Ala Thr Gly Ser Leu Val Gly Ala Pro Val Ala Ala Leu Val Ser Ala Ile
Thr Gly Ile Ile Ser Gly Ile Leu Asp Ala Ser Lys Gln Ala Ile Phe Glu Arg Val Ala Thr
Lys Leu Ala Asn Lys Ile Asp Glu Trp Glu Lys Lys His Gly Lys Asn Tyr Phe Glu Asn Gly
Tyr Asp Ala Arg His Ser Ala Phe Leu Glu Asp Thr Phe Glu Leu Leu Ser Gln Tyr Asn Lys
Glu Tyr Ser Val Glu Arg Val Val Ala Ile Thr Gln Gln Arg Trp Asp Val Asn Ile Gly Glu
Leu Ala Gly Ile Thr Arg Lys Gly Ala Asp Ala Lys Ser Gly Lys Ala Tyr Val Asp Phe Phe
Glu Glu Gly Lys Leu Leu Glu Lys Asp Pro Asp Arg Phe Asp Lys Lys Val Phe Asp Pro Leu
Glu Gly Lys Ile Asp Leu Ser Ser Ile Asn Lys Thr Thr Leu Leu Lys Phe Ile Thr Pro Val
Phe Thr Ala Gly Glu Glu Ile Arg Glu Arg Lys Gln Thr Gly Ala Tyr Glu Tyr Met Thr Glu
Leu Phe Val Lys Gly Lys Glu Lys Trp Val Val Thr Gly Val Gln Ser His Asn Ala Ile Tyr
Asp Tyr Thr Asn Leu Ile Gln Leu Ala Ile Asp Lys Lys Gly Glu Lys Arg Gln Val Thr Ile
Glu Ser His Leu Gly Glu Lys Asn Asp Arg Ile Tyr Leu Ser Ser Gly Ser Ser Ile Val Tyr
Ala Gly Asn Gly His Asp Val Ala Tyr Tyr Asp Lys Thr Asp Thr Gly Tyr Leu Thr Phe Asp
Gly Gln Ser Ala Gln Lys Ala Gly Glu Tyr Ile Val Thr Lys Glu Leu Lys Ala Asp Val Lys
Val Leu Lys Glu Val Val Lys Thr Gln Asp Ile Ser Val Gly Ala Arg Ser Glu Lys Leu Glu
Tyr Arg Asp Tyr Glu Leu Ser Pro Phe Glu Leu Gly Asn Gly Ile Arg Ala Lys Asp Glu Leu
His Ser Val Glu Glu Ile Ile Gly Ser Asn Arg Lys Asp Lys Phe Phe Gly Ser Arg Phe Thr
Asp Ile Phe His Gly Ala Lys Gly Asp Asp Glu Ile Tyr Gly Asn Asp Gly His Asp Ile Leu
Tyr Gly Asp Asp Gly Asn Asp Val Ile His Gly Gly Asp Gly Asn Asp His Leu Val Gly Gly
Asn Gly Asn Asp Arg Leu Ile Gly Gly Lys Gly Asn Asn Phe Leu Asn Gly Gly Asp Gly Asp
Asp Glu Leu Gln Val Phe Glu Gly Gln Tyr Asn Val Leu Leu Gly Gly Ala Gly Asn Asp Ile
Leu Tyr Gly Ser Asp Gly Thr Asn Leu Phe Asp Gly Gly Val Gly Asn Asp Lys Ile Tyr Gly
Gly Leu Gly Lys Asp Ile Tyr Arg Tyr Ser Lys Glu Tyr Gly Arg His Ile Ile Ile Glu Lys
Gly Gly Asp Asp Asp Thr Leu Leu Leu Ser Asp Leu Ser Phe Lys Asp Val Gly Phe Ile Arg
Ile Gly Asp Asp Leu Leu Val Asn Lys Arg Ile Gly Gly Thr Leu Tyr Tyr His Glu Asp Tyr
Asn Gly Asn Ala Leu Thr Ile Lys Asp Trp Phe Lys Glu Gly Lys Glu Gly Gln Asn Asn Lys
Ile Glu Lys Ile Val Asp Lys Asp Gly Ala Tyr Val Leu Ser Gln Tyr Leu Thr Glu Leu Thr
Ala Pro Gly Arg Gly Ile Asn Tyr Phe Asn Gly Leu Glu Glu Lys Leu Tyr Tyr Gly Glu Gly
Tyr Asn Ala Leu Pro Gln Leu Arg Lys Asp Ile Glu Gln Ile Ile Ser Ser Thr Gly Ala Leu
Thr Gly Glu His Gly Gln Val Leu Val Gly Ala Gly Gly Pro Leu Ala Tyr Ser Asn Ser Pro
Asn Ser Ile Pro Asn Ala Phe Ser Asn Tyr Leu Thr Gln Ser Ala
ApxIIA S148G K557A N687A
SEQ ID NO: 5
Met Ser Lys Ile Thr Leu Ser Ser Leu Lys Ser Ser Leu Gln Gln Gly Leu Lys Asn Gly Lys
Asn Lys Leu Asn Gln Ala Gly Thr Thr Leu Lys Asn Gly Leu Thr Gln Thr Gly His Ser Leu
Gln Asn Gly Ala Lys Lys Leu Ile Leu Tyr Ile Pro Gln Gly Tyr Asp Ser Gly Gln Gly Asn
Gly Val Gln Asp Leu Val Lys Ala Ala Asn Asp Leu Gly Ile Glu Val Trp Arg Glu Glu Arg
Ser Asn Leu Asp Ile Ala Lys Thr Ser Phe Asp Thr Thr Gln Lys Ile Leu Gly Phe Thr Asp
Arg Gly Ile Val Leu Phe Ala Pro Gln Leu Asp Asn Leu Leu Lys Lys Asn Pro Lys Ile Gly
Asn Thr Leu Gly Ser Ala Ser Ser Ile Ser Gln Asn Ile Gly Lys Ala Asn Thr Val Leu Gly
Gly Ile Gln Ser Ile Leu Gly Ser Val Leu Ser Gly Val Asn Leu Asn Glu Leu Leu Gln Asn
Lys Asp Pro Asn Gln Leu Glu Leu Ala Lys Ala Gly Leu Glu Leu Thr Asn Glu Leu Val Gly
Asn Ile Ala Ser Ser Val Gln Thr Val Asp Ala Phe Ala Glu Gln Ile Ser Lys Leu Gly Ser
His Leu Gln Asn Val Lys Gly Leu Gly Gly Leu Ser Asn Lys Leu Gln Asn Leu Pro Asp Leu
Gly Lys Ala Ser Leu Gly Leu Asp Ile Ile Ser Gly Leu Leu Ser Gly Ala Ser Ala Gly Leu
Ile Leu Ala Asp Lys Glu Ala Ser Thr Glu Lys Lys Ala Ala Ala Gly Val Glu Phe Ala Asn
Gln Ile Ile Gly Asn Val Thr Lys Ala Val Ser Ser Tyr Ile Leu Ala Gln Arg Val Ala Ser
Gly Leu Ser Ser Thr Gly Pro Val Ala Ala Leu Ile Ala Ser Thr Val Ala Leu Ala Val Ser
Pro Leu Ser Phe Leu Asn Val Ala Asp Lys Phe Lys Gln Ala Asp Leu Ile Lys Ser Tyr Ser
Glu Arg Phe Gln Lys Leu Gly Tyr Asp Gly Asp Arg Leu Leu Ala Asp Phe His Arg Glu Thr
Gly Thr Ile Asp Ala Ser Val Thr Thr Ile Asn Thr Ala Leu Ala Ala Ile Ser Gly Gly Val
Gly Ala Ala Ser Ala Gly Ser Leu Val Gly Ala Pro Val Ala Leu Leu Val Ala Gly Val Thr
Gly Leu Ile Thr Thr Ile Leu Glu Tyr Ser Lys Gln Ala Met Phe Glu His Val Ala Asn Lys
Val His Asp Arg Ile Val Glu Trp Glu Lys Lys His Asn Lys Asn Tyr Phe Glu Gln Gly Tyr
Asp Ser Arg His Leu Ala Asp Leu Gln Asp Asn Met Lys Phe Leu Ile Asn Leu Asn Lys Glu
Leu Gln Ala Glu Arg Val Val Ala Ile Thr Gln Gln Arg Trp Asp Asn Gln Ile Gly Asp Leu
Ala Ala Ile Ser Arg Arg Thr Asp Lys Ile Ser Ser Gly Lys Ala Tyr Val Asp Ala Phe Glu
Glu Gly Gln His Gln Ser Tyr Asp Ser Ser Val Gln Leu Asp Asn Lys Asn Gly Ile Ile Asn
Ile Ser Asn Thr Asn Arg Lys Thr Gln Ser Val Leu Phe Arg Thr Pro Leu Leu Thr Pro Gly
Glu Glu Asn Arg Glu Arg Ile Gln Glu Gly Ala Asn Ser Tyr Ile Thr Lys Leu His Ile Gln
Arg Val Asp Ser Trp Thr Val Thr Asp Gly Asp Ala Ser Ser Ser Val Asp Phe Thr Asn Val
Val Gln Arg Ile Ala Val Lys Phe Asp Asp Ala Gly Asn Ile Ile Glu Ser Lys Asp Thr Lys
Ile Ile Ala Asn Leu Gly Ala Gly Asn Asp Asn Val Phe Val Gly Ser Ser Thr Thr Val Ile
Asp Gly Gly Asp Gly His Asp Arg Val His Tyr Ser Arg Gly Glu Tyr Gly Ala Leu Val Ile
Asp Ala Thr Ala Glu Thr Glu Lys Gly Ser Tyr Ser Val Lys Arg Tyr Val Gly Asp Ser Lys
Ala Leu His Glu Thr Ile Ala Thr His Gln Thr Asn Val Gly Ala Arg Glu Glu Lys Ile Glu
Tyr Arg Arg Glu Asp Asp Arg Phe His Thr Gly Tyr Thr Val Thr Asp Ser Leu Lys Ser Val
Glu Glu Ile Ile Gly Ser Gln Phe Asn Asp Ile Phe Lys Gly Ser Gln Phe Asp Asp Val Phe
His Gly Gly Asn Gly Val Asp Thr Ile Asp Gly Asn Asp Gly Asp Asp His Leu Phe Gly Gly
Ala Gly Asp Asp Val Ile Asp Gly Gly Asn Gly Asn Asn Phe Leu Val Gly Gly Thr Gly Asn
Asp Ile Ile Ser Gly Gly Lys Asp Asn Asp Ile Tyr Val His Lys Thr Gly Asp Gly Asn Asp
Ser Ile Thr Asp Ser Gly Gly Gln Asp Lys Leu Ala Phe Ser Asp Val Asn Leu Lys Asp Leu
Thr Phe Lys Lys Val Asp Ser Ser Leu Glu Ile Ile Asn Gln Lys Gly Glu Lys Val Arg Ile
Gly Asn Trp Phe Leu Glu Asp Asp Leu Ala Ser Thr Val Ala Asn Tyr Lys Ala Thr Asn Asp
Arg Lys Ile Glu Glu Ile Ile Gly Lys Gly Gly Glu Arg Ile Thr Ser Glu Gln Val Asp Lys
Leu Ile Lys Glu Gly Asn Asn Gln Ile Ser Ala Glu Ala Leu Ser Lys Val Val Asn Asp Tyr
Asn Thr Ser Lys Asp Arg Gln Asn Val Ser Asn Ser Leu Ala Lys Leu Ile Ser Ser Val Gly
Ser Phe Thr Ser Ser Ser Asp Phe Arg Asn Asn Leu Gly Thr Tyr Val Pro Ser Ser Ile Asp
Val Ser Asn Asn Ile Gln Leu Ala Arg Ala Ala
APP ApxIIIA K571A K702A
SEQ ID NO: 6
Met Ser Thr Trp Ser Ser Met Leu Ala Asp Leu Lys Lys Arg Ala Glu Glu Ala Lys Arg Gln
Ala Lys Lys Gly Tyr Asp Val Thr Lys Asn Gly Leu Gln Tyr Gly Val Ser Gln Ala Lys Leu
Gln Ala Leu Ala Ala Gly Lys Ala Val Gln Lys Tyr Gly Asn Lys Leu Val Leu Val Ile Pro
Lys Glu Tyr Asp Gly Ser Val Gly Asn Gly Phe Phe Asp Leu Val Lys Ala Ala Glu Glu Leu
Gly Ile Gln Val Lys Tyr Val Asn Arg Asn Glu Leu Glu Val Ala His Lys Ser Leu Gly Thr
Ala Asp Gln Phe Leu Gly Leu Thr Glu Arg Gly Leu Thr Leu Phe Ala Pro Gln Leu Asp Gln
Phe Leu Gln Lys His Ser Lys Ile Ser Asn Val Val Gly Ser Ser Thr Gly Asp Ala Val Ser
Lys Leu Ala Lys Ser Gln Thr Ile Ile Ser Gly Ile Gln Ser Val Leu Gly Thr Val Leu Ala
Gly Ile Asn Leu Asn Glu Ala Ile Ile Ser Gly Gly Ser Glu Leu Glu Leu Ala Glu Ala Gly
Val Ser Leu Ala Ser Glu Leu Val Ser Asn Ile Ala Lys Gly Thr Thr Thr Ile Asp Ala Phe
Thr Thr Gln Ile Gln Asn Phe Gly Lys Leu Val Glu Asn Ala Lys Gly Leu Gly Gly Val Gly
Arg Gln Leu Gln Asn Ile Ser Gly Ser Ala Leu Ser Lys Thr Gly Leu Gly Leu Asp Ile Ile
Ser Ser Leu Leu Ser Gly Val Thr Ala Ser Phe Ala Leu Ala Asn Lys Asn Ala Ser Thr Ser
Thr Lys Val Ala Ala Gly Phe Glu Leu Ser Asn Gln Val Ile Gly Gly Ile Thr Lys Ala Val
Ser Ser Tyr Ile Leu Ala Gln Arg Leu Ala Ala Gly Leu Ser Thr Thr Gly Pro Ala Ala Ala
Leu Ile Ala Ser Ser Ile Ser Leu Ala Ile Ser Pro Leu Ala Phe Leu Arg Val Ala Asp Asn
Phe Asn Arg Ser Lys Glu Ile Gly Glu Phe Ala Glu Arg Phe Lys Lys Leu Gly Tyr Asp Gly
Asp Lys Leu Leu Ser Glu Phe Tyr His Glu Ala Gly Thr Ile Asp Ala Ser Ile Thr Thr Ile
Ser Thr Ala Leu Ser Ala Ile Ala Ala Gly Thr Ala Ala Ala Ser Ala Gly Ala Leu Val Gly
Ala Pro Ile Thr Leu Leu Val Thr Gly Ile Thr Gly Leu Ile Ser Gly Ile Leu Glu Phe Ser
Lys Gln Pro Met Leu Asp His Val Ala Ser Lys Ile Gly Asn Lys Ile Asp Glu Trp Glu Lys
Lys Tyr Gly Lys Asn Tyr Phe Glu Asn Gly Tyr Asp Ala Arg His Lys Ala Phe Leu Glu Asp
Ser Phe Ser Leu Leu Ser Ser Phe Asn Lys Gln Tyr Glu Thr Glu Arg Ala Val Leu Ile Thr
Gln Gln Arg Trp Asp Glu Tyr Ile Gly Glu Leu Ala Gly Ile Thr Gly Lys Gly Asp Lys Leu
Ser Ser Gly Lys Ala Tyr Val Asp Tyr Phe Gln Glu Gly Lys Leu Leu Glu Lys Lys Pro Asp
Asp Phe Ser Lys Val Val Phe Asp Pro Thr Lys Gly Glu Ile Asp Ile Ser Asn Ser Gln Thr
Ser Thr Leu Leu Lys Phe Val Thr Pro Leu Leu Thr Pro Gly Thr Glu Ser Arg Glu Arg Thr
Gln Thr Gly Ala Tyr Glu Tyr Ile Thr Lys Leu Val Val Lys Gly Lys Asp Lys Trp Val Val
Asn Gly Val Lys Asp Lys Gly Ala Val Tyr Asp Tyr Thr Asn Leu Ile Gln His Ala His Ile
Ser Ser Ser Val Ala Arg Gly Glu Glu Tyr Arg Glu Val Arg Leu Val Ser His Leu Gly Asn
Gly Asn Asp Lys Val Phe Leu Ala Ala Gly Ser Ala Glu Ile His Ala Gly Glu Gly His Asp
Val Val Tyr Tyr Asp Lys Thr Asp Thr Gly Leu Leu Val Ile Asp Gly Thr Lys Ala Thr Glu
Gln Gly Arg Tyr Ser Val Thr Arg Glu Leu Ser Gly Ala Thr Lys Ile Leu Arg Glu Val Ile
Lys Asn Gln Lys Ser Ala Val Gly Ala Arg Glu Glu Thr Leu Glu Tyr Arg Asp Tyr Glu Leu
Thr Gln Ser Gly Asn Ser Asn Leu Lys Ala His Asp Glu Leu His Ser Val Glu Glu Ile Ile
Gly Ser Asn Gln Arg Asp Glu Phe Lys Gly Ser Lys Phe Arg Asp Ile Phe His Gly Ala Asp
Gly Asp Asp Leu Leu Asn Gly Asn Asp Gly Asp Asp Ile Leu Tyr Gly Asp Lys Gly Asn Asp
Glu Leu Arg Gly Asp Asn Gly Asn Asp Gln Leu Tyr Gly Gly Glu Gly Asn Asp Lys Leu Leu
Gly Gly Asn Gly Asn Asn Tyr Leu Ser Gly Gly Asp Gly Asn Asp Glu Leu Gln Val Leu Gly
Asn Gly Phe Asn Val Leu Arg Gly Gly Lys Gly Asp Asp Lys Leu Tyr Gly Ser Ser Gly Ser
Asp Leu Leu Asp Gly Gly Glu Gly Asn Asp Tyr Leu Glu Gly Gly Asp Gly Ser Asp Phe Tyr
Val Tyr Arg Ser Thr Ser Gly Asn His Thr Ile Tyr Asp Gln Gly Lys Ser Ser Asp Leu Asp
Lys Leu Tyr Leu Ser Asp Phe Ser Phe Asp Arg Leu Leu Val Glu Lys Val Asp Asp Asn Leu
Val Leu Arg Ser Asn Glu Ser Ser His Asn Asn Gly Val Leu Thr Ile Lys Asp Trp Phe Lys
Glu Gly Asn Lys Tyr Asn His Lys Ile Glu Gln Ile Val Asp Lys Asn Gly Arg Lys Leu Thr
Ala Glu Asn Leu Gly Thr Tyr Phe Lys Asn Ala Pro Lys Ala Asp Asn Leu Leu Asn Tyr Ala
Thr Lys Glu Asp Gln Asn Glu Ser Asn Leu Ser Ser Leu Lys Thr Glu Leu Ser Lys Ile Ile
Thr Asn Ala Gly Asn Phe Gly Val Ala Lys Gln Gly Asn Thr Gly Ile Asn Thr Ala Ala Leu
Asn Asn Glu Val Asn Lys Ile Ile Ser Ser Ala Asn Thr Phe Ala Thr Ser Gln Leu Gly Gly
Ser Gly Met Gly Thr Leu Pro Ser Thr Asn Val Asn Ser Met Met Leu Gly Asn Leu Ala
Arg Ala Ala
APP truncated ApxIIA
SEQ ID NO: 7
Gln Gly Thr Asp Ser Arg His Leu Ala Asp Leu Gln Asp Asn Met Lys Phe Leu Ile Asn Leu
Asn Lys Glu Leu Gln Ala Glu Arg Val Val Ala Ile Thr Gln Gln Arg Trp Asp Asn Gln Ile
Gly Asp Leu Ala Ala Ile Ser Arg Arg Thr Asp Lys Ile Ser Ser Gly Lys Ala Tyr Val Asp
Ala Phe Glu Glu Gly Gln His Gln Ser Tyr Asp Ser Ser Val Gln Leu Asp Asn Lys Asn Gly
Ile Ile Asn Ile Ser Asn Thr Asn Arg Lys Thr Gln Ser Val Leu Phe Arg Thr Pro Leu Leu
Thr Pro Gly Glu Glu Asn Arg Glu Arg Ile Gln Glu Gly Lys Asn Ser Tyr Ile Thr Lys Leu
His Ile Gln Arg Val Asp Ser Trp Thr Val Thr Asp Gly Asp Ala Ser Ser Ser Val Asp Phe
Thr Asn Val Val Gln Arg Ile Ala Val Lys Phe Asp Asp Ala Gly Asn Ile Ile Glu Ser Lys
Asp Thr Lys Ile Ile Ala Asn Leu Gly Ala Gly Asn Asp Asn Val Phe Val Gly Ser Ser Thr
Thr Val Ile Asp Gly Gly Asp Gly His Asp Arg Val His Tyr Ser Arg Gly Glu Tyr Gly Ala
Leu Val Ile Asp Ala Thr Ala Glu Thr Glu Lys Gly Ser Tyr Ser Val Lys Arg Tyr Val Gly
Asp Ser Lys Ala Leu His Glu Thr Ile Ala Thr His Gln Thr Asn Val Gly Asn Arg Glu Glu
Lys Ile Glu Tyr Arg Arg Glu Asp Asp Arg Phe His Thr Gly Tyr Thr Val Thr Asp Ser Leu
Lys Ser Val Glu Glu Ile Ile Gly Ser Gln Phe Asn Asp Ile Phe Lys Gly Ser Gln Phe Asp
Asp Val Phe His Gly Gly Asn Gly Val Asp Thr Ile Asp Gly Asn Asp Gly Asp Asp His Leu
Phe Gly Gly Ala Gly Asp Asp Val Ile Asp Gly Gly Asn Gly Asn Asn Phe Leu Val Gly Gly
Thr Gly Asn Asp Ile Ile Ser Gly Gly Lys Asp Asn Asp Ile Tyr Val His Lys Thr Gly Asp
Gly Asn Asp Ser Ile Thr
APP truncated ApxIIA K557A N687A
SEQ ID NO: 8
Gln Gly Tyr Asp Ser Arg His Leu Ala Asp Leu Gln Asp Asn Met Lys Phe Leu Ile Asn Leu
Asn Lys Glu Leu Gln Ala Glu Arg Val Val Ala Ile Thr Gln Gln Arg Trp Asp Asn Gln Ile
Gly Asp Leu Ala Ala Ile Ser Arg Arg Thr Asp Lys Ile Ser Ser Gly Lys Ala Tyr Val Asp
Ala Phe Glu Glu Gly Gln His Gln Ser Tyr Asp Ser Ser Val Gln Leu Asp Asn Lys Asn Gly
Ile Ile Asn Ile Ser Asn Thr Asn Arg Lys Thr Gln Ser Val Leu Phe Arg Thr Pro Leu Leu
Thr Pro Gly Glu Glu Asn Arg Glu Arg Ile Gln Glu Gly Ala Asn Ser Tyr Ile Thr Lys Leu
His Ile Gln Arg Val Asp Ser Trp Thr Val Thr Asp Gly Asp Ala Ser Ser Ser Val Asp Phe
Thr Asn Val Val Gln Arg Ile Ala Val Lys Phe Asp Asp Ala Gly Asn Ile Ile Glu Ser Lys
Asp Thr Lys Ile Ile Ala Asn Leu Gly Ala Gly Asn Asp Asn Val Phe Val Gly Ser Ser Thr
Thr Val Ile Asp Gly Gly Asp Gly His Asp Arg Val His Tyr Ser Arg Gly Glu Tyr Gly Ala
Leu Val Ile Asp Ala Thr Ala Glu Thr Glu Lys Gly Ser Tyr Ser Val Lys Arg Tyr Val Gly
Asp Ser Lys Ala Leu His Glu Thr Ile Ala Thr His Gln Thr Asn Val Gly Ala Arg Glu Glu
Lys Ile Glu Tyr Arg Arg Glu Asp Asp Arg Phe His Thr Gly Tyr Thr Val Thr Asp Ser Leu
Lys Ser Val Glu Glu Ile Ile Gly Ser Gln Phe Asn Asp Ile Phe Lys Gly Ser Gln Phe Asp
Asp Val Phe His Gly Gly Asn Gly Val Asp Thr Ile Asp Gly Asn Asp Gly Asp Asp His Leu
Phe Gly Gly Ala Gly Asp Asp Val Ile Asp Gly Gly Asn Gly Asn Asn Phe Leu Val Gly Gly
Thr Gly Asn Asp Ile Ile Ser Gly Gly Lys Asp Asn Asp Ile Tyr Val His Lys Thr Gly Asp
Gly Asn Asp Ser Ile Thr
ApxIA wild type
SEQ ID NO: 9
atggctaactctcagctcgatagagtcaaaggattgattgattcacttaatcaacatacaaaaagtgcagctaaatcaggtgc
cggcgcattaaaaaatggtttgggacaggtgaagcaagcagggcagaaattaattttatatattccgaaagattatcaagcta
gtaccggctcaagtcttaatgatttagtgaaagcggcggaggctttagggatcgaagtacatcgctcggaaaaaaacggtacc
gcactagcgaaagaattattcggtacaacggaaaaactattaggtttctcggaacgaggcatcgcattatttgcacctcagtt
tgataagttactgaataagaaccaaaaattaagtaaatcgctcggcggttcatcggaagcattaggacaacgtttaaataaaa
cgcaaacggcactttcagccttacaaagtttcttaggtacggctattgcgggtatggatcttgatagcctgcttcgtcgccgt
agaaacggtgaggacgtcagtggttcggaattagctaaagcgggtgtggatctagccgctcagttagtggataacattgcaag
tgcaacgggtacggtggatgcgtttgccgaacaattaggtaaattgggcaatgccttatctaacactcgcttaagcggtttag
caagtaagttaaataaccttccagatttaagccttgcaggacctgggtttgatgccgtatcaggtatcttatctgttgtttcg
gcttcattcattttaagtaataaagatgccgatgcaggtacaaaagcggcggcaggtattgaaatctcaactaaaatcttagg
caatatcggtaaagcggtttctcaatatattattgcgcaacgtgtggcggcaggcttatccacaactgcggcaaccggtggtt
taatcggttcggtcgtagcattagcgattagcccgctttcgttcttaaatgttgcggataagtttgaacgtgcgaaacagctt
gaacaatattcggagcgctttaaaaagttcggttatgaaggtgatagtttattagcttcattctaccgtgaaaccggtgcgat
tgaagcggcattaaccacgattaacagtgtgttaagtgcggcttccgcaggtgttggggctgctgcaaccggctcattagtcg
gtgcgccggtagcagctttagttagtgcaatcaccggtattatttcaggtattttagatgcttctaaacaggcaatcttcgaa
cgagttgcaacgaaattagcgaataagattgacgaatgggagaaaaaacacggtaaaaactattttgaaaacggttatgacgc
ccgccattccgcattcttagaagatacctttgaattgttatcacaatacaataaagagtattcggtagagcgtgtcgttgcta
ttacgcaacaacgttgggatgtcaatatcggggaacttgccggtatcacgcgtaaaggtgcggatgcgaaaagcggtaaggct
tatgtcgatttctttgaagaaggaaaattgttagagaaagatccggatcgttttgataaaaaagtgtttgatccgcttgaagg
caaaatcgacctttcttcaattaacaaaaccactttattgaaatttattacaccggtttttaccgcaggtgaagagattcgtg
agcgtaagcaaaccggtaaatacgaatatatgaccgaattattcgttaaaggtaaagaaaaatgggtggtaaccggtgtgcag
tcacataatgcgatttatgactatacgaatcttatccaattagcgatagataaaaaaggtgaaaaacgtcaagtgaccattga
atctcatttgggtgagaaaaatgatcgtatatatctttcatccggttcatctatcgtatatgcgggtaacggacatgatgtag
catattacgataaaaccgatacaggttacttaacatttgacggacaaagtgcacagaaagccggtgaatatattgtcactaaa
gaacttaaagctgatgtaaaagttttaaaagaagtggttaaaactcaggatatttcagttggaaaacgcagtgaaaaattaga
atatcgtgattatgagttaagcccattcgaacttgggaacggtatcagagctaaagatgaattacattctgttgaagaaatta
tcggtagtaatcgtaaagacaaattctttggtagtcgctttaccgatattttccatggtgcgaaaggcgatgatgaaatctac
ggtaatgacggccacgatatcttatacggagacgacggtaatgatgtaatccatggcggtgacggtaacgaccatcttgttgg
tcgtaacggaaacgaccgattaatcggcggaaaaggtaataatttccttaatggcggtgatggtgacgatgagttgcaggtct
ttgagggtcaatacaacgtattattaggtggtgcgggtaatgacattctgtatggcagcgatggtactaacttatttgacggt
ggtgtaggcaatgacaaaatctacggtggtttaggtaaggatatttatcgctacagtaaggagtacggtcgtcatatcattat
tgagaaaggcggtgatgatgatacgttattgttatcggatcttagttttaaagatgtaggatttatcagaatcggtgatgatc
ttcttgtgaataaaagaatcggaggaacactgtattaccatgaagattacaatgggaatgcgctcacgattaaagattggttc
aaggaaggtaaagaaggacaaaataataaaattgaaaaaatcgttgataaagatggagcttatgttttaagccaatatctgac
tgaactgacagctcctggaagaggtatcaattactttaatgggttagaagaaaaattgtattatggagaaggatataatgcac
ttcctcaactcagaaaagatattgaacaaatcatttcatctactggtgcacttaccggtgaacacggacaagttttagtggga
gcaggcggtccattagcttacagcaattcaccgaatagcataccgaatgctttcagtaattatttaacacaatctgcttaa
ApxIIA wild type
SEQ ID NO: 10
atgtcaaaaatcactttgtcatcattaaaatcgtccttacaacaaggattgacaaatgggaaaaacaagttaaatcaagcagg
tacaacactgaagaatggtttaactcaaactggtcattctctacagaatggggctaaaaaattaatcttatatattcctcaag
gctatgattcgggtcaaggaaatggaattcaagatttagttaaagctgctaatgatttaggtattgaagtatggcgagaagaa
cgcagcaatttggacattgcaaaaactagctttgatacaactcagaaaattctaggttttactgatagaggaattgtattatt
tgcacctcagctagataatttattaaagaagaatcctaaaattggcaatacattaggaagtgcttctagcatctcacaaaata
taggtaaagccaatactgtattaggtggtattcaatctattttaggatctgttttatctggagtaaatctgaatgaattactt
caaaataaagatcctaatcaattagaacttgcaaaagcagggctagaactgactaatgaattagttggtaatattgctagctc
ggtgcaaactgtagatgcatttgcagaacaaatatctaaactaggttcacatttacagaatgtgaaaggattaggaggattga
gtaataaattacaaaatctaccagatctaggaaaagcaagtttaggtttggacattatctctggtttactttctggagcatct
gcaggtctcattttagcagataaagaggcttcaacagaaaagaaagctgccgcaggtgtagaatttgctaaccaaattatagg
taatgtaacaaaagcggtctcatcttacattcttgcccaacgagtcgcttcaggtttgtcttcaactggtcctgtcgctgcat
taatcgcatctacagttgcactagctgttagccctctttcattcttaaatgtagctgataagtttaaacaagctgatttaatc
aaatcatattctgaacgcttccaaaaattaggatatgatggagatcgtttattagctgattttcaccgtgagacaggaactat
tgatgcttctgtaacaacaattaacactgctttagcagctatctccggtggagttggagctgcaagcgcgggttctctagtcg
gagctccagttgcgttactcgttgctggtgttacgggacttattacaactattctagaatattctaaacaagccatgtttgaa
catgttgcaaataaggttcatgacagaatagttgaatgggagaaaaaacataataaaaactattttgagcaaggttatgattc
tcgtcatttagctgatttacaagacaatatgaagtttcttatcaatttaaataaagaacttcaggctgaacgcgtagtagcta
ttacccaacaaagatgggataaccaaattggagacctagcggcaattagccgtagaacggataaaatttccagtggaaaagct
tatgtggatgcttttgaggaggggcaacaccagtcctacgattcatccgtacagctagataacaaaaacggtattattaatat
tagtaatacaaatagaaagacacaaagtgttttattcagaactccattactaactccaggtgaagagaatcgggaacgtattc
aggaaggtaaaaattcttatattacaaaattacatatacaaagagttgacagttggactgtaacagatggtgatgctagctca
agcgtagatttcactaatgtagtacaacgaatcgctgtgaaatttgatgatgcaggtaacattatcgaatctaaagatactaa
aattatcgcaaatttaggtgctggtaacgataatgtatttgttgggtcaagtactaccgttattgatggcggggacggacatg
atcgagttcactacagtagaggagaatatggcgcattagttattgatgctacagccgagacagaaaaaggctcatattcagta
aaacgctatgtcggagacagtaaagcattacatgaaacaattgccacccaccaaacaaatgttggtaatcgtgaagaaaaaat
tgaatatcgtcgtgaagatgatcgttttcatactggttatactgtgacggactcactcaaatcagttgaagagatcattggtt
cacaatttaatgatattttcaaaggaagccaatttgatgatgtgttccatggtggtaatggtgtagacactattgatggtaac
gatggtgacgatcatttatttggtggcgcaggcgatgatgttatcgatggaggaaacggtaacaatttccttgttggaggaac
cggtaatgatattatctcgggaggtaaagataatgatatttatgtccataaaacaggcgatggaaatgattctattacagact
ctggcggacaagataaactggcattttcggatgtaaatcttaaagacctcacctttaagaaagtagattcttctctcgaaatc
attaatcaaaaaggagaaaaagttcgtattgggaattggttcttagaagatgatttggctagcacagttgctaactataaagc
tacgaatgaccgaaaaattgaggaaattattggtaaaggaggagaacgtattacatcagaacaagttgataaactgattaagg
agggtaacaatcaaatctctgcagaagcattatccaaagttgtgaatgattacaatacgagtaaagatagacagaacgtatct
aatagcttagcaaaattgatttcttcagtcgggagctttacgtcttcctcagactttaggaataatttaggaacatatgttcc
ttcatcaatagatgtctcgaataatattcaattagctagagccgcttaa
ApxIIIA wild type
SEQ ID NO: 11
atgagtacttggtcaagcatgttagccgacttaaaaaaacgggctgaagaagccaaaagacaagccaaaaaaggctacgatgt
aactaaaaatggtttgcaatatggggtgagtcaagcaaaattacaagcattagcagctggtaaagccgttcaaaagtacggta
ataaattagttttagttattccaaaagagtatgacggaagtgttggtaacggtttctttgatttagtaaaagcagctgaggaa
ttaggcattcaagttaaatatgttaaccgtaatgaattggaagttgcccataaaagtttaggtaccgcagaccaattcttggg
tttaacagaacgtggacttactttatttgcaccgcaactagatcagttcttacaaaaacattcaaaaatttctaacgtagtgg
gcagttctactggtgatgcagtaagtaaacttgctaagagtcaaactattatttcaggaattcaatctgtattaggtactgta
ttagcaggtattaatcttaatgaagctattattagtggcggttcagagctcgaattagctgaagctggtgtttctttagcctc
tgagctcgttagtaatattgctaaaggtacaacaacaatagatgctttcactacacaaatccagaactttgggaaattagtgg
aaaatgctaaagggttaggtggtgttggccgccaattacagaatatttcaggttctgcattaagcaaaactggattaggtttg
gatattatctcaagcttactttcaggagtaactgcaagttttgctttagcgaataagaatgcttcaacaagcactaaagttgc
tgctggctttgaactctcaaatcaagtaattggtggtattacgaaagcagtatcaagctatattcttgcacagcgtttagctg
ctggtttatcaacgacaggtcctgctgcagcactaattgcgtctagtatttctttagcaatcagtccattggcgtttttacgt
gtagctgataattttaatcgttctaaagaaattggcgaatttgctgaacgtttcaaaaaattgggctatgacggcgataaact
actttcagagttttatcacgaagctggtactattgatgcctcaattactacaattagtacagcactttctgctatcgcagctg
gaacggccgccgcgagtgcaggtgcattagttggcgcaccaattactttgttggttactggtatcacaggattaatttctggt
attttagagttctctaaacaaccaatgttagatcatgttgcatcgaaaattggtaacaaaattgacgaatgggagaaaaaata
cggtaaaaattacttcgagaatggctatgatgctcgtcataaagctttcttagaagattcattctcattattgtctagtttta
ataaacaatatgaaactgaaagagctgttttaattacacaacaacgttgggatgaatatattggcgaacttgcgggtattact
ggcaaaggtgacaaactctctagtggtaaggcgtatgtagattactttcaagaaggtaaattattagagaaaaaacctgatga
ctttagcaaagtagttttcgatccaactaagggcgaaattgatatttcaaatagccaaacgtcaacgttgttaaaatttgtta
cgccattattaacaccaggtacagagtcacgtgaaagaactcaaacaggtaaatatgaatatatcacgaagttagttgtaaaa
ggtaaagataaatgggttgttaatggcgttaaagataaaggtgccgtttatgattatactaatttaattcaacatgctcatat
tagttcatcagtagcacgtggtgaagaataccgtgaagttcgtttggtatctcatctaggcaatggtaatgacaaagtgttct
tagctgcgggttccgcagaaattcacgctggtgaaggtcatgatgtggtttattatgataaaaccgatacaggtcttttagta
attgatggaaccaaagcgactgaacaagggcgttattctgttacgcgcgaattgagtggtgctacaaaaatcctgagagaagt
aataaaaaatcaaaaatctgctgttggtaaacgtgaagaaaccttggaatatcgtgattatgaattaacgcaatcaggtaata
gtaacctaaaagcacatgatgaattacattcagtagaagaaattattggaagtaatcagagagacgaatttaaaggtagtaaa
ttcagagatattttccatggtgccgatggtgatgatctattaaatggtaatgatggggatgatattctatacggtgataaagg
taacgatgagttaagaggtgataatggtaacgaccaactttatggtggtgaaggtaatgacaaactattaggaggtaatggca
ataattacctcagtggtggtgatggcaatgatgagcttcaagtcttaggcaatggttttaatgtgcttcgtggcggtaaaggc
gatgataaactttatggtagctcaggttctgatttacttgatggtggagaaggtaatgattatctagaaggaggcgatggtag
cgatttttatgtttatcgttccacttcaggtaatcatactatttatgatcaaggtaaatctagtgatttagataaactatatt
tgtctgatttttccttcgatcgtcttcttgttgagaaagttgatgataaccttgtacttagaagtaatgaaagtagtcataat
aatggagtactcacaatcaaagactggtttaaagaagggaataaatataaccataaaattgaacaaattgttgataaaaatgg
tagaaaattgacagcagagaatttaggaacttatttcaaaaatgctccaaaagctgacaatttgcttaattatgcaactaaag
aagatcagaatgaaagcaatttatcttcacttaaaactgaattaagtaaaattattactaatgcaggtaattttggtgtggca
aaacaaggtaatactggaatcaatacagctgccttgaacaatgaagtgaataaaatcatttcttctgctaatacctttgctac
ttcacaattgggtggctcagggatgggaacattaccatcaacgaatgtaaattcaatgatgctaggtaacctagctagagcag
cttaa
APP ApxIA K560A K686A
SEQ ID NO: 12
atggctaactctcagctcgatagagtcaaaggattgattgattcacttaatcaacatacaaaaagtgcagctaaatcaggtgc
cggcgcattaaaaaatggtttgggacaggtgaagcaagcagggcagaaattaattttatatattccgaaagattatcaagcta
gtaccggctcaagtcttaatgatttagtgaaagcggcggaggctttagggatcgaagtacatcgctcggaaaaaaacggtacc
gcactagcgaaagaattattcggtacaacggaaaaactattaggtttctcggaacgaggcatcgcattatttgcacctcagtt
tgataagttactgaataagaaccaaaaattaagtaaatcgctcggcggttcatcggaagcattaggacaacgtttaaataaaa
cgcaaacggcactttcagccttacaaagtttcttaggtacggctattgcgggtatggatcttgatagcctgcttcgtcgccgt
agaaacggtgaggacgtcagtggttcggaattagctaaagcgggtgtggatctagccgctcagttagtggataacattgcaag
tgcaacgggtacggtggatgcgtttgccgaacaattaggtaaattgggcaatgccttatctaacactcgcttaagcggtttag
caagtaagttaaataaccttccagatttaagccttgcaggacctgggtttgatgccgtatcaggtatcttatctgttgtttcg
gcttcattcattttaagtaataaagatgccgatgcaggtacaaaagcggcggcaggtattgaaatctcaactaaaatcttagg
caatatcggtaaagcggtttctcaatatattattgcgcaacgtgtggcggcaggcttatccacaactgcggcaaccggtggtt
taatcggttcggtcgtagcattagcgattagcccgctttcgttcttaaatgttgcggataagtttgaacgtgcgaaacagctt
gaacaatattcggagcgctttaaaaagttcggttatgaaggtgatagtttattagcttcattctaccgtgaaaccggtgcgat
tgaagcggcattaaccacgattaacagtgtgttaagtgcggcttccgcaggtgttggggctgctgcaaccggctcattagtcg
gtgcgccggtagcagctttagttagtgcaatcaccggtattatttcaggtattttagatgcttctaaacaggcaatcttcgaa
cgagttgcaacgaaattagcgaataagattgacgaatgggagaaaaaacacggtaaaaactattttgaaaacggttatgacgc
ccgccattccgcattcttagaagatacctttgaattgttatcacaatacaataaagagtattcggtagagcgtgtcgttgcta
ttacgcaacaacgttgggatgtcaatatcggggaacttgccggtatcacgcgtaaaggtgcggatgcgaaaagcggtaaggct
tatgtcgatttctttgaagaaggaaaattgttagagaaagatccggatcgttttgataaaaaagtgtttgatccgcttgaagg
caaaatcgacctttcttcaattaacaaaaccactttattgaaatttattacaccggtttttaccgcaggtgaagagattcgtg
agcgtaagcaaaccggtgcatacgaatatatgaccgaattattcgttaaaggtaaagaaaaatgggtggtaaccggtgtgcag
tcacataatgcgatttatgactatacgaatcttatccaattagcgatagataaaaaaggtgaaaaacgtcaagtgaccattga
atctcatttgggtgagaaaaatgatcgtatatatctttcatccggttcatctatcgtatatgcgggtaacggacatgatgtag
catattacgataaaaccgatacaggttacttaacatttgacggacaaagtgcacagaaagccggtgaatatattgtcactaaa
gaacttaaagctgatgtaaaagttttaaaagaagtggttaaaactcaggatatttcagttggagcacgcagtgaaaaattaga
atatcgtgattatgagttaagcccattcgaacttgggaacggtatcagagctaaagatgaattacattctgttgaagaaatta
tcggtagtaatcgtaaagacaaattctttggtagtcgctttaccgatattttccatggtgcgaaaggcgatgatgaaatctac
ggtaatgacggccacgatatcttatacggagacgacggtaatgatgtaatccatggcggtgacggtaacgaccatcttgttgg
tggtaacggaaacgaccgattaatcggcggaaaaggtaataatttccttaatggcggtgatggtgacgatgagttgcaggtct
ttgagggtcaatacaacgtattattaggtggtgcgggtaatgacattctgtatggcagcgatggtactaacttatttgacggt
ggtgtaggcaatgacaaaatctacggtggtttaggtaaggatatttatcgctacagtaaggagtacggtcgtcatatcattat
tgagaaaggcggtgatgatgatacgttattgttatcggatcttagttttaaagatgtaggatttatcagaatcggtgatgatc
ttcttgtgaataaaagaatcggaggaacactgtattaccatgaagattacaatgggaatgcgctcacgattaaagattggttc
aaggaaggtaaagaaggacaaaataataaaattgaaaaaatcgttgataaagatggagcttatgttttaagccaatatctgac
tgaactgacagctcctggaagaggtatcaattactttaatgggttagaagaaaaattgtattatggagaaggatataatgcac
ttcctcaactcagaaaagatattgaacaaatcatttcatctactggtgcacttaccggtgaacacggacaagttttagtggga
gcaggcggtccattagcttacagcaattcaccgaatagcataccgaatgctttcagtaattatttaacacaatctgcttaa
APP ApxIIA S148A K557A K686A
SEQ ID NO: 13
atgtcaaaaatcactttgtcatcattaaaatcgtccttacaacaaggattgacaaatgggaaaaacaagttaaatcaagcagg
tacaacactgaagaatggtttaactcaaactggtcattctctacagaatggggctaaaaaattaatcttatatattcctcaag
gctatgattcgggtcaaggaaatggaattcaagatttagttaaagctgctaatgatttaggtattgaagtatggcgagaagaa
cgcagcaatttggacattgcaaaaactagctttgatacaactcagaaaattctaggttttactgatagaggaattgtattatt
tgcacctcagctagataatttattaaagaagaatcctaaaattggcaatacattaggaagtgcttctagcatctcacaaaata
taggtaaagccaatactgtattaggtggtattcaatctattttaggatctgttttatctggagtaaatctgaatgaattactt
caaaataaagatcctaatcaattagaacttgcaaaagcagggctagaactgactaatgaattagttggtaatattgctagctc
ggtgcaaactgtagatgcatttgcagaacaaatatctaaactaggttcacatttacagaatgtgaaaggattaggaggattga
gtaataaattacaaaatctaccagatctaggaaaagcaagtttaggtttggacattatctctggtttactttctggagcatct
gcaggtctcattttagcagataaagaggcttcaacagaaaagaaagctgccgcaggtgtagaatttgctaaccaaattatagg
taatgtaacaaaagcggtctcatcttacattcttgcccaacgagtcgcttcaggtttgtcttcaactggtcctgtcgctgcat
taatcgcatctacagttgcactagctgttagccctctttcattcttaaatgtagctgataagtttaaacaagctgatttaatc
aaatcatattctgaacgcttccaaaaattaggatatgatggagatcgtttattagctgattttcaccgtgagacaggaactat
tgatgcttctgtaacaacaattaacactgctttagcagctatctccggtggagttggagctgcaagcgcgggttctctagtcg
gagctccagttgcgttactcgttgctggtgttacgggacttattacaactattctagaatattctaaacaagccatgtttgaa
catgttgcaaataaggttcatgacagaatagttgaatgggagaaaaaacataataaaaactattttgagcaaggttatgattc
tcgtcatttagctgatttacaagacaatatgaagtttcttatcaatttaaataaagaacttcaggctgaacgcgtagtagcta
ttacccaacaaagatgggataaccaaattggagacctagcggcaattagccgtagaacggataaaatttccagtggaaaagct
tatgtggatgcttttgaggaggggcaacaccagtcctacgattcatccgtacagctagataacaaaaacggtattattaatat
tagtaatacaaatagaaagacacaaagtgttttattcagaactccattactaactccaggtgaagagaatcgggaacgtattc
aggaaggtgcaaattcttatattacaaaattacatatacaaagagttgacagttggactgtaacagatggtgatgctagctca
agcgtagatttcactaatgtagtacaacgaatcgctgtgaaatttgatgatgcaggtaacattatcgaatctaaagatactaa
aattatcgcaaatttaggtgctggtaacgataatgtatttgttgggtcaagtactaccgttattgatggcggggacggacatg
atcgagttcactacagtagaggagaatatggcgcattagttattgatgctacagccgagacagaaaaaggctcatattcagta
aaacgctatgtcggagacagtaaagcattacatgaaacaattgccacccaccaaacaaatgttggtgctcgtgaagaaaaaat
tgaatatcgtcgtgaagatgatcgttttcatactggttatactgtgacggactcactcaaatcagttgaagagatcattggtt
cacaatttaatgatattttcaaaggaagccaatttgatgatgtgttccatggtggtaatggtgtagacactattgatggtaac
gatggtgacgatcatttatttggtggcgcaggcgatgatgttatcgatggaggaaacggtaacaatttccttgttggaggaac
cggtaatgatattatctcgggaggtaaagataatgatatttatgtccataaaacaggcgatggaaatgattctattacagact
ctggcggacaagataaactggcattttcggatgtaaatcttaaagacctcacctttaagaaagtagattcttctctcgaaatc
attaatcaaaaaggagaaaaagttcgtattgggaattggttcttagaagatgatttggctagcacagttgctaactataaagc
tacgaatgaccgaaaaattgaggaaattattggtaaaggaggagaacgtattacatcagaacaagttgataaactgattaagg
agggtaacaatcaaatctctgcagaagcattatccaaagttgtgaatgattacaatacgagtaaagatagacagaacgtatct
aatagcttagcaaaattgatttcttcagtcgggagctttacgtcttcctcagactttaggaataatttaggaacatatgttcc
ttcatcaatagatgtctcgaataatattcaattagctagagccgcttaa
APP ApxIIIA K571A K702A
SEQ ID NO: 14
atgagtacttggtcaagcatgttagccgacttaaaaaaacgggctgaagaagccaaaagacaagccaaaaaaggctacgatgt
aactaaaaatggtttgcaatatggggtgagtcaagcaaaattacaagcattagcagctggtaaagccgttcaaaagtacggta
ataaattagttttagttattccaaaagagtatgacggaagtgttggtaacggtttctttgatttagtaaaagcagctgaggaa
ttaggcattcaagttaaatatgttaaccgtaatgaattggaagttgcccataaaagtttaggtaccgcagaccaattcttggg
tttaacagaacgtggacttactttatttgcaccgcaactagatcagttcttacaaaaacattcaaaaatttctaacgtagtgg
gcagttctactggtgatgcagtaagtaaacttgctaagagtcaaactattatttcaggaattcaatctgtattaggtactgta
ttagcaggtattaatcttaatgaagctattattagtggcggttcagagctcgaattagctgaagctggtgtttctttagcctc
tgagctcgttagtaatattgctaaaggtacaacaacaatagatgctttcactacacaaatccagaactttgggaaattagtgg
aaaatgctaaagggttaggtggtgttggccgccaattacagaatatttcaggttctgcattaagcaaaactggattaggtttg
gatattatctcaagcttactttcaggagtaactgcaagttttgctttagcgaataagaatgcttcaacaagcactaaagttgc
tgctggctttgaactctcaaatcaagtaattggtggtattacgaaagcagtatcaagctatattcttgcacagcgtttagctg
ctggtttatcaacgacaggtcctgctgcagcactaattgcgtctagtatttctttagcaatcagtccattggcgtttttacgt
gtagctgataattttaatcgttctaaagaaattggcgaatttgctgaacgtttcaaaaaattgggctatgacggcgataaact
actttcagagttttatcacgaagctggtactattgatgcctcaattactacaattagtacagcactttctgctatcgcagctg
gaacggccgccgcgagtgcaggtgcattagttggcgcaccaattactttgttggttactggtatcacaggattaatttctggt
attttagagttctctaaacaaccaatgttagatcatgttgcatcgaaaattggtaacaaaattgacgaatgggagaaaaaata
cggtaaaaattacttcgagaatggctatgatgctcgtcataaagctttcttagaagattcattctcattattgtctagtttta
ataaacaatatgaaactgaaagagctgttttaattacacaacaacgttgggatgaatatattggcgaacttgcgggtattact
ggcaaaggtgacaaactctctagtggtaaggcgtatgtagattactttcaagaaggtaaattattagagaaaaaacctgatga
ctttagcaaagtagttttcgatccaactaagggcgaaattgatatttcaaatagccaaacgtcaacgttgttaaaatttgtta
cgccattattaacaccaggtacagagtcacgtgaaagaactcaaacaggtgcatatgaatatatcacgaagttagttgtaaaa
ggtaaagataaatgggttgttaatggcgttaaagataaaggtgccgtttatgattatactaatttaattcaacatgctcatat
tagttcatcagtagcacgtggtgaagaataccgtgaagttcgtttggtatctcatctaggcaatggtaatgacaaagtgttct
tagctgcgggttccgcagaaattcacgctggtgaaggtcatgatgtggtttattatgataaaaccgatacaggtcttttagta
attgatggaaccaaagcgactgaacaagggcgttattctgttacgcgcgaattgagtggtgctacaaaaatcctgagagaagt
aataaaaaatcaaaaatctgctgttggtgcacgtgaagaaaccttggaatatcgtgattatgaattaacgcaatcaggtaata
gtaacctaaaagcacatgatgaattacattcagtagaagaaattattggaagtaatcagagagacgaatttaaaggtagtaaa
ttcagagatattttccatggtgccgatggtgatgatctattaaatggtaatgatggggatgatattctatacggtgataaagg
taacgatgagttaagaggtgataatggtaacgaccaactttatggtggtgaaggtaatgacaaactattaggaggtaatggca
ataattacctcagtggtggtgatggcaatgatgagcttcaagtcttaggcaatggttttaatgtgcttcgtggcggtaaaggc
gatgataaactttatggtagctcaggttctgatttacttgatggtggagaaggtaatgattatctagaaggaggcgatggtag
cgatttttatgtttatcgttccacttcaggtaatcatactatttatgatcaaggtaaatctagtgatttagataaactatatt
tgtctgatttttccttcgatcgtcttcttgttgagaaagttgatgataaccttgtacttagaagtaatgaaagtagtcataat
aatggagtactcacaatcaaagactggtttaaagaagggaataaatataaccataaaattgaacaaattgttgataaaaatgg
tagaaaattgacagcagagaatttaggaacttatttcaaaaatgctccaaaagctgacaatttgcttaattatgcaactaaag
aagatcagaatgaaagcaatttatcttcacttaaaactgaattaagtaaaattattactaatgcaggtaattttggtgtggca
aaacaaggtaatactggaatcaatacagctgccttgaacaatgaagtgaataaaatcatttcttctgctaatacctttgctac
ttcacaattgggtggctcagggatgggaacattaccatcaacgaatgtaaattcaatgatgctaggtaacctagctagagcag
cttaa
Plasmid pEX-A258
SEQ ID NO: 15
gtggcagctctagagctagcgaattctttggtgaaattgttatccgctcacaattccacacaacatacgagccggaagcataa
agtgtaaagcctggggtgcctaatgagtgagctaactcacattaattgcgttgcgctcactgcccgctttccagtcgggaaac
ctgtcgtgccagctgcattaatgaatcggccaacgcgcggggagaggcggtttgcgtattgggcgctcttccgcttcctcgct
cactgactcgctgcgctcggtcgttcggctgcggcgagcggtatcagctcactcaaaggcggtaatacggttatccacagaat
caggggataacgcaggaaagaacatgtgagcaaaaggccagcaaaaggccaggaaccgtaaaaaggccgcgttgctggcgttt
ttccataggctccgcccccctgacgagcatcacaaaaatcgacgctcaagtcagaggtggcgaaacccgacaggactataaag
ataccaggcgtttccccctggaagctccctcgtgcgctctcctgttccgaccctgccgcttaccggatacctgtccgcctttc
tcccttcgggaagcgtggcgctttctcatagctcacgctgtaggtatctcagttcggtgtaggtcgttcgctccaagctgggc
tgtgtgcacgaaccccccgttcagcccgaccgctgcgccttatccggtaactatcgtcttgagtccaacccggtaagacacga
cttatcgccactggcagcagccactggtaacaggattagcagagcgaggtatgtaggcggtgctacagagttcttgaagtggt
ggcctaactacggctacactagaagaacagtatttggtatctgcgctctgctgaagccagttaccttcggaaaaagagttggt
agctcttgatccggcaaacaaaccaccgctggtagcggtggtttttttgtttgcaagcagcagattacgcgcagaaaaaaagg
atctcaagaagatcctttgatcttttctacggggtctgacgctcagtggaacgaaaactcacgttaagggattttggtcatga
gattatcaaaaaggatcttcacctagatccttttaaattaaaaatgaagttttaaatcaatctaaagtatatatgagtaaact
tggtctgacagttaccaatgcttaatcagtgaggcacctatctcagcgatctgtctatttcgttcatccatagttgcctgact
ccccgtcgtgtagataactacgatacgggagggcttaccatctggccccagtgctgcaatgataccgcgcgaaccacgctcac
cggctccagatttatcagcaataaaccagccagccggaagggccgagcgcagaagtggtcctgcaactttatccgcctccatc
cagtctattaattgttgccgggaagctagagtaagtagttcgccagttaatagtttgcgcaacgttgttgccattgctacagg
catcgtggtgtcacgctcgtcgtttggtatggcttcattcagctccggttcccaacgatcaaggcgagttacatgatccccca
tgttgtgcaaaaaagcggttagctccttcggtcctccgatcgttgtcagaagtaagttggccgcagtgttatcactcatggtt
atggcagcactgcataattctcttactgtcatgccatccgtaagatgcttttctgtgactggtgagtactcaaccaagtcatt
ctgagaatagtgtatgcggcgaccgagttgctcttgcccggcgtcaatacgggataataccgcgccacatagcagaactttaa
aagtgctcatcattggaaaacgttcttcggggcgaaaactctcaaggatcttaccgctgttgagatccagttcgatgtaaccc
actcgtgcacccaactgatcttcagcatcttttactttcaccagcgtttctgggtgagcaaaaacaggaaggcaaaatgccgc
aaaaaagggaataagggcgacacggaaatgttgaatactcatactcttcctttttcaatattattgaagcatttatcagggtt
attgtctcatgagcggatacatatttgaatgtatttagaaaaataaacaaataggggttccgcgcacatttccccgaaaagtg
ccacctgacgtctaagaaaccattattatcatgacattaacctataaaaataggcgtatcacgaggccctttcgtctcgcgcg
tttcggtgatgacggtgaaaacctctgacacatgcagctcccggagacggtcacagcttgtctgtaagcggatgccgggagca
gacaagcccgtcagggcgcgtcagcgggtgttggcgggtgtcggggctggcttaactatgcggcatcagagcagattgtactg
agagtttggcaattggtcgacctcgagggcgcgcccgta
Plasmid pQE-80L
SEQ ID NO: 16
ctcgagaaatcataaaaaatttatttgctttgtgagcggataacaattataatagattcaattgtgagcggataacaatttca
cacagaattcattaaagaggagaaattaactatgagaggatcgcatcaccatcaccatcacggatccgcatgcgagctcggta
ccccgggtcgacctgcagccaagcttaattagctgagcttggactcctgttgatagatccagtaatgacctcagaactccatc
tggatttgttcagaacgctcggttgccgccgggcgttttttattggtgagaatccaagctagcttggcgagattttcaggagc
taaggaagctaaaatggagaaaaaaatcactggatataccaccgttgatatatcccaatggcatcgtaaagaacattttgagg
catttcagtcagttgctcaatgtacctataaccagaccgttcagctggatattacggcctttttaaagaccgtaaagaaaaat
aagcacaagttttatccggcctttattcacattcttgcccgcctgatgaatgctcatccggaatttcgtatggcaatgaaaga
cggtgagctggtgatatgggatagtgttcacccttgttacaccgttttccatgagcaaactgaaacgttttcatcgctctgga
gtgaataccacgacgatttccggcagtttctacacatatattcgcaagatgtggcgtgttacggtgaaaacctggcctatttc
cctaaagggtttattgagaatatgtttttcgtctcagccaatccctgggtgagtttcaccagttttgatttaaacgtggccaa
tatggacaacttcttcgcccccgttttcaccatgggcaaatattatacgcaaggcgacaaggtgctgatgccgctggcgattc
aggttcatcatgccgtttgtgatggcttccatgtcggcagaatgcttaatgaattacaacagtactgcgatgagtggcagggc
ggggcgtaatttttttaaggcagttattggtgcccttaaacgcctggggtaatgactctctagcttgaggcatcaaataaaac
gaaaggctcagtcgaaagactgggcctttcgttttatctgttgtttgtcggtgaacgctctcctgagtaggacaaatccgccc
tctagattacgtgcagtcgatgataagctgtcaaacatgagaattgtgcctaatgagtgagctaacttacattaattgcgttg
cgctcactgcccgctttccagtcgggaaacctgtcgtgccagctgcattaatgaatcggccaacgcgcggggagaggcggttt
gcgtattgggcgccagggtggtttttcttttcaccagtgagacgggcaacagctgattgcccttcaccgcctggccctgagag
agttgcagcaagcggtccacgctggtttgccccagcaggcgaaaatcctgtttgatggtggttaacggcgggatataacatga
gctgtcttcggtatcgtcgtatcccactaccgagatatccgcaccaacgcgcagcccggactcggtaatggcgcgcattgcgc
ccagcgccatctgatcgttggcaaccagcatcgcagtgggaacgatgccctcattcagcatttgcatggtttgttgaaaaccg
gacatggcactccagtcgccttcccgttccgctatcggctgaatttgattgcgagtgagatatttatgccagccagccagacg
cagacgcgccgagacagaacttaatgggcccgctaacagcgcgatttgctggtgacccaatgcgaccagatgctccacgccca
gtcgcgtaccgtcttcatgggagaaaataatactgttgatgggtgtctggtcagagacatcaagaaataacgccggaacatta
gtgcaggcagcttccacagcaatggcatcctggtcatccagcggatagttaatgatcagcccactgacgcgttgcgcgagaag
attgtgcaccgccgctttacaggcttcgacgccgcttcgttctaccatcgacaccaccacgctggcacccagttgatcggcgc
gagatttaatcgccgcgacaatttgcgacggcgcgtgcagggccagactggaggtggcaacgccaatcagcaacgactgtttg
cccgccagttgttgtgccacgcggttgggaatgtaattcagctccgccatcgccgcttccactttttcccgcgttttcgcaga
aacgtggctggcctggttcaccacgcgggaaacggtctgataagagacaccggcatactctgcgacatcgtataacgttactg
gtttcacattcaccaccctgaattgactctcttccgggcgctatcatgccataccgcgaaaggttttgcaccattcgatggtg
tcggaatttcgggcagcgttgggtcctggccacgggtgcgcatgatctagagctgcctcgcgcgtttcggtgatgacggtgaa
aacctctgacacatgcagctcccggagacggtcacagcttgtctgtaagcggatgccgggagcagacaagcccgtcagggcgc
gtcagcgggtgttggcgggtgtcggggcgcagccatgacccagtcacgtagcgatagcggagtgtatactggcttaactatgc
ggcatcagagcagattgtactgagagtgcaccatatgcggtgtgaaataccgcacagatgcgtaaggagaaaataccgcatca
ggcgctcttccgcttcctcgctcactgactcgctgcgctcggtcgttcggctgcggcgagcggtatcagctcactcaaaggcg
gtaatacggttatccacagaatcaggggataacgcaggaaagaacatgtgagcaaaaggccagcaaaaggccaggaaccgtaa
aaaggccgcgttgctggcgtttttccataggctccgcccccctgacgagcatcacaaaaatcgacgctcaagtcagaggtggc
gaaacccgacaggactataaagataccaggcgtttccccctggaagctccctcgtgcgctctcctgttccgaccctgccgctt
accggatacctgtccgcctttctcccttcgggaagcgtggcgctttctcatagctcacgctgtaggtatctcagttcggtgta
ggtcgttcgctccaagctgggctgtgtgcacgaaccccccgttcagcccgaccgctgcgccttatccggtaactatcgtcttg
agtccaacccggtaagacacgacttatcgccactggcagcagccactggtaacaggattagcagagcgaggtatgtaggcggt
gctacagagttcttgaagtggtggcctaactacggctacactagaaggacagtatttggtatctgcgctctgctgaagccagt
taccttcggaaaaagagttggtagctcttgatccggcaaacaaaccaccgctggtagcggtggtttttttgtttgcaagcagc
agattacgcgcagaaaaaaaggatctcaagaagatcctttgatcttttctacggggtctgacgctcagtggaacgaaaactca
cgttaagggattttggtcatgagattatcaaaaaggatcttcacctagatccttttaaattaaaaatgaagttttaaatcaat
ctaaagtatatatgagtaaacttggtctgacagttaccaatgcttaatcagtgaggcacctatctcagcgatctgtctatttc
gttcatccatagttgcctgactccccgtcgtgtagataactacgatacgggagggcttaccatctggccccagtgctgcaatg
ataccgcgagacccacgctcaccggctccagatttatcagcaataaaccagccagccggaagggccgagcgcagaagtggtcc
tgcaactttatccgcctccatccagtctattaattgttgccgggaagctagagtaagtagttcgccagttaatagtttgcgca
acgttgttgccattgctacaggcatcgtggtgtcacgctcgtcgtttggtatggcttcattcagctccggttcccaacgatca
aggcgagttacatgatcccccatgttgtgcaaaaaagcggttagctccttcggtcctccgatcgttgtcagaagtaagttggc
cgcagtgttatcactcatggttatggcagcactgcataattctcttactgtcatgccatccgtaagatgcttttctgtgactg
gtgagtactcaaccaagtcattctgagaatagtgtatgcggcgaccgagttgctcttgcccggcgtcaatacgggataatacc
gcgccacatagcagaactttaaaagtgctcatcattggaaaacgttcttcggggcgaaaactctcaaggatcttaccgctgtt
gagatccagttcgatgtaacccactcgtgcacccaactgatcttcagcatcttttactttcaccagcgtttctgggtgagcaa
aaacaggaaggcaaaatgccgcaaaaaagggaataagggcgacacggaaatgttgaatactcatactcttcctttttcaatat
tattgaagcatttatcagggttattgtctcatgagcggatacatatttgaatgtatttagaaaaataaacaaataggggttcc
gcgcacatttccccgaaaagtgccacctgacgtctaagaaaccattattatcatgacattaacctataaaaataggcgtatca
cgaggccctttcgtcttcac
Plasmid pQE-60
SEQ ID NO: 17
ctcgagaaatcataaaaaatttatttgctttgtgagcggataacaattataatagattcaattgtgagcggataacaatttca
cacagaattcattaaagaggagaaattaaccatgggaggatccagatctcatcaccatcaccatcactaagcttaattagctg
agcttggactcctgttgatagatccagtaatgacctcagaactccatctggatttgttcagaacgctcggttgccgccgggcg
ttttttattggtgagaatccaagctagcttggcgagattttcaggagctaaggaagctaaaatggagaaaaaaatcactggat
ataccaccgttgatatatcccaatggcatcgtaaagaacattttgaggcatttcagtcagttgctcaatgtacctataaccag
accgttcagctggatattacggcctttttaaagaccgtaaagaaaaataagcacaagttttatccggcctttattcacattct
tgcccgcctgatgaatgctcatccggaatttcgtatggcaatgaaagacggtgagctggtgatatgggatagtgttcaccctt
gttacaccgttttccatgagcaaactgaaacgttttcatcgctctggagtgaataccacgacgatttccggcagtttctacac
atatattcgcaagatgtggcgtgttacggtgaaaacctggcctatttccctaaagggtttattgagaatatgtttttcgtctc
agccaatccctgggtgagtttcaccagttttgatttaaacgtggccaatatggacaacttcttcgcccccgttttcaccatgc
atgggcaaatattatacgcaaggcgacaaggtgctgatgccgctggcgattcaggttcatcatgccgtctgtgatggcttcca
tgtcggcagaatgcttaatgaattacaacagtactgcgatgagtggcagggcggggcgtaatttttttaaggcagttattggt
gcccttaaacgcctggggtaatgactctctagcttgaggcatcaaataaaacgaaaggctcagtcgaaagactgggcctttcg
ttttatctgttgtttgtcggtgaacgctctcctgagtaggacaaatccgccgctctagagctgcctcgcgcgtttcggtgatg
acggtgaaaacctctgacacatgcagctcccggagacggtcacagcttgtctgtaagcggatgccgggagcagacaagcccgt
cagggcgcgtcagcgggtgttggcgggtgtcggggcgcagccatgacccagtcacgtagcgatagcggagtgtatactggctt
aactatgcggcatcagagcagattgtactgagagtgcaccatatgcggtgtgaaataccgcacagatgcgtaaggagaaaata
ccgcatcaggcgctcttccgcttcctcgctcactgactcgctgcgctcggtcgttcggctgcggcgagcggtatcagctcact
caaaggcggtaatacggttatccacagaatcaggggataacgcaggaaagaacatgtgagcaaaaggccagcaaaaggccagg
aaccgtaaaaaggccgcgttgctggcgtttttccataggctccgcccccctgacgagcatcacaaaaatcgacgctcaagtca
gaggtggcgaaacccgacaggactataaagataccaggcgtttccccctggaagctccctcgtgcgctctcctgttccgaccc
tgccgcttaccggatacctgtccgcctttctcccttcgggaagcgtggcgctttctcatagctcacgctgtaggtatctcagt
tcggtgtaggtcgttcgctccaagctgggctgtgtgcacgaaccccccgttcagcccgaccgctgcgccttatccggtaacta
tcgtcttgagtccaacccggtaagacacgacttatcgccactggcagcagccactggtaacaggattagcagagcgaggtatg
taggcggtgctacagagttcttgaagtggtggcctaactacggctacactagaaggacagtatttggtatctgcgctctgctg
aagccagttaccttcggaaaaagagttggtagctcttgatccggcaaacaaaccaccgctggtagcggtggtttttttgtttg
caagcagcagattacgcgcagaaaaaaaggatctcaagaagatcctttgatcttttctacggggtctgacgctcagtggaacg
aaaactcacgttaagggattttggtcatgagattatcaaaaaggatcttcacctagatccttttaaattaaaaatgaagtttt
aaatcaatctaaagtatatatgagtaaacttggtctgacagttaccaatgcttaatcagtgaggcacctatctcagcgatctg
tctatttcgttcatccatagttgcctgactccccgtcgtgtagataactacgatacgggagggcttaccatctggccccagtg
ctgcaatgataccgcgagacccacgctcaccggctccagatttatcagcaataaaccagccagccggaagggccgagcgcaga
agtggtcctgcaactttatccgcctccatccagtctattaattgttgccgggaagctagagtaagtagttcgccagttaatag
tttgcgcaacgttgttgccattgctacaggcatcgtggtgtcacgctcgtcgtttggtatggcttcattcagctccggttccc
aacgatcaaggcgagttacatgatcccccatgttgtgcaaaaaagcggttagctccttcggtcctccgatcgttgtcagaagt
aagttggccgcagtgttatcactcatggttatggcagcactgcataattctcttactgtcatgccatccgtaagatgcttttc
tgtgactggtgagtactcaaccaagtcattctgagaatagtgtatgcggcgaccgagttgctcttgcccggcgtcaatacggg
ataataccgcgccacatagcagaactttaaaagtgctcatcattggaaaacgttcttcggggcgaaaactctcaaggatctta
ccgctgttgagatccagttcgatgtaacccactcgtgcacccaactgatcttcagcatcttttactttcaccagcgtttctgg
gtgagcaaaaacaggaaggcaaaatgccgcaaaaaagggaataagggcgacacggaaatgttgaatactcatactcttccttt
ttcaatattattgaagcatttatcagggttattgtctcatgagcggatacatatttgaatgtatttagaaaaataaacaaata
ggggttccgcgcacatttccccgaaaagtgccacctgacgtctaagaaaccattattatcatgacattaacctataaaaatag
gcgtatcacgaggccctttcgtcttcac
dfrA14sacB cassette
SEQ ID NO: 18
gttaatgccgtctgaagtgcgaagcggcatcagagcagattgtactgagagtgcaccatatggtcgacctcgagttaattaac
gtatgcggccgctttagactatttaaataatattatttaaattctttactatagtgtacaatacacacagtccattaaccaaa
ataaaaggaggaattaggatgagaaccttgaaagtatcattgatagctgcgaaagcgaaaaacggcgtgattggttgcggtcc
agacataccctggtccgcgaaaggggagcagctactttttaaagcattgacctacaatcagtggcttctggtgggtcgcaaga
cgtttgaatctatgggcgcactccccaataggaaatacgcggtcgttacccgctcaggttggacatcaaatgatgacaatgta
gttgtatttcagtcaatcgaagaggccatggacaggctagctgaattcaccggtcacgttatagtgtctggtggcggagaaat
ttaccgagaaacattacccatggcctctacgctccacttatcgacgatcgacatcgagccagagggggatgttttcttcccga
gtattccaaataccttcgaagttgtttttgagcaacactttacttcaaacattaactattgctatcaaatttggaaaaagggt
taatgccgtctgaagtgcggtacaagcggtagaacctgccccgttagttgaaaccgcttgttatgcatgcatgggatccgcga
atcccgcggccatggcggccgggagcatgcgacgtcgggcccattgggatccgcttttacagcgattgcagaatgattgaatt
gtaaactttagagctttatattttgtttaatggtattatatttacttatatttatgattcttagtttttattgtaaattaaag
tgtttatttattgtattttaagtataagatcctttttaacccatcacatatacctgccgttcactattatttagtgaaatgag
atattatgatattttctgaattgtgattaaaaaggcaactttatgcccatgcaacagaaactataaaaaatacagagaatgaa
aagaaacagatagattttttagttctttaggcccgtagtctgcaaatccttttatgattttctatcaaacaaaagaggaaaat
agaccagttgcaatccaaacgagagtctaatagaatgaggtcgaaaagtaaatcgcgcgggtttgttactgataaagcaggca
agacctaaaatgtgtaaagggcaaagtgtatactttggcgtcaccccttacatattttaggtctttttttattgtgcgtaact
aacttgccatcttcaaacaggagggctggaagaagcagaccgctaacacagtacataaaaaaggagacatgaacgatgaacat
caaaaagtttgcaaaacaagcaacagtattaacctttactaccgcactgctggcaggaggcgcaactcaagcgtttgcgaaag
aaacgaaccaaaagccatataaggaaacatacggcatttcccatattacacgccatgatatgctgcaaatccctgaacagcaa
aaaaatgaaaaatatcaagttcctgaattcgattcgtccacaattaaaaatatctcttctgcaaaaggcctggacgtttggga
cagctggccattacaaaacgctgacggcactgtcgcaaactatcacggctaccacatcgtctttgcattagccggagatccta
aaaatgcggatgacacatcgatttacatgttctatcaaaaagtcggcgaaacttctattgacagctggaaaaacgctggccgc
gtctttaaagacagcgacaaattcgatgcaaatgattctatcctaaaagaccaaacacaagaatggtcaggttcagccacatt
tacatctgacggaaaaatccgtttattctacactgatttctccggtaaacattacggcaaacaaacactgacaactgcacaag
ttaacgtatcagcatcagacagctctttgaacatcaacggtgtagaggattataaatcaatctttgacggtgacggaaaaacg
tatcaaaatgtacagcagttcatcgatgaaggcaactacagctcaggcgacaaccatacgctgagagatcctcactacgtaga
agataaaggccacaaatacttagtatttgaagcaaacactggaactgaagatggctaccaaggcgaagaatctttatttaaca
aagcatactatggcaaaagcacatcattcttccgtcaagaaagtcaaaaacttctgcaaagcgataaaaaacgcacggctgag
ttagcaaacggcgctctcggtatgattgagctaaacgatgattacacactgaaaaaagtgatgaaaccgctgattgcatctaa
cacagtaacagatgaaattgaacgcgcgaacgtctttaaaatgaacggcaaatggtacctgttcactgactcccgcggatcaa
aaatgacgattgacggcattacgtctaacgatatttacatgcttggttatgtttctaattctttaactggcccatacaagccg
ctgaacaaaactggccttgtgttaaaaatggatcttgatcctaacgatgtaacctttacttactcacacttcgctgtacctca
agcgaaaggaaacaatgtcgtgattacaagctatatgacaaacagaggattctacgcagacaaacaatcaacgtttgcgccaa
gcttcctgctgaacatcaaaggcaagaaaacatctgttgtcaaagacagcatccttgaacaaggacaattaacagttaacaaa
taaaaacgcaaaagaaaatgccgatatcctattggcattttcttttatttcttatcaacataaaggtgaatcccatacctaga
gctgcacgcgagacatgaacgtgcaactgcttc
linker tri OE for
SEQ ID NO: 19
gttaatgccg tctgaagtgc gaag
linker sac OE rev
SEQ ID NO: 20
gaagcagttg cacgttcatg tctc
left flank for primer
SEQ ID NO: 21
attgggtacc gagctcgc
tri OE rev primer
SEQ ID NO: 22
cttcgcactt cagacggcat taac
sac OE for primer
SEQ ID NO: 23
gagacatgaa cgtgcaactg cttc
right flank rev primer
SEQ ID NO: 24
ccatttcaca caggaattcg gatc
for primer
SEQ ID NO: 25
aaacaagcgg tccggatctt ggaatttcgg c
rev primer
SEQ ID NO: 26
tgccttcaag cggatcaaac ac
for primer
SEQ ID NO: 27
tcgaacttgg gaacggtatc ag
rev primer
SEQ ID NO: 28
ttacaagcgg tactttgcca gcttacctac gatg
apxIA mut for OE
SEQ ID NO: 29
gtgtttgatc cgcttgaagg ca
apxIA mut rev OE
SEQ ID NO: 30
ctgataccgt tcccaagttc ga
left flank for USS
SEQ ID NO: 31
atccacaagc ggtcatctgg c
sxy TS LF for
SEQ ID NO: 32
gtaccgcttg ttaaatgatt acacc
Sxy TS LF rev1
SEQ ID NO: 33
ggcattaact tagttagcct gtgagatagc
Sxy TS LF rev2
SEQ ID NO: 34
cttcgcactt cagacggcat taacttagtt agcctgtgag
delta apxIA dfrA14sacB
SEQ ID NO: 35
attgggtaccgagctcgcggccgcaccggctcattagtcggtgcgccggtagcagctttagttagtgcaatcaccggtattat
ttcaggtattttagatgcttctaaacaggcaatcttcgaacgagttgcaacgaaattagcgaataagattgacgaatgggaga
aaaaacacggtaaaaactattttgaaaacggttatgacgcccgccattccgcattcttagaagatacctttgaattgttatca
caatacaataaagagtattcggtagagcgtgtcgttgctattacgcaacaacgttgggatgtcaatatcggggaacttgccgg
tatcacgcgtaaaggtgcggatgcgaaaagcggtaaggcttatgtcgatttctttgaagaaggaaaattgttagagaaagatc
cggatcgttttgataaaaaagtgtttgatccgcttgaaggcaaaatcgacctttcttcaattaacaaaaccactttattgaaa
tttattacaccggtttttaccgcaggtgttaatgccgtctgaagtgcgaagcggcatcagagcagattgtactgagagtgcac
catatggtcgacctcgagttaattaacgtatgcggccgctttagactatttaaataatattatttaaattctttactatagtg
tacaatacacacagtccattaaccaaaataaaaggaggaattaggatgagaaccttgaaagtatcattgatagctgcgaaagc
gaaaaacggcgtgattggttgcggtccagacataccctggtccgcgaaaggggagcagctactttttaaagcattgacctaca
atcagtggcttctggtgggtcgcaagacgtttgaatctatgggcgcactccccaataggaaatacgcggtcgttacccgctca
ggttggacatcaaatgatgacaatgtagttgtatttcagtcaatcgaagaggccatggacaggctagctgaattcaccggtca
cgttatagtgtctggtggcggagaaatttaccgagaaacattacccatggcctctacgctccacttatcgacgatcgacatcg
agccagagggggatgttttcttcccgagtattccaaataccttcgaagttgtttttgagcaacactttacttcaaacattaac
tattgctatcaaatttggaaaaagggttaatgccgtctgaagtgcggtacaagcggtagaacctgccccgttagttgaaaccg
cttgttatgcatgcatgggatccgcgaatcccgcggccatggcggccgggagcatgcgacgtcgggcccattgggatccgctt
ttacagcgattgcagaatgattgaattgtaaactttagagctttatattttgtttaatggtattatatttacttatatttatg
attcttagtttttattgtaaattaaagtgtttatttattgtattttaagtataagatcctttttaacccatcacatatacctg
ccgttcactattatttagtgaaatgagatattatgatattttctgaattgtgattaaaaaggcaactttatgcccatgcaaca
gaaactataaaaaatacagagaatgaaaagaaacagatagattttttagttctttaggcccgtagtctgcaaatccttttatg
attttctatcaaacaaaagaggaaaatagaccagttgcaatccaaacgagagtctaatagaatgaggtcgaaaagtaaatcgc
gcgggtttgttactgataaagcaggcaagacctaaaatgtgtaaagggcaaagtgtatactttggcgtcaccccttacatatt
ttaggtctttttttattgtgcgtaactaacttgccatcttcaaacaggagggctggaagaagcagaccgctaacacagtacat
aaaaaaggagacatgaacgatgaacatcaaaaagtttgcaaaacaagcaacagtattaacctttactaccgcactgctggcag
gaggcgcaactcaagcgtttgcgaaagaaacgaaccaaaagccatataaggaaacatacggcatttcccatattacacgccat
gatatgctgcaaatccctgaacagcaaaaaaatgaaaaatatcaagttcctgaattcgattcgtccacaattaaaaatatctc
ttctgcaaaaggcctggacgtttgggacagctggccattacaaaacgctgacggcactgtcgcaaactatcacggctaccaca
tcgtctttgcattagccggagatcctaaaaatgcggatgacacatcgatttacatgttctatcaaaaagtcggcgaaacttct
attgacagctggaaaaacgctggccgcgtctttaaagacagcgacaaattcgatgcaaatgattctatcctaaaagaccaaac
acaagaatggtcaggttcagccacatttacatctgacggaaaaatccgtttattctacactgatttctccggtaaacattacg
gcaaacaaacactgacaactgcacaagttaacgtatcagcatcagacagctctttgaacatcaacggtgtagaggattataaa
tcaatctttgacggtgacggaaaaacgtatcaaaatgtacagcagttcatcgatgaaggcaactacagctcaggcgacaacca
tacgctgagagatcctcactacgtagaagataaaggccacaaatacttagtatttgaagcaaacactggaactgaagatggct
accaaggcgaagaatctttatttaacaaagcatactatggcaaaagcacatcattcttccgtcaagaaagtcaaaaacttctg
caaagcgataaaaaacgcacggctgagttagcaaacggcgctctcggtatgattgagctaaacgatgattacacactgaaaaa
agtgatgaaaccgctgattgcatctaacacagtaacagatgaaattgaacgcgcgaacgtctttaaaatgaacggcaaatggt
acctgttcactgactcccgcggatcaaaaatgacgattgacggcattacgtctaacgatatttacatgcttggttatgtttct
aattctttaactggcccatacaagccgctgaacaaaactggccttgtgttaaaaatggatcttgatcctaacgatgtaacctt
tacttactcacacttcgctgtacctcaagcgaaaggaaacaatgtcgtgattacaagctatatgacaaacagaggattctacg
cagacaaacaatcaacgtttgcgccaagcttcctgctgaacatcaaaggcaagaaaacatctgttgtcaaagacagcatcctt
gaacaaggacaattaacagttaacaaataaaaacgcaaaagaaaatgccgatatcctattggcattttcttttatttcttatc
aacataaaggtgaatcccatacctagagctgcacgcgagacatgaacgtgcaactgcttcccattcgaacttgggaacggtat
cagagctaaagatgaattacattctgttgaagaaattatcggtagtaatcgtaaagacaaattctttggtagtcgctttaccg
atattttccatggtgcgaaaggcgatgatgaaatctacggtaatgacggccacgatatcttatacggagacgacggtaatgat
gtaatccatggcggtgacggtaacgaccatcttgttggtggtaacggaaacgaccgattaatcggcggaaaaggtaataattt
ccttaatggcggtgatggtgacgatgagttgcaggtctttgagggtcaatacaacgtattattaggtggtgcgggtaatgaca
ttctgtatggcagcgatggtactaacttatttgacggtggtgtaggcaatgacaaaatctacggtggtttaggtaaggatatt
tatcgctacagtaaggagtacggtcgtcatatcattattgagaaaggcggtgatgatgatacggatccgaattcctgtgtgaa
atgg
apxIAmut
SEQ ID NO: 36
gaccgcggccgcaccggctcattagtcggtgcgccggtagcagctttagttagtgcaatcaccggtattatttcaggtatttt
agatgcttctaaacaggcaatcttcgaacgagttgcaacgaaattagcgaataagattgacgaatgggagaaaaaacacggta
aaaactattttgaaaacggttatgacgcccgccattccgcattcttagaagatacctttgaattgttatcacaatacaataaa
gagtattcggtagagcgtgtcgttgctattacgcaacaacgttgggatgtcaatatcggggaacttgccggtatcacgcgtaa
aggtgcggatgcgaaaagcggtaaggcttatgtcgatttctttgaagaaggaaaattgttagagaaagatccggatcgttttg
ataaaaaagtgtttgatccgcttgaaggcaaaatcgacctttcttcaattaacaaaaccactttattgaaatttattacaccg
gtttttaccgcaggtgaagagattcgtgagcgtaagcaaaccggtgcatacgaatatatgaccgaattattcgttaaaggtaa
agaaaaatgggtggtaaccggtgtgcagtcacataatgcgatttatgactatacgaatcttatccaattagcgatagataaaa
aaggtgaaaaacgtcaagtgaccattgaatctcatttgggtgagaaaaatgatcgtatatatctttcatccggttcatctatc
gtatatgcgggtaacggacatgatgtagcatattacgataaaaccgatacaggttacttaacatttgacggacaaagtgcaca
gaaagccggtgaatatattgtcactaaagaacttaaagctgatgtaaaagttttaaaagaagtggttaaaactcaggatattt
cagttggagcacgcagtgaaaaattagaatatcgtgattatgagttaagcccattcgaacttgggaacggtatcagagctaaa
gatgaattacattctgttgaagaaattatcggtagtaatcgtaaagacaaattctttggtagtcgctttaccgatattttcca
tggtgcgaaaggcgatgatgaaatctacggtaatgacggccacgatatcttatacggagacgacggtaatgatgtaatccatg
gcggtgacggtaacgaccatcttgttggtggtaacggaaacgaccgattaatcggcggaaaaggtaataatttccttaatggc
ggtgatggtgacgatgagttgcaggtctttgagggtcaatacaacgtattattaggtggtgcgggtaatgacattctgtatgg
cagcgatggtactaacttatttgacggtggtgtaggcaatgacaaaatctacggtggtttaggtaaggatatttatcgctaca
gtaaggagtacggtcgtcatatcattattgagaaaggcggtgatgatgatacaagcggtttg
delta apxIIA dfrA14sacB
SEQ ID NO: 37
attgggtaccgagctcgcggccgcgctgcaagcgcgggttctctagtcggagctccagttgcgttactcgttgctggtgttac
gggacttattacaactattctagaatattctaaacaagccatgtttgaacatgttgcaaataaggttcatgacagaatagttg
aatgggagaaaaaacataataaaaactattttgagcaaggttatgattctcgtcatttagctgatttacaagacaatatgaag
tttcttatcaatttaaataaagaacttcaggctgaacgcgtagtagctattacccaacaaagatgggataaccaaattggaga
cctagcggcaattagccgtagaacggataaaatttccagtggaaaagcttatgtggatgcttttgaggaggggcaacaccagt
cctacgattcatccgtacagctagataacaaaaacggtattattaatattagtaatacaaatagaaagacacaaagtgtttta
ttcagaactccattactaactccaggtgttaatgccgtctgaagtgcgaagcggcatcagagcagattgtactgagagtgcac
catatggtcgacctcgagttaattaacgtatgcggccgctttagactatttaaataatattatttaaattctttactatagtg
tacaatacacacagtccattaaccaaaataaaaggaggaattaggatgagaaccttgaaagtatcattgatagctgcgaaagc
gaaaaacggcgtgattggttgcggtccagacataccctggtccgcgaaaggggagcagctactttttaaagcattgacctaca
atcagtggcttctggtgggtcgcaagacgtttgaatctatgggcgcactccccaataggaaatacgcggtcgttacccgctca
ggttggacatcaaatgatgacaatgtagttgtatttcagtcaatcgaagaggccatggacaggctagctgaattcaccggtca
cgttatagtgtctggtggcggagaaatttaccgagaaacattacccatggcctctacgctccacttatcgacgatcgacatcg
agccagagggggatgttttcttcccgagtattccaaataccttcgaagttgtttttgagcaacactttacttcaaacattaac
tattgctatcaaatttggaaaaagggttaatgccgtctgaagtgcggtacaagcggtagaacctgccccgttagttgaaaccg
cttgttatgcatgcatgggatccgcgaatcccgcggccatggcggccgggagcatgcgacgtcgggcccattgggatccgctt
ttacagcgattgcagaatgattgaattgtaaactttagagctttatattttgtttaatggtattatatttacttatatttatg
attcttagtttttattgtaaattaaagtgtttatttattgtattttaagtataagatcctttttaacccatcacatatacctg
ccgttcactattatttagtgaaatgagatattatgatattttctgaattgtgattaaaaaggcaactttatgcccatgcaaca
gaaactataaaaaatacagagaatgaaaagaaacagatagattttttagttctttaggcccgtagtctgcaaatccttttatg
attttctatcaaacaaaagaggaaaatagaccagttgcaatccaaacgagagtctaatagaatgaggtcgaaaagtaaatcgc
gcgggtttgttactgataaagcaggcaagacctaaaatgtgtaaagggcaaagtgtatactttggcgtcaccccttacatatt
ttaggtctttttttattgtgcgtaactaacttgccatcttcaaacaggagggctggaagaagcagaccgctaacacagtacat
aaaaaaggagacatgaacgatgaacatcaaaaagtttgcaaaacaagcaacagtattaacctttactaccgcactgctggcag
gaggcgcaactcaagcgtttgcgaaagaaacgaaccaaaagccatataaggaaacatacggcatttcccatattacacgccat
gatatgctgcaaatccctgaacagcaaaaaaatgaaaaatatcaagttcctgaattcgattcgtccacaattaaaaatatctc
ttctgcaaaaggcctggacgtttgggacagctggccattacaaaacgctgacggcactgtcgcaaactatcacggctaccaca
tcgtctttgcattagccggagatcctaaaaatgcggatgacacatcgatttacatgttctatcaaaaagtcggcgaaacttct
attgacagctggaaaaacgctggccgcgtctttaaagacagcgacaaattcgatgcaaatgattctatcctaaaagaccaaac
acaagaatggtcaggttcagccacatttacatctgacggaaaaatccgtttattctacactgatttctccggtaaacattacg
gcaaacaaacactgacaactgcacaagttaacgtatcagcatcagacagctctttgaacatcaacggtgtagaggattataaa
tcaatctttgacggtgacggaaaaacgtatcaaaatgtacagcagttcatcgatgaaggcaactacagctcaggcgacaacca
tacgctgagagatcctcactacgtagaagataaaggccacaaatacttagtatttgaagcaaacactggaactgaagatggct
accaaggcgaagaatctttatttaacaaagcatactatggcaaaagcacatcattcttccgtcaagaaagtcaaaaacttctg
caaagcgataaaaaacgcacggctgagttagcaaacggcgctctcggtatgattgagctaaacgatgattacacactgaaaaa
agtgatgaaaccgctgattgcatctaacacagtaacagatgaaattgaacgcgcgaacgtctttaaaatgaacggcaaatggt
acctgttcactgactcccgcggatcaaaaatgacgattgacggcattacgtctaacgatatttacatgcttggttatgtttct
aattctttaactggcccatacaagccgctgaacaaaactggccttgtgttaaaaatggatcttgatcctaacgatgtaacctt
tacttactcacacttcgctgtacctcaagcgaaaggaaacaatgtcgtgattacaagctatatgacaaacagaggattctacg
cagacaaacaatcaacgtttgcgccaagcttcctgctgaacatcaaaggcaagaaaacatctgttgtcaaagacagcatcctt
gaacaaggacaattaacagttaacaaataaaaacgcaaaagaaaatgccgatatcctattggcattttcttttatttcttatc
aacataaaggtgaatcccatacctagagctgcacgcgagacatgaacgtgcaactgcttcgttttcatactggttatactgtg
acggactcactcaaatcagttgaagagatcattggttcacaatttaatgatattttcaaaggaagccaatttgatgatgtgtt
ccatggtggtaatggtgtagacactattgatggtaacgatggtgacgatcatttatttggtggcgcaggcgatgatgttatcg
atggaggaaacggtaacaatttccttgttggaggaaccggtaatgatattatctcgggaggtaaagataatgatatttatgtc
cataaaacaggcgatggaaatgattctattacagactctggcggacaagataaactggcattttcggatgtaaatcttaaaga
cctcacctttaagaaagtagattcttctctcgaaatcattaatcaaaaaggagaaaaagttcgtattgggaattggttcttag
aagatgatttggctagcacagttgctaactataaagctacgaatgaccgaaaaattgaggaagatccgaattcctgtgtgaaa
tgg
apxIIAmut
SEQ ID NO: 38
gaccgcggccgcgctgcaagcgcgggttctctagtcggagctccagttgcgttactcgttgctggtgttacgggacttattac
aactattctagaatattctaaacaagccatgtttgaacatgttgcaaataaggttcatgacagaatagttgaatgggagaaaa
aacataataaaaactattttgagcaaggttatgattctcgtcatttagctgatttacaagacaatatgaagtttcttatcaat
ttaaataaagaacttcaggctgaacgcgtagtagctattacccaacaaagatgggataaccaaattggagacctagcggcaat
tagccgtagaacggataaaatttccagtggaaaagcttatgtggatgcttttgaggaggggcaacaccagtcctacgattcat
ccgtacagctagataacaaaaacggtattattaatattagtaatacaaatagaaagacacaaagtgttttattcagaactcca
ttactaactccaggtgaagagaatcgggaacgtattcaggaaggtgcaaattcttatattacaaaattacatatacaaagagt
tgacagttggactgtaacagatggtgatgctagctcaagcgtagatttcactaatgtagtacaacgaatcgctgtgaaatttg
atgatgcaggtaacattatcgaatctaaagatactaaaattatcgcaaatttaggtgctggtaacgataatgtatttgttggg
tcaagtactaccgttattgatggcggggacggacatgatcgagttcactacagtagaggagaatatggcgcattagttattga
tgctacagccgagacagaaaaaggctcatattcagtaaaacgctatgtcggagacagtaaagcattacatgaaacaattgcca
cccaccaaacaaatgttggtgctcgtgaagaaaaaattgaatatcgtcgtgaagatgatcgttttcatactggttatactgtg
acggactcactcaaatcagttgaagagatcattggttcacaatttaatgatattttcaaaggaagccaatttgatgatgtgtt
ccatggtggtaatggtgtagacactattgatggtaacgatggtgacgatcatttatttggtggcgcaggcgatgatgttatcg
atggaggaaacggtaacaatttccttgttggaggaaccggtaatgatattatctcgggaggtaaagataatgatatttatgtc
cataaaacaggcgatggaaatgattctattacagactctggcggacaagataaactggcattttcggatgtaaatcttaaaga
cctcacctttaagaaagtagattcttctctcgaaatcattaatcaaaaaggagaaaaagttcgtattgggaattggttcttag
aagatgatttggctagcacagttgctaactataaagctacgaatgaccgaaaaattgaggacaagcggtttg
delta apxIIIA dfrA14sacB
SEQ ID NO: 39
attgggtaccgagctcgcggccgcgcaggtgcattagttggcgcaccaattactttgttggttactggtatcacaggattaat
ttctggtattttagagttctctaaacaaccaatgttagatcatgttgcatcgaaaattggtaacaaaattgacgaatgggaga
aaaaatacggtaaaaattacttcgagaatggctatgatgctcgtcataaagctttcttagaagattcattctcattattgtct
agttttaataaacaatatgaaactgaaagagctgttttaattacacaacaacgttgggatgaatatattggcgaacttgcggg
tattactggcaaaggtgacaaactctctagtggtaaggcgtatgtagattactttcaagaaggtaaattattagagaaaaaac
ctgatgactttagcaaagtagttttcgatccaactaagggcgaaattgatatttcaaatagccaaacgtcaacgttgttaaaa
tttgttacgccattattaacaccaggtgttaatgccgtctgaagtgcgaagcggcatcagagcagattgtactgagagtgcac
catatggtcgacctcgagttaattaacgtatgcggccgctttagactatttaaataatattatttaaattctttactatagtg
tacaatacacacagtccattaaccaaaataaaaggaggaattaggatgagaaccttgaaagtatcattgatagctgcgaaagc
gaaaaacggcgtgattggttgcggtccagacataccctggtccgcgaaaggggagcagctactttttaaagcattgacctaca
atcagtggcttctggtgggtcgcaagacgtttgaatctatgggcgcactccccaataggaaatacgcggtcgttacccgctca
ggttggacatcaaatgatgacaatgtagttgtatttcagtcaatcgaagaggccatggacaggctagctgaattcaccggtca
cgttatagtgtctggtggcggagaaatttaccgagaaacattacccatggcctctacgctccacttatcgacgatcgacatcg
agccagagggggatgttttcttcccgagtattccaaataccttcgaagttgtttttgagcaacactttacttcaaacattaac
tattgctatcaaatttggaaaaagggttaatgccgtctgaagtgcggtacaagcggtagaacctgccccgttagttgaaaccg
cttgttatgcatgcatgggatccgcgaatcccgcggccatggcggccgggagcatgcgacgtcgggcccattgggatccgctt
ttacagcgattgcagaatgattgaattgtaaactttagagctttatattttgtttaatggtattatatttacttatatttatg
attcttagtttttattgtaaattaaagtgtttatttattgtattttaagtataagatcctttttaacccatcacatatacctg
ccgttcactattatttagtgaaatgagatattatgatattttctgaattgtgattaaaaaggcaactttatgcccatgcaaca
gaaactataaaaaatacagagaatgaaaagaaacagatagattttttagttctttaggcccgtagtctgcaaatccttttatg
attttctatcaaacaaaagaggaaaatagaccagttgcaatccaaacgagagtctaatagaatgaggtcgaaaagtaaatcgc
gcgggtttgttactgataaagcaggcaagacctaaaatgtgtaaagggcaaagtgtatactttggcgtcaccccttacatatt
ttaggtctttttttattgtgcgtaactaacttgccatcttcaaacaggagggctggaagaagcagaccgctaacacagtacat
aaaaaaggagacatgaacgatgaacatcaaaaagtttgcaaaacaagcaacagtattaacctttactaccgcactgctggcag
gaggcgcaactcaagcgtttgcgaaagaaacgaaccaaaagccatataaggaaacatacggcatttcccatattacacgccat
gatatgctgcaaatccctgaacagcaaaaaaatgaaaaatatcaagttcctgaattcgattcgtccacaattaaaaatatctc
ttctgcaaaaggcctggacgtttgggacagctggccattacaaaacgctgacggcactgtcgcaaactatcacggctaccaca
tcgtctttgcattagccggagatcctaaaaatgcggatgacacatcgatttacatgttctatcaaaaagtcggcgaaacttct
attgacagctggaaaaacgctggccgcgtctttaaagacagcgacaaattcgatgcaaatgattctatcctaaaagaccaaac
acaagaatggtcaggttcagccacatttacatctgacggaaaaatccgtttattctacactgatttctccggtaaacattacg
gcaaacaaacactgacaactgcacaagttaacgtatcagcatcagacagctctttgaacatcaacggtgtagaggattataaa
tcaatctttgacggtgacggaaaaacgtatcaaaatgtacagcagttcatcgatgaaggcaactacagctcaggcgacaacca
tacgctgagagatcctcactacgtagaagataaaggccacaaatacttagtatttgaagcaaacactggaactgaagatggct
accaaggcgaagaatctttatttaacaaagcatactatggcaaaagcacatcattcttccgtcaagaaagtcaaaaacttctg
caaagcgataaaaaacgcacggctgagttagcaaacggcgctctcggtatgattgagctaaacgatgattacacactgaaaaa
agtgatgaaaccgctgattgcatctaacacagtaacagatgaaattgaacgcgcgaacgtctttaaaatgaacggcaaatggt
acctgttcactgactcccgcggatcaaaaatgacgattgacggcattacgtctaacgatatttacatgcttggttatgtttct
aattctttaactggcccatacaagccgctgaacaaaactggccttgtgttaaaaatggatcttgatcctaacgatgtaacctt
tacttactcacacttcgctgtacctcaagcgaaaggaaacaatgtcgtgattacaagctatatgacaaacagaggattctacg
cagacaaacaatcaacgtttgcgccaagcttcctgctgaacatcaaaggcaagaaaacatctgttgtcaaagacagcatcctt
gaacaaggacaattaacagttaacaaataaaaacgcaaaagaaaatgccgatatcctattggcattttcttttatttcttatc
aacataaaggtgaatcccatacctagagctgcacgcgagacatgaacgtgcaactgcttctcaggtaatagtaacctaaaagc
acatgatgaattacattcagtagaagaaattattggaagtaatcagagagacgaatttaaaggtagtaaattcagagatattt
tccatggtgccgatggtgatgatctattaaatggtaatgatggggatgatattctatacggtgataaaggtaacgatgagtta
agaggtgataatggtaacgaccaactttatggtggtgaaggtaatgacaaactattaggaggtaatggcaataattacctcag
tggtggtgatggcaatgatgagcttcaagtcttaggcaatggttttaatgtgcttcgtggcggtaaaggcgatgataaacttt
atggtagctcaggttctgatttacttgatggtggagaaggtaatgattatctagaaggaggcgatggtagcgatttttatgtt
tatcgttccacttcaggtaatcatactatttatgatcaaggtaaatctagtgatttagataaagatccgaattcctgtgtgaa
atgg
apxIIIAmut
SEQ ID NO: 40
gaccgcggccgcgcaggtgcattagttggcgcaccaattactttgttggttactggtatcacaggattaatttctggtatttt
agagttctctaaacaaccaatgttagatcatgttgcatcgaaaattggtaacaaaattgacgaatgggagaaaaaatacggta
aaaattacttcgagaatggctatgatgctcgtcataaagctttcttagaagattcattctcattattgtctagttttaataaa
caatatgaaactgaaagagctgttttaattacacaacaacgttgggatgaatatattggcgaacttgcgggtattactggcaa
aggtgacaaactctctagtggtaaggcgtatgtagattactttcaagaaggtaaattattagagaaaaaacctgatgacttta
gcaaagtagttttcgatccaactaagggcgaaattgatatttcaaatagccaaacgtcaacgttgttaaaatttgttacgcca
ttattaacaccaggtacagagtcacgtgaaagaactcaaacaggtgcatatgaatatatcacgaagttagttgtaaaaggtaa
agataaatgggttgttaatggcgttaaagataaaggtgccgtttatgattatactaatttaattcaacatgctcatattagtt
catcagtagcacgtggtgaagaataccgtgaagttcgtttggtatctcatctaggcaatggtaatgacaaagtgttcttagct
tggaaccaaagcgactgaacaagggcgttattctgttacgcgcgaattgagtggtgctacaaaaatcctgagagaagtaataa
aaaatcaaaaatctgctgttggtgcacgtgaagaaaccttggaatatcgtgattatgaattaacgcaatcaggtaatagtaac
ctaaaagcacatgatgaattacattcagtagaagaaattattggaagtaatcagagagacgaatttaaaggtagtaaattcag
agatattttccatggtgccgatggtgatgatctattaaatggtaatgatggggatgatattctatacggtgataaaggtaacg
atgagttaagaggtgataatggtaacgaccaactttatggtggtgaaggtaatgacaaactattaggaggtaatggcaataat
tacctcagtggtggtgatggcaatgatgagcttcaagtcttaggcaatggttttaatgtgcttcgtggcggtaaaggcgatga
taaactttatggtagctcaggttctgatttacttgatggtggagaaggtaatgattatctagaaggaggcgatggtagcgatt
tttatgtttatcgttccacttcaggtaatcatactatttatgatcaaggtaaatctagtgatttagatacaagcggtttg
delta apxIA trunc dfrA14sacB
SEQ ID NO: 41
attgggtaccgagctcgcggccgcctaattcacccgcttgcgattgcgggtctaaagtaccgccgtaccaaacgtccgcttgc
ggattatttttttccgcttcgatttttgcaaaggtactgccggaaccgttgcggataaaagaggttttcacatcatatttttg
ttcgaatgtttttgccgcattctcacacatcacattggtcgcactacagtaaatcactaaacgtccttttgcctgagccgccg
aactgaacattaagcccgcaccaagtaatgcggttgaaaccgctaaagaaagttttccaaatttcataatcaaagcctcatat
tgagcataaatcaataaaatgccgcgaatataatcgaaagcatttttcttattggaactaatttaccgtaattgaataaaaaa
taccgtgaagcagttcacaaaatacgagattaatgagcgatattgttataaaatcataatgtaaacctcatttgtaatgaatt
ggtaaattatataaataatcaaaaaacttacttttttttatttttatcggtaagtatttacaatcaagtcagacaaacagtaa
gattgaaggttaatgccgtctgaagtgcgaagcggcatcagagcagattgtactgagagtgcaccatatggtcgacctcgagt
taattaacgtatgcggccgctttagactatttaaataatattatttaaattctttactatagtgtacaatacacacagtccat
taaccaaaataaaaggaggaattaggatgagaaccttgaaagtatcattgatagctgcgaaagcgaaaaacggcgtgattggt
tgcggtccagacataccctggtccgcgaaaggggagcagctactttttaaagcattgacctacaatcagtggcttctggtggg
tcgcaagacgtttgaatctatgggcgcactccccaataggaaatacgcggtcgttacccgctcaggttggacatcaaatgatg
acaatgtagttgtatttcagtcaatcgaagaggccatggacaggctagctgaattcaccggtcacgttatagtgtctggtggc
ggagaaatttaccgagaaacattacccatggcctctacgctccacttatcgacgatcgacatcgagccagagggggatgtttt
cttcccgagtattccaaataccttcgaagttgtttttgagcaacactttacttcaaacattaactattgctatcaaatttgga
aaaagggttaatgccgtctgaagtgcggtacaagcggtagaacctgccccgttagttgaaaccgcttgttatgcatgcatggg
atccgcgaatcccgcggccatggcggccgggagcatgcgacgtcgggcccattgggatccgcttttacagcgattgcagaatg
attgaattgtaaactttagagctttatattttgtttaatggtattatatttacttatatttatgattcttagtttttattgta
aattaaagtgtttatttattgtattttaagtataagatcctttttaacccatcacatatacctgccgttcactattatttagt
gaaatgagatattatgatattttctgaattgtgattaaaaaggcaactttatgcccatgcaacagaaactataaaaaatacag
agaatgaaaagaaacagatagattttttagttctttaggcccgtagtctgcaaatccttttatgattttctatcaaacaaaag
aggaaaatagaccagttgcaatccaaacgagagtctaatagaatgaggtcgaaaagtaaatcgcgcgggtttgttactgataa
agcaggcaagacctaaaatgtgtaaagggcaaagtgtatactttggcgtcaccccttacatattttaggtctttttttattgt
gcgtaactaacttgccatcttcaaacaggagggctggaagaagcagaccgctaacacagtacataaaaaaggagacatgaacg
atgaacatcaaaaagtttgcaaaacaagcaacagtattaacctttactaccgcactgctggcaggaggcgcaactcaagcgtt
tgcgaaagaaacgaaccaaaagccatataaggaaacatacggcatttcccatattacacgccatgatatgctgcaaatccctg
aacagcaaaaaaatgaaaaatatcaagttcctgaattcgattcgtccacaattaaaaatatctcttctgcaaaaggcctggac
gtttgggacagctggccattacaaaacgctgacggcactgtcgcaaactatcacggctaccacatcgtctttgcattagccgg
agatcctaaaaatgcggatgacacatcgatttacatgttctatcaaaaagtcggcgaaacttctattgacagctggaaaaacg
ctggccgcgtctttaaagacagcgacaaattcgatgcaaatgattctatcctaaaagaccaaacacaagaatggtcaggttca
gccacatttacatctgacggaaaaatccgtttattctacactgatttctccggtaaacattacggcaaacaaacactgacaac
tgcacaagttaacgtatcagcatcagacagctctttgaacatcaacggtgtagaggattataaatcaatctttgacggtgacg
gaaaaacgtatcaaaatgtacagcagttcatcgatgaaggcaactacagctcaggcgacaaccatacgctgagagatcctcac
tacgtagaagataaaggccacaaatacttagtatttgaagcaaacactggaactgaagatggctaccaaggcgaagaatcttt
atttaacaaagcatactatggcaaaagcacatcattcttccgtcaagaaagtcaaaaacttctgcaaagcgataaaaaacgca
cggctgagttagcaaacggcgctctcggtatgattgagctaaacgatgattacacactgaaaaaagtgatgaaaccgctgatt
gcatctaacacagtaacagatgaaattgaacgcgcgaacgtctttaaaatgaacggcaaatggtacctgttcactgactcccg
cggatcaaaaatgacgattgacggcattacgtctaacgatatttacatgcttggttatgtttctaattctttaactggcccat
acaagccgctgaacaaaactggccttgtgttaaaaatggatcttgatcctaacgatgtaacctttacttactcacacttcgct
gtacctcaagcgaaaggaaacaatgtcgtgattacaagctatatgacaaacagaggattctacgcagacaaacaatcaacgtt
tgcgccaagcttcctgctgaacatcaaaggcaagaaaacatctgttgtcaaagacagcatccttgaacaaggacaattaacag
ttaacaaataaaaacgcaaaagaaaatgccgatatcctattggcattttcttttatttcttatcaacataaaggtgaatccca
tacctagagctgcacgcgagacatgaacgtgcaactgcttcgatagttatttttagatgataaatagcaatcctatatatatt
aggtgtgtaggattgctattttatttatggaggagcaaatggatttttatcgggaagaagactacggattatacgcactgacg
attttagcccagtaccataatattgctgtaaatccggaagaactaaaacataaattcgaccttgaaggaaaaggcttagatct
aaccgcttggctattagccgcaaaatcattagaacttaaagcaaaacaagtaaaaaaagcgattgatcgtttggcgtttatcg
cactaccggcacttgtatggcgagaagacggtaaacattttattttgactaaaattgataatgaagcaaaaaaatatttaatt
tttgatttggaaacgcataatcctcgcattttggaacaagcggaattcgagagcttataccaaggaaaactgattttagttgc
atcaagagcttccatcgtaggtaagctggcaaagtttgacttgatccgaattcctgtgtgaaatgg
apxIAmut long
SEQ ID NO: 42
aaacaagcggtccggatcttggaatttcggcataatttgatcgatattcggcgaacgatacgcttctaataagcctaattcac
ccgcttgcgattgcgggtctaaagtaccgccgtaccaaacgtccgcttgcggattatttttttccgcttcgatttttgcaaag
gtactgccggaaccgtttcggataaaagaggttttcacatcatatttttgttcgaatgtttttgccgcattctcacacatcac
attggtcgcactacagtaaatcactaaacgtccttttgcctgagccgccgaactgaacattaagcccgcaccaagtaatgcgg
ttgaaaccgctaaagaaagttttccaaatttcataatcaaagcctcatattgagcataaatcaacaaaatgccgcgaatataa
tcgaaagcatttttcttattggaactaatttaccgtaattgaataaaaaataccgtgaagcagttcacaaaatacgagattaa
tgagcgatattgttataaaatcataatgtaaacctcatttgtaatgaattggtaaattatataaataatcaaaaaacttactt
ttttttatttttatcggtaagtatttacaatcaagtcagacaaacggcaatattgttataaatctggggggatgaatgagtaa
aaaaattaatggatttgaggttttaggagaggtggcatggttatgggcaagttctcctttacatcgaaagtggccgctttctt
tgttagcaattaatgtgctacctgcgattgagagtaatcaatatgttttgttaaagcgtgacggttttcctattgcattttgt
agctgggcaaatttgaatttggaaaatgaaattaaataccttgatgatgttgcctcgctagttgcggatgattggacttccgg
cgatcgtcgatggtttatagattggatagcaccgttcggagacagtgccgcattatacaaacatatgcgagataacttcccga
atgagctgtttagggctattcgagttgatccggactctcgagtagggaaaatttcagaatttcatggaggaaaaattgataag
aaactggcaagtaaaatttttcaacaatatcactttgaattaatgagtgagctaaaaaataaacaaaattttaaattttcatt
agtaaatagctaaggagacaacatggctaactctcagctcgatagagtcaaaggattgattgattcacttaatcaacatacaa
aaagtgcagctaaatcaggtgccggcgcattaaaaaatggtttgggacaggtgaagcaagcagggcagaaattaattttatat
attccgaaagattatcaagctagtaccggctcaagtcttaatgatttagtgaaagcggcggaggctttagggatcgaagtaca
tcgctcggaaaaaaacggtaccgcactagcgaaagaattattcggtacaacggaaaaactattaggtttctcggaacgaggca
tcgcattatttgcacctcagtttgataagttactgaataagaaccaaaaattaagtaaatcgctcggcggttcatcggaagca
ttaggacaacgtttaaataaaacgcaaacggcactttcagccttacaaagtttcttaggtacggctattgcgggtatggatct
tgatagcctgcttcgtcgccgtagaaacggtgaggacgtcagtggttcggaattagctaaagcaggtgtggatctagccgctc
agttagtggataacattgcaagtgcaacgggtacggtggatgcgtttgccgaacaattaggtaaattgggcaatgccttatct
aacactcgcttaagcggtttagcaagtaagttaaataaccttccagatttaagccttgcaggacctgggtttgatgccgtatc
aggtatcttatctgttgtttcggcttcattcattttaagtaataaagatgccgatgcaggtacaaaagcggcggcaggtattg
aaatctcaactaaaatcttaggcaatatcggtaaagcggtttctcaatatattattgcgcaacgtgtggcggcaggcttatcc
acaactgcggcaaccggtggtttaatcggttcggtcgtagcattagcgattagcccgctttcgttcttaaatgttgcggataa
gtttgaacgtgcgaaacagcttgaacaatattcggagcgctttaaaaagttcggttatgaaggtgatagtttattagcttcat
tctaccgtgaaaccggtgcgattgaagcggcattaaccacgattaacagtgtgttaagtgcggcttccgcaggtgttggggct
gctgcaaccggctcattagtcggtgcgccggtagcagctttagttagtgcaatcaccggtattatttcaggtattttagatgc
ttctaaacaggcaatcttcgaacgagttgcaacgaaattagcgaataagattgacgaatgggagaaaaaacacggtaaaaact
attttgaaaacggttatgacgcccgccattccgcattcttagaagatacctttgaattgttatcacaatacaataaagagtat
tcggtagagcgtgtcgttgctattacgcaacagcgttgggatgtcaatatcggtgaacttgccggcattactcgcaaaggttc
tgatacgaaaagcggtaaagcttacgttgatttctttgaagaaggaaaacttttagagaaagaaccggatcgttttgataaaa
aagtgtttgatccgcttgaaggcaaaatcgacctttcttcaattaacaaaaccactttattgaaatttattacaccggttttt
accgcaggtgaagagattcgtgagcgtaagcaaaccggtgcatacgaatatatgaccgaattattcgttaaaggtaaagaaaa
atgggtggtaaccggtgtgcagtcacataatgcgatttatgactatacgaatcttatccaattagcgatagataaaaaaggtg
aaaaacgtcaagtgaccattgaatctcatttgggtgagaaaaatgatcgtatatatctttcatccggttcatctatcgtatat
gcgggtaacggacatgatgtagcatattacgataaaaccgatacaggttacttaacatttgacggacaaagtgcacagaaagc
cggtgaatatattgtcactaaagaacttaaagctgatgtaaaagttttaaaagaagtggttaaaactcaggatatttcagttg
gagcacgcagtgaaaaattagaatatcgtgattatgagttaagcccattcgaacttgggaacggtatcagagctaaagatgaa
ttacattctgttgaagaaattatcggtagtaatcgtaaagacaaattctttggtagtcgctttaccgatattttccatggtgc
gaaaggcgatgatgaaatctacggtaatgacggccacgatatcttatacggagacgacggtaatgatgtaatccatggcggtg
acggtaacgaccatcttgttggtggtaacggaaacgaccgattaatcggcggaaaaggtaataatttccttaatggcggtgat
ggtgacgatgagttgcaggtctttgagggtcaatacaacgtattattaggtggtgcgggtaatgacattctgtatggcagcga
tggtactaacttatttgacggtggtgtaggcaatgacaaaatctacggtggtttaggtaaggatatttatcgctacagtaagg
agtacggtcgtcatatcattattgagaaaggcggtgatgatgatacgttattgttatcggatcttagttttaaagatgtagga
tttatcagaatcggtgatgatcttcttgtgaataaaagaatcggaggaacactgtattaccatgaagattacaatgggaatgc
gctcacgattaaagattggttcaaggaaggtaaagaaggacaaaataataaaattgaaaaaatcgttgataaagatggagctt
atgttttaagccaatatctgactgaactgacagctcctggaagaggtatcaattactttaatgggttagaagaaaaattgtat
tatggagaaggatataatgcacttcctcaactcagaaaagatattgaacaaatcatttcatctacgggtgcatttaccggtga
tcacggaaaagtatctgtaggctcaggcggaccgttagtctataataactcagctaacaatgtagcaaattctttgagttatt
ctttagcacaagcagcttaagatagttatttttagatgataaatagcaatcctatatatattaggtgtgtaggattgctattt
tatttatggaggagcaaatggatttttatcgggaagaagactacggattatacgcactgacgattttagcccagtaccataat
attgctgtaaatccggaagaactaaaacataaattcgaccttgaaggaaaaggcttagatctaaccgcttggctattagccgc
aaaatcattagaacttaaagcaaaacaagtaaaaaaagcgattgatcgtttggcgtttatcgcactaccggcacttgtatggc
gagaagacggtaaacattttattttgactaaaattgataatgaagcaaaaaaatatttaatttttgatttggaaacgcataat
cctcgcattttggaacaagcggaattcgagagcttataccaaggaaaactgattttagttgcatcaagagcttccatcgtagg
taagctggcaaagtaccgcttgtaa
delta apxIVA dfrA14sacB
SEQ ID NO: 43
atccacaagcggtcatctggcgcgaatagagaacctgaacaatgggaaaattacatagtatttgataattgcagtggaattaa
agaaagacaccaactgtattaaaaatagattagaaggagacaacacgatgacaaaactaactatgcaagatgtgactaattta
tatttatataagcaaagaactttacctacggataggttagatgattcgcttattagcaaaacaggaaaaggggaaaatattga
taaaaaggaatttatggcggggccgggacgttttgtgacggccgataattttagtgttgtaaaagacttttttactgcaaagg
attcattaataaacctaagcttgcagactcgtatattagcgaatttaaagccgggcaaatattccaaagcgcagatattagaa
atgttgggctatacgaaaaatggagaaaaggtagatggcatgtttaccggtgaagtccagacattaggcttttatgacgatgg
caaaggggatttactcgaacgcgttaatgccgtctgaagtgcgaagcggcatcagagcagattgtactgagagtgcaccatat
ggtcgacctcgagttaattaacgtatgcggccgctttagactatttaaataatattatttaaattctttactatagtgtacaa
tacacacagtccattaaccaaaataaaaggaggaattaggatgagaaccttgaaagtatcattgatagctgcgaaagcgaaaa
acggcgtgattggttgcggtccagacataccctggtccgcgaaaggggagcagctactttttaaagcattgacctacaatcag
tggcttctggtgggtcgcaagacgtttgaatctatgggcgcactccccaataggaaatacgcggtcgttacccgctcaggttg
gacatcaaatgatgacaatgtagttgtatttcagtcaatcgaagaggccatggacaggctagctgaattcaccggtcacgtta
tagtgtctggtggcggagaaatttaccgagaaacattacccatggcctctacgctccacttatcgacgatcgacatcgagcca
gagggggatgttttcttcccgagtattccaaataccttcgaagttgtttttgagcaacactttacttcaaacattaactattg
ctatcaaatttggaaaaagggttaatgccgtctgaagtgcggtacaagcggtagaacctgccccgttagttgaaaccgcttgt
tatgcatgcatgggatccgcgaatcccgcggccatggcggccgggagcatgcgacgtcgggcccattgggatccgcttttaca
gcgattgcagaatgattgaattgtaaactttagagctttatattttgtttaatggtattatatttacttatatttatgattct
tagtttttattgtaaattaaagtgtttatttattgtattttaagtataagatcctttttaacccatcacatatacctgccgtt
cactattatttagtgaaatgagatattatgatattttctgaattgtgattaaaaaggcaactttatgcccatgcaacagaaac
tataaaaaatacagagaatgaaaagaaacagatagattttttagttctttaggcccgtagtctgcaaatccttttatgatttt
ctatcaaacaaaagaggaaaatagaccagttgcaatccaaacgagagtctaatagaatgaggtcgaaaagtaaatcgcgcggg
tttgttactgataaagcaggcaagacctaaaatgtgtaaagggcaaagtgtatactttggcgtcaccccttacatattttagg
tctttttttattgtgcgtaactaacttgccatcttcaaacaggagggctggaagaagcagaccgctaacacagtacataaaaa
aggagacatgaacgatgaacatcaaaaagtttgcaaaacaagcaacagtattaacctttactaccgcactgctggcaggaggc
gcaactcaagcgtttgcgaaagaaacgaaccaaaagccatataaggaaacatacggcatttcccatattacacgccatgatat
gctgcaaatccctgaacagcaaaaaaatgaaaaatatcaagttcctgaattcgattcgtccacaattaaaaatatctcttctg
caaaaggcctggacgtttgggacagctggccattacaaaacgctgacggcactgtcgcaaactatcacggctaccacatcgtc
tttgcattagccggagatcctaaaaatgcggatgacacatcgatttacatgttctatcaaaaagtcggcgaaacttctattga
cagctggaaaaacgctggccgcgtctttaaagacagcgacaaattcgatgcaaatgattctatcctaaaagaccaaacacaag
aatggtcaggttcagccacatttacatctgacggaaaaatccgtttattctacactgatttctccggtaaacattacggcaaa
caaacactgacaactgcacaagttaacgtatcagcatcagacagctctttgaacatcaacggtgtagaggattataaatcaat
ctttgacggtgacggaaaaacgtatcaaaatgtacagcagttcatcgatgaaggcaactacagctcaggcgacaaccatacgc
tgagagatcctcactacgtagaagataaaggccacaaatacttagtatttgaagcaaacactggaactgaagatggctaccaa
ggcgaagaatctttatttaacaaagcatactatggcaaaagcacatcattcttccgtcaagaaagtcaaaaacttctgcaaag
cgataaaaaacgcacggctgagttagcaaacggcgctctcggtatgattgagctaaacgatgattacacactgaaaaaagtga
tgaaaccgctgattgcatctaacacagtaacagatgaaattgaacgcgcgaacgtctttaaaatgaacggcaaatggtacctg
ttcactgactcccgcggatcaaaaatgacgattgacggcattacgtctaacgatatttacatgcttggttatgtttctaattc
tttaactggcccatacaagccgctgaacaaaactggccttgtgttaaaaatggatcttgatcctaacgatgtaacctttactt
actcacacttcgctgtacctcaagcgaaaggaaacaatgtcgtgattacaagctatatgacaaacagaggattctacgcagac
aaacaatcaacgtttgcgccaagcttcctgctgaacatcaaaggcaagaaaacatctgttgtcaaagacagcatccttgaaca
aggacaattaacagttaacaaataaaaacgcaaaagaaaatgccgatatcctattggcattttcttttatttcttatcaacat
aaaggtgaatcccatacctagagctgcacgcgagacatgaacgtgcaactgcttccaattcgaagggaaatgggtaaccgatt
attctcgtactgaagccttatttaactctacttttaaacaatcgcctgaaaatgcattatatgatttaagcgaatacctttct
ttctttaacgatcctacggaatggaaagaagggctattactgttaagccgttatatagattatgctaaagcacaaggatttta
tgaaaactgggcggctacttctaacttaactattgcccgtttaagagaggctggagtaatttttgcagaatcgacggatttaa
aaggcgatgaaaaaaataatattttgttaggtagccaaaaagataataacttatcgggtagtgcaggtgatgatctacttatc
ggcggagagggtaatgatacgttaaaaggcagctacggtgcggacacctatatctttagcaaaggacacggacaggatatcgt
ttatgaagataccaataatgataaccgagcaagagatatcgacaccttaaaatttggatccgaattcctgtgtgaaatgg
apxIV int del
SEQ ID NO: 44
atccacaagcggtcatctggcgcgaatagagaacctgaacaatgggaaaattacatagtatttgataattgcagtggaattaa
agaaagacaccaactgtattaaaaatagattagaaggagacaacacgatgacaaaactaactatgcaagatgtgactaattta
tatttatataagcaaagaactttacctacggataggttagatgattcgcttattagcaaaacaggaaaaggggaaaatattga
taaaaaggaatttatggcggggccgggacgttttgtgacggccgataattttagtgttgtaaaagacttttttactgcaaagg
attcattaataaacctaagcttgcagactcgtatattagcgaatttaaagccgggcaaatattccaaagcgcagatattagaa
atgttgggctatacgaaaaatggagaaaaggtagatggcatgtttaccggtgaagtccagacattaggcttttatgacgatgg
caaaggggatttactcgaacgccaattcgaagggaaatgggtaaccgattattctcgtactgaagccttatttaactctactt
ttaaacaatcgcctgaaaatgcattatatgatttaagcgaatacctttctttctttaacgatcctacggaatggaaagaaggg
ctattactgttaagccgttatatagattatgctaaagcacaaggattttatgaaaactgggcggctacttctaacttaactat
tgcccgtttaagagaggctggagtaatttttgcagaatcgacggatttaaaaggcgatgaaaaaaataatattttgttaggta
gccaaaaagataataacttatcgggtagtgcaggtgatgatctacttatcggcggagagggtaatgatacgttaaaaggcagc
tacggtgcggacacctatatctttagcaaaggacacggacaggatatcgtttatgaagataccaataatgataaccgagcaag
agatatcgacaccttaaaatttggatccgaattcctgtgtgaaatgg
sxy dfrA14sacB insert
SEQ ID NO: 45
gtaccgcttgttaaatgattacaccaagcgactctaaaaatcttcgtatctatatcataaatacgatgagcgttaaagtggcg
atattctagtgtaaaaaacacttaaaagcaagatttaattttatttttctaaaaaatatagtttcaaacgaatcggacatatt
tttaccctttattatatttacattattgacattaaataatttattttgcaaaatatacataaatttcgctcattaaaaaataa
tcatatataaaaaaggagaaacataatggcaatatccccaaaaaagttccaatatcttaaggagatttttagtcctcttggag
aaattaacttcaaaagctatttttcttacttaggaatatttaaagacgatactatgttcgccctctatgatcataaaaacgat
cgattatacttaagaaaatccgctcaattttatccggatattataagaacaataccgatacattttttaattgatcgtcgtat
cggtaagcaacaatctcatattttttatcttataccttcttctattattcacaatcttcatttatatactcattggattctct
ctgctatcgaagaatatcaaactgcaaaggccaaattgatttctcaaaataaaaataaaattcgtctgcttcccaatttgaat
atcaatatagaaagattattggcacgtattgagatttataccgtagatgatttaaaaaacgtaggcgtgattaatgcgtttgt
aaaactgataatgctaggcttggaagtaaccgaattactcctcttcaaactctacgctgcgctcgaacataaatatatctata
tgttatccaagcaagaaaaacaatccctattaattgaagccgatttatctctctataacgcaggcctacgtaaacgcttcgct
atctcacaggctaactaagttaatgccgtctgaagtgcgaagcggcatcagagcagattgtactgagagtgcaccatatggtc
gacctcgagttaattaacgtatgcggccgctttagactatttaaataatattatttaaattctttactatagtgtacaataca
cacagtccattaaccaaaataaaaggaggaattaggatgagaaccttgaaagtatcattgatagctgcgaaagcgaaaaacgg
cgtgattggttgcggtccagacataccctggtccgcgaaaggggagcagctactttttaaagcattgacctacaatcagtggc
ttctggtgggtcgcaagacgtttgaatctatgggcgcactccccaataggaaatacgcggtcgttacccgctcaggttggaca
tcaaatgatgacaatgtagttgtatttcagtcaatcgaagaggccatggacaggctagctgaattcaccggtcacgttatagt
gtctggtggcggagaaatttaccgagaaacattacccatggcctctacgctccacttatcgacgatcgacatcgagccagagg
gggatgttttcttcccgagtattccaaataccttcgaagttgtttttgagcaacactttacttcaaacattaactattgctat
caaatttggaaaaagggttaatgccgtctgaagtgcggtacaagcggtagaacctgccccgttagttgaaaccgcttgttatg
catgcatgggatccgcgaatcccgcggccatggcggccgggagcatgcgacgtcgggcccattgggatccgcttttacagcga
ttgcagaatgattgaattgtaaactttagagctttatattttgtttaatggtattatatttacttatatttatgattcttagt
ttttattgtaaattaaagtgtttatttattgtattttaagtataagatcctttttaacccatcacatatacctgccgttcact
attatttagtgaaatgagatattatgatattttctgaattgtgattaaaaaggcaactttatgcccatgcaacagaaactata
aaaaatacagagaatgaaaagaaacagatagattttttagttctttaggcccgtagtctgcaaatccttttatgattttctat
caaacaaaagaggaaaatagaccagttgcaatccaaacgagagtctaatagaatgaggtcgaaaagtaaatcgcgcgggtttg
ttactgataaagcaggcaagacctaaaatgtgtaaagggcaaagtgtatactttggcgtcaccccttacatattttaggtctt
tttttattgtgcgtaactaacttgccatcttcaaacaggagggctggaagaagcagaccgctaacacagtacataaaaaagga
gacatgaacgatgaacatcaaaaagtttgcaaaacaagcaacagtattaacctttactaccgcactgctggcaggaggcgcaa
ctcaagcgtttgcgaaagaaacgaaccaaaagccatataaggaaacatacggcatttcccatattacacgccatgatatgctg
caaatccctgaacagcaaaaaaatgaaaaatatcaagttcctgaattcgattcgtccacaattaaaaatatctcttctgcaaa
aggcctggacgtttgggacagctggccattacaaaacgctgacggcactgtcgcaaactatcacggctaccacatcgtctttg
cattagccggagatcctaaaaatgcggatgacacatcgatttacatgttctatcaaaaagtcggcgaaacttctattgacagc
tggaaaaacgctggccgcgtctttaaagacagcgacaaattcgatgcaaatgattctatcctaaaagaccaaacacaagaatg
gtcaggttcagccacatttacatctgacggaaaaatccgtttattctacactgatttctccggtaaacattacggcaaacaaa
cactgacaactgcacaagttaacgtatcagcatcagacagctctttgaacatcaacggtgtagaggattataaatcaatcttt
gacggtgacggaaaaacgtatcaaaatgtacagcagttcatcgatgaaggcaactacagctcaggcgacaaccatacgctgag
agatcctcactacgtagaagataaaggccacaaatacttagtatttgaagcaaacactggaactgaagatggctaccaaggcg
aagaatctttatttaacaaagcatactatggcaaaagcacatcattcttccgtcaagaaagtcaaaaacttctgcaaagcgat
aaaaaacgcacggctgagttagcaaacggcgctctcggtatgattgagctaaacgatgattacacactgaaaaaagtgatgaa
accgctgattgcatctaacacagtaacagatgaaattgaacgcgcgaacgtctttaaaatgaacggcaaatggtacctgttca
ctgactcccgcggatcaaaaatgacgattgacggcattacgtctaacgatatttacatgcttggttatgtttctaattcttta
actggcccatacaagccgctgaacaaaactggccttgtgttaaaaatggatcttgatcctaacgatgtaacctttacttactc
acacttcgctgtacctcaagcgaaaggaaacaatgtcgtgattacaagctatatgacaaacagaggattctacgcagacaaac
aatcaacgtttgcgccaagcttcctgctgaacatcaaaggcaagaaaacatctgttgtcaaagacagcatccttgaacaagga
caattaacagttaacaaataaaaacgcaaaagaaaatgccgatatcctattggcattttcttttatttcttatcaacataaag
gtgaatcccatacctagagctgcacgcgagacatgaacgtgcaactgcttcgtaagccggttcctttctgattatctcaatgc
taccatcctacctataacttgttagtttatttaagtgaaatctacttttatccataggagaacacaatggaatttcgtattga
aaaagataccatgggcgaagttcaagtacctgccaatcgttattgggcggcacaaacagagcgttcacgcaataattttaaaa
tcggtcccgaagcgtcaatgcctaaagaaattattgaagcgttcggttacttgaaaaaagcagcggcatttgccaacacagat
ttaggcgtattacctgcggaaaaacgtgatttaatcgctcaagcctgtgatgaaatccttgccggtaaattaaacgaagaatt
cccgcttgtaatctggcaaaccggttccggtacgcaatccaatatgaacttaaacgaagttattgcaaaccgtgcgcatgtta
ttcacggcggtaaattaggtgaaaaatcggtaattcacccgaatgatgaggatccgaattcctgtgtgaaatgg
Sxy del
SEQ ID NO: 46
atccacaagcggtcatctggctcacgtgtggagaaatcaatacagtaaaacgttctttacgagttggtaaaggaattggacca
cgaacttgtgcaccagtacgtttagctgtttctacgatctccgcagtagattgatcaattaaacgatgatcaaatgcttttaa
gcggatacggattctttggttctgcattagaccagagctccaattaaaatttagctaataaaaaaaccgaactaccacttaag
ccacatagcataagggagcgcagttatacctatatagtttccaaatcggaaacattgtatgtactacaatatctgtagtaccg
cttgataaatgattacaccaagcgactctaaaaatcttcgtatctatatcataaatacgatgagcgttaaagtggcgatattc
tagtgtaaaaaacacttaaaagcaagatttaattttatttttctaaaaaatatagtttcaaacgaatcggacatatttttacc
ctttattatatttacattgtaagccggttcctttctgattatctcaatgctaccatcctacctataacttgttagtttattta
agtgaaatctacttttatccataggagaacacaatggaatttcgtattgaaaaagataccatgggcgaagttcaagtacctgc
caatcgttattgggcggcacaaacagagcgttcacgcaataattttaaaatcggtcccgaagcgtcaatgcctaaagaaatta
ttgaagcgttcggttacttgaaaaaagcagcggcatttgccaacacagatttaggcgtattacctgcggaaaaacgtgattta
atcgctcaagcctgtgatgaaatccttgccggtaaattaaacgaagaattcccgcttgtaatctggcaaaccggttccggtac
gcaatccaatatgaacttaaacgaagttattgcaaaccgtgcgcatgttattcacggcggtaaattaggtgaaaaatcggtaa
ttcacccgaatgatgaggatccgaattcctgtgtgaaatgg
APP ApxIV
SEQ ID NO: 47
Met Thr Lys Leu Thr Met Gln Asp Val Thr Asn Leu Tyr Leu Tyr Lys Gln Arg Thr Leu Pro
Thr Asp Arg Leu Asp Asp Ser Leu Ile Ser Lys Thr Gly Lys Gly Glu Asn Ile Asp Lys Lys
Glu Phe Met Ala Gly Pro Gly Arg Phe Val Thr Ala Asp Asn Phe Ser Val Val Lys Asp Phe
Phe Thr Ala Lys Asp Ser Leu Ile Asn Leu Ser Leu Gln Thr Arg Ile Leu Ala Asn Leu Lys
Pro Gly Lys Tyr Ser Lys Ala Gln Ile Leu Glu Met Leu Gly Tyr Thr Lys Asn Gly Glu Lys
Val Asp Gly Met Phe Thr Gly Glu Val Gln Thr Leu Gly Phe Tyr Asp Asp Gly Lys Gly Asp
Leu Leu Glu Arg Ala Tyr Ile Trp Asn Thr Thr Gly Phe Lys Met Ser Asp Asn Ala Phe Phe
Val Ile Glu Glu Ser Gly Lys Arg Tyr Ile Glu Asn Phe Gly Ile Glu Pro Leu Gly Lys Gln
Glu Asp Phe Asp Phe Val Gly Gly Phe Trp Ser Asn Leu Val Asn Arg Gly Leu Glu Ser Ile
Ile Asp Pro Ser Gly Ile Gly Gly Thr Val Asn Leu Asn Phe Thr Gly Glu Val Glu Thr Tyr
Thr Leu Asp Glu Thr Arg Phe Lys Ala Glu Ala Ala Lys Lys Ser His Trp Ser Leu Val Asn
Ala Ala Lys Val Tyr Gly Gly Leu Asp Gln Ile Ile Lys Lys Leu Trp Asp Ser Gly Ser Ile
Lys His Leu Tyr Gln Asp Lys Asp Thr Gly Lys Leu Lys Pro Ile Ile Tyr Gly Thr Ala Gly
Asn Asp Ser Lys Ile Glu Gly Thr Lys Ile Thr Arg Arg Ile Ala Gly Lys Glu Val Thr Leu
Asp Ile Ala Asn Gln Lys Ile Glu Lys Gly Val Leu Glu Lys Leu Gly Leu Ser Val Ser Gly
Ser Asp Ile Ile Lys Leu Leu Phe Gly Ala Leu Thr Pro Thr Leu Asn Arg Met Leu Leu Ser
Gln Leu Ile Gln Ser Phe Ser Asp Ser Leu Ala Lys Leu Asp Asn Pro Leu Ala Pro Tyr Thr
Lys Asn Gly Val Val Tyr Val Thr Gly Lys Gly Asn Asp Val Leu Lys Gly Thr Glu His Glu
Asp Leu Phe Leu Gly Gly Glu Gly Asn Asp Thr Tyr Tyr Ala Arg Val Gly Asp Thr Ile Glu
Asp Ala Asp Gly Lys Gly Lys Val Tyr Phe Val Arg Glu Lys Gly Ile Pro Lys Ala Asp Pro
Lys Arg Val Glu Phe Ser Lys Tyr Ile Thr Glu Glu Glu Ile Lys Glu Val Glu Lys Gly Leu
Leu Thr Tyr Ala Val Leu Glu Asn Tyr Asn Trp Glu Glu Lys Thr Ala Thr Phe Ala His Ala
Thr Met Leu Asn Glu Leu Phe Thr Asp Tyr Thr Asn Tyr Arg Tyr Lys Val Lys Gly Leu Lys
Leu Pro Ala Val Lys Lys Leu Lys Ser Pro Leu Val Glu Phe Thr Ala Asp Leu Leu Thr Val
Thr Pro Ile Asp Glu Asn Gly Lys Ala Leu Ser Glu Lys Ser Ile Thr Val Lys Asn Phe Lys
Asn Gly Asp Leu Gly Ile Arg Leu Leu Asp Pro Asn Ser Tyr Tyr Tyr Phe Leu Glu Gly Gln
Asp Thr Gly Phe Tyr Gly Pro Ala Phe Tyr Ile Glu Arg Lys Asn Gly Gly Gly Ala Lys Asn
Asn Ser Ser Gly Ala Gly Asn Ser Lys Asp Trp Gly Gly Asn Gly His Gly Asn His Arg Asn
Asn Ala Ser Asp Leu Asn Lys Pro Asp Gly Asn Asn Gly Asn Asn Gln Asn Asn Gly Ser Asn
Gln Asp Asn His Ser Asp Val Asn Ala Pro Asn Asn Pro Gly Arg Asn Tyr Asp Ile Tyr Asp
Pro Leu Ala Leu Asp Leu Asp Gly Asp Gly Leu Glu Thr Val Ser Met Asn Gly Arg Gln Gly
Ala Leu Phe Asp His Glu Gly Lys Gly Ile Arg Thr Ala Thr Gly Trp Leu Ala Ala Asp Asp
Gly Phe Leu Val Leu Asp Arg Asn Gln Asp Gly Ile Ile Asn Asp Ile Ser Glu Leu Phe Ser
Asn Lys Asn Gln Leu Ser Asp Gly Ser Ile Ser Ala His Gly Phe Ala Thr Leu Ala Asp Leu
Asp Thr Asn Gln Asp Gln Arg Ile Asp Gln Asn Asp Lys Leu Phe Ser Lys Leu Gln Ile Trp
Arg Asp Leu Asn Gln Asn Gly Phe Ser Glu Ala Asn Glu Leu Phe Ser Leu Glu Ser Leu Asn
Ile Lys Ser Leu His Thr Ala Tyr Glu Glu Arg Asn Asp Phe Leu Ala Gly Asn Asn Ile Leu
Ala Gln Leu Gly Lys Tyr Glu Lys Thr Asp Gly Thr Phe Ala Gln Met Gly Asp Leu Asn Phe
Ser Phe Asn Pro Phe Tyr Ser Arg Phe Thr Glu Ala Leu Asn Leu Thr Glu Gln Gln Arg Arg
Thr Ile Asn Leu Thr Gly Thr Gly Arg Val Arg Asp Leu Arg Glu Ala Ala Ala Leu Ser Glu
Glu Leu Ala Ala Leu Leu Gln Gln Tyr Thr Lys Ala Ser Asp Phe Gln Ala Gln Arg Glu Leu
Leu Pro Ala Ile Leu Asp Lys Trp Ala Ala Thr Asp Leu Gln Tyr Gln His Tyr Asp Lys Thr
Leu Leu Lys Thr Val Glu Ser Thr Asp Ser Ser Ala Ser Val Val Arg Val Thr Pro Ser Gln
Leu Ser Ser Ile Arg Asn Ala Lys His Asp Pro Thr Val Met Gln Asn Phe Glu Gln Ser Lys
Ala Lys Ile Ala Thr Leu Asn Ser Leu Tyr Gly Leu Asn Ile Asp Gln Leu Tyr Tyr Thr Thr
Asp Lys Asp Ile Arg Tyr Ile Thr Asp Lys Val Asn Asn Met Tyr Gln Thr Thr Val Glu Leu
Ala Tyr Arg Ser Leu Leu Leu Gln Thr Arg Leu Lys Lys Tyr Val Tyr Ser Val Asn Ala Lys
Gln Phe Glu Gly Lys Trp Val Thr Asp Tyr Ser Arg Thr Glu Ala Leu Phe Asn Ser Thr Phe
Lys Gln Ser Pro Glu Asn Ala Leu Tyr Asp Leu Ser Glu Tyr Leu Ser Phe Phe Asn Asp
Pro Thr Glu Trp Lys Glu Gly Leu Leu Leu Leu Ser Arg Tyr Ile Asp Tyr Ala Lys Ala
Gln Gly Phe Tyr Glu Asn Trp Ala Ala Thr Ser Asn Leu Thr Ile Ala Arg Leu Arg Glu
Ala Gly Val Ile Phe Ala Glu Ser Thr Asp Leu Lys Gly Asp Glu Lys Asn Asn Ile Leu
Leu Gly Ser Gln Lys Asp Asn Asn Leu Ser Gly Ser Ala Gly Asp Asp Leu Leu Ile Gly
Gly Glu Gly Asn Asp Thr Leu Lys Gly Ser Tyr Gly Ala Asp Thr Tyr Ile Phe Ser Lys
Gly His Gly Gln Asp Ile Val Tyr Glu Asp Thr Asn Asn Asp Asn Arg Ala Arg Asp Ile
Asp Thr Leu Lys Phe Thr Asp Val Asn Tyr Ala Glu Val Lys Phe Arg Arg Val Asp Asn
Asp Leu Met Leu Phe Gly Tyr His Asp Thr Asp Ser Val Thr Val Lys Ser Phe Tyr Asn
His Glu Tyr Tyr Gln Phe Glu Lys Leu Glu Phe Ala Asp Arg Ser Ile Thr Arg Asp Glu
Leu Gly Lys Gln Gly Met Ala Leu Phe Gly Thr Asp Gly Asp Asp Asp Ile Asn Asp Trp
Gly Arg Asn Ser Val Ile Asp Ala Gly Ala Gly Asn Asp Thr Ile Asn Gly Ser Tyr Gly
Asp Asp Thr Leu Ile Gly Gly Thr Gly Asn Asp Ile Leu Lys Gly Ser Tyr Gly Ala Asp
Thr Tyr Leu Phe Ser Lys Gly His Gly Gln Asp Val Ile Tyr Glu Tyr Ser Asp Ser Ala
Asn Ser Lys Arg Asp Ile Asp Thr Leu Lys Phe Thr Asp Val Asn Tyr Ala Glu Val Lys
Phe Arg Arg Val Gly Asp Asp Leu Met Leu Phe Gly Tyr His Asp Thr Asp Ser Val Thr
Val Lys Ser Phe Tyr Asp His Glu Tyr Tyr Gln Phe Glu Lys Leu Glu Phe Ala Asp Arg
Ser Ile Ser Arg Asp Glu Leu Ile Lys Ala Gly Leu His Leu Tyr Gly Thr Asp Gly Asn
Asp Glu Ile Asn Asp His Ala Asp Trp Asp Ser Ile Leu Glu Gly Gly Lys Gly Asn Asp
Ile Leu Arg Gly Ser Tyr Gly Ala Asp Thr Tyr Leu Phe Ser Lys Gly His Gly Gln Asp
Val Ile Tyr Glu Tyr Ser Asp Ser Ala Asn Ser Lys Ser Asp Ile Asp Thr Leu Lys Phe
Thr Asp Ile Asn Tyr Ala Glu Val Lys Phe Arg Arg Val Gly Asp Asp Leu Met Leu Phe
Gly Tyr His Asp Thr Asp Ser Val Thr Val Lys Ser Phe Tyr Asn His Glu Tyr Tyr Gln
Phe Glu Lys Leu Glu Phe Ala Asp Arg Ser Ile Thr Arg Asp Glu Leu Gly Lys Gln Gly
Met Ala Leu Phe Gly Thr Asp Gly Asp Asp Asp Ile Asn Asp Trp Gly Arg Asn Ser Val
Ile Asp Ala Gly Ala Gly Asn Asp Thr Ile Asn Gly Ser Tyr Gly Asp Asp Thr Leu Ile
Gly Gly Thr Gly Asn Asp Ile Leu Lys Gly Ser Tyr Gly Ala Asp Thr Tyr Leu Phe Ser
Lys Gly His Gly Gln Asp Val Ile Tyr Glu Tyr Ser Asp Ser Ala Asn Ser Lys Arg Asp
Ile Asp Thr Leu Lys Phe Thr Asp Val Asn Tyr Ala Glu Val Lys Phe Arg Arg Val Gly
Asp Asp Leu Met Leu Phe Gly Tyr His Asp Thr Asp Ser Val Thr Val Lys Ser Phe Tyr
Ser His Glu Tyr Tyr Gln Phe Glu Lys Leu Glu Phe Ala Asp Arg Ser Ile Ser Arg Asp
Glu Leu Ile Lys Ala Gly Leu His Leu Tyr Gly Thr Asp Gly Asn Asp Glu Ile Asn Asp
His Ala Asp Trp Asp Ser Ile Leu Glu Gly Gly Lys Gly Asn Asp Ile Leu Arg Gly Ser
Tyr Gly Ala Asp Thr Tyr Ile Phe Ser Lys Gly His Gly Gln Asp Val Ile Tyr Glu Tyr
Ser Asp Ser Ala Asn Ser Lys Arg Asp Ile Asp Thr Leu Lys Phe Thr Asp Val Asn Tyr
Ala Glu Val Lys Phe Arg Arg Val Gly Asp Asp Leu Met Leu Phe Gly Tyr His Asp Thr
Asp Ser Val Thr Val Lys Ser Phe Tyr Asp His Glu Tyr Tyr Gln Phe Glu Lys Leu Glu
Phe Ala Asp Arg Ser Ile Thr Arg Asp Glu Leu Gly Lys Gln Gly Met Ala Leu Phe Gly
Thr Asp Gly Asp Asp Asp Ile Asn Asp Trp Gly Arg Asn Ser Val Ile Asp Ala Gly Ala
Gly Asn Asp Thr Ile Asn Gly Gly Tyr Gly Asp Asp Thr Leu Ile Gly Gly Lys Gly Asn
Asp Ile Leu Lys Gly Ser Tyr Gly Ala Asp Thr Tyr Leu Phe Ser Lys Gly His Gly Gln
Asp Val Ile Tyr Glu Tyr Ser Asp Ser Ala Ser Ser Lys Ser Asp Ile Asp Thr Leu Lys
Phe Thr Asp Ile Gly Leu Ser Glu Leu Trp Phe Ser Arg Glu Asn Asn Asp Leu Ile Ile
Lys Ser Leu Leu Ser Glu Asp Lys Val Thr Val Gln Asn Trp Tyr Ser His Gln Asp His
Lys Ile Glu Asn Ile Arg Leu Ser Asn Glu Gln Met Leu Val Ser Thr Gln Val Glu Lys
Met Val Glu Ser Met Ala Gly Phe Ala Gln Gln His Gly Gly Glu Ile Ser Leu Val Pro
Arg Glu Glu Val Lys Gln Tyr Ile Asn Ser Leu Thr Ala Ala Leu
APP ApxIV with N-terminal in-frame deletion
SEQ ID NO: 48
Met Thr Lys Leu Thr Met Gln Asp Val Thr Asn Leu Tyr Leu Tyr Lys Gln Arg Thr Leu Pro
Thr Asp Arg Leu Asp Asp Ser Leu Ile Ser Lys Thr Gly Lys Gly Glu Asn Ile Asp Lys Lys
Glu Phe Met Ala Gly Pro Gly Arg Phe Val Thr Ala Asp Asn Phe Ser Val Val Lys Asp Phe
Phe Thr Ala Lys Asp Ser Leu Ile Asn Leu Ser Leu Gln Thr Arg Ile Leu Ala Asn Leu Lys
Pro Gly Lys Tyr Ser Lys Ala Gln Ile Leu Glu Met Leu Gly Tyr Thr Lys Asn Gly Glu Lys
Val Asp Gly Met Phe Thr Gly Glu Val Gln Thr Leu Gly Phe Tyr Asp Asp Gly Lys Gly Asp
Leu Leu Glu Arg Gln Phe Glu Gly Lys Trp Val Thr Asp Tyr Ser Arg Thr Glu Ala Leu Phe
Asn Ser Thr Phe Lys Gln Ser Pro Glu Asn Ala Leu Tyr Asp Leu Ser Glu Tyr Leu Ser Phe
Phe Asn Asp Pro Thr Glu Trp Lys Glu Gly Leu Leu Leu Leu Ser Arg Tyr Ile Asp Tyr Ala
Lys Ala Gln Gly Phe Tyr Glu Asn Trp Ala Ala Thr Ser Asn Leu Thr Ile Ala Arg Leu Arg
Glu Ala Gly Val Ile Phe Ala Glu Ser Thr Asp Leu Lys Gly Asp Glu Lys Asn Asn Ile Leu
Leu Gly Ser Gln Lys Asp Asn Asn Leu Ser Gly Ser Ala Gly Asp Asp Leu Leu Ile Gly Gly
Glu Gly Asn Asp Thr Leu Lys Gly Ser Tyr Gly Ala Asp Thr Tyr Ile Phe Ser Lys Gly His
Gly Gln Asp Ile Val Tyr Glu Asp Thr Asn Asn Asp Asn Arg Ala Arg Asp Ile Asp Thr Leu
Lys Phe Thr Asp Val Asn Tyr Ala Glu Val Lys Phe Arg Arg Val Asp Asn Asp Leu Met Leu
Phe Gly Tyr His Asp Thr Asp Ser Val Thr Val Lys Ser Phe Tyr Asn His Glu Tyr Tyr Gln
Phe Glu Lys Leu Glu Phe Ala Asp Arg Ser Ile Thr Arg Asp Glu Leu Gly Lys Gln Gly Met
Ala Leu Phe Gly Thr Asp Gly Asp Asp Asp Ile Asn Asp Trp Gly Arg Asn Ser Val Ile Asp
Ala Gly Ala Gly Asn Asp Thr Ile Asn Gly Ser Tyr Gly Asp Asp Thr Leu Ile Gly Gly Thr
Gly Asn Asp Ile Leu Lys Gly Ser Tyr Gly Ala Asp Thr Tyr Leu Phe Ser Lys Gly His Gly
Gln Asp Val Ile Tyr Glu Tyr Ser Asp Ser Ala Asn Ser Lys Arg Asp Ile Asp Thr Leu Lys
Phe Thr Asp Val Asn Tyr Ala Glu Val Lys Phe Arg Arg Val Gly Asp Asp Leu Met Leu Phe
Gly Tyr His Asp Thr Asp Ser Val Thr Val Lys Ser Phe Tyr Asp His Glu Tyr Tyr Gln Phe
Glu Lys Leu Glu Phe Ala Asp Arg Ser Ile Ser Arg Asp Glu Leu Ile Lys Ala Gly Leu His
Leu Tyr Gly Thr Asp Gly Asn Asp Glu Ile Asn Asp His Ala Asp Trp Asp Ser Ile Leu Glu
Gly Gly Lys Gly Asn Asp Ile Leu Arg Gly Ser Tyr Gly Ala Asp Thr Tyr Leu Phe Ser Lys
Gly His Gly Gln Asp Val Ile Tyr Glu Tyr Ser Asp Ser Ala Asn Ser Lys Ser Asp Ile Asp
Thr Leu Lys Phe Thr Asp Ile Asn Tyr Ala Glu Val Lys Phe Arg Arg Val Gly Asp Asp Leu
Met Leu Phe Gly Tyr His Asp Thr Asp Ser Val Thr Val Lys Ser Phe Tyr Asn His Glu Tyr
Tyr Gln Phe Glu Lys Leu Glu Phe Ala Asp Arg Ser Ile Thr Arg Asp Glu Leu Gly Lys Gln
Gly Met Ala Leu Phe Gly Thr Asp Gly Asp Asp Asp Ile Asn Asp Trp Gly Arg Asn Ser Val
Ile Asp Ala Gly Ala Gly Asn Asp Thr Ile Asn Gly Ser Tyr Gly Asp Asp Thr Leu Ile Gly
Gly Thr Gly Asn Asp Ile Leu Lys Gly Ser Tyr Gly Ala Asp Thr Tyr Leu Phe Ser Lys Gly
His Gly Gln Asp Val Ile Tyr Glu Tyr Ser Asp Ser Ala Asn Ser Lys Arg Asp Ile Asp Thr
Leu Lys Phe Thr Asp Val Asn Tyr Ala Glu Val Lys Phe Arg Arg Val Gly Asp Asp Leu Met
Leu Phe Gly Tyr His Asp Thr Asp Ser Val Thr Val Lys Ser Phe Tyr Ser His Glu Tyr Tyr
Gln Phe Glu Lys Leu Glu Phe Ala Asp Arg Ser Ile Ser Arg Asp Glu Leu Ile Lys Ala Gly
Leu His Leu Tyr Gly Thr Asp Gly Asn Asp Glu Ile Asn Asp His Ala Asp Trp Asp Ser Ile
Leu Glu Gly Gly Lys Gly Asn Asp Ile Leu Arg Gly Ser Tyr Gly Ala Asp Thr Tyr Ile Phe
Ser Lys Gly His Gly Gln Asp Val Ile Tyr Glu Tyr Ser Asp Ser Ala Asn Ser Lys Arg Asp
Ile Asp Thr Leu Lys Phe Thr Asp Val Asn Tyr Ala Glu Val Lys Phe Arg Arg Val Gly Asp
Asp Leu Met Leu Phe Gly Tyr His Asp Thr Asp Ser Val Thr Val Lys Ser Phe Tyr Asp His
Glu Tyr Tyr Gln Phe Glu Lys Leu Glu Phe Ala Asp Arg Ser Ile Thr Arg Asp Glu Leu Gly
Lys Gln Gly Met Ala Leu Phe Gly Thr Asp Gly Asp Asp Asp Ile Asn Asp Trp Gly Arg Asn
Ser Val Ile Asp Ala Gly Ala Gly Asn Asp Thr Ile Asn Gly Gly Tyr Gly Asp Asp Thr Leu
Ile Gly Gly Lys Gly Asn Asp Ile Leu Lys Gly Ser Tyr Gly Ala Asp Thr Tyr Leu Phe Ser
Lys Gly His Gly Gln Asp Val Ile Tyr Glu Tyr Ser Asp Ser Ala Ser Ser Lys Ser Asp Ile
Asp Thr Leu Lys Phe Thr Asp Ile Gly Leu Ser Glu Leu Trp Phe Ser Arg Glu Asn Asn Asp
Leu Ile Ile Lys Ser Leu Leu Ser Glu Asp Lys Val Thr Val Gln Asn Trp Tyr Ser His
Gln Asp His Lys Ile Glu Asn Ile Arg Leu Ser Asn Glu Gln Met Leu Val Ser Thr Gln
Val Glu Lys Met Val Glu Ser Met Ala Gly Phe Ala Gln Gln His Gly Gly Glu Ile Ser
Leu Val Pro Arg Glu Glu Val Lys Gln Tyr Ile Asn Ser Leu Thr Ala Ala Leu

The invention will now be illustrated by the following non-limiting examples. The Examples that follow are illustrative of specific embodiments of the disclosure, and various uses thereof. They are set forth for explanatory purposes only and should not be construed as limiting the scope of the disclosure in any way.

### EXAMPLES

### Example 1: Generation of a Microorganism Expressing Inactive ApxIIA and ApxIIIA from an A. pleuropneumoniae Strain which Endogenously Expresses Wild-Type ApxIIA and ApxIIIA

Unmarked mutations have been introduced to an APP strain via use of two-step natural transformation method, resulting in the systematic alteration of the codons for both acylation sites in each of the endogenous apxIIA and apxIIIA genes present in said strain. Subsequently, generation of an unmarked in-frame deletion of an N-terminal immunogenic domain sequence in the apxIVA gene was used to generate DIVA vaccine candidates. Finally, an unmarked deletion of the competence regulatory gene, sxy, may be generated to render the strains unable to undergo further natural transformation, and so eliminate the most likely source of reversion to wild-type for any of the introduced mutations.

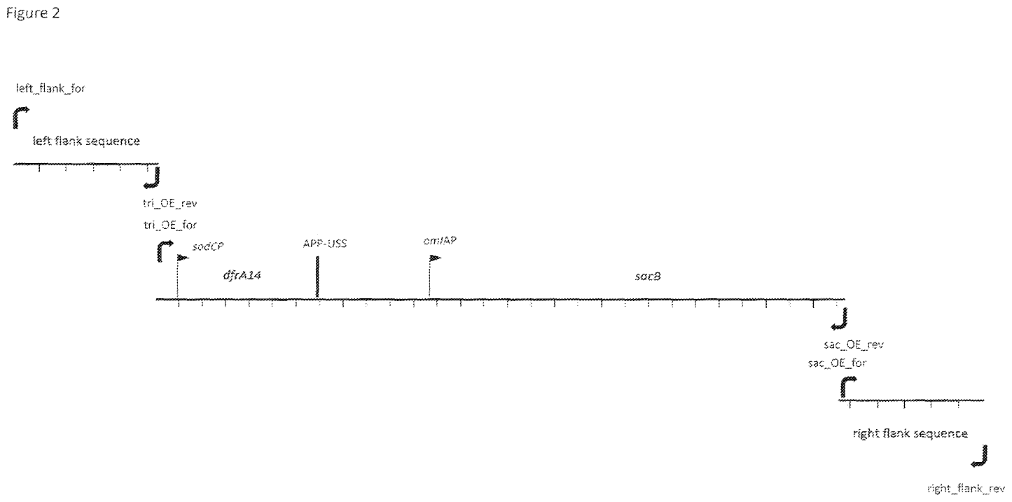

The inventors have previously described the use of a catsacB cassette (encoding a promoterless chloramphenicol resistance gene and sucrose sensitivity gene transcribed from the promoter of the omlA gene of APP) for generation of successive unmarked mutations in APP (Bossé et al. 2014 PLoS ONE 9(11): e111252). In the present example, a more refined dfrA14sacB cassette, encoding the trimethoprim resistance allele dfrA14, identified in endogenous APP plasmids (Bosse et al. (2015) J Antimicrob Chemother 70(8):2217-2222), and the sucrose sensitivity gene, sacB. The dfrA14sacB cassette was generated by overlap extension-PCR (OE-PCR) to combine a synthetic trimethoprim selection cassette (generated by Eurofins Genomics and consisting of the dfrA14 gene, preceded by the promoter for the sodC gene of APP, known to be active under all tested conditions, and followed by the 9-bp sequence required for uptake of DNA during natural transformation by APP) to the sacB gene PCR amplified from the previous catsacB cassette. Linker sequences, tri_OE_for (GTTAATGCCGTCTGAAGTGCGAAG, SEQ ID NO: 19) and sac_OE_rev (GAAGCAGTTGCACGTTCATGTCTC, SEQ ID NO: 20) were added on either end of the dfrA14sacB cassette to facilitate addition of all synthetically generated gene-specific left and right flanking sequences (comprising approx. 500 bases to either side of the region to be mutated) to which the complementary linker sequences (tri_OE_rev or sac_OE_for, as appropriate, described below) are added—see FIG. 2. The dfrA14sacB cassette is shown in SEQ ID NO: 18.

For generation of unmarked acylation site mutations, gene replacement constructs (to be used in the second round of natural transformation to remove the dfrA14sacB cassette added in the first round natural transformation—see below) were synthesised by Eurofins Genomics consisting of approx. 1500 bases of sequence comprising the full left and right flanking regions (see below) as well as the central acylation-site containing region in which the acylation site codons were altered (K to A and N to A for the respective acylation sites in ApxIIA; K to A for both acylation sites in each of ApxIA and ApxIIIA). These mutation constructs were synthesised with the 9 bp uptake signal sequence (USS) required for efficient natural transformation into APP (Redfield et al., 2006. BMC Evolutionary Biology 2006, 6:82) at the 3′, and were supplied already cloned into a pEX4K vector (with the resulting plasmids designated pExapxIAmut, pExapxIIAmut, and pExapxIIIAmut), each of which was linearised with XhoI prior to use in natural transformation for removal of the dfrA14sacB in the respective toxin gene mutants described below.

For sequential mutation of the acylation sites in the endogenous apxIIA and apxIIIA genes in the naturally transformable strains of serotype 8 and serotype 15, synthetic left and right flanking sequences (approximately 500 bases to the left and right of a central region of approximately 465 bp containing both acylation sites) were synthesised for both apxIIA and apxIIIA by Eurofins Genomics such that both left flank sequences were flanked on the 5′ end with a 24 bp left_flank_for priming site (ATTGGGTACCGAGCTCGC, SEQ ID NO: 21), and on the 3′ end with a 24 bp tri_OE_rev priming site (CTTCGCACTTCAGACGGCATTAAC, SEQ ID NO: 22) complementary to the linker sequence present at the 5′ end of the dfrA14sacB cassette. Similarly, both apxIIA and apxIIIA right flank sequences were synthesised by Eurofins Genomics such that the 5′ ends contained a 24 bp sac_OE_for primer sequence (GAGACATGAACGTGCAACTGCTTC, SEQ ID NO: 23) complementary to the 24 bp linker present at the 3′ end of the dfrA14sacB cassette, and the 3′ ends contained a 24 bp right_flank_rev primer site (CCATTTCACACAGGAATTCGGATC, SEQ ID NO: 24). In this way, the same primer pairs, i.e. left_flank_forward/tri_OE_rev were used to reamplify all synthetic left flank sequences, and sac_OE_for/right_flank_rev were used to reamplify all synthetic right flank sequences, prior to OE-PCR fusion of the flanks to the central dfrA14sacB cassette which had been amplified using tri_OE_for/sac_OE_rev primers. All PCR amplifications were performed using the CloneAmp proof-reading polymerase and, where necessary, template DNA was subsequently removed by treatment with DpnI.

Overlap extension PCRs were performed by combining roughly equimolar concentrations of the left and right flanking sequences (used as primers) and the dfrA14sacB in a total volume of 15 μl of 1× CloneAmp PCR mix. Following an initial amplification of 12 cycles of 98° C./10 sec, 60° C./10 sec, 72° C./3 min, a 1 μl aliquot of the fused overlap product was then used as template for a subsequent 15 cycle PCR amplification (using the same cycling conditions) with the terminal left_flank_forward/right_flank_rev primers (at a final concentration of 1 μM each) in a total volume of 30 μl 1× CloneAmp mix. The resulting fused gene replacement constructs were cleaned using the Qiamp PCR cleanup kit and used directly as template DNA for natural transformation (or, alternatively, were A-tailed and cloned into pGEMT, with the resulting verified clones linearised by digestion with SpeI prior to use in natural transformation) for replacement of the central approx. 465 bp sequences of the respective toxin genes (containing the acylation sites) with the dfrA14sacB cassette. Transformants were selected on Columbia agar plates containing 0.01% nicotinamide adenine dinucleotide and 10 μg/ml trimethoprim (Col-NAD-Tri10) and subsequently screened for sensitivity to sucrose on salt-free LB agar supplemented with 10% sucrose, 5% horse serum and 0.01% NAD (LB-SSN). Sequencing was used to confirm correct insertion of the dfrA14sacB cassette replacing the respective toxin gene regions. Gene replacement constructs delta_apxIIA_dfrA14sacB and delta_apxIIIA_dfrA14sacB are shown in SEQ ID Nos: 37 and 39 respectively.

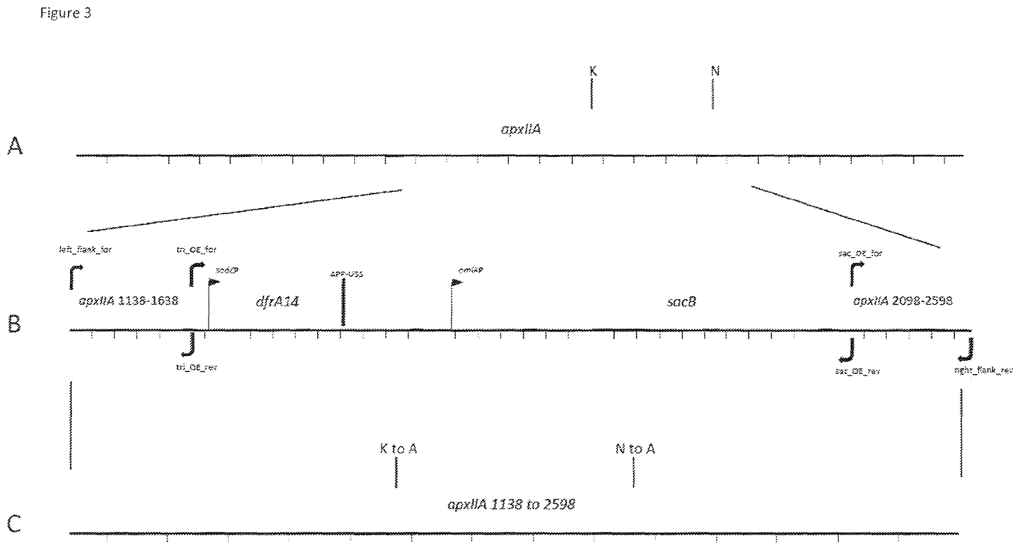

The dfrA14sacB cassette was subsequently removed in a second round of natural transformation using the appropriate mutation constructs (i.e. apxIIA_mut or apxIIIA_mut, as required; see SEQ ID NOs: 38 and 40, respectively) to leave unmarked mutants, selected for sucrose resistance by plating on LB-SSN plates. Sensitivity to trimethoprim following loss of the dfrA14sacB cassette was confirmed by plating onto Col-NAD-Tri10 plates and selected clones were sequenced across the modified region of the respective toxin sequence to confirm the presence of both modified acylation sites and no other mutation. See FIG. 3 for an example (using the apxIIA constructs) of the two-step natural transformation system for removal of acylation sites.

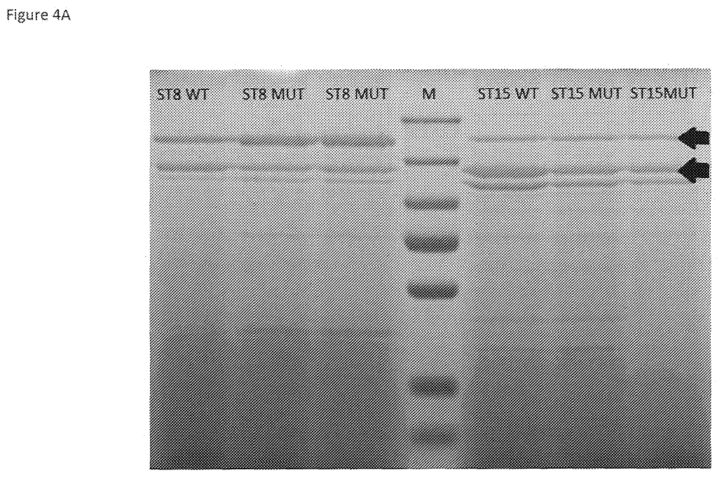

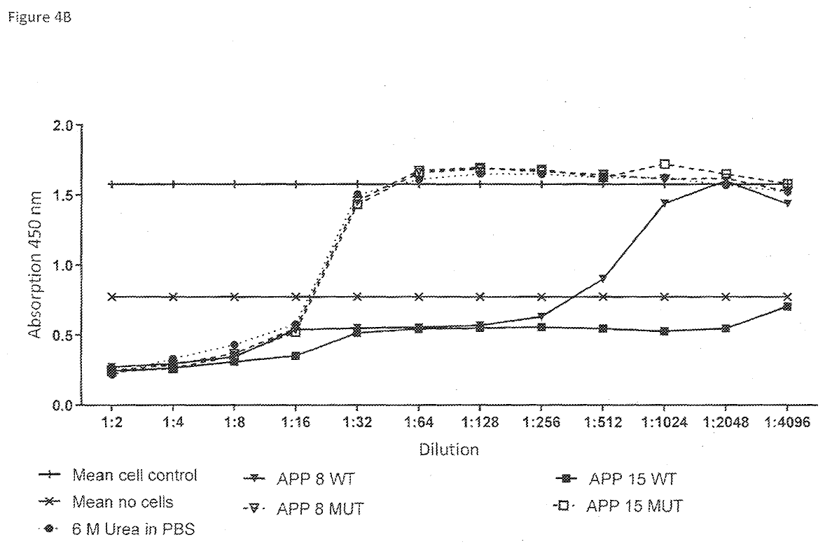

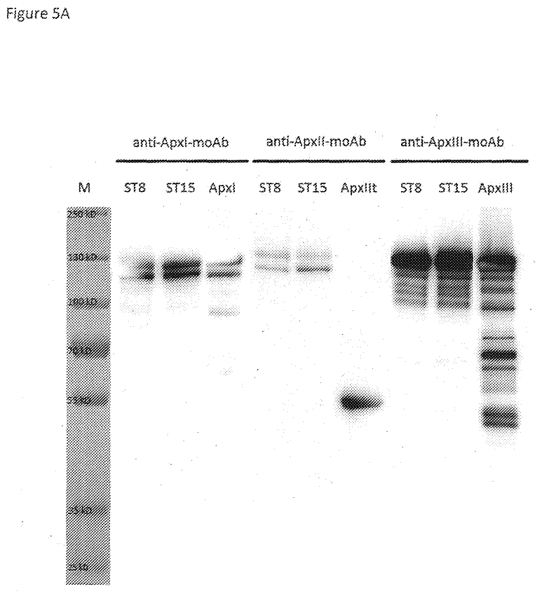

Unmarked apxIIA mutants were generated first, followed by mutation of apxIIIA, to generate double toxin mutants (apxIIA_mut/apxIIIA_mut) in which both acylation sites were altered in each of the ApxII and ApxIII proteins. Secretion of immunogenic non-toxic ApxIIA and ApxIIIA proteins was confirmed by SDS-PAGE analysis of cell free culture supernatants and cytotoxicity assays using cultured BL3 cells, with wild-type active ApxII and ApxIII toxins used as positive controls. As shown in FIG. 4A, no difference in the expression level of the wild-type ApxIIA and ApxIIIA polypeptides was observed. However, when the cytotoxicity of the wild-type and mutant ApxIIA and ApxIIIA polypeptides were tested in a BL3 cell-based assay, the mutant ApxIIA and ApxIIIA polypeptides did not induce cell death at a level above the urea control. In contrast, wild-type ApxIIA and ApxIIIA remained cytotoxic even at dilutions of 1:1024 and above (FIG. 4B).

### Example 2: Generation of a Microorganism Expressing all Three of ApxIA, ApxIIA and ApxIIIA in an Inactive Form from an A. pleuropneumoniae Strain which Endogenously Expresses Wild-Type ApxIIA and ApxIIIA

An alternative to having two different strains to produce the full complement of detoxified Apx proteins is to generate a single strain secreting all three proteins. To do this, a mutated apxIA gene (along with the apxIC gene) was introduced to replace the truncated apxIA sequence present in isolates in which the endogenous apxIIA and apxIIIA genes were already mutated to remove acylation sites, as described in Example 1 above. The resulting mutants therefore expressed all three ApxI-ApxIII proteins, each of which is an inactive toxin.

Briefly, a dfrA14sacB-containing construct (i.e. delta_apxIA_trunc_dfrA14sacB; SEQ ID NO: 41) was generated to replace the existing truncated apxIA sequence (see FIG. 2) in a manner similar to generation of the apxIIA and apxIIIA constructs described in Example 1 (i.e., appropriate left and right flanking sequences were synthesised with linkers to allow OE-PCR fusion to the dfrA14sacB cassette). This dfrA14sacB-containing construct was introduced by natural transformation into the apxIIA_mut/apxIII_mut double toxin mutants generated in Example 1, with selection of transformants on Col-NAD-Tri10 plates and confirmation of sucrose sensitivity on LB-SSN plates.

The correct gene replacement/insertion of the dfrA14sacB cassette (replacing the truncated apxIA) was confirmed by sequencing. To remove the dfrA14sacB cassette in the mutant, an extended 4.7 kb unmarked mutation (apxIAmut_long; SEQ ID NO: 42), capable of reconstituting an intact apxI operon (in which both acylation sites of the apxIA gene are mutated) was then generated as follows. Extended left (2773 bp) and right (1481 bp) flanking sequences were PCR amplified from genomic DNA extracted from Shope 4074 (serotype 1 strain with complete apxI operon) using primers aaacaagcggtCCGGATCTTGGAATTTCGGC (SEQ ID NO: 25)/TGCCTTCAAGCGGATCAAACAC (SEQ ID NO: 26) for the left flank, and TCGAACTTGGGAACGGTATCAG (SEQ ID NO: 27)/ttacaagcggtACTTTGCCAGCTTACCTACGATG (SEQ ID NO: 28) for the right flank. Copies of the 9 bp USS was appended to the 5′ ends of both the left flank forward and right flank reverse primers (shown underlined in both sequences); the left flank reverse and right flank forward primers were complementary to those used to amplify 566 bp of the apxIA sequence (containing both mutated acylation sites) from the synthetic construct in the plasmid pExapxIAmut, i.e. primers apxIA_mut_for_OE (GTGTTTGATCCGCTTGAAGGCA, SEQ ID NO: 29) and apxIA_mut_rev_OE (CTGATACCGTTCCCAAGTTCGA, SEQ ID NO: 30). The left and right flanks were combined in equimolar ratios with the central 566 bp amplicon and fused by OE-PCR as described in Example 1. The resulting fusion product was cleaned, A-tailed and cloned into pGEMT. Sequencing was used to confirm the correct gene replacement construct in the resulting plasmid pTapxIAmut_long, which was linearised with SpeI prior to use in the second round of natural transformation to remove the dfrA14sacB cassette and leave a reconstituted, but mutated, apxI operon in the chromosome, with selection of transformants on LB-SSN plates. Confirmation of trimethoprim sensitivity due to loss of the dfrA14sacB cassette was assessed by sub-culturing onto Col-NAD-Tri10 plates. Sequencing across the insertion site confirmed reconstitution of the mutated apxI operon (i.e., insertion of the apxICA genes, with the apxIA gene having both acylation sites mutated).

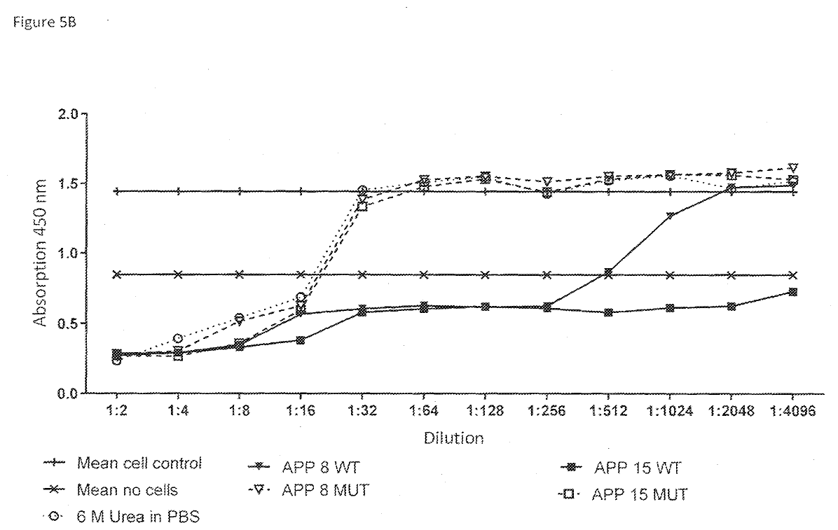

Secretion of all three immunogenic non-toxic proteins in the resulting triple toxin mutants was confirmed by Western blot (FIG. 5A) using monoclonal antibodies specific for the ApxII and ApxIII proteins, and cytotoxicity assays (FIG. 5B), as in Example 1 above. As shown in FIG. 5A, a single ST8 strain was capable of producing inactive forms of all three of ApxIA, ApxIIA and ApxIIIA. Similarly, a single ST15 strain was also capable of producing inactive forms of all three of ApxIA, ApxIIA and ApxIIIA. Supernatants from these ST8 and ST15 strains (each expressing inactive forms of all three of ApxIA, ApxIIA and ApxIIIA) were assayed for cytotoxicity compared with the respective wild-type ST8 and ST15 strains in a BL3 cell-based assay. As shown in FIG. 5B, the supernatant from both the ST8 and ST15 strains expressing mutant/inactive ApxIA, ApxIIA and ApxIIIA polypeptides did not induce cell death at a level above the urea control. In contrast, the wild-type ST8 and ST15 strains expressing wild-type ApxIA, ApxIIA and ApxIIIA remained cytotoxic even at dilutions of 1:1024 and above (FIG. 5B).

### Example 3: Introduction of an apxIVA Mutation into the Triple ApxIA/ApxIIA/ApxIIIA Mutant to Produce a DIVA Strain

Following confirmation of creation of triple toxin mutants in Example 2, a 2586 bp in-frame N-terminal deletion in the apxIVA gene was generated. Similar to creation of the acylation site mutations described in Examples 1 and 2 above, synthetic left and right flanking sequences of approx. 500 bp (to either side of the 2586 bp region to be deleted) were generated by Eurofins Genomics. In this instance, the left_flank_for_USS primer sequence (ATCC\sqrt{ACAAGCGGT}CATCTGGC, SEQ ID NO: 31) added to the 5′ end of the apxIV left flank construct was modified to incorporate the 9 bp USS (underlined) required for natural transformation; all other linker priming sites for the left and right flank sequences were as used in Examples 1 and 2. The gene replacement construct delta_apxIVA_dfrA14sacB is shown in SEQ ID NO: 43. A 1043 bp deletion construct (apxIV_int_del, SEQ ID NO: 44), consisting of the left and right flanking regions fused together at the site of the 2586 bp in-frame deletion was generated by Eurofins Genomics, with the same terminal left_flank_for USS and right_flank_rev priming sites to allow re-amplification of the synthetic strand.

Following amplification of the synthetic left and right flank constructs using left_flank_for_USS/tri_OE_rev and sac_OE_for/right_flank_rev, and the dfrA14sacB cassette using tri_OE_for/sac_OE_rev, the three cleaned sequences were fused by OE-PCR, as above. This construct was introduced into the triple toxin mutants by natural transformation with selection on Col-NAD-Tri10 plates. Following confirmation of the correct insertion site, the dfrA14sacB cassette was removed using the amplified apxIV_int_del sequence in a second round of natural transformation, with selection of transformants on LB-SSN plates. Loss of the dfrA14sacB cassette was confirmed by plating on Col-NAD-Tri10 plates to show trimethoprim sensitivity, and by PCR to show the 2586 bp in-frame N-terminal deletion.

### Example 4: Introduction of a Sxy Mutation into Triple ApxIA/ApxIIA/ApxIIIA DIVA (Internal ApxIV Deletion) Mutant to Eliminate Further Natural Transformation

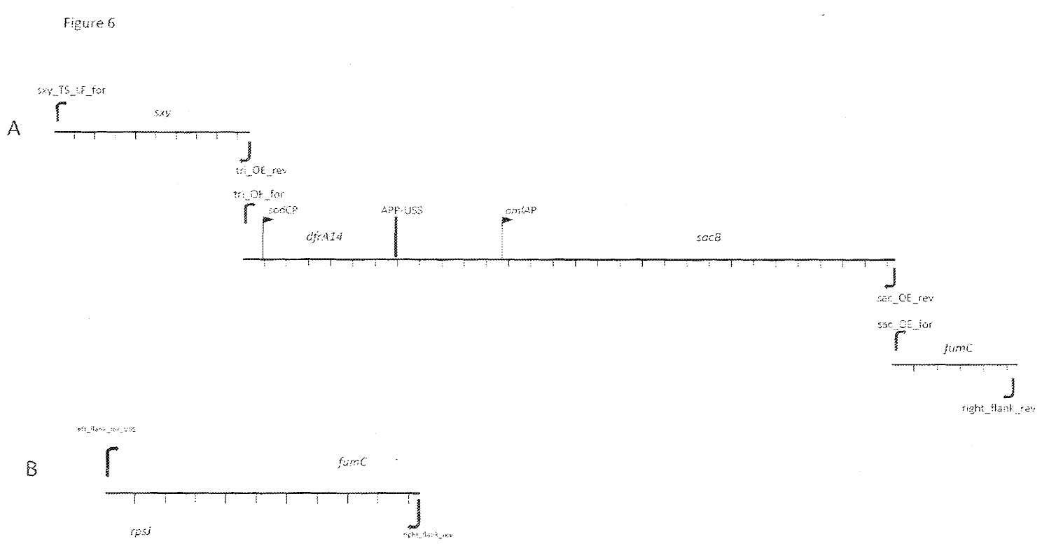

Following confirmation of creation of the DIVA (internal ApxIV deletion) triple toxin mutants in Example 3, the sxy gene is deleted using a modified version of the method for generating the in-frame apxIV deletion. As the Sxy protein is required for the second round of natural transformation, the construct for the first round of natural transformation to introduce the dfra14sacB cassette is not designed using flanking regions that would replace the sxy gene with the cassette, but rather to introduce the cassette immediately downstream of the sxy gene. The construct to remove the dfra14sacB cassette in the second round of transformation is designed to also remove the entire sxy gene, leaving an unmarked deletion (FIG. 6). The gene replacement construct sxy_dfrA14sacB_insert is shown in SEQ ID NO: 45. The sxy deletion construct is shown in SEQ ID NO: 46.

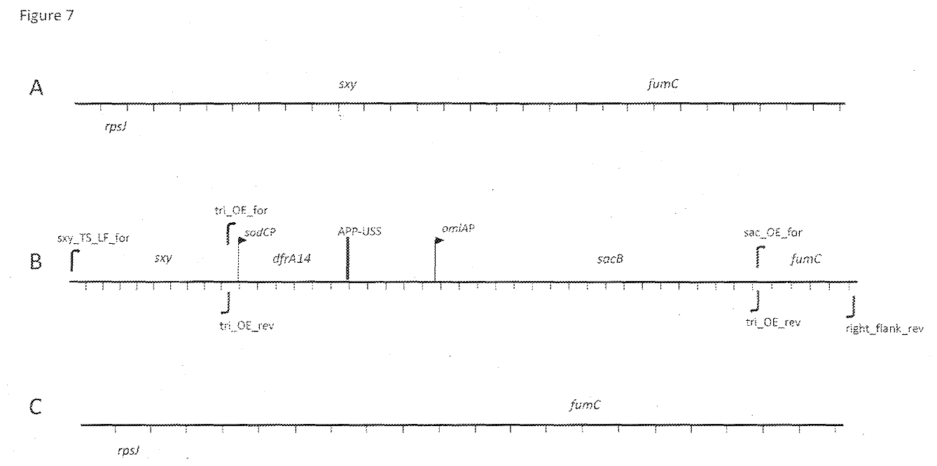

Due to complex secondary structure of the sequence upstream of the sxy gene, generation of a synthetic left flank was not possible. In order to PCR amplify an appropriate left flank sequence that would be amenable for use in natural transformation, the sequence upstream of sxy was analysed for the presence of a natural USS (FIG. 7). A sequence differing at one base from the 9 bp USS was identified 264 bp upstream of sxy and a forward primer was designed with a 1 bp mismatch in order to generate a perfect USS near the 5′ end of the primer (sxy_TS_LF_for GTACCGCTTGtTAAATGATTACACC, SEQ ID NO: 32; the USS is underlined with the single mismatched base in lower case). Two reverse primers, Sxy_TS_LF_rev1 (ggcAtTAAcTTAGTTAGCCTGTGAGATAGC, SEQ ID NO: 33; the lower case letters indicate bases not matching the endogenous APP sequence but required for addition of a portion of the linker) and Sxy_TS_LF_rev2 (CTTCGCACTTCAGACGGCATTAACTTAGTTAGCCTGTGAG, SEQ ID NO: 34; the bases indicated in bold match the sequence of the tri_OE_rev primer) were designed to allow sequential addition, by two successive rounds of PCR with the sxy_TS_LF_for used as forward primer in both reactions, of sequence to generate the linker required for fusion of the left flank to the dfra14sacB cassette. The resulting 952 bp left flank product comprised the entire sxy gene, along with approximately 270 bp upstream sequence, and a 3′ end complimentary to the 5′ end of the dfra14sacB cassette.

A right flank sequence, comprising approximately 500 bp of sequence downstream of the sxy gene was synthesised by Eurofins, with the appropriate sac_OE_for and right_flank_rev priming sites at the 5′ and 3′ ends, respectively, as described in Example 1. Following amplification of both the left and right flanking sequences, as well as the dfra14sacB cassette, the three fragments were fused by OE-PCR, as described in Example 1. The product of this OE-PCR is used in the first round of natural transformation, to introduce the dfra14sacB cassette downstream of the sxy gene, with selection on Col-NAD-Tri10 plates.

A sxy deletion construct was synthesised by Eurofins, comprising the same 500 bp downstream of sxy used in generating the right flank sequence fused on the 5′ end to approximately 500 bp sequence upstream of sxy, bounded by the left_flank_for_USS and right_flank_rev priming sites, as described in Examples 3 and 1, respectively, to allow re-amplification of the synthetic construct. Following confirmation of the correct insertion site in the mutants generated by the first round of natural transformation, the dfrA14sacB cassette is removed along with the entire sxy gene, by transformation with the amplified sxy deletion construct. Transformants are selected on LB-SSN plates. Loss of the dfrA14sacB cassette is confirmed by plating on Col-NAD-Tri10 plates to show trimethoprim sensitivity, and by PCR and sequencing to confirm the clean deletion of the sxy gene.

### Example 5: Generation of a Microorganism Expressing all Three of ApxIA, ApxIIA and ApxIIIA in an Inactive Form from an A. pleuropneumoniae Strain which Endogenously Expresses Wild-Type ApxIA and ApxIIA

In the event of identification of a naturally transformable isolate encoding ApxI and ApxII, the structural genes for these two toxins is similarly be inactivated using the same protocol as described in Example 1. Following mutation of the apxIIA gene, as described in Example 1, the apxIA gene is similarly mutated using the gene replacement and mutation constructs delta_apxIA_dfrA14sacB and the apxIA_mut, respectively, shown in SEQ ID Nos: 35 and 36. This is followed by the introduction of genes encoding for inactive ApxIIIA, amplified from a suitable apxIIIA mutant as generated in Example 1. Lastly, this is followed by introduction of the apxIVA and sxy mutations (as per Examples 2 and 4).

1. A microorganism comprising:
(a) a nucleic acid sequence encoding ApxIA of Actinobacillus pleuropneumoniae;
(b) a nucleic acid sequence encoding ApxIIA of A. pleuropneumoniae; and
(c) a nucleic acid sequence encoding ApxIIIA of A. pleuropneumoniae.

2. The microorganism of claim 1, wherein the nucleic acid sequences of (a), (b) and/or (c) are:
(i) comprised within the genome of the microorganism; or
(ii) comprised extra-chromosomally.

3. The microorganism of claim 1 or 2, wherein the ApxIA, ApxIIA and ApxIIIA are:
(a) inactive ApxIA, ApxIIA and ApxIIIA which have common antigenic cross-reactivity with wild-type ApxIA, ApxIIA and ApxIIIA; or
(b) wild-type ApxIA, ApxIIA and ApxIIIA.

4. The microorganism of claim 3, wherein:
(a) (i) the inactive ApxIA has an amino acid sequence corresponding to the wild-type ApxIA amino acid sequence of SEQ ID NO: 1, modified in at least one amino acid selected from the group consisting of K560 and K686, or a variant or fragment thereof which is at least 90% homologous to said inactive ApxIA amino acid sequence, said fragment comprising at least 30% of the consecutive amino acids of said inactive ApxIA amino acid sequence, wherein said variant or fragment comprises the at least one modified amino acid;
(ii) the inactive ApxIIA has an amino acid sequence corresponding to the wild-type ApxIIA amino acid sequence of SEQ ID NO: 2, modified in at least one amino acid selected from the group consisting of K557 and N687, or a variant or fragment thereof which is at least 90% homologous to said inactive ApxIIA amino acid sequence, said fragment comprising at least 30% of the consecutive amino acids of said inactive ApxIIA amino acid sequence, wherein said variant or fragment comprises the at least one modified amino acid; and
(iii) the inactive ApxIIIA has an amino acid sequence corresponding to the wild-type ApxIIIA amino acid sequence of SEQ ID NO: 3, modified in at least one amino acid selected from the group consisting of K571 and K702, or a variant or fragment thereof which is at least 90% homologous to said inactive ApxIIIA amino acid sequence, said fragment comprising at least 30% of the consecutive amino acids of said inactive ApxIIIA amino acid sequence, wherein said variant or fragment comprises the at least one modified amino acid;
and the at least one modified amino acid is substituted by an amino acid not susceptible to acylation; or
(b) (i) the inactive ApxIA has an amino acid sequence corresponding to the wild-type ApxIA amino acid sequence of SEQ ID NO: 1, containing deletions comprising at least one amino acid selected from the group consisting of K560 and K686, or a variant or fragment thereof which is at least 90% homologous to said inactive ApxIA amino acid sequence, said fragment comprising at least 30% of the consecutive amino acids of said inactive ApxIA amino acid sequence, wherein said variant or fragment comprises the deletion;
(ii) the inactive ApxIIA has an amino acid sequence corresponding to the wild-type ApxIIA amino acid sequence of SEQ ID NO: 2, containing deletions comprising at least one amino acid selected from the group consisting of K557 and N687, or a variant or fragment thereof which is at least 90% homologous to said inactive ApxIIA amino acid sequence, said fragment comprising at least 30% of the consecutive amino acids of said inactive ApxIIA amino acid sequence, wherein said variant or fragment comprises the deletion; and
(iii) the inactive ApxIIIA has an amino acid sequence corresponding to the wild-type ApxIIIA amino acid sequence of SEQ ID NO: 3, containing deletions comprising at least one amino acid selected from the group consisting of K571 and K702, or a variant or fragment thereof which is at least 90% homologous to said inactive ApxIIIA amino acid sequence, said fragment comprising at least 30% of the consecutive amino acids of said inactive ApxIIIA amino acid sequence, wherein said variant or fragment comprises the deletion.

5. The microorganism of claim 3 or 4, wherein:
(a) each amino acid not susceptible to acylation is independently selected from the group consisting of alanine, glycine, isoleucine, leucine, methionine, valine, serine, threonine, asparagine, glutamine, aspartic acid, histidine, aspartic acid, cysteine, proline, phenylalanine, tyrosine, tryptophan and glutamic acid; preferably selected from the group consisting of alanine, glycine, serine, isoleucine and leucine, valine and threonine; most preferably selected from the group consisting of alanine, glycine and serine; and/or
(b) (i) the inactive ApxIA has substitutions at both K560 and K686;
(ii) the inactive ApxIIA has substitutions at both K557 and N687; and
(iii) the inactive ApxIIIA has substitutions at both K571 and K702; and/or
(c) (i) the inactive ApxIA comprises the amino acid sequence of SEQ ID NO: 4;
(ii) the inactive ApxIIA comprises the amino acid sequence of SEQ ID NO: 5; and
(iii) the inactive ApxIIIA comprises the amino acid sequence of SEQ ID NO: 6.

6. The microorganism of claim 3 or 4, wherein:
(i) the inactive ApxIA has deletions at both K560 and K686;
(ii) the inactive ApxIIA has deletions at both K557 and N687; and
(iii) the inactive ApxIIIA has deletions at both K571 and K702.

7. The microorganism of claim 3, wherein:
(a) the wild-type ApxIA has an amino acid sequence corresponding to SEQ ID NO: 1, or a variant or fragment thereof which is at least 90% homologous to said wild-type ApxIA amino acid sequence, said fragment comprising at least 30% of the consecutive amino acids of said wild-type ApxIA amino acid sequence;
(b) the wild-type ApxIIA has an amino acid sequence corresponding to SEQ ID NO: 2, or a variant or fragment thereof which is at least 90% homologous to said wild-type ApxIIA amino acid sequence, said fragment comprising at least 30% of the consecutive amino acids of said wild-type ApxIIA amino acid sequence; and
(c) the wild-type ApxIIIA has an amino acid sequence corresponding to SEQ ID NO: 3, or a variant or fragment thereof which is at least 90% homologous to said wild-type ApxIIIA amino acid sequence, said fragment comprising at least 30% of the consecutive amino acids of said wild-type ApxIIIA amino acid sequence.

8. The microorganism of any one of the preceding claims which is an Escherichia coli strain or an Actinobacillus strain, preferably an Actinobacillus pleuropneumoniae strain.

9. The microorganism of claim 8, wherein the A. pleuropneumoniae strain is produced from:
(a) an A. pleuropneumoniae strain which expresses an endogenous ApxIIA and ApxIIIA, preferably a serotype 2, 8, or 15 strain; or
(b) an A. pleuropneumoniae strain which expresses an endogenous ApxIA and ApxIIA, preferably a serotype 1, 5 or 9 strain.

10. The microorganism of claim 8 or 9, which is an A. pleuropneumoniae strain in which at least one additional gene is modified, wherein preferably:
(a) said one or more additional gene is selected from the group consisting of apxIVA, sxy, nlpD and/or ssrA; and/or
(b) said modification results in the inactivation of said one or more additional gene.

11. The microorganism of any one of claims 8 to 10, which is an A. pleuropneumoniae strain in which the at least one additional gene which is modified is (i) apxIVA; (ii) sxy; or (iii) apxIVA and sxy, wherein preferably:
(a) the apxIVA gene is modified by an unmarked in-frame deletion of an N-terminal immunogenic domain sequence; and/or
(b) the sxy gene is deleted.

12. A vaccine composition comprises a microorganism as defined in any one of the preceding claims and at least a pharmaceutical carrier, a diluent and/or an adjuvant.

13. The vaccine composition of claim 12, which is a live vaccine, wherein preferably:
(a) the microorganism is an Actinobacillus pleuropneumoniae strain; and/or
(b) the ApxIA, ApxIIA and ApxIIIA are inactive ApxIA, ApxIIA and ApxIIIA which have common antigenic cross-reactivity with wild-type ApxIA, ApxIIA and ApxIIIA.

14. The vaccine composition of claim 12, which is an inactivated vaccine, wherein preferably:
(a) the microorganism is an Actinobacillus pleuropneumoniae strain; and/or
(b) the ApxIA, ApxIIA and ApxIIIA are wild-type ApxIA, ApxIIA and ApxIIIA which have been subsequently inactivated, preferably by chemical and/or heat treatment.

15. A method of producing a live vaccine composition as defined in claim 13, comprising:
(a) culturing a microorganism as defined in any one of claims 1, 2, 3 to 5 or 7 to 10, wherein the ApxIA, ApxIIA and ApxIIIA are inactive ApxIA, ApxIIA and ApxIIIA which have common antigenic cross-reactivity with wild-type ApxIA, ApxIIA and ApxIIIA;
(b) isolating the microorganism; and
(c) formulating the microorganism with a pharmaceutical carrier, a diluent and/or an adjuvant.

16. A method of producing an inactivated vaccine composition as defined in claim 14, comprising:
(a) culturing a microorganism as defined in any one of claims 1, 2 or 6 to 10, wherein the ApxIA, ApxIIA and ApxIIIA are wild-type ApxIA, ApxIIA and ApxIIIA;
(b) isolating the microorganism;
(c) inactivating the microorganism, preferably by chemical and/or heat treatment; and
(d) formulating the inactivated microorganism with a pharmaceutical carrier, a diluent and/or an adjuvant.

17. A method of producing a subunit vaccine composition, comprising:
(a) (i) culturing a microorganism as defined in any one of claims 1, 2, 3 to 5 or 7 to 10, wherein the ApxIA, ApxIIA and ApxIIIA are inactive ApxIA, ApxIIA and ApxIIIA which have common antigenic cross-reactivity with wild-type ApxIA, ApxIIA and ApxIIIA;
(ii) isolating the inactive ApxIA, ApxIIA and ApxIIIA from the cultured microorganism; and
(iii) formulating the inactive ApxIA, ApxIIA and ApxIIIA with a pharmaceutical carrier, a diluent and/or an adjuvant; or
(b) (i) culturing a microorganism as defined in any one of claims 1, 2 or 6 to 10, wherein the ApxIA, ApxIIA and ApxIIIA are wild-type ApxIA, ApxIIA and ApxIIIA;
(ii) isolating the wild-type ApxIA, ApxIIA and ApxIIIA from the cultured microorganism;
(iii) inactivating the wild-type ApxIA, ApxIIA and ApxIIIA, preferably by chemical and/or heat treatment; and
(iv) formulating the inactivated wild-type ApxIA, ApxIIA and ApxIIIA with a pharmaceutical carrier, a diluent and/or an adjuvant.

18. A vaccine composition as defined in any one of claims 12 to 14 for use in a method of prophylactic, metaphylactic or therapeutic treatment of a pneumonia, a pleurisy or a pleuropneumonia, in particular, of a pneumonia, a pleurisy or a pleuropneumonia caused by Actinobacillus pleuropneumoniae, wherein optionally the vaccine composition is to be administered intramuscularly, intradermally, intravenously, subcutaneously, or by mucosal administration.

19. An expression system comprising a microorganism as defined in any one of claims 1 to 11, further comprising at least one additional nucleic acid which encodes one or more additional swine pathogen antigen, wherein preferably the at least one additional nucleic acid is comprised within the genome of the microorganism.

20. A vector or set of vectors comprising nucleic acids encoding for:
(a) wild-type ApxIA, ApxIIA and ApxIIIA as defined in any one of claim 3 or 7; or
(b) inactive ApxIA, ApxIIA and ApxIIIA which have common antigenic cross-reactivity with wild-type ApxIA, ApxIIA and ApxIIIA as defined in any one of claims 3 to 6.


SEQUENCE LISTING (48 sequences)

SEQ ID NO: 1 - protein, 1022 aa, Actinobacillus pleuropneumoniae
MANSQLDRVKGLIDSLNQHTKSAAKSGAGALKNGLGQVKQAGQKLILYIPKDYQASTGSSLNDLVKAAEALGIEVHRSEKNGTALAKELFGTTEKLLGFSERGIALFAPQFDKLLNKNQKLSKSLGGSSEALGQRLNKTQTALSALQSFLGTAIAGMDLDSLLRRRRNGEDVSGSELAKAGVDLAAQLVDNIASATGTVDAFAEQLGKLGNALSNTRLSGLASKLNNLPDLSLAGPGFDAVSGILSVVSASFILSNKDADAGTKAAAGIEISTKILGNIGKAVSQYIIAQRVAAGLSTTAATGGLIGSVVALAISPLSFLNVADKFERAKQLEQYSERFKKFGYEGDSLLASFYRETGAIEAALTTINSVLSAASAGVGAAATGSLVGAPVAALVSAITGIISGILDASKQAIFERVATKLANKIDEWEKKHGKNYFENGYDARHSAFLEDTFELLSQYNKEYSVERVVAITQQRWDVNIGELAGITRKGADAKSGKAYVDFFEEGKLLEKDPDRFDKKVFDPLEGKIDLSSINKTTLLKFITPVFTAGEEIRERKQTGKYEYMTELFVKGKEKWVVTGVQSHNAIYDYTNLIQLAIDKKGEKRQVTIESHLGEKNDRIYLSSGSSIVYAGNGHDVAYYDKTDTGYLTFDGQSAQKAGEYIVTKELKADVKVLKEVVKTQDISVGKRSEKLEYRDYELSPFELGNGIRAKDELHSVEEIIGSNRKDKFFGSRFTDIFHGAKGDDEIYGNDGHDILYGDDGNDVIHGGDGNDHLVGGNGNDRLIGGKGNNFLNGGDGDDELQVFEGQYNVLLGGAGNDILYGSDGTNLFDGGVGNDKIYGGLGKDIYRYSKEYGRHIIIEKGGDDDTLLLSDLSFKDVGFIRIGDDLLVNKRIGGTLYYHEDYNGNALTIKDWFKEGKEGQNNKIEKIVDKDGAYVLSQYLTELTAPGRGINYFNGLEEKLYYGEGYNALPQLRKDIEQIISSTGALTGEHGQVLVGAGGPLAYSNSPNSIPNAFSNYLTQSA

SEQ ID NO: 2 - protein, 956 aa, Actinobacillus pleuropneumoniae
MSKITLSSLKSSLQQGLKNGKNKLNQAGTTLKNGLTQTGHSLQNGAKKLILYIPQGYDSGQGNGVQDLVKAANDLGIEVWREERSNLDIAKTSFDTTQKILGFTDRGIVLFAPQLDNLLKKNPKIGNTLGSASSISQNIGKANTVLGGIQSILGSVLSGVNLNELLQNKDPNQLELAKAGLELTNELVGNIASSVQTVDAFAEQISKLGSHLQNVKGLGGLSNKLQNLPDLGKASLGLDIISGLLSGASAGLILADKEASTEKKAAAGVEFANQIIGNVTKAVSSYILAQRVASGLSSTGPVAALIASTVALAVSPLSFLNVADKFKQADLIKSYSERFQKLGYDGDRLLADFHRETGTIDASVTTINTALAAISGGVGAASAGSLVGAPVALLVAGVTGLITTILEYSKQAMFEHVANKVHDRIVEWEKKHNKNYFEQGYDSRHLADLQDNMKFLINLNKELQAERVVAITQQRWDNQIGDLAAISRRTDKISSGKAYVDAFEEGQHQSYDSSVQLDNKNGIINISNTNRKTQSVLFRTPLLTPGEENRERIQEGKNSYITKLHIQRVDSWTVTDGDASSSVDFTNVVQRIAVKFDDAGNIIESKDTKIIANLGAGNDNVFVGSSTTVIDGGDGHDRVHYSRGEYGALVIDATAETEKGSYSVKRYVGDSKALHETIATHQTNVGNREEKIEYRREDDRFHTGYTVTDSLKSVEEIIGSQFNDIFKGSQFDDVFHGGNGVDTIDGNDGDDHLFGGAGDDVIDGGNGNNFLVGGTGNDIISGGKDNDIYVHKTGDGNDSITDSGGQDKLAFSDVNLKDLTFKKVDSSLEIINQKGEKVRIGNWFLEDDLASTVANYKATNDRKIEEIIGKGGERITSEQVDKLIKEGNNQISAEALSKVVNDYNTSKDRQNVSNSLAKLISSVGSFTSSSDFRNNLGTYVPSSIDVSNNIQLARAA

SEQ ID NO: 3 - protein, 1052 aa, Actinobacillus pleuropneumoniae
MSTWSSMLADLKKRAEEAKRQAKKGYDVTKNGLQYGVSQAKLQALAAGKAVQKYGNKLVLVIPKEYDGSVGNGFFDLVKAAEELGIQVKYVNRNELEVAHKSLGTADQFLGLTERGLTLFAPQLDQFLQKHSKISNVVGSSTGDAVSKLAKSQTIISGIQSVLGTVLAGINLNEAIISGGSELELAEAGVSLASELVSNIAKGTTTIDAFTTQIQNFGKLVENAKGLGGVGRQLQNISGSALSKTGLGLDIISSLLSGVTASFALANKNASTSTKVAAGFELSNQVIGGITKAVSSYILAQRLAAGLSTTGPAAALIASSISLAISPLAFLRVADNFNRSKEIGEFAERFKKLGYDGDKLLSEFYHEAGTIDASITTISTALSAIAAGTAAASAGALVGAPITLLVTGITGLISGILEFSKQPMLDHVASKIGNKIDEWEKKYGKNYFENGYDARHKAFLEDSFSLLSSFNKQYETERAVLITQQRWDEYIGELAGITGKGDKLSSGKAYVDYFQEGKLLEKKPDDFSKVVFDPTKGEIDISNSQTSTLLKFVTPLLTPGTESRERTQTGKYEYITKLVVKGKDKWVVNGVKDKGAVYDYTNLIQHAHISSSVARGEEYREVRLVSHLGNGNDKVFLAAGSAEIHAGEGHDVVYYDKTDTGLLVIDGTKATEQGRYSVTRELSGATKILREVIKNQKSAVGKREETLEYRDYELTQSGNSNLKAHDELHSVEEIIGSNQRDEFKGSKFRDIFHGADGDDLLNGNDGDDILYGDKGNDELRGDNGNDQLYGGEGNDKLLGGNGNNYLSGGDGNDELQVLGNGFNVLRGGKGDDKLYGSSGSDLLDGGEGNDYLEGGDGSDFYVYRSTSGNHTIYDQGKSSDLDKLYLSDFSFDRLLVEKVDDNLVLRSNESSHNNGVLTIKDWFKEGNKYNHKIEQIVDKNGRKLTAENLGTYFKNAPKADNLLNYATKEDQNESNLSSLKTELSKIITNAGNFGVAKQGNTGINTAALNNEVNKIISSANTFATSQLGGSGMGTLPSTNVNSMMLGNLARAA

SEQ ID NO: 4 - protein, 1022 aa, Artificial Sequence
  APP ApxIA K560A K686A
MANSQLDRVKGLIDSLNQHTKSAAKSGAGALKNGLGQVKQAGQKLILYIPKDYQASTGSSLNDLVKAAEALGIEVHRSEKNGTALAKELFGTTEKLLGFSERGIALFAPQFDKLLNKNQKLSKSLGGSSEALGQRLNKTQTALSALQSFLGTAIAGMDLDSLLRRRRNGEDVSGSELAKAGVDLAAQLVDNIASATGTVDAFAEQLGKLGNALSNTRLSGLASKLNNLPDLSLAGPGFDAVSGILSVVSASFILSNKDADAGTKAAAGIEISTKILGNIGKAVSQYIIAQRVAAGLSTTAATGGLIGSVVALAISPLSFLNVADKFERAKQLEQYSERFKKFGYEGDSLLASFYRETGAIEAALTTINSVLSAASAGVGAAATGSLVGAPVAALVSAITGIISGILDASKQAIFERVATKLANKIDEWEKKHGKNYFENGYDARHSAFLEDTFELLSQYNKEYSVERVVAITQQRWDVNIGELAGITRKGADAKSGKAYVDFFEEGKLLEKDPDRFDKKVFDPLEGKIDLSSINKTTLLKFITPVFTAGEEIRERKQTGAYEYMTELFVKGKEKWVVTGVQSHNAIYDYTNLIQLAIDKKGEKRQVTIESHLGEKNDRIYLSSGSSIVYAGNGHDVAYYDKTDTGYLTFDGQSAQKAGEYIVTKELKADVKVLKEVVKTQDISVGARSEKLEYRDYELSPFELGNGIRAKDELHSVEEIIGSNRKDKFFGSRFTDIFHGAKGDDEIYGNDGHDILYGDDGNDVIHGGDGNDHLVGGNGNDRLIGGKGNNFLNGGDGDDELQVFEGQYNVLLGGAGNDILYGSDGTNLFDGGVGNDKIYGGLGKDIYRYSKEYGRHIIIEKGGDDDTLLLSDLSFKDVGFIRIGDDLLVNKRIGGTLYYHEDYNGNALTIKDWFKEGKEGQNNKIEKIVDKDGAYVLSQYLTELTAPGRGINYFNGLEEKLYYGEGYNALPQLRKDIEQIISSTGALTGEHGQVLVGAGGPLAYSNSPNSIPNAFSNYLTQSA

SEQ ID NO: 5 - protein, 956 aa, Artificial Sequence
  APP ApxIIA S148G K557A N687A
MSKITLSSLKSSLQQGLKNGKNKLNQAGTTLKNGLTQTGHSLQNGAKKLILYIPQGYDSGQGNGVQDLVKAANDLGIEVWREERSNLDIAKTSFDTTQKILGFTDRGIVLFAPQLDNLLKKNPKIGNTLGSASSISQNIGKANTVLGGIQSILGSVLSGVNLNELLQNKDPNQLELAKAGLELTNELVGNIASSVQTVDAFAEQISKLGSHLQNVKGLGGLSNKLQNLPDLGKASLGLDIISGLLSGASAGLILADKEASTEKKAAAGVEFANQIIGNVTKAVSSYILAQRVASGLSSTGPVAALIASTVALAVSPLSFLNVADKFKQADLIKSYSERFQKLGYDGDRLLADFHRETGTIDASVTTINTALAAISGGVGAASAGSLVGAPVALLVAGVTGLITTILEYSKQAMFEHVANKVHDRIVEWEKKHNKNYFEQGYDSRHLADLQDNMKFLINLNKELQAERVVAITQQRWDNQIGDLAAISRRTDKISSGKAYVDAFEEGQHQSYDSSVQLDNKNGIINISNTNRKTQSVLFRTPLLTPGEENRERIQEGANSYITKLHIQRVDSWTVTDGDASSSVDFTNVVQRIAVKFDDAGNIIESKDTKIIANLGAGNDNVFVGSSTTVIDGGDGHDRVHYSRGEYGALVIDATAETEKGSYSVKRYVGDSKALHETIATHQTNVGAREEKIEYRREDDRFHTGYTVTDSLKSVEEIIGSQFNDIFKGSQFDDVFHGGNGVDTIDGNDGDDHLFGGAGDDVIDGGNGNNFLVGGTGNDIISGGKDNDIYVHKTGDGNDSITDSGGQDKLAFSDVNLKDLTFKKVDSSLEIINQKGEKVRIGNWFLEDDLASTVANYKATNDRKIEEIIGKGGERITSEQVDKLIKEGNNQISAEALSKVVNDYNTSKDRQNVSNSLAKLISSVGSFTSSSDFRNNLGTYVPSSIDVSNNIQLARAA

SEQ ID NO: 6 - protein, 1052 aa, Artificial Sequence
  APP ApxIIIA K571A K702A
MSTWSSMLADLKKRAEEAKRQAKKGYDVTKNGLQYGVSQAKLQALAAGKAVQKYGNKLVLVIPKEYDGSVGNGFFDLVKAAEELGIQVKYVNRNELEVAHKSLGTADQFLGLTERGLTLFAPQLDQFLQKHSKISNVVGSSTGDAVSKLAKSQTIISGIQSVLGTVLAGINLNEAIISGGSELELAEAGVSLASELVSNIAKGTTTIDAFTTQIQNFGKLVENAKGLGGVGRQLQNISGSALSKTGLGLDIISSLLSGVTASFALANKNASTSTKVAAGFELSNQVIGGITKAVSSYILAQRLAAGLSTTGPAAALIASSISLAISPLAFLRVADNFNRSKEIGEFAERFKKLGYDGDKLLSEFYHEAGTIDASITTISTALSAIAAGTAAASAGALVGAPITLLVTGITGLISGILEFSKQPMLDHVASKIGNKIDEWEKKYGKNYFENGYDARHKAFLEDSFSLLSSFNKQYETERAVLITQQRWDEYIGELAGITGKGDKLSSGKAYVDYFQEGKLLEKKPDDFSKVVFDPTKGEIDISNSQTSTLLKFVTPLLTPGTESRERTQTGAYEYITKLVVKGKDKWVVNGVKDKGAVYDYTNLIQHAHISSSVARGEEYREVRLVSHLGNGNDKVFLAAGSAEIHAGEGHDVVYYDKTDTGLLVIDGTKATEQGRYSVTRELSGATKILREVIKNQKSAVGAREETLEYRDYELTQSGNSNLKAHDELHSVEEIIGSNQRDEFKGSKFRDIFHGADGDDLLNGNDGDDILYGDKGNDELRGDNGNDQLYGGEGNDKLLGGNGNNYLSGGDGNDELQVLGNGFNVLRGGKGDDKLYGSSGSDLLDGGEGNDYLEGGDGSDFYVYRSTSGNHTIYDQGKSSDLDKLYLSDFSFDRLLVEKVDDNLVLRSNESSHNNGVLTIKDWFKEGNKYNHKIEQIVDKNGRKLTAENLGTYFKNAPKADNLLNYATKEDQNESNLSSLKTELSKIITNAGNFGVAKQGNTGINTAALNNEVNKIISSANTFATSQLGGSGMGTLPSTNVNSMMLGNLARAA

SEQ ID NO: 7 - protein, 363 aa, Actinobacillus pleuropneumoniae
QGYDSRHLADLQDNMKFLINLNKELQAERVVAITQQRWDNQIGDLAAISRRTDKISSGKAYVDAFEEGQHQSYDSSVQLDNKNGIINISNTNRKTQSVLFRTPLLTPGEENRERIQEGKNSYITKLHIQRVDSWTVTDGDASSSVDFTNVVQRIAVKFDDAGNIIESKDTKIIANLGAGNDNVFVGSSTTVIDGGDGHDRVHYSRGEYGALVIDATAETEKGSYSVKRYVGDSKALHETIATHQTNVGNREEKIEYRREDDRFHTGYTVTDSLKSVEEIIGSQFNDIFKGSQFDDVFHGGNGVDTIDGNDGDDHLFGGAGDDVIDGGNGNNFLVGGTGNDIISGGKDNDIYVHKTGDGNDSIT

SEQ ID NO: 8 - protein, 363 aa, Artificial Sequence
  APP truncated ApxIIA K557A N687A
QGYDSRHLADLQDNMKFLINLNKELQAERVVAITQQRWDNQIGDLAAISRRTDKISSGKAYVDAFEEGQHQSYDSSVQLDNKNGIINISNTNRKTQSVLFRTPLLTPGEENRERIQEGANSYITKLHIQRVDSWTVTDGDASSSVDFTNVVQRIAVKFDDAGNIIESKDTKIIANLGAGNDNVFVGSSTTVIDGGDGHDRVHYSRGEYGALVIDATAETEKGSYSVKRYVGDSKALHETIATHQTNVGAREEKIEYRREDDRFHTGYTVTDSLKSVEEIIGSQFNDIFKGSQFDDVFHGGNGVDTIDGNDGDDHLFGGAGDDVIDGGNGNNFLVGGTGNDIISGGKDNDIYVHKTGDGNDSIT

SEQ ID NO: 9 - DNA, 3069 bp, Actinobacillus pleuropneumoniae
atggctaactctcagctcgatagagtcaaaggattgattgattcacttaatcaacatacaaaaagtgcagctaaatcaggtgccggcgcattaaaaaatggtttgggacaggtgaagcaagcagggcagaaattaattttatatattccgaaagattatcaagctagtaccggctcaagtcttaatgatttagtgaaagcggcggaggctttagggatcgaagtacatcgctcggaaaaaaacggtaccgcactagcgaaagaattattcggtacaacggaaaaactattaggtttctcggaacgaggcatcgcattatttgcacctcagtttgataagttactgaataagaaccaaaaattaagtaaatcgctcggcggttcatcggaagcattaggacaacgtttaaataaaacgcaaacggcactttcagccttacaaagtttcttaggtacggctattgcgggtatggatcttgatagcctgcttcgtcgccgtagaaacggtgaggacgtcagtggttcggaattagctaaagcgggtgtggatctagccgctcagttagtggataacattgcaagtgcaacgggtacggtggatgcgtttgccgaacaattaggtaaattgggcaatgccttatctaacactcgcttaagcggtttagcaagtaagttaaataaccttccagatttaagccttgcaggacctgggtttgatgccgtatcaggtatcttatctgttgtttcggcttcattcattttaagtaataaagatgccgatgcaggtacaaaagcggcggcaggtattgaaatctcaactaaaatcttaggcaatatcggtaaagcggtttctcaatatattattgcgcaacgtgtggcggcaggcttatccacaactgcggcaaccggtggtttaatcggttcggtcgtagcattagcgattagcccgctttcgttcttaaatgttgcggataagtttgaacgtgcgaaacagcttgaacaatattcggagcgctttaaaaagttcggttatgaaggtgatagtttattagcttcattctaccgtgaaaccggtgcgattgaagcggcattaaccacgattaacagtgtgttaagtgcggcttccgcaggtgttggggctgctgcaaccggctcattagtcggtgcgccggtagcagctttagttagtgcaatcaccggtattatttcaggtattttagatgcttctaaacaggcaatcttcgaacgagttgcaacgaaattagcgaataagattgacgaatgggagaaaaaacacggtaaaaactattttgaaaacggttatgacgcccgccattccgcattcttagaagatacctttgaattgttatcacaatacaataaagagtattcggtagagcgtgtcgttgctattacgcaacaacgttgggatgtcaatatcggggaacttgccggtatcacgcgtaaaggtgcggatgcgaaaagcggtaaggcttatgtcgatttctttgaagaaggaaaattgttagagaaagatccggatcgttttgataaaaaagtgtttgatccgcttgaaggcaaaatcgacctttcttcaattaacaaaaccactttattgaaatttattacaccggtttttaccgcaggtgaagagattcgtgagcgtaagcaaaccggtaaatacgaatatatgaccgaattattcgttaaaggtaaagaaaaatgggtggtaaccggtgtgcagtcacataatgcgatttatgactatacgaatcttatccaattagcgatagataaaaaaggtgaaaaacgtcaagtgaccattgaatctcatttgggtgagaaaaatgatcgtatatatctttcatccggttcatctatcgtatatgcgggtaacggacatgatgtagcatattacgataaaaccgatacaggttacttaacatttgacggacaaagtgcacagaaagccggtgaatatattgtcactaaagaacttaaagctgatgtaaaagttttaaaagaagtggttaaaactcaggatatttcagttggaaaacgcagtgaaaaattagaatatcgtgattatgagttaagcccattcgaacttgggaacggtatcagagctaaagatgaattacattctgttgaagaaattatcggtagtaatcgtaaagacaaattctttggtagtcgctttaccgatattttccatggtgcgaaaggcgatgatgaaatctacggtaatgacggccacgatatcttatacggagacgacggtaatgatgtaatccatggcggtgacggtaacgaccatcttgttggtggtaacggaaacgaccgattaatcggcggaaaaggtaataatttccttaatggcggtgatggtgacgatgagttgcaggtctttgagggtcaatacaacgtattattaggtggtgcgggtaatgacattctgtatggcagcgatggtactaacttatttgacggtggtgtaggcaatgacaaaatctacggtggtttaggtaaggatatttatcgctacagtaaggagtacggtcgtcatatcattattgagaaaggcggtgatgatgatacgttattgttatcggatcttagttttaaagatgtaggatttatcagaatcggtgatgatcttcttgtgaataaaagaatcggaggaacactgtattaccatgaagattacaatgggaatgcgctcacgattaaagattggttcaaggaaggtaaagaaggacaaaataataaaattgaaaaaatcgttgataaagatggagcttatgttttaagccaatatctgactgaactgacagctcctggaagaggtatcaattactttaatgggttagaagaaaaattgtattatggagaaggatataatgcacttcctcaactcagaaaagatattgaacaaatcatttcatctactggtgcacttaccggtgaacacggacaagttttagtgggagcaggcggtccattagcttacagcaattcaccgaatagcataccgaatgctttcagtaattatttaacacaatctgcttaa

SEQ ID NO: 10 - DNA, 2871 bp, Actinobacillus pleuropneumoniae
atgtcaaaaatcactttgtcatcattaaaatcgtccttacaacaaggattgacaaatgggaaaaacaagttaaatcaagcaggtacaacactgaagaatggtttaactcaaactggtcattctctacagaatggggctaaaaaattaatcttatatattcctcaaggctatgattcgggtcaaggaaatggaattcaagatttagttaaagctgctaatgatttaggtattgaagtatggcgagaagaacgcagcaatttggacattgcaaaaactagctttgatacaactcagaaaattctaggttttactgatagaggaattgtattatttgcacctcagctagataatttattaaagaagaatcctaaaattggcaatacattaggaagtgcttctagcatctcacaaaatataggtaaagccaatactgtattaggtggtattcaatctattttaggatctgttttatctggagtaaatctgaatgaattacttcaaaataaagatcctaatcaattagaacttgcaaaagcagggctagaactgactaatgaattagttggtaatattgctagctcggtgcaaactgtagatgcatttgcagaacaaatatctaaactaggttcacatttacagaatgtgaaaggattaggaggattgagtaataaattacaaaatctaccagatctaggaaaagcaagtttaggtttggacattatctctggtttactttctggagcatctgcaggtctcattttagcagataaagaggcttcaacagaaaagaaagctgccgcaggtgtagaatttgctaaccaaattataggtaatgtaacaaaagcggtctcatcttacattcttgcccaacgagtcgcttcaggtttgtcttcaactggtcctgtcgctgcattaatcgcatctacagttgcactagctgttagccctctttcattcttaaatgtagctgataagtttaaacaagctgatttaatcaaatcatattctgaacgcttccaaaaattaggatatgatggagatcgtttattagctgattttcaccgtgagacaggaactattgatgcttctgtaacaacaattaacactgctttagcagctatctccggtggagttggagctgcaagcgcgggttctctagtcggagctccagttgcgttactcgttgctggtgttacgggacttattacaactattctagaatattctaaacaagccatgtttgaacatgttgcaaataaggttcatgacagaatagttgaatgggagaaaaaacataataaaaactattttgagcaaggttatgattctcgtcatttagctgatttacaagacaatatgaagtttcttatcaatttaaataaagaacttcaggctgaacgcgtagtagctattacccaacaaagatgggataaccaaattggagacctagcggcaattagccgtagaacggataaaatttccagtggaaaagcttatgtggatgcttttgaggaggggcaacaccagtcctacgattcatccgtacagctagataacaaaaacggtattattaatattagtaatacaaatagaaagacacaaagtgttttattcagaactccattactaactccaggtgaagagaatcgggaacgtattcaggaaggtaaaaattcttatattacaaaattacatatacaaagagttgacagttggactgtaacagatggtgatgctagctcaagcgtagatttcactaatgtagtacaacgaatcgctgtgaaatttgatgatgcaggtaacattatcgaatctaaagatactaaaattatcgcaaatttaggtgctggtaacgataatgtatttgttgggtcaagtactaccgttattgatggcggggacggacatgatcgagttcactacagtagaggagaatatggcgcattagttattgatgctacagccgagacagaaaaaggctcatattcagtaaaacgctatgtcggagacagtaaagcattacatgaaacaattgccacccaccaaacaaatgttggtaatcgtgaagaaaaaattgaatatcgtcgtgaagatgatcgttttcatactggttatactgtgacggactcactcaaatcagttgaagagatcattggttcacaatttaatgatattttcaaaggaagccaatttgatgatgtgttccatggtggtaatggtgtagacactattgatggtaacgatggtgacgatcatttatttggtggcgcaggcgatgatgttatcgatggaggaaacggtaacaatttccttgttggaggaaccggtaatgatattatctcgggaggtaaagataatgatatttatgtccataaaacaggcgatggaaatgattctattacagactctggcggacaagataaactggcattttcggatgtaaatcttaaagacctcacctttaagaaagtagattcttctctcgaaatcattaatcaaaaaggagaaaaagttcgtattgggaattggttcttagaagatgatttggctagcacagttgctaactataaagctacgaatgaccgaaaaattgaggaaattattggtaaaggaggagaacgtattacatcagaacaagttgataaactgattaaggagggtaacaatcaaatctctgcagaagcattatccaaagttgtgaatgattacaatacgagtaaagatagacagaacgtatctaatagcttagcaaaattgatttcttcagtcgggagctttacgtcttcctcagactttaggaataatttaggaacatatgttccttcatcaatagatgtctcgaataatattcaattagctagagccgcttaa

SEQ ID NO: 11 - DNA, 3159 bp, Actinobacillus pleuropneumoniae
atgagtacttggtcaagcatgttagccgacttaaaaaaacgggctgaagaagccaaaagacaagccaaaaaaggctacgatgtaactaaaaatggtttgcaatatggggtgagtcaagcaaaattacaagcattagcagctggtaaagccgttcaaaagtacggtaataaattagttttagttattccaaaagagtatgacggaagtgttggtaacggtttctttgatttagtaaaagcagctgaggaattaggcattcaagttaaatatgttaaccgtaatgaattggaagttgcccataaaagtttaggtaccgcagaccaattcttgggtttaacagaacgtggacttactttatttgcaccgcaactagatcagttcttacaaaaacattcaaaaatttctaacgtagtgggcagttctactggtgatgcagtaagtaaacttgctaagagtcaaactattatttcaggaattcaatctgtattaggtactgtattagcaggtattaatcttaatgaagctattattagtggcggttcagagctcgaattagctgaagctggtgtttctttagcctctgagctcgttagtaatattgctaaaggtacaacaacaatagatgctttcactacacaaatccagaactttgggaaattagtggaaaatgctaaagggttaggtggtgttggccgccaattacagaatatttcaggttctgcattaagcaaaactggattaggtttggatattatctcaagcttactttcaggagtaactgcaagttttgctttagcgaataagaatgcttcaacaagcactaaagttgctgctggctttgaactctcaaatcaagtaattggtggtattacgaaagcagtatcaagctatattcttgcacagcgtttagctgctggtttatcaacgacaggtcctgctgcagcactaattgcgtctagtatttctttagcaatcagtccattggcgtttttacgtgtagctgataattttaatcgttctaaagaaattggcgaatttgctgaacgtttcaaaaaattgggctatgacggcgataaactactttcagagttttatcacgaagctggtactattgatgcctcaattactacaattagtacagcactttctgctatcgcagctggaacggccgccgcgagtgcaggtgcattagttggcgcaccaattactttgttggttactggtatcacaggattaatttctggtattttagagttctctaaacaaccaatgttagatcatgttgcatcgaaaattggtaacaaaattgacgaatgggagaaaaaatacggtaaaaattacttcgagaatggctatgatgctcgtcataaagctttcttagaagattcattctcattattgtctagttttaataaacaatatgaaactgaaagagctgttttaattacacaacaacgttgggatgaatatattggcgaacttgcgggtattactggcaaaggtgacaaactctctagtggtaaggcgtatgtagattactttcaagaaggtaaattattagagaaaaaacctgatgactttagcaaagtagttttcgatccaactaagggcgaaattgatatttcaaatagccaaacgtcaacgttgttaaaatttgttacgccattattaacaccaggtacagagtcacgtgaaagaactcaaacaggtaaatatgaatatatcacgaagttagttgtaaaaggtaaagataaatgggttgttaatggcgttaaagataaaggtgccgtttatgattatactaatttaattcaacatgctcatattagttcatcagtagcacgtggtgaagaataccgtgaagttcgtttggtatctcatctaggcaatggtaatgacaaagtgttcttagctgcgggttccgcagaaattcacgctggtgaaggtcatgatgtggtttattatgataaaaccgatacaggtcttttagtaattgatggaaccaaagcgactgaacaagggcgttattctgttacgcgcgaattgagtggtgctacaaaaatcctgagagaagtaataaaaaatcaaaaatctgctgttggtaaacgtgaagaaaccttggaatatcgtgattatgaattaacgcaatcaggtaatagtaacctaaaagcacatgatgaattacattcagtagaagaaattattggaagtaatcagagagacgaatttaaaggtagtaaattcagagatattttccatggtgccgatggtgatgatctattaaatggtaatgatggggatgatattctatacggtgataaaggtaacgatgagttaagaggtgataatggtaacgaccaactttatggtggtgaaggtaatgacaaactattaggaggtaatggcaataattacctcagtggtggtgatggcaatgatgagcttcaagtcttaggcaatggttttaatgtgcttcgtggcggtaaaggcgatgataaactttatggtagctcaggttctgatttacttgatggtggagaaggtaatgattatctagaaggaggcgatggtagcgatttttatgtttatcgttccacttcaggtaatcatactatttatgatcaaggtaaatctagtgatttagataaactatatttgtctgatttttccttcgatcgtcttcttgttgagaaagttgatgataaccttgtacttagaagtaatgaaagtagtcataataatggagtactcacaatcaaagactggtttaaagaagggaataaatataaccataaaattgaacaaattgttgataaaaatggtagaaaattgacagcagagaatttaggaacttatttcaaaaatgctccaaaagctgacaatttgcttaattatgcaactaaagaagatcagaatgaaagcaatttatcttcacttaaaactgaattaagtaaaattattactaatgcaggtaattttggtgtggcaaaacaaggtaatactggaatcaatacagctgccttgaacaatgaagtgaataaaatcatttcttctgctaatacctttgctacttcacaattgggtggctcagggatgggaacattaccatcaacgaatgtaaattcaatgatgctaggtaacctagctagagcagcttaa

SEQ ID NO: 12 - DNA, 3069 bp, Artificial Sequence
  APP ApxIA K560A K686A
atggctaactctcagctcgatagagtcaaaggattgattgattcacttaatcaacatacaaaaagtgcagctaaatcaggtgccggcgcattaaaaaatggtttgggacaggtgaagcaagcagggcagaaattaattttatatattccgaaagattatcaagctagtaccggctcaagtcttaatgatttagtgaaagcggcggaggctttagggatcgaagtacatcgctcggaaaaaaacggtaccgcactagcgaaagaattattcggtacaacggaaaaactattaggtttctcggaacgaggcatcgcattatttgcacctcagtttgataagttactgaataagaaccaaaaattaagtaaatcgctcggcggttcatcggaagcattaggacaacgtttaaataaaacgcaaacggcactttcagccttacaaagtttcttaggtacggctattgcgggtatggatcttgatagcctgcttcgtcgccgtagaaacggtgaggacgtcagtggttcggaattagctaaagcgggtgtggatctagccgctcagttagtggataacattgcaagtgcaacgggtacggtggatgcgtttgccgaacaattaggtaaattgggcaatgccttatctaacactcgcttaagcggtttagcaagtaagttaaataaccttccagatttaagccttgcaggacctgggtttgatgccgtatcaggtatcttatctgttgtttcggcttcattcattttaagtaataaagatgccgatgcaggtacaaaagcggcggcaggtattgaaatctcaactaaaatcttaggcaatatcggtaaagcggtttctcaatatattattgcgcaacgtgtggcggcaggcttatccacaactgcggcaaccggtggtttaatcggttcggtcgtagcattagcgattagcccgctttcgttcttaaatgttgcggataagtttgaacgtgcgaaacagcttgaacaatattcggagcgctttaaaaagttcggttatgaaggtgatagtttattagcttcattctaccgtgaaaccggtgcgattgaagcggcattaaccacgattaacagtgtgttaagtgcggcttccgcaggtgttggggctgctgcaaccggctcattagtcggtgcgccggtagcagctttagttagtgcaatcaccggtattatttcaggtattttagatgcttctaaacaggcaatcttcgaacgagttgcaacgaaattagcgaataagattgacgaatgggagaaaaaacacggtaaaaactattttgaaaacggttatgacgcccgccattccgcattcttagaagatacctttgaattgttatcacaatacaataaagagtattcggtagagcgtgtcgttgctattacgcaacaacgttgggatgtcaatatcggggaacttgccggtatcacgcgtaaaggtgcggatgcgaaaagcggtaaggcttatgtcgatttctttgaagaaggaaaattgttagagaaagatccggatcgttttgataaaaaagtgtttgatccgcttgaaggcaaaatcgacctttcttcaattaacaaaaccactttattgaaatttattacaccggtttttaccgcaggtgaagagattcgtgagcgtaagcaaaccggtgcatacgaatatatgaccgaattattcgttaaaggtaaagaaaaatgggtggtaaccggtgtgcagtcacataatgcgatttatgactatacgaatcttatccaattagcgatagataaaaaaggtgaaaaacgtcaagtgaccattgaatctcatttgggtgagaaaaatgatcgtatatatctttcatccggttcatctatcgtatatgcgggtaacggacatgatgtagcatattacgataaaaccgatacaggttacttaacatttgacggacaaagtgcacagaaagccggtgaatatattgtcactaaagaacttaaagctgatgtaaaagttttaaaagaagtggttaaaactcaggatatttcagttggagcacgcagtgaaaaattagaatatcgtgattatgagttaagcccattcgaacttgggaacggtatcagagctaaagatgaattacattctgttgaagaaattatcggtagtaatcgtaaagacaaattctttggtagtcgctttaccgatattttccatggtgcgaaaggcgatgatgaaatctacggtaatgacggccacgatatcttatacggagacgacggtaatgatgtaatccatggcggtgacggtaacgaccatcttgttggtggtaacggaaacgaccgattaatcggcggaaaaggtaataatttccttaatggcggtgatggtgacgatgagttgcaggtctttgagggtcaatacaacgtattattaggtggtgcgggtaatgacattctgtatggcagcgatggtactaacttatttgacggtggtgtaggcaatgacaaaatctacggtggtttaggtaaggatatttatcgctacagtaaggagtacggtcgtcatatcattattgagaaaggcggtgatgatgatacgttattgttatcggatcttagttttaaagatgtaggatttatcagaatcggtgatgatcttcttgtgaataaaagaatcggaggaacactgtattaccatgaagattacaatgggaatgcgctcacgattaaagattggttcaaggaaggtaaagaaggacaaaataataaaattgaaaaaatcgttgataaagatggagcttatgttttaagccaatatctgactgaactgacagctcctggaagaggtatcaattactttaatgggttagaagaaaaattgtattatggagaaggatataatgcacttcctcaactcagaaaagatattgaacaaatcatttcatctactggtgcacttaccggtgaacacggacaagttttagtgggagcaggcggtccattagcttacagcaattcaccgaatagcataccgaatgctttcagtaattatttaacacaatctgcttaa

SEQ ID NO: 13 - DNA, 2871 bp, Artificial Sequence
  APP ApxIIA S148A K557A K686A
atgtcaaaaatcactttgtcatcattaaaatcgtccttacaacaaggattgacaaatgggaaaaacaagttaaatcaagcaggtacaacactgaagaatggtttaactcaaactggtcattctctacagaatggggctaaaaaattaatcttatatattcctcaaggctatgattcgggtcaaggaaatggaattcaagatttagttaaagctgctaatgatttaggtattgaagtatggcgagaagaacgcagcaatttggacattgcaaaaactagctttgatacaactcagaaaattctaggttttactgatagaggaattgtattatttgcacctcagctagataatttattaaagaagaatcctaaaattggcaatacattaggaagtgcttctagcatctcacaaaatataggtaaagccaatactgtattaggtggtattcaatctattttaggatctgttttatctggagtaaatctgaatgaattacttcaaaataaagatcctaatcaattagaacttgcaaaagcagggctagaactgactaatgaattagttggtaatattgctagctcggtgcaaactgtagatgcatttgcagaacaaatatctaaactaggttcacatttacagaatgtgaaaggattaggaggattgagtaataaattacaaaatctaccagatctaggaaaagcaagtttaggtttggacattatctctggtttactttctggagcatctgcaggtctcattttagcagataaagaggcttcaacagaaaagaaagctgccgcaggtgtagaatttgctaaccaaattataggtaatgtaacaaaagcggtctcatcttacattcttgcccaacgagtcgcttcaggtttgtcttcaactggtcctgtcgctgcattaatcgcatctacagttgcactagctgttagccctctttcattcttaaatgtagctgataagtttaaacaagctgatttaatcaaatcatattctgaacgcttccaaaaattaggatatgatggagatcgtttattagctgattttcaccgtgagacaggaactattgatgcttctgtaacaacaattaacactgctttagcagctatctccggtggagttggagctgcaagcgcgggttctctagtcggagctccagttgcgttactcgttgctggtgttacgggacttattacaactattctagaatattctaaacaagccatgtttgaacatgttgcaaataaggttcatgacagaatagttgaatgggagaaaaaacataataaaaactattttgagcaaggttatgattctcgtcatttagctgatttacaagacaatatgaagtttcttatcaatttaaataaagaacttcaggctgaacgcgtagtagctattacccaacaaagatgggataaccaaattggagacctagcggcaattagccgtagaacggataaaatttccagtggaaaagcttatgtggatgcttttgaggaggggcaacaccagtcctacgattcatccgtacagctagataacaaaaacggtattattaatattagtaatacaaatagaaagacacaaagtgttttattcagaactccattactaactccaggtgaagagaatcgggaacgtattcaggaaggtgcaaattcttatattacaaaattacatatacaaagagttgacagttggactgtaacagatggtgatgctagctcaagcgtagatttcactaatgtagtacaacgaatcgctgtgaaatttgatgatgcaggtaacattatcgaatctaaagatactaaaattatcgcaaatttaggtgctggtaacgataatgtatttgttgggtcaagtactaccgttattgatggcggggacggacatgatcgagttcactacagtagaggagaatatggcgcattagttattgatgctacagccgagacagaaaaaggctcatattcagtaaaacgctatgtcggagacagtaaagcattacatgaaacaattgccacccaccaaacaaatgttggtgctcgtgaagaaaaaattgaatatcgtcgtgaagatgatcgttttcatactggttatactgtgacggactcactcaaatcagttgaagagatcattggttcacaatttaatgatattttcaaaggaagccaatttgatgatgtgttccatggtggtaatggtgtagacactattgatggtaacgatggtgacgatcatttatttggtggcgcaggcgatgatgttatcgatggaggaaacggtaacaatttccttgttggaggaaccggtaatgatattatctcgggaggtaaagataatgatatttatgtccataaaacaggcgatggaaatgattctattacagactctggcggacaagataaactggcattttcggatgtaaatcttaaagacctcacctttaagaaagtagattcttctctcgaaatcattaatcaaaaaggagaaaaagttcgtattgggaattggttcttagaagatgatttggctagcacagttgctaactataaagctacgaatgaccgaaaaattgaggaaattattggtaaaggaggagaacgtattacatcagaacaagttgataaactgattaaggagggtaacaatcaaatctctgcagaagcattatccaaagttgtgaatgattacaatacgagtaaagatagacagaacgtatctaatagcttagcaaaattgatttcttcagtcgggagctttacgtcttcctcagactttaggaataatttaggaacatatgttccttcatcaatagatgtctcgaataatattcaattagctagagccgcttaa

SEQ ID NO: 14 - DNA, 3159 bp, Artificial Sequence
  APP ApxIIIA K571A K702A
atgagtacttggtcaagcatgttagccgacttaaaaaaacgggctgaagaagccaaaagacaagccaaaaaaggctacgatgtaactaaaaatggtttgcaatatggggtgagtcaagcaaaattacaagcattagcagctggtaaagccgttcaaaagtacggtaataaattagttttagttattccaaaagagtatgacggaagtgttggtaacggtttctttgatttagtaaaagcagctgaggaattaggcattcaagttaaatatgttaaccgtaatgaattggaagttgcccataaaagtttaggtaccgcagaccaattcttgggtttaacagaacgtggacttactttatttgcaccgcaactagatcagttcttacaaaaacattcaaaaatttctaacgtagtgggcagttctactggtgatgcagtaagtaaacttgctaagagtcaaactattatttcaggaattcaatctgtattaggtactgtattagcaggtattaatcttaatgaagctattattagtggcggttcagagctcgaattagctgaagctggtgtttctttagcctctgagctcgttagtaatattgctaaaggtacaacaacaatagatgctttcactacacaaatccagaactttgggaaattagtggaaaatgctaaagggttaggtggtgttggccgccaattacagaatatttcaggttctgcattaagcaaaactggattaggtttggatattatctcaagcttactttcaggagtaactgcaagttttgctttagcgaataagaatgcttcaacaagcactaaagttgctgctggctttgaactctcaaatcaagtaattggtggtattacgaaagcagtatcaagctatattcttgcacagcgtttagctgctggtttatcaacgacaggtcctgctgcagcactaattgcgtctagtatttctttagcaatcagtccattggcgtttttacgtgtagctgataattttaatcgttctaaagaaattggcgaatttgctgaacgtttcaaaaaattgggctatgacggcgataaactactttcagagttttatcacgaagctggtactattgatgcctcaattactacaattagtacagcactttctgctatcgcagctggaacggccgccgcgagtgcaggtgcattagttggcgcaccaattactttgttggttactggtatcacaggattaatttctggtattttagagttctctaaacaaccaatgttagatcatgttgcatcgaaaattggtaacaaaattgacgaatgggagaaaaaatacggtaaaaattacttcgagaatggctatgatgctcgtcataaagctttcttagaagattcattctcattattgtctagttttaataaacaatatgaaactgaaagagctgttttaattacacaacaacgttgggatgaatatattggcgaacttgcgggtattactggcaaaggtgacaaactctctagtggtaaggcgtatgtagattactttcaagaaggtaaattattagagaaaaaacctgatgactttagcaaagtagttttcgatccaactaagggcgaaattgatatttcaaatagccaaacgtcaacgttgttaaaatttgttacgccattattaacaccaggtacagagtcacgtgaaagaactcaaacaggtgcatatgaatatatcacgaagttagttgtaaaaggtaaagataaatgggttgttaatggcgttaaagataaaggtgccgtttatgattatactaatttaattcaacatgctcatattagttcatcagtagcacgtggtgaagaataccgtgaagttcgtttggtatctcatctaggcaatggtaatgacaaagtgttcttagctgcgggttccgcagaaattcacgctggtgaaggtcatgatgtggtttattatgataaaaccgatacaggtcttttagtaattgatggaaccaaagcgactgaacaagggcgttattctgttacgcgcgaattgagtggtgctacaaaaatcctgagagaagtaataaaaaatcaaaaatctgctgttggtgcacgtgaagaaaccttggaatatcgtgattatgaattaacgcaatcaggtaatagtaacctaaaagcacatgatgaattacattcagtagaagaaattattggaagtaatcagagagacgaatttaaaggtagtaaattcagagatattttccatggtgccgatggtgatgatctattaaatggtaatgatggggatgatattctatacggtgataaaggtaacgatgagttaagaggtgataatggtaacgaccaactttatggtggtgaaggtaatgacaaactattaggaggtaatggcaataattacctcagtggtggtgatggcaatgatgagcttcaagtcttaggcaatggttttaatgtgcttcgtggcggtaaaggcgatgataaactttatggtagctcaggttctgatttacttgatggtggagaaggtaatgattatctagaaggaggcgatggtagcgatttttatgtttatcgttccacttcaggtaatcatactatttatgatcaaggtaaatctagtgatttagataaactatatttgtctgatttttccttcgatcgtcttcttgttgagaaagttgatgataaccttgtacttagaagtaatgaaagtagtcataataatggagtactcacaatcaaagactggtttaaagaagggaataaatataaccataaaattgaacaaattgttgataaaaatggtagaaaattgacagcagagaatttaggaacttatttcaaaaatgctccaaaagctgacaatttgcttaattatgcaactaaagaagatcagaatgaaagcaatttatcttcacttaaaactgaattaagtaaaattattactaatgcaggtaattttggtgtggcaaaacaaggtaatactggaatcaatacagctgccttgaacaatgaagtgaataaaatcatttcttctgctaatacctttgctacttcacaattgggtggctcagggatgggaacattaccatcaacgaatgtaaattcaatgatgctaggtaacctagctagagcagcttaa

SEQ ID NO: 15 - DNA, 2446 bp, Artificial Sequence
  Plasmid pEX-A258
gtggcagctctagagctagcgaattctttggtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgcgaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtttggcaattggtcgacctcgagggcgcgcccgta

SEQ ID NO: 16 - DNA, 4751 bp, Artificial Sequence
  Plasmid pQE-80L
ctcgagaaatcataaaaaatttatttgctttgtgagcggataacaattataatagattcaattgtgagcggataacaatttcacacagaattcattaaagaggagaaattaactatgagaggatcgcatcaccatcaccatcacggatccgcatgcgagctcggtaccccgggtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccctctagattacgtgcagtcgatgataagctgtcaaacatgagaattgtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcggaatttcgggcagcgttgggtcctggccacgggtgcgcatgatctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 17 - DNA, 3431 bp, Artificial Sequence
  Plasmid pQE-60
ctcgagaaatcataaaaaatttatttgctttgtgagcggataacaattataatagattcaattgtgagcggataacaatttcacacagaattcattaaagaggagaaattaaccatgggaggatccagatctcatcaccatcaccatcactaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 18 - DNA, 2938 bp, Artificial Sequence
  dfrA14sacB cassette
gttaatgccgtctgaagtgcgaagcggcatcagagcagattgtactgagagtgcaccatatggtcgacctcgagttaattaacgtatgcggccgctttagactatttaaataatattatttaaattctttactatagtgtacaatacacacagtccattaaccaaaataaaaggaggaattaggatgagaaccttgaaagtatcattgatagctgcgaaagcgaaaaacggcgtgattggttgcggtccagacataccctggtccgcgaaaggggagcagctactttttaaagcattgacctacaatcagtggcttctggtgggtcgcaagacgtttgaatctatgggcgcactccccaataggaaatacgcggtcgttacccgctcaggttggacatcaaatgatgacaatgtagttgtatttcagtcaatcgaagaggccatggacaggctagctgaattcaccggtcacgttatagtgtctggtggcggagaaatttaccgagaaacattacccatggcctctacgctccacttatcgacgatcgacatcgagccagagggggatgttttcttcccgagtattccaaataccttcgaagttgtttttgagcaacactttacttcaaacattaactattgctatcaaatttggaaaaagggttaatgccgtctgaagtgcggtacaagcggtagaacctgccccgttagttgaaaccgcttgttatgcatgcatgggatccgcgaatcccgcggccatggcggccgggagcatgcgacgtcgggcccattgggatccgcttttacagcgattgcagaatgattgaattgtaaactttagagctttatattttgtttaatggtattatatttacttatatttatgattcttagtttttattgtaaattaaagtgtttatttattgtattttaagtataagatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatatcctattggcattttcttttatttcttatcaacataaaggtgaatcccatacctagagctgcacgcgagacatgaacgtgcaactgcttc

SEQ ID NO: 19 - DNA, 24 bp, Artificial Sequence
  linker tri_OE_for
gttaatgccgtctgaagtgcgaag

SEQ ID NO: 20 - DNA, 24 bp, Artificial Sequence
  linker sac_OE_rev
gaagcagttgcacgttcatgtctc

SEQ ID NO: 21 - DNA, 18 bp, Artificial Sequence
  left_flank_for primer
attgggtaccgagctcgc

SEQ ID NO: 22 - DNA, 24 bp, Artificial Sequence
  tri_OE_rev primer
cttcgcacttcagacggcattaac

SEQ ID NO: 23 - DNA, 24 bp, Artificial Sequence
  sac_OE_for primer
gagacatgaacgtgcaactgcttc

SEQ ID NO: 24 - DNA, 24 bp, Artificial Sequence
  right_flank_rev primer
ccatttcacacaggaattcggatc

SEQ ID NO: 25 - DNA, 31 bp, Artificial Sequence
  for primer
aaacaagcggtccggatcttggaatttcggc

SEQ ID NO: 26 - DNA, 22 bp, Artificial Sequence
  rev primer
tgccttcaagcggatcaaacac

SEQ ID NO: 27 - DNA, 22 bp, Artificial Sequence
  for primer
tcgaacttgggaacggtatcag

SEQ ID NO: 28 - DNA, 34 bp, Artificial Sequence
  rev primer
ttacaagcggtactttgccagcttacctacgatg

SEQ ID NO: 29 - DNA, 22 bp, Artificial Sequence
  apxIA_mut_for_OE
gtgtttgatccgcttgaaggca

SEQ ID NO: 30 - DNA, 22 bp, Artificial Sequence
  apxIA_mut_rev_OE
ctgataccgttcccaagttcga

SEQ ID NO: 31 - DNA, 21 bp, Artificial Sequence
  left_flank_for_USS
atccacaagcggtcatctggc

SEQ ID NO: 32 - DNA, 25 bp, Artificial Sequence
  sxy_TS_LF_for
gtaccgcttgttaaatgattacacc

SEQ ID NO: 33 - DNA, 30 bp, Artificial Sequence
  Sxy_TS_LF_rev1
ggcattaacttagttagcctgtgagatagc

SEQ ID NO: 34 - DNA, 40 bp, Artificial Sequence
  Sxy_TS_LF_rev2
cttcgcacttcagacggcattaacttagttagcctgtgag

SEQ ID NO: 35 - DNA, 3988 bp, Artificial Sequence
  delta_apxIA_dfrA14sacB
attgggtaccgagctcgcggccgcaccggctcattagtcggtgcgccggtagcagctttagttagtgcaatcaccggtattatttcaggtattttagatgcttctaaacaggcaatcttcgaacgagttgcaacgaaattagcgaataagattgacgaatgggagaaaaaacacggtaaaaactattttgaaaacggttatgacgcccgccattccgcattcttagaagatacctttgaattgttatcacaatacaataaagagtattcggtagagcgtgtcgttgctattacgcaacaacgttgggatgtcaatatcggggaacttgccggtatcacgcgtaaaggtgcggatgcgaaaagcggtaaggcttatgtcgatttctttgaagaaggaaaattgttagagaaagatccggatcgttttgataaaaaagtgtttgatccgcttgaaggcaaaatcgacctttcttcaattaacaaaaccactttattgaaatttattacaccggtttttaccgcaggtgttaatgccgtctgaagtgcgaagcggcatcagagcagattgtactgagagtgcaccatatggtcgacctcgagttaattaacgtatgcggccgctttagactatttaaataatattatttaaattctttactatagtgtacaatacacacagtccattaaccaaaataaaaggaggaattaggatgagaaccttgaaagtatcattgatagctgcgaaagcgaaaaacggcgtgattggttgcggtccagacataccctggtccgcgaaaggggagcagctactttttaaagcattgacctacaatcagtggcttctggtgggtcgcaagacgtttgaatctatgggcgcactccccaataggaaatacgcggtcgttacccgctcaggttggacatcaaatgatgacaatgtagttgtatttcagtcaatcgaagaggccatggacaggctagctgaattcaccggtcacgttatagtgtctggtggcggagaaatttaccgagaaacattacccatggcctctacgctccacttatcgacgatcgacatcgagccagagggggatgttttcttcccgagtattccaaataccttcgaagttgtttttgagcaacactttacttcaaacattaactattgctatcaaatttggaaaaagggttaatgccgtctgaagtgcggtacaagcggtagaacctgccccgttagttgaaaccgcttgttatgcatgcatgggatccgcgaatcccgcggccatggcggccgggagcatgcgacgtcgggcccattgggatccgcttttacagcgattgcagaatgattgaattgtaaactttagagctttatattttgtttaatggtattatatttacttatatttatgattcttagtttttattgtaaattaaagtgtttatttattgtattttaagtataagatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatatcctattggcattttcttttatttcttatcaacataaaggtgaatcccatacctagagctgcacgcgagacatgaacgtgcaactgcttcccattcgaacttgggaacggtatcagagctaaagatgaattacattctgttgaagaaattatcggtagtaatcgtaaagacaaattctttggtagtcgctttaccgatattttccatggtgcgaaaggcgatgatgaaatctacggtaatgacggccacgatatcttatacggagacgacggtaatgatgtaatccatggcggtgacggtaacgaccatcttgttggtggtaacggaaacgaccgattaatcggcggaaaaggtaataatttccttaatggcggtgatggtgacgatgagttgcaggtctttgagggtcaatacaacgtattattaggtggtgcgggtaatgacattctgtatggcagcgatggtactaacttatttgacggtggtgtaggcaatgacaaaatctacggtggtttaggtaaggatatttatcgctacagtaaggagtacggtcgtcatatcattattgagaaaggcggtgatgatgatacggatccgaattcctgtgtgaaatgg

SEQ ID NO: 36 - DNA, 1473 bp, Artificial Sequence
  apxIAmut
gaccgcggccgcaccggctcattagtcggtgcgccggtagcagctttagttagtgcaatcaccggtattatttcaggtattttagatgcttctaaacaggcaatcttcgaacgagttgcaacgaaattagcgaataagattgacgaatgggagaaaaaacacggtaaaaactattttgaaaacggttatgacgcccgccattccgcattcttagaagatacctttgaattgttatcacaatacaataaagagtattcggtagagcgtgtcgttgctattacgcaacaacgttgggatgtcaatatcggggaacttgccggtatcacgcgtaaaggtgcggatgcgaaaagcggtaaggcttatgtcgatttctttgaagaaggaaaattgttagagaaagatccggatcgttttgataaaaaagtgtttgatccgcttgaaggcaaaatcgacctttcttcaattaacaaaaccactttattgaaatttattacaccggtttttaccgcaggtgaagagattcgtgagcgtaagcaaaccggtgcatacgaatatatgaccgaattattcgttaaaggtaaagaaaaatgggtggtaaccggtgtgcagtcacataatgcgatttatgactatacgaatcttatccaattagcgatagataaaaaaggtgaaaaacgtcaagtgaccattgaatctcatttgggtgagaaaaatgatcgtatatatctttcatccggttcatctatcgtatatgcgggtaacggacatgatgtagcatattacgataaaaccgatacaggttacttaacatttgacggacaaagtgcacagaaagccggtgaatatattgtcactaaagaacttaaagctgatgtaaaagttttaaaagaagtggttaaaactcaggatatttcagttggagcacgcagtgaaaaattagaatatcgtgattatgagttaagcccattcgaacttgggaacggtatcagagctaaagatgaattacattctgttgaagaaattatcggtagtaatcgtaaagacaaattctttggtagtcgctttaccgatattttccatggtgcgaaaggcgatgatgaaatctacggtaatgacggccacgatatcttatacggagacgacggtaatgatgtaatccatggcggtgacggtaacgaccatcttgttggtggtaacggaaacgaccgattaatcggcggaaaaggtaataatttccttaatggcggtgatggtgacgatgagttgcaggtctttgagggtcaatacaacgtattattaggtggtgcgggtaatgacattctgtatggcagcgatggtactaacttatttgacggtggtgtaggcaatgacaaaatctacggtggtttaggtaaggatatttatcgctacagtaaggagtacggtcgtcatatcattattgagaaaggcggtgatgatgatacaagcggtttg

SEQ ID NO: 37 - DNA, 3987 bp, Artificial Sequence
  delta_apxIIA_dfrA14sacB
attgggtaccgagctcgcggccgcgctgcaagcgcgggttctctagtcggagctccagttgcgttactcgttgctggtgttacgggacttattacaactattctagaatattctaaacaagccatgtttgaacatgttgcaaataaggttcatgacagaatagttgaatgggagaaaaaacataataaaaactattttgagcaaggttatgattctcgtcatttagctgatttacaagacaatatgaagtttcttatcaatttaaataaagaacttcaggctgaacgcgtagtagctattacccaacaaagatgggataaccaaattggagacctagcggcaattagccgtagaacggataaaatttccagtggaaaagcttatgtggatgcttttgaggaggggcaacaccagtcctacgattcatccgtacagctagataacaaaaacggtattattaatattagtaatacaaatagaaagacacaaagtgttttattcagaactccattactaactccaggtgttaatgccgtctgaagtgcgaagcggcatcagagcagattgtactgagagtgcaccatatggtcgacctcgagttaattaacgtatgcggccgctttagactatttaaataatattatttaaattctttactatagtgtacaatacacacagtccattaaccaaaataaaaggaggaattaggatgagaaccttgaaagtatcattgatagctgcgaaagcgaaaaacggcgtgattggttgcggtccagacataccctggtccgcgaaaggggagcagctactttttaaagcattgacctacaatcagtggcttctggtgggtcgcaagacgtttgaatctatgggcgcactccccaataggaaatacgcggtcgttacccgctcaggttggacatcaaatgatgacaatgtagttgtatttcagtcaatcgaagaggccatggacaggctagctgaattcaccggtcacgttatagtgtctggtggcggagaaatttaccgagaaacattacccatggcctctacgctccacttatcgacgatcgacatcgagccagagggggatgttttcttcccgagtattccaaataccttcgaagttgtttttgagcaacactttacttcaaacattaactattgctatcaaatttggaaaaagggttaatgccgtctgaagtgcggtacaagcggtagaacctgccccgttagttgaaaccgcttgttatgcatgcatgggatccgcgaatcccgcggccatggcggccgggagcatgcgacgtcgggcccattgggatccgcttttacagcgattgcagaatgattgaattgtaaactttagagctttatattttgtttaatggtattatatttacttatatttatgattcttagtttttattgtaaattaaagtgtttatttattgtattttaagtataagatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatatcctattggcattttcttttatttcttatcaacataaaggtgaatcccatacctagagctgcacgcgagacatgaacgtgcaactgcttcgttttcatactggttatactgtgacggactcactcaaatcagttgaagagatcattggttcacaatttaatgatattttcaaaggaagccaatttgatgatgtgttccatggtggtaatggtgtagacactattgatggtaacgatggtgacgatcatttatttggtggcgcaggcgatgatgttatcgatggaggaaacggtaacaatttccttgttggaggaaccggtaatgatattatctcgggaggtaaagataatgatatttatgtccataaaacaggcgatggaaatgattctattacagactctggcggacaagataaactggcattttcggatgtaaatcttaaagacctcacctttaagaaagtagattcttctctcgaaatcattaatcaaaaaggagaaaaagttcgtattgggaattggttcttagaagatgatttggctagcacagttgctaactataaagctacgaatgaccgaaaaattgaggaagatccgaattcctgtgtgaaatgg

SEQ ID NO: 38 - DNA, 1483 bp, Artificial Sequence
  apxIIAmut
gaccgcggccgcgctgcaagcgcgggttctctagtcggagctccagttgcgttactcgttgctggtgttacgggacttattacaactattctagaatattctaaacaagccatgtttgaacatgttgcaaataaggttcatgacagaatagttgaatgggagaaaaaacataataaaaactattttgagcaaggttatgattctcgtcatttagctgatttacaagacaatatgaagtttcttatcaatttaaataaagaacttcaggctgaacgcgtagtagctattacccaacaaagatgggataaccaaattggagacctagcggcaattagccgtagaacggataaaatttccagtggaaaagcttatgtggatgcttttgaggaggggcaacaccagtcctacgattcatccgtacagctagataacaaaaacggtattattaatattagtaatacaaatagaaagacacaaagtgttttattcagaactccattactaactccaggtgaagagaatcgggaacgtattcaggaaggtgcaaattcttatattacaaaattacatatacaaagagttgacagttggactgtaacagatggtgatgctagctcaagcgtagatttcactaatgtagtacaacgaatcgctgtgaaatttgatgatgcaggtaacattatcgaatctaaagatactaaaattatcgcaaatttaggtgctggtaacgataatgtatttgttgggtcaagtactaccgttattgatggcggggacggacatgatcgagttcactacagtagaggagaatatggcgcattagttattgatgctacagccgagacagaaaaaggctcatattcagtaaaacgctatgtcggagacagtaaagcattacatgaaacaattgccacccaccaaacaaatgttggtgctcgtgaagaaaaaattgaatatcgtcgtgaagatgatcgttttcatactggttatactgtgacggactcactcaaatcagttgaagagatcattggttcacaatttaatgatattttcaaaggaagccaatttgatgatgtgttccatggtggtaatggtgtagacactattgatggtaacgatggtgacgatcatttatttggtggcgcaggcgatgatgttatcgatggaggaaacggtaacaatttccttgttggaggaaccggtaatgatattatctcgggaggtaaagataatgatatttatgtccataaaacaggcgatggaaatgattctattacagactctggcggacaagataaactggcattttcggatgtaaatcttaaagacctcacctttaagaaagtagattcttctctcgaaatcattaatcaaaaaggagaaaaagttcgtattgggaattggttcttagaagatgatttggctagcacagttgctaactataaagctacgaatgaccgaaaaattgaggacaagcggtttg

SEQ ID NO: 39 - DNA, 3988 bp, Artificial Sequence
  delta_apxIIIA_dfrA14sacB
attgggtaccgagctcgcggccgcgcaggtgcattagttggcgcaccaattactttgttggttactggtatcacaggattaatttctggtattttagagttctctaaacaaccaatgttagatcatgttgcatcgaaaattggtaacaaaattgacgaatgggagaaaaaatacggtaaaaattacttcgagaatggctatgatgctcgtcataaagctttcttagaagattcattctcattattgtctagttttaataaacaatatgaaactgaaagagctgttttaattacacaacaacgttgggatgaatatattggcgaacttgcgggtattactggcaaaggtgacaaactctctagtggtaaggcgtatgtagattactttcaagaaggtaaattattagagaaaaaacctgatgactttagcaaagtagttttcgatccaactaagggcgaaattgatatttcaaatagccaaacgtcaacgttgttaaaatttgttacgccattattaacaccaggtgttaatgccgtctgaagtgcgaagcggcatcagagcagattgtactgagagtgcaccatatggtcgacctcgagttaattaacgtatgcggccgctttagactatttaaataatattatttaaattctttactatagtgtacaatacacacagtccattaaccaaaataaaaggaggaattaggatgagaaccttgaaagtatcattgatagctgcgaaagcgaaaaacggcgtgattggttgcggtccagacataccctggtccgcgaaaggggagcagctactttttaaagcattgacctacaatcagtggcttctggtgggtcgcaagacgtttgaatctatgggcgcactccccaataggaaatacgcggtcgttacccgctcaggttggacatcaaatgatgacaatgtagttgtatttcagtcaatcgaagaggccatggacaggctagctgaattcaccggtcacgttatagtgtctggtggcggagaaatttaccgagaaacattacccatggcctctacgctccacttatcgacgatcgacatcgagccagagggggatgttttcttcccgagtattccaaataccttcgaagttgtttttgagcaacactttacttcaaacattaactattgctatcaaatttggaaaaagggttaatgccgtctgaagtgcggtacaagcggtagaacctgccccgttagttgaaaccgcttgttatgcatgcatgggatccgcgaatcccgcggccatggcggccgggagcatgcgacgtcgggcccattgggatccgcttttacagcgattgcagaatgattgaattgtaaactttagagctttatattttgtttaatggtattatatttacttatatttatgattcttagtttttattgtaaattaaagtgtttatttattgtattttaagtataagatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatatcctattggcattttcttttatttcttatcaacataaaggtgaatcccatacctagagctgcacgcgagacatgaacgtgcaactgcttctcaggtaatagtaacctaaaagcacatgatgaattacattcagtagaagaaattattggaagtaatcagagagacgaatttaaaggtagtaaattcagagatattttccatggtgccgatggtgatgatctattaaatggtaatgatggggatgatattctatacggtgataaaggtaacgatgagttaagaggtgataatggtaacgaccaactttatggtggtgaaggtaatgacaaactattaggaggtaatggcaataattacctcagtggtggtgatggcaatgatgagcttcaagtcttaggcaatggttttaatgtgcttcgtggcggtaaaggcgatgataaactttatggtagctcaggttctgatttacttgatggtggagaaggtaatgattatctagaaggaggcgatggtagcgatttttatgtttatcgttccacttcaggtaatcatactatttatgatcaaggtaaatctagtgatttagataaagatccgaattcctgtgtgaaatgg

SEQ ID NO: 40 - DNA, 1491 bp, Artificial Sequence
  apxIIIAmut
gaccgcggccgcgcaggtgcattagttggcgcaccaattactttgttggttactggtatcacaggattaatttctggtattttagagttctctaaacaaccaatgttagatcatgttgcatcgaaaattggtaacaaaattgacgaatgggagaaaaaatacggtaaaaattacttcgagaatggctatgatgctcgtcataaagctttcttagaagattcattctcattattgtctagttttaataaacaatatgaaactgaaagagctgttttaattacacaacaacgttgggatgaatatattggcgaacttgcgggtattactggcaaaggtgacaaactctctagtggtaaggcgtatgtagattactttcaagaaggtaaattattagagaaaaaacctgatgactttagcaaagtagttttcgatccaactaagggcgaaattgatatttcaaatagccaaacgtcaacgttgttaaaatttgttacgccattattaacaccaggtacagagtcacgtgaaagaactcaaacaggtgcatatgaatatatcacgaagttagttgtaaaaggtaaagataaatgggttgttaatggcgttaaagataaaggtgccgtttatgattatactaatttaattcaacatgctcatattagttcatcagtagcacgtggtgaagaataccgtgaagttcgtttggtatctcatctaggcaatggtaatgacaaagtgttcttagctgcgggttccgcagaaattcacgctggtgaaggtcatgatgtggtttattatgataaaaccgatacaggtcttttagtaattgatggaaccaaagcgactgaacaagggcgttattctgttacgcgcgaattgagtggtgctacaaaaatcctgagagaagtaataaaaaatcaaaaatctgctgttggtgcacgtgaagaaaccttggaatatcgtgattatgaattaacgcaatcaggtaatagtaacctaaaagcacatgatgaattacattcagtagaagaaattattggaagtaatcagagagacgaatttaaaggtagtaaattcagagatattttccatggtgccgatggtgatgatctattaaatggtaatgatggggatgatattctatacggtgataaaggtaacgatgagttaagaggtgataatggtaacgaccaactttatggtggtgaaggtaatgacaaactattaggaggtaatggcaataattacctcagtggtggtgatggcaatgatgagcttcaagtcttaggcaatggttttaatgtgcttcgtggcggtaaaggcgatgataaactttatggtagctcaggttctgatttacttgatggtggagaaggtaatgattatctagaaggaggcgatggtagcgatttttatgtttatcgttccacttcaggtaatcatactatttatgatcaaggtaaatctagtgatttagatacaagcggtttg

SEQ ID NO: 41 - DNA, 4050 bp, Artificial Sequence
  delta_apxIA_trunc_dfrA14sacB
attgggtaccgagctcgcggccgcctaattcacccgcttgcgattgcgggtctaaagtaccgccgtaccaaacgtccgcttgcggattatttttttccgcttcgatttttgcaaaggtactgccggaaccgttgcggataaaagaggttttcacatcatatttttgttcgaatgtttttgccgcattctcacacatcacattggtcgcactacagtaaatcactaaacgtccttttgcctgagccgccgaactgaacattaagcccgcaccaagtaatgcggttgaaaccgctaaagaaagttttccaaatttcataatcaaagcctcatattgagcataaatcaataaaatgccgcgaatataatcgaaagcatttttcttattggaactaatttaccgtaattgaataaaaaataccgtgaagcagttcacaaaatacgagattaatgagcgatattgttataaaatcataatgtaaacctcatttgtaatgaattggtaaattatataaataatcaaaaaacttacttttttttatttttatcggtaagtatttacaatcaagtcagacaaacagtaagattgaaggttaatgccgtctgaagtgcgaagcggcatcagagcagattgtactgagagtgcaccatatggtcgacctcgagttaattaacgtatgcggccgctttagactatttaaataatattatttaaattctttactatagtgtacaatacacacagtccattaaccaaaataaaaggaggaattaggatgagaaccttgaaagtatcattgatagctgcgaaagcgaaaaacggcgtgattggttgcggtccagacataccctggtccgcgaaaggggagcagctactttttaaagcattgacctacaatcagtggcttctggtgggtcgcaagacgtttgaatctatgggcgcactccccaataggaaatacgcggtcgttacccgctcaggttggacatcaaatgatgacaatgtagttgtatttcagtcaatcgaagaggccatggacaggctagctgaattcaccggtcacgttatagtgtctggtggcggagaaatttaccgagaaacattacccatggcctctacgctccacttatcgacgatcgacatcgagccagagggggatgttttcttcccgagtattccaaataccttcgaagttgtttttgagcaacactttacttcaaacattaactattgctatcaaatttggaaaaagggttaatgccgtctgaagtgcggtacaagcggtagaacctgccccgttagttgaaaccgcttgttatgcatgcatgggatccgcgaatcccgcggccatggcggccgggagcatgcgacgtcgggcccattgggatccgcttttacagcgattgcagaatgattgaattgtaaactttagagctttatattttgtttaatggtattatatttacttatatttatgattcttagtttttattgtaaattaaagtgtttatttattgtattttaagtataagatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatatcctattggcattttcttttatttcttatcaacataaaggtgaatcccatacctagagctgcacgcgagacatgaacgtgcaactgcttcgatagttatttttagatgataaatagcaatcctatatatattaggtgtgtaggattgctattttatttatggaggagcaaatggatttttatcgggaagaagactacggattatacgcactgacgattttagcccagtaccataatattgctgtaaatccggaagaactaaaacataaattcgaccttgaaggaaaaggcttagatctaaccgcttggctattagccgcaaaatcattagaacttaaagcaaaacaagtaaaaaaagcgattgatcgtttggcgtttatcgcactaccggcacttgtatggcgagaagacggtaaacattttattttgactaaaattgataatgaagcaaaaaaatatttaatttttgatttggaaacgcataatcctcgcattttggaacaagcggaattcgagagcttataccaaggaaaactgattttagttgcatcaagagcttccatcgtaggtaagctggcaaagtttgacttgatccgaattcctgtgtgaaatgg

SEQ ID NO: 42 - DNA, 4756 bp, Artificial Sequence
  apxIAmut_long
aaacaagcggtccggatcttggaatttcggcataatttgatcgatattcggcgaacgatacgcttctaataagcctaattcacccgcttgcgattgcgggtctaaagtaccgccgtaccaaacgtccgcttgcggattatttttttccgcttcgatttttgcaaaggtactgccggaaccgtttcggataaaagaggttttcacatcatatttttgttcgaatgtttttgccgcattctcacacatcacattggtcgcactacagtaaatcactaaacgtccttttgcctgagccgccgaactgaacattaagcccgcaccaagtaatgcggttgaaaccgctaaagaaagttttccaaatttcataatcaaagcctcatattgagcataaatcaacaaaatgccgcgaatataatcgaaagcatttttcttattggaactaatttaccgtaattgaataaaaaataccgtgaagcagttcacaaaatacgagattaatgagcgatattgttataaaatcataatgtaaacctcatttgtaatgaattggtaaattatataaataatcaaaaaacttacttttttttatttttatcggtaagtatttacaatcaagtcagacaaacggcaatattgttataaatctggggggatgaatgagtaaaaaaattaatggatttgaggttttaggagaggtggcatggttatgggcaagttctcctttacatcgaaagtggccgctttctttgttagcaattaatgtgctacctgcgattgagagtaatcaatatgttttgttaaagcgtgacggttttcctattgcattttgtagctgggcaaatttgaatttggaaaatgaaattaaataccttgatgatgttgcctcgctagttgcggatgattggacttccggcgatcgtcgatggtttatagattggatagcaccgttcggagacagtgccgcattatacaaacatatgcgagataacttcccgaatgagctgtttagggctattcgagttgatccggactctcgagtagggaaaatttcagaatttcatggaggaaaaattgataagaaactggcaagtaaaatttttcaacaatatcactttgaattaatgagtgagctaaaaaataaacaaaattttaaattttcattagtaaatagctaaggagacaacatggctaactctcagctcgatagagtcaaaggattgattgattcacttaatcaacatacaaaaagtgcagctaaatcaggtgccggcgcattaaaaaatggtttgggacaggtgaagcaagcagggcagaaattaattttatatattccgaaagattatcaagctagtaccggctcaagtcttaatgatttagtgaaagcggcggaggctttagggatcgaagtacatcgctcggaaaaaaacggtaccgcactagcgaaagaattattcggtacaacggaaaaactattaggtttctcggaacgaggcatcgcattatttgcacctcagtttgataagttactgaataagaaccaaaaattaagtaaatcgctcggcggttcatcggaagcattaggacaacgtttaaataaaacgcaaacggcactttcagccttacaaagtttcttaggtacggctattgcgggtatggatcttgatagcctgcttcgtcgccgtagaaacggtgaggacgtcagtggttcggaattagctaaagcaggtgtggatctagccgctcagttagtggataacattgcaagtgcaacgggtacggtggatgcgtttgccgaacaattaggtaaattgggcaatgccttatctaacactcgcttaagcggtttagcaagtaagttaaataaccttccagatttaagccttgcaggacctgggtttgatgccgtatcaggtatcttatctgttgtttcggcttcattcattttaagtaataaagatgccgatgcaggtacaaaagcggcggcaggtattgaaatctcaactaaaatcttaggcaatatcggtaaagcggtttctcaatatattattgcgcaacgtgtggcggcaggcttatccacaactgcggcaaccggtggtttaatcggttcggtcgtagcattagcgattagcccgctttcgttcttaaatgttgcggataagtttgaacgtgcgaaacagcttgaacaatattcggagcgctttaaaaagttcggttatgaaggtgatagtttattagcttcattctaccgtgaaaccggtgcgattgaagcggcattaaccacgattaacagtgtgttaagtgcggcttccgcaggtgttggggctgctgcaaccggctcattagtcggtgcgccggtagcagctttagttagtgcaatcaccggtattatttcaggtattttagatgcttctaaacaggcaatcttcgaacgagttgcaacgaaattagcgaataagattgacgaatgggagaaaaaacacggtaaaaactattttgaaaacggttatgacgcccgccattccgcattcttagaagatacctttgaattgttatcacaatacaataaagagtattcggtagagcgtgtcgttgctattacgcaacagcgttgggatgtcaatatcggtgaacttgccggcattactcgcaaaggttctgatacgaaaagcggtaaagcttacgttgatttctttgaagaaggaaaacttttagagaaagaaccggatcgttttgataaaaaagtgtttgatccgcttgaaggcaaaatcgacctttcttcaattaacaaaaccactttattgaaatttattacaccggtttttaccgcaggtgaagagattcgtgagcgtaagcaaaccggtgcatacgaatatatgaccgaattattcgttaaaggtaaagaaaaatgggtggtaaccggtgtgcagtcacataatgcgatttatgactatacgaatcttatccaattagcgatagataaaaaaggtgaaaaacgtcaagtgaccattgaatctcatttgggtgagaaaaatgatcgtatatatctttcatccggttcatctatcgtatatgcgggtaacggacatgatgtagcatattacgataaaaccgatacaggttacttaacatttgacggacaaagtgcacagaaagccggtgaatatattgtcactaaagaacttaaagctgatgtaaaagttttaaaagaagtggttaaaactcaggatatttcagttggagcacgcagtgaaaaattagaatatcgtgattatgagttaagcccattcgaacttgggaacggtatcagagctaaagatgaattacattctgttgaagaaattatcggtagtaatcgtaaagacaaattctttggtagtcgctttaccgatattttccatggtgcgaaaggcgatgatgaaatctacggtaatgacggccacgatatcttatacggagacgacggtaatgatgtaatccatggcggtgacggtaacgaccatcttgttggtggtaacggaaacgaccgattaatcggcggaaaaggtaataatttccttaatggcggtgatggtgacgatgagttgcaggtctttgagggtcaatacaacgtattattaggtggtgcgggtaatgacattctgtatggcagcgatggtactaacttatttgacggtggtgtaggcaatgacaaaatctacggtggtttaggtaaggatatttatcgctacagtaaggagtacggtcgtcatatcattattgagaaaggcggtgatgatgatacgttattgttatcggatcttagttttaaagatgtaggatttatcagaatcggtgatgatcttcttgtgaataaaagaatcggaggaacactgtattaccatgaagattacaatgggaatgcgctcacgattaaagattggttcaaggaaggtaaagaaggacaaaataataaaattgaaaaaatcgttgataaagatggagcttatgttttaagccaatatctgactgaactgacagctcctggaagaggtatcaattactttaatgggttagaagaaaaattgtattatggagaaggatataatgcacttcctcaactcagaaaagatattgaacaaatcatttcatctacgggtgcatttaccggtgatcacggaaaagtatctgtaggctcaggcggaccgttagtctataataactcagctaacaatgtagcaaattctttgagttattctttagcacaagcagcttaagatagttatttttagatgataaatagcaatcctatatatattaggtgtgtaggattgctattttatttatggaggagcaaatggatttttatcgggaagaagactacggattatacgcactgacgattttagcccagtaccataatattgctgtaaatccggaagaactaaaacataaattcgaccttgaaggaaaaggcttagatctaaccgcttggctattagccgcaaaatcattagaacttaaagcaaaacaagtaaaaaaagcgattgatcgtttggcgtttatcgcactaccggcacttgtatggcgagaagacggtaaacattttattttgactaaaattgataatgaagcaaaaaaatatttaatttttgatttggaaacgcataatcctcgcattttggaacaagcggaattcgagagcttataccaaggaaaactgattttagttgcatcaagagcttccatcgtaggtaagctggcaaagtaccgcttgtaa

SEQ ID NO: 43 - DNA, 3981 bp, Artificial Sequence
  delta_apxIVA_ dfrA14sacB
atccacaagcggtcatctggcgcgaatagagaacctgaacaatgggaaaattacatagtatttgataattgcagtggaattaaagaaagacaccaactgtattaaaaatagattagaaggagacaacacgatgacaaaactaactatgcaagatgtgactaatttatatttatataagcaaagaactttacctacggataggttagatgattcgcttattagcaaaacaggaaaaggggaaaatattgataaaaaggaatttatggcggggccgggacgttttgtgacggccgataattttagtgttgtaaaagacttttttactgcaaaggattcattaataaacctaagcttgcagactcgtatattagcgaatttaaagccgggcaaatattccaaagcgcagatattagaaatgttgggctatacgaaaaatggagaaaaggtagatggcatgtttaccggtgaagtccagacattaggcttttatgacgatggcaaaggggatttactcgaacgcgttaatgccgtctgaagtgcgaagcggcatcagagcagattgtactgagagtgcaccatatggtcgacctcgagttaattaacgtatgcggccgctttagactatttaaataatattatttaaattctttactatagtgtacaatacacacagtccattaaccaaaataaaaggaggaattaggatgagaaccttgaaagtatcattgatagctgcgaaagcgaaaaacggcgtgattggttgcggtccagacataccctggtccgcgaaaggggagcagctactttttaaagcattgacctacaatcagtggcttctggtgggtcgcaagacgtttgaatctatgggcgcactccccaataggaaatacgcggtcgttacccgctcaggttggacatcaaatgatgacaatgtagttgtatttcagtcaatcgaagaggccatggacaggctagctgaattcaccggtcacgttatagtgtctggtggcggagaaatttaccgagaaacattacccatggcctctacgctccacttatcgacgatcgacatcgagccagagggggatgttttcttcccgagtattccaaataccttcgaagttgtttttgagcaacactttacttcaaacattaactattgctatcaaatttggaaaaagggttaatgccgtctgaagtgcggtacaagcggtagaacctgccccgttagttgaaaccgcttgttatgcatgcatgggatccgcgaatcccgcggccatggcggccgggagcatgcgacgtcgggcccattgggatccgcttttacagcgattgcagaatgattgaattgtaaactttagagctttatattttgtttaatggtattatatttacttatatttatgattcttagtttttattgtaaattaaagtgtttatttattgtattttaagtataagatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatatcctattggcattttcttttatttcttatcaacataaaggtgaatcccatacctagagctgcacgcgagacatgaacgtgcaactgcttccaattcgaagggaaatgggtaaccgattattctcgtactgaagccttatttaactctacttttaaacaatcgcctgaaaatgcattatatgatttaagcgaatacctttctttctttaacgatcctacggaatggaaagaagggctattactgttaagccgttatatagattatgctaaagcacaaggattttatgaaaactgggcggctacttctaacttaactattgcccgtttaagagaggctggagtaatttttgcagaatcgacggatttaaaaggcgatgaaaaaaataatattttgttaggtagccaaaaagataataacttatcgggtagtgcaggtgatgatctacttatcggcggagagggtaatgatacgttaaaaggcagctacggtgcggacacctatatctttagcaaaggacacggacaggatatcgtttatgaagataccaataatgataaccgagcaagagatatcgacaccttaaaatttggatccgaattcctgtgtgaaatgg

SEQ ID NO: 44 - DNA, 1043 bp, Artificial Sequence
  apxIV_int_del
atccacaagcggtcatctggcgcgaatagagaacctgaacaatgggaaaattacatagtatttgataattgcagtggaattaaagaaagacaccaactgtattaaaaatagattagaaggagacaacacgatgacaaaactaactatgcaagatgtgactaatttatatttatataagcaaagaactttacctacggataggttagatgattcgcttattagcaaaacaggaaaaggggaaaatattgataaaaaggaatttatggcggggccgggacgttttgtgacggccgataattttagtgttgtaaaagacttttttactgcaaaggattcattaataaacctaagcttgcagactcgtatattagcgaatttaaagccgggcaaatattccaaagcgcagatattagaaatgttgggctatacgaaaaatggagaaaaggtagatggcatgtttaccggtgaagtccagacattaggcttttatgacgatggcaaaggggatttactcgaacgccaattcgaagggaaatgggtaaccgattattctcgtactgaagccttatttaactctacttttaaacaatcgcctgaaaatgcattatatgatttaagcgaatacctttctttctttaacgatcctacggaatggaaagaagggctattactgttaagccgttatatagattatgctaaagcacaaggattttatgaaaactgggcggctacttctaacttaactattgcccgtttaagagaggctggagtaatttttgcagaatcgacggatttaaaaggcgatgaaaaaaataatattttgttaggtagccaaaaagataataacttatcgggtagtgcaggtgatgatctacttatcggcggagagggtaatgatacgttaaaaggcagctacggtgcggacacctatatctttagcaaaggacacggacaggatatcgtttatgaagataccaataatgataaccgagcaagagatatcgacaccttaaaatttggatccgaattcctgtgtgaaatgg

SEQ ID NO: 45 - DNA, 4390 bp, Artificial Sequence
  sxy_dfrA14sacB_insert
gtaccgcttgttaaatgattacaccaagcgactctaaaaatcttcgtatctatatcataaatacgatgagcgttaaagtggcgatattctagtgtaaaaaacacttaaaagcaagatttaattttatttttctaaaaaatatagtttcaaacgaatcggacatatttttaccctttattatatttacattattgacattaaataatttattttgcaaaatatacataaatttcgctcattaaaaaataatcatatataaaaaaggagaaacataatggcaatatccccaaaaaagttccaatatcttaaggagatttttagtcctcttggagaaattaacttcaaaagctatttttcttacttaggaatatttaaagacgatactatgttcgccctctatgatcataaaaacgatcgattatacttaagaaaatccgctcaattttatccggatattataagaacaataccgatacattttttaattgatcgtcgtatcggtaagcaacaatctcatattttttatcttataccttcttctattattcacaatcttcatttatatactcattggattctctctgctatcgaagaatatcaaactgcaaaggccaaattgatttctcaaaataaaaataaaattcgtctgcttcccaatttgaatatcaatatagaaagattattggcacgtattgagatttataccgtagatgatttaaaaaacgtaggcgtgattaatgcgtttgtaaaactgataatgctaggcttggaagtaaccgaattactcctcttcaaactctacgctgcgctcgaacataaatatatctatatgttatccaagcaagaaaaacaatccctattaattgaagccgatttatctctctataacgcaggcctacgtaaacgcttcgctatctcacaggctaactaagttaatgccgtctgaagtgcgaagcggcatcagagcagattgtactgagagtgcaccatatggtcgacctcgagttaattaacgtatgcggccgctttagactatttaaataatattatttaaattctttactatagtgtacaatacacacagtccattaaccaaaataaaaggaggaattaggatgagaaccttgaaagtatcattgatagctgcgaaagcgaaaaacggcgtgattggttgcggtccagacataccctggtccgcgaaaggggagcagctactttttaaagcattgacctacaatcagtggcttctggtgggtcgcaagacgtttgaatctatgggcgcactccccaataggaaatacgcggtcgttacccgctcaggttggacatcaaatgatgacaatgtagttgtatttcagtcaatcgaagaggccatggacaggctagctgaattcaccggtcacgttatagtgtctggtggcggagaaatttaccgagaaacattacccatggcctctacgctccacttatcgacgatcgacatcgagccagagggggatgttttcttcccgagtattccaaataccttcgaagttgtttttgagcaacactttacttcaaacattaactattgctatcaaatttggaaaaagggttaatgccgtctgaagtgcggtacaagcggtagaacctgccccgttagttgaaaccgcttgttatgcatgcatgggatccgcgaatcccgcggccatggcggccgggagcatgcgacgtcgggcccattgggatccgcttttacagcgattgcagaatgattgaattgtaaactttagagctttatattttgtttaatggtattatatttacttatatttatgattcttagtttttattgtaaattaaagtgtttatttattgtattttaagtataagatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatatcctattggcattttcttttatttcttatcaacataaaggtgaatcccatacctagagctgcacgcgagacatgaacgtgcaactgcttcgtaagccggttcctttctgattatctcaatgctaccatcctacctataacttgttagtttatttaagtgaaatctacttttatccataggagaacacaatggaatttcgtattgaaaaagataccatgggcgaagttcaagtacctgccaatcgttattgggcggcacaaacagagcgttcacgcaataattttaaaatcggtcccgaagcgtcaatgcctaaagaaattattgaagcgttcggttacttgaaaaaagcagcggcatttgccaacacagatttaggcgtattacctgcggaaaaacgtgatttaatcgctcaagcctgtgatgaaatccttgccggtaaattaaacgaagaattcccgcttgtaatctggcaaaccggttccggtacgcaatccaatatgaacttaaacgaagttattgcaaaccgtgcgcatgttattcacggcggtaaattaggtgaaaaatcggtaattcacccgaatgatgaggatccgaattcctgtgtgaaatgg

SEQ ID NO: 46 - DNA, 1037 bp, Artificial Sequence
  Sxy_del
atccacaagcggtcatctggctcacgtgtggagaaatcaatacagtaaaacgttctttacgagttggtaaaggaattggaccacgaacttgtgcaccagtacgtttagctgtttctacgatctccgcagtagattgatcaattaaacgatgatcaaatgcttttaagcggatacggattctttggttctgcattagaccagagctccaattaaaatttagctaataaaaaaaccgaactaccacttaagccacatagcataagggagcgcagttatacctatatagtttccaaatcggaaacattgtatgtactacaatatctgtagtaccgcttgataaatgattacaccaagcgactctaaaaatcttcgtatctatatcataaatacgatgagcgttaaagtggcgatattctagtgtaaaaaacacttaaaagcaagatttaattttatttttctaaaaaatatagtttcaaacgaatcggacatatttttaccctttattatatttacattgtaagccggttcctttctgattatctcaatgctaccatcctacctataacttgttagtttatttaagtgaaatctacttttatccataggagaacacaatggaatttcgtattgaaaaagataccatgggcgaagttcaagtacctgccaatcgttattgggcggcacaaacagagcgttcacgcaataattttaaaatcggtcccgaagcgtcaatgcctaaagaaattattgaagcgttcggttacttgaaaaaagcagcggcatttgccaacacagatttaggcgtattacctgcggaaaaacgtgatttaatcgctcaagcctgtgatgaaatccttgccggtaaattaaacgaagaattcccgcttgtaatctggcaaaccggttccggtacgcaatccaatatgaacttaaacgaagttattgcaaaccgtgcgcatgttattcacggcggtaaattaggtgaaaaatcggtaattcacccgaatgatgaggatccgaattcctgtgtgaaatgg

SEQ ID NO: 47 - protein, 1943 aa, Actinobacillus pleuropneumoniae
MTKLTMQDVTNLYLYKQRTLPTDRLDDSLISKTGKGENIDKKEFMAGPGRFVTADNFSVVKDFFTAKDSLINLSLQTRILANLKPGKYSKAQILEMLGYTKNGEKVDGMFTGEVQTLGFYDDGKGDLLERAYIWNTTGFKMSDNAFFVIEESGKRYIENFGIEPLGKQEDFDFVGGFWSNLVNRGLESIIDPSGIGGTVNLNFTGEVETYTLDETRFKAEAAKKSHWSLVNAAKVYGGLDQIIKKLWDSGSIKHLYQDKDTGKLKPIIYGTAGNDSKIEGTKITRRIAGKEVTLDIANQKIEKGVLEKLGLSVSGSDIIKLLFGALTPTLNRMLLSQLIQSFSDSLAKLDNPLAPYTKNGVVYVTGKGNDVLKGTEHEDLFLGGEGNDTYYARVGDTIEDADGKGKVYFVREKGIPKADPKRVEFSKYITEEEIKEVEKGLLTYAVLENYNWEEKTATFAHATMLNELFTDYTNYRYKVKGLKLPAVKKLKSPLVEFTADLLTVTPIDENGKALSEKSITVKNFKNGDLGIRLLDPNSYYYFLEGQDTGFYGPAFYIERKNGGGAKNNSSGAGNSKDWGGNGHGNHRNNASDLNKPDGNNGNNQNNGSNQDNHSDVNAPNNPGRNYDIYDPLALDLDGDGLETVSMNGRQGALFDHEGKGIRTATGWLAADDGFLVLDRNQDGIINDISELFSNKNQLSDGSISAHGFATLADLDTNQDQRIDQNDKLFSKLQIWRDLNQNGFSEANELFSLESLNIKSLHTAYEERNDFLAGNNILAQLGKYEKTDGTFAQMGDLNFSFNPFYSRFTEALNLTEQQRRTINLTGTGRVRDLREAAALSEELAALLQQYTKASDFQAQRELLPAILDKWAATDLQYQHYDKTLLKTVESTDSSASVVRVTPSQLSSIRNAKHDPTVMQNFEQSKAKIATLNSLYGLNIDQLYYTTDKDIRYITDKVNNMYQTTVELAYRSLLLQTRLKKYVYSVNAKQFEGKWVTDYSRTEALFNSTFKQSPENALYDLSEYLSFFNDPTEWKEGLLLLSRYIDYAKAQGFYENWAATSNLTIARLREAGVIFAESTDLKGDEKNNILLGSQKDNNLSGSAGDDLLIGGEGNDTLKGSYGADTYIFSKGHGQDIVYEDTNNDNRARDIDTLKFTDVNYAEVKFRRVDNDLMLFGYHDTDSVTVKSFYNHEYYQFEKLEFADRSITRDELGKQGMALFGTDGDDDINDWGRNSVIDAGAGNDTINGSYGDDTLIGGTGNDILKGSYGADTYLFSKGHGQDVIYEYSDSANSKRDIDTLKFTDVNYAEVKFRRVGDDLMLFGYHDTDSVTVKSFYDHEYYQFEKLEFADRSISRDELIKAGLHLYGTDGNDEINDHADWDSILEGGKGNDILRGSYGADTYLFSKGHGQDVIYEYSDSANSKSDIDTLKFTDINYAEVKFRRVGDDLMLFGYHDTDSVTVKSFYNHEYYQFEKLEFADRSITRDELGKQGMALFGTDGDDDINDWGRNSVIDAGAGNDTINGSYGDDTLIGGTGNDILKGSYGADTYLFSKGHGQDVIYEYSDSANSKRDIDTLKFTDVNYAEVKFRRVGDDLMLFGYHDTDSVTVKSFYSHEYYQFEKLEFADRSISRDELIKAGLHLYGTDGNDEINDHADWDSILEGGKGNDILRGSYGADTYIFSKGHGQDVIYEYSDSANSKRDIDTLKFTDVNYAEVKFRRVGDDLMLFGYHDTDSVTVKSFYDHEYYQFEKLEFADRSITRDELGKQGMALFGTDGDDDINDWGRNSVIDAGAGNDTINGGYGDDTLIGGKGNDILKGSYGADTYLFSKGHGQDVIYEYSDSASSKSDIDTLKFTDIGLSELWFSRENNDLIIKSLLSEDKVTVQNWYSHQDHKIENIRLSNEQMLVSTQVEKMVESMAGFAQQHGGEISLVPREEVKQYINSLTAAL

SEQ ID NO: 48 - protein, 1086 aa, Artificial Sequence
  APP ApxIV with N-terminal in-frame deletion
MTKLTMQDVTNLYLYKQRTLPTDRLDDSLISKTGKGENIDKKEFMAGPGRFVTADNFSVVKDFFTAKDSLINLSLQTRILANLKPGKYSKAQILEMLGYTKNGEKVDGMFTGEVQTLGFYDDGKGDLLERQFEGKWVTDYSRTEALFNSTFKQSPENALYDLSEYLSFFNDPTEWKEGLLLLSRYIDYAKAQGFYENWAATSNLTIARLREAGVIFAESTDLKGDEKNNILLGSQKDNNLSGSAGDDLLIGGEGNDTLKGSYGADTYIFSKGHGQDIVYEDTNNDNRARDIDTLKFTDVNYAEVKFRRVDNDLMLFGYHDTDSVTVKSFYNHEYYQFEKLEFADRSITRDELGKQGMALFGTDGDDDINDWGRNSVIDAGAGNDTINGSYGDDTLIGGTGNDILKGSYGADTYLFSKGHGQDVIYEYSDSANSKRDIDTLKFTDVNYAEVKFRRVGDDLMLFGYHDTDSVTVKSFYDHEYYQFEKLEFADRSISRDELIKAGLHLYGTDGNDEINDHADWDSILEGGKGNDILRGSYGADTYLFSKGHGQDVIYEYSDSANSKSDIDTLKFTDINYAEVKFRRVGDDLMLFGYHDTDSVTVKSFYNHEYYQFEKLEFADRSITRDELGKQGMALFGTDGDDDINDWGRNSVIDAGAGNDTINGSYGDDTLIGGTGNDILKGSYGADTYLFSKGHGQDVIYEYSDSANSKRDIDTLKFTDVNYAEVKFRRVGDDLMLFGYHDTDSVTVKSFYSHEYYQFEKLEFADRSISRDELIKAGLHLYGTDGNDEINDHADWDSILEGGKGNDILRGSYGADTYIFSKGHGQDVIYEYSDSANSKRDIDTLKFTDVNYAEVKFRRVGDDLMLFGYHDTDSVTVKSFYDHEYYQFEKLEFADRSITRDELGKQGMALFGTDGDDDINDWGRNSVIDAGAGNDTINGGYGDDTLIGGKGNDILKGSYGADTYLFSKGHGQDVIYEYSDSASSKSDIDTLKFTDIGLSELWFSRENNDLIIKSLLSEDKVTVQNWYSHQDHKIENIRLSNEQMLVSTQVEKMVESMAGFAQQHGGEISLVPREEVKQYINSLTAAL